(19) United States
(12) Patent Application Publication
(10) Pub. No.: US 2022/0064146 A1
(43) Pub. Date: Mar. 3, 2022

(54) KINASE INHIBITOR COMPOUNDS AND COMPOSITIONS AND METHODS OF USE

(71) Applicant: ICAHN SCHOOL OF MEDICINE AT MOUNT SINAI, New York, NY (US)

(72) Inventors: Robert J. DEVITA, New York, NY (US); Andrew F. STEWART, New York, NY (US); Chalada SUEBSUWONG, New York, NY (US); Kunal KUMAR, New York, NY (US); Peng WANG, New York, NY (US); Roberto J. SANCHEZ, New York, NY (US); Hui WANG, New York, NY (US)

(21) Appl. No.: 17/418,607

(22) PCT Filed: Dec. 31, 2019
(86) PCT No.: PCT/US19/69059

§ 371 Date: Jun. 25, 2021

Related U.S. Application Data
(60) Provisional application No. 62/786,991, filed on Dec. 31, 2018.

(51) Int. Cl. C07D 403/04; C07D 405/14; C07D 409/14; C07D 403/14; C07D 413/14; C07D 401/14; A61K 45/06

(52) U.S. Cl. CPC C07D 403/04; C07D 405/14; C07D 409/14; A61K 45/06; C07D 413/14; C07D 401/14; C07D 403/14

(57) ABSTRACT

Disclosed are kinase inhibitor compounds having the following structure: (I), or a stereoisomer, pharmaceutically acceptable salt, oxide, or solvate thereof, where R¹, R², R³, R⁴, R⁵, R⁶, N—Ar, X, Y, Z, and AA are as defined herein. Also disclosed are compositions containing the kinase inhibitor compounds, methods of inhibiting activity of a kinase in a cell, methods of increasing cell proliferation in a population of pancreatic beta cells, methods of treating a subject for a condition associated with insufficient insulin secretion, and methods of treating a subject for a neurological disorder.

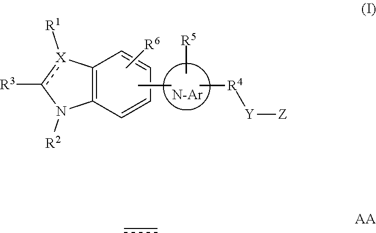

This application claims the priority benefit of U.S. Provisional Patent Application Ser. No. 62/786,991, filed Dec. 31, 2018, which is hereby incorporated by reference in its entirety.

This invention was made with government support under grant number DK015015 and DK116904 awarded by the National Institutes of Health. The government has certain rights in the invention.

### FIELD OF THE INVENTION

The present invention relates to kinase inhibitor compounds and compositions and methods of use thereof.

### BACKGROUND OF THE INVENTION

The Dual-Specificity Tyrosine-Regulated kinases (“DYRKs”) belong to the CMCG family of eukaryotic protein kinases which include the CDK-like kinases (CLKs), Glycogen Synthase Kinase 3 (GSK3), Cyclin Dependent Kinases (CDKs), and Mitogen-Activated Protein Kinases (MAPKs). DYRK family proteins self-activate by autophosphorylation of the conserved tyrosine residue in the activation loop, then subsequently phosphorylate substrates only on serine and threonine residues (Lochhead et al., “Activation-Loop Autophosphorylation is Mediated by a Novel Transitional Intermediate Form of DYRKs,” Cell 121(6):925-936 (2005); Walte et al., “Mechanism of Dual Specificity Kinase Activity of DYRK1A,” FEBS J. 280(18):4495-4511 (2013); and Becker et al., “Activation, Regulation, and Inhibition of DYRK1A,” FEBS 278(2):246-256 (2011)). The DYRK family consists of five subtypes, including 1A, 1B, 2, 3 and 4. Among them, DYRK1A is the most extensively studied subtype. It is ubiquitously expressed and has been shown to play an important role in brain development and function (Becker et al., “DYRK1A: A Potential Drug Target for Multiple Down Syndrome Neuropathologies,” CNS Neurol. Disord.: Drug Targets 13(1):26-33 (2014)), neurodegenerative diseases (Wegiel et al., “The Role of DYRK1A in Neurodegenerative Diseases,” FEBS J. 278(2):236-245 (2011) and Smith et al., “Recent Advances in the Design, Synthesis, and Biological Evaluation of Selective DYRK1A Inhibitors: A New Avenue for a Disease Modifying Treatment of Alzheimer's?,” ACS Chem. Neurosci. 3(11):857-872 (2012)), tumorigenesis, apoptosis (Ionescu et al., “DYRK1A Kinase Inhibitors With Emphasis on Cancer,” Mini-Rev. Med. Chem. 12(13):1315-1329 (2012) and Fernandez-Martinez et al., “DYRK1A: The Double-Edged Kinase as a Protagonist in Cell Growth and Tumorigenesis,” Mol. Cell. Oncol. 2(1):e970048 (2015)), and human pancreatic β-cell proliferation (Wang et al., “A High-Throughput Chemical Screen Reveals That Harmine-Mediated Inhibition of DYRK1A Increases Human Pancreatic Beta Cell Replication,” Nat. Med. 21(4):383-388 (2015); Shen et al., “Inhibition of DYRK1A and GSK3B Induces Human β-cell Proliferation,” Nat. Commun. 6:8372 (2015); Rachdi et al., “Dyrk1A Induces Pancreatic β Cell Mass Expansion and Improves Glucose Tolerance,” Cell Cycle 13(14):2221-2229 (2014); and Dirice et al., “Inhibition of DYRK1A Stimulates Human Beta-Cell Proliferation,” Diabetes 65:(6):1660-1671 (2016)).

Regulated expression of DYRK1A during fetal, postnatal life, as well as in adults, is essential for normal neuronal development and brain function. DYRK1A is located in the Down Syndrome Critical region (“DSCR”) on human chromosome 21, a genomic region that has an important role in pathogenesis of Down Syndrome (“DS”), one of the most common and frequent human genetic disorders (Becker et al., “Activation, Regulation, and Inhibition of DYRK1A,” FEBS J. 278(2):246-256 (2011) and Becker et al., “Structural and Functional Characteristics of Dyrk, a Novel Subfamily of Protein Kinases With Dual Specificity,” Prog. Nucleic Acid Res. Mol. Biol. 62:1-17 (1999)). Overexpression of DYRK1A in mouse and drosophila models mimics the neurodevelopmental abnormalities associated with DS (Becker et al., “DYRK1A: A Potential Drug Target for Multiple Down Syndrome Neuropathologies,” CNS Neurol. Disord.: Drug Targets 13(1):26-33 (2014); Wegiel et al., “The Role of DYRK1A in Neurodegenerative Diseases,” FEBS J. 278(2):236-245 (2011); Park et al., “Function and Regulation of Dyrkl A: Towards Understanding Down Syndrome,” Cell. Mol. Life Sci. 66(20):3235-3240 (2009); and Ogawa et al., “Development of a Novel Selective Inhibitor of the Down Syndrome-Related Kinase Dyrkl A,” Nat. Commun. 1: Article Number 86 (2010)).

Recent evidences has also implicated DYRK1A in the tau dysfunction and tau pathology of Alzheimer's disease (“AD”), dementia with Lewy bodies, and Parkinson's disease (Wegiel et al., “The Role of DYRK1A in Neurodegenerative Diseases,” FEBS J. 278(2):236-245 (2011); Smith et al., “Recent Advances in the Design, Synthesis, and Biological Evaluation of Selective DYRK1A Inhibitors: A New Avenue for a Disease Modifying Treatment of Alzheimer's?,” ACS Chem. Neurosci. 3(11):857-872 (2012); and Stotani et al., “DYRK1A Inhibition as Potential Treatment for Alzheimer's Disease,” Future Med. Chem. 8(6):681-696 (2016)). It has been reported that DYRK1A is overexpressed in various tumors such as, ovarian cancer, colon cancer, lung cancer, and pancreatic cancer, signifying its role in tumorigenesis and uncontrolled cell proliferation (Ionescu et al., “DYRK1A Kinase Inhibitors With Emphasis on Cancer,” Mini-Rev. Med. Chem. 12(13):1315-1329 (2012) and Fernandez-Martinez et al., “DYRK1A: The Double-Edged Kinase as a Protagonist in Cell Growth and Tumorigenesis,” Mol. Cell. Oncol. 2(1):e970048 (2015)). Inhibition of DYRK1A leads to destabilized EGFR and reduced EGFR-dependent tumor growth in glioblastoma (Pozo et al., “Inhibition of DYRK1A Destabilizes EGFR and Reduces EGFR-Dependent Glioblastoma Growth,” J. Clin. Invest. 123(6):2475-2487 (2013)). Also, DYRK1A inhibition induces activation of caspase-9 which leads to massive apoptosis in specific cancer cell types (Seifert et al., “DYRK1A Phosphorylates Caspase 9 at an Inhibitory Site and is Potently Inhibited in Human Cells by Harmine,” FEBS J. 275(24):6268-6280 (2008)). Recently, DYRK1A has been shown to be involved in molecular pathways relevant to human β-cell proliferation, making it a potential therapeutic target for β-cell regeneration in Type 1 and Type 2 diabetes (Wang et al., “A High-throughput Chemical Screen Reveals That Harmine-Mediated Inhibition of DYRK1A Increases Human Pancreatic Beta Cell Replication,” Nat. Med. 21(4):383-388 (2015); Shen et al., “Inhibition of DYRK1A and GSK3B Induces Human β-cell Proliferation,” Nat. Commun. 6:8372 (2015); Rachdi et al., “Dyrkl A Induces Pancreatic β Cell Mass Expansion and Improves Glucose Tolerance,” Cell Cycle 13(14):2221-2229 (2014); and Dirice et al., “Inhibition of DYRK1A Stimulates Human Beta-cell Proliferation,” Diabetes 65:(6):1660-1671 (2016)). DYRK1A inhibition has been proposed to drive β-cell proliferation by inducing translocation of the nuclear factor of activated T cells (“NFAT”) family of transcription factors to the nucleus, allowing access to the promoters of genes which subsequently activate human β-cell proliferation (Wang et al., “A High-throughput Chemical Screen Reveals That Harmine-Mediated Inhibition of DYRK1A Increases Human Pancreatic Beta Cell Replication,” Nat. Med. 21(4):383-388 (2015) and Rachdi et al., “Dyrkl A Induces Pancreatic β Cell Mass Expansion and Improves Glucose Tolerance,” Cell Cycle 13(14):2221-2229 (2014)).

Because of its involvement in neurodegenerative disease, cancer, and diabetes, DYRK1A has attracted increasing interest as a potential therapeutic target. A significant amount of work has been carried out to not only understand its underlying role in diseases, but also in identifying novel DYRK1A inhibitors (Becker et al., “Activation, Regulation, and Inhibition of DYRK1A,” FEBS J. 278(2):246-256 (2011); Becker et al., “DYRK1A: A Potential Drug Target for Multiple Down Syndrome Neuropathologies,” CNS Neurol. Disord.: Drug Targets 13(1):26-33 (2014); Wegiel et al., “The Role of DYRK1A in Neurodegenerative Diseases,” FEBS J. 278(2):236-245 (2011); Smith et al., “Recent Advances in the Design, Synthesis, and Biological Evaluation of Selective DYRK1A Inhibitors: A New Avenue for a Disease Modifying Treatment of Alzheimer's?,” ACS Chem. Neurosci. 3(11):857-872 (2012); Ionescu et al., “DYRK1A Kinase Inhibitors with Emphasis on Cancer,” Mini-Rev. Med. Chem. 12(13):1315-1329 (2012); Fernandez-Martinez et al., “DYRK1A: The Double-Edged Kinase as a Protagonist in Cell Growth and Tumorigenesis,” Mol. Cell. Oncol. 2(1):e970048 (2015); Wang et al., “A High-throughput Chemical Screen Reveals That Harmine-Mediated Inhibition of DYRK1A Increases Human Pancreatic Beta Cell Replication,” Nat. Med. 21(4):383-388 (2015); Shen et al., “Inhibition of DYRK1A and GSK3B Induces Human β-cell Proliferation,” Nat. Commun. 6:8372 (2015); and Dirice et al., “Inhibition of DYRK1A Stimulates Human Beta-cell Proliferation,” Diabetes 65:(6):1660-1671 (2016)).

Several DYRK1A inhibitors from natural sources as well as small molecule drug discovery programs have been identified and characterized. Among all the DYRK1A inhibitors, harmine and its analogues (β-carbolines) are the most commonly studied and remain the most potent and orally bioavailable class of inhibitors covered to date (Becker et al., “Activation, Regulation, and Inhibition of DYRK1A,” FEBS J. 278(2):246-256 (2011) and Smith et al., “Recent Advances in the Design, Synthesis, and Biological Evaluation of Selective DYRK1A Inhibitors: A New Avenue for a Disease Modifying Treatment of Alzheimer's?,” ACS Chem. Neurosci. 3(11):857-872 (2012)).

Apart from harmine, EGCg and other flavan-3-ols (Guedj et al., “Green Tea Polyphenols Rescue of Brain Defects Induced by Overexpression of DYRK1A,” PLoS One 4(2):e4606 (2009) and Bain et al., “The Specificities of Protein Kinase Inhibitors: An Update,” Biochem. J. 371(1):199-204 (2003)), leucettines (Tahtouh et al., “Selectivity, Cocrystal Structures, and Neuroprotective Properties of Leucettines, a Family of Protein Kinase Inhibitors Derived from the Marine Sponge Alkaloid Leucettamine B,” J. Med. Chem. 55(21):9312-9330 (2012) and Naert et al., “Leucettine L41, a DYRK1A-preferential DYRKs/CLKs Inhibitor, Prevents Memory Impairments and Neurotoxicity Induced by Oligomeric Aβ25-35 Peptide Administration in Mice,” Eur. Neuropsychopharmacol. 25(11):2170-2182 (2015)), quinalizarine (Cozza et al., “Quinalizarin as a Potent, Selective and Cell-permeable Inhibitor of Protein Kinase CK2,” Biochem. J. 421(3):387-395 (2009)), peltogynoids Acanilol A and B (Ahmadu et al, “Two New Peltogynoids from Acacia nilotica Delile with Kinase Inhibitory Activity,” Planta Med. 76(5):458-460 (2010)), benzocoumarins (dNBC) (Sarno et al., “Structural Features Underlying the Selectivity of the Kinase Inhibitors NBC and dNBC: Role of a Nitro Group that Discriminates Between CK2 and DYRK1A,” Cell. Mol. Life Sci. 69(3):449-460 (2012)), and indolocarbazoles (Starosporine, rebeccamycin and their analogues) (Sanchez et al., “Generation of Potent and Selective Kinase Inhibitors by Combinatorial Biosynthesis of Glycosylated Indolocarbazoles,” Chem. Commun. 27:4118-4120 (2009), are other natural products that have been shown to inhibit DYRK1A and other kinases.

Among the other scaffolds identified from small molecule drug discovery attempts, INDY (Ogawa et al., “Development of a Novel Selective Inhibitor of the Down Syndrome-Related Kinase Dyrkl A,” Nat. Commun. 1: Article Number 86 (2010)), DANDY (Gourdain et al., “Development of DANDYs, New 3,5-Diaryl-7-Azaindoles Demonstrating Potent DYRK1A Kinase Inhibitory Activity,” J. Med. Chem. 56(23):9569-9585 (2013)), and FINDY (Kii et al., “Selective Inhibition of the Kinase DYRK1A by Targeting its Folding Process,” Nat. Commun. 7:11391 (2016)), pyrazolidine-diones (Koo et al., “QSAR Analysis of Pyrazolidine-3,5-Diones Derivatives as Dyrk1A Inhibitors,” Bioorg. Med. Chem. Lett. 19(8):2324-2328 (2009); Kim et al., “Putative Therapeutic Agents for the Learning and Memory Deficits of People with Down Syndrome,” Bioorg. Med. Chem. Lett. 16(14):3772-3776 (2006)), amino-quinazolines (Rosenthal et al., “Potent and Selective Small Molecule Inhibitors of Specific Isoforms of Cdc2-Like Kinases (Clk) and Dual Specificity Tyrosine-Phosphorylation-Regulated Kinases (Dyrk),” Bioorg. Med. Chem. Lett. 21(10):3152-3158 (2011)), meriolins (Giraud et al., “Synthesis, Protein Kinase Inhibitory Potencies, and In Vitro Antiproliferative Activities of Meridianin Derivatives,” J. Med. Chem. 54(13):4474-4489 (2011); Echalier et al., “Meriolins (3-(Pyrimidin-4-yl)-7-Azaindoles): Synthesis, Kinase Inhibitory Activity, Cellular Effects, and Structure of a CDK2/Cyclin A/Meriolin Complex,” J. Med. Chem. 51(4):737-751 (2008); and Akue-Gedu et al., “Synthesis and Biological Activities of Aminopyrimidyl-Indoles Structurally Related to Meridianins,” Bioorg. Med. Chem. 17(13):4420-4424 (2009)), pyridine and pyrazines (Kassis et al., “Synthesis and Biological Evaluation of New 3-(6-hydroxyindo1-2-yl)-5-(Phenyl) Pyridine or Pyrazine V-Shaped Molecules as Kinase Inhibitors and Cytotoxic Agents,” Eur. J. Med. Chem. 46(11):5416-5434 (2011)), chromenoidoles (Neagoie et al., “Synthesis of Chromeno[3,4-b]indoles as Lamellarin D Analogues: A Novel DYRK1A Inhibitor Class,” Eur. J. Med. Chem. 49:379-396 (2012)), 11H-indolo[3,2-c]quinoline-6-carboxylic acids,37 thiazolo[5,4-f]quinazolines (EHT 5372) (Foucourt et al., “Design and Synthesis of Thiazolo[5,4-f]quinazolines as DYRK1A Inhibitors, Part I.,” Molecules 19(10):15546-15571 (2014) and Coutadeur et al., “A Novel DYRK1A (Dual Specificity Tyrosine Phosphorylation-Regulated Kinase 1A) Inhibitor for the Treatment of Alzheimer's Disease: Effect on Tau and Amyloid Pathologies In Vitro,” J. Neurochem. 133(3):440-451 (2015)), and 5-iodotubercidin (Dirice et al., “Inhibition of DYRK1A Stimulates Human Beta-cell Proliferation,” Diabetes 65:(6):1660-1671 (2016) and Annes et al., “Adenosine Kinase Inhibition Selectively Promotes Rodent and Porcine Islet β-cell Replication,” Proc. Natl. Acad. Sci. 109(10):3915-3920 (2012)) showed potent DYRK1A activity with varying degrees of kinase selectivity.

Most of these compounds are non-selective inhibitors of DYRK1A and exhibit pharmacological side effects, such as CNS activity or apoptosis, thereby limiting their therapeutic utility and potential for pharmaceutical development. This non-selectivity may be attributed to the fact that all these DYRK1A inhibitors are Type I kinase inhibitors, which bind to a highly conserved ATP binding pocket.

The present invention is directed to overcoming deficiencies in the art.

### SUMMARY OF THE INVENTION

One aspect of the present invention relates to a compound of formula (I) having the following structure:

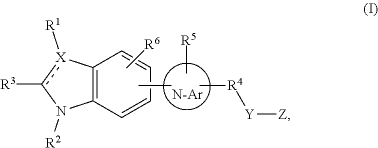

or a stereoisomer, pharmaceutically acceptable salt, oxide, or solvate thereof, wherein

R¹ and R⁶ are independently optionally present, and when present, each is independently selected from H, substituted or unsubstituted C₁-C₆ alkyl, halogen, —CF₃, —OCF₃, or a substituted or unsubstituted cycloalkyl;

R² is selected from H, substituted or unsubstituted C₁-C₆ alkyl, halogen, —CF₃, —OCF₃, substituted or unsubstituted cycloalkyl, or substituted or unsubstituted aryl or heteroaryl;

R³ is optionally present, and when present is an oxygen that forms a carbonyl, or a substituted or unsubstituted C₁-C₆ alkyl;

R⁴ is NH, carbonyl, or branched or unbranched C₁-C₆ alkyl;

R⁵ is optionally present, and when present is a substituted or unsubstituted C₁-C₆ alkyl, halogen, —CF₃, or —OCF₃;

[symbol] is a single or double bond;

X is C, CH, O, or N;

N—Ar is selected from pyridazine, pyrazine, and pyrimidine;

Y is a bond, NH, or branched or linear C₁-C₆ substituted or unsubstituted alkyl; and

Z is H or a substituted or unsubstituted aryl, biaryl, heteroaryl, cycloalkyl, heterocycle, or alkyl, wherein said substituent is selected from hydroxyl, —CF₃, —OCF₃, halogen, nitrile, aryl, C₁-C₆ alkoxy, amide, amino, alkyl, aminocarboxamide, substituted or unsubstituted carboxamide, or a C₁-C₆ alkyl ester.

Another aspect of the present invention relates to a method of inhibiting activity of a kinase in a cell. This method involves contacting the cell with a compound of formula (I) of the present invention under conditions effective to inhibit activity of the kinase in the cell.

A further aspect of the present invention relates to a method of increasing cell proliferation in a population of pancreatic beta cells. This method involves contacting a population of pancreatic beta cells with a compound of formula (I) according to the present invention under conditions effective to increase cell proliferation in the population of pancreatic beta cells.

Another aspect of the present invention relates to a composition comprising a compound of formula (I) according to the present invention and a carrier.

An additional aspect of the present invention relates to a method of treating a subject for a condition associated with insufficient insulin secretion. This method involves administering to a subject in need of treatment for a condition associated with an insufficient level of insulin secretion a compound or composition of the present invention.

A further aspect of the present invention relates to a method of treating a subject for a neurological disorder. This method involves administering to a subject in need of treatment for a neurological disorder a compound of formula (I) according to the present invention under conditions effective to treat the subject for the condition.

Although efforts have been made toward the discovery of potent and selective DYRK1A inhibitors, most of them are still in early stages of lead identification.

Described herein infra is the identification and evaluation of a highly potent and novel class of indole-, benzimidazole- and benzimidazolone pyridazine, pyrazine, and pyrimidine analogue inhibitors.

### BRIEF DESCRIPTION OF THE DRAWINGS

FIG. 1 is a schematic illustration showing the synthesis of N-benzyl-haloanilines intermediate compounds.

FIG. 2 is a schematic illustration showing the synthesis of Indole-, Oxindole- and Benzimidazolone-boronic acid pinacol ester intermediate compounds.

FIG. 3 is a schematic illustration showing the synthesis of Indole-, Benzimidazole- and Benzimidazolone benzyl heterocyclic amine compounds.

FIG. 4 is a schematic illustration showing the synthesis of N-benzylpyrimidin-2-amines or N-(naphthalenylmethyl)pyrimidin-2-amines intermediate compounds.

FIG. 5 is a schematic illustration showing the synthesis of Benzimidazolone benzyl or naphthyl heterocyclic amine compounds.

FIG. 6 is a schematic illustration showing the synthesis of Benzimidazole pyrimidin-2-yl amino methylbenzamide compounds.

FIG. 7 is a schematic illustration showing the synthesis of 5-Bromo-N-phenylpyrimidine-2-carboxamide and N-((5-Bromopyrimidin-2-yl)methyl)aniline compounds.

FIG. 8 is a schematic illustration showing the synthesis of Benzimidazolone benzyl heterocyclic amine compounds.

FIG. 9 is a schematic illustration showing the synthesis of 5-(2-(Benzylamino)pyrimidin-5-yl)-methyl-1H-benzo[d]imidazol-2(3H)-one compounds.

FIG. 10 is a schematic illustration showing the synthesis of 5-(2-(Phenylamino)pyrimidin-5-yl)-1H-benzo[d]imidazol-2(3H)-one compounds.

FIG. 11 is a schematic illustration showing the synthesis of heterocyclic methylamino pyrimidinyl-1H-benzo[d]imidazol-2(3H)-one compounds.

FIG. 12 is a schematic illustration showing the synthesis of substituted Benzo[d]imidazol-2(3H)-one pyrimidyl(benzylamine) compounds.

FIG. 13 is a schematic illustration showing the synthesis of 5-(2-((1-Phenylethyl)amino)pyrimidin-5-yl)-1H-benzo[d]imidazol-2(3H)-one compounds.

FIG. 14 is a schematic illustration showing the synthesis of 5-(2-((3-(2-Methoxyethoxy)benzyl)amino)pyrimidin-5-yl)-1H-benzo[d]imidazol-2(3H)-one (62).

### DETAILED DESCRIPTION OF THE INVENTION

One aspect of the present invention relates to a compound of formula (I) having the following structure:

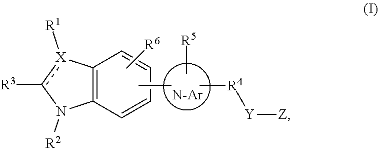

or a stereoisomer, pharmaceutically acceptable salt, oxide, or solvate thereof, where

R¹ and R⁶ are independently optionally present, and when present, each is independently selected from H, substituted or unsubstituted C₁-C₆ alkyl, halogen, —CF₃, —OCF₃, or a substituted or unsubstituted cycloalkyl;

R² is selected from H, substituted or unsubstituted C₁-C₆ alkyl, halogen, —CF₃, —OCF₃, substituted or unsubstituted cycloalkyl, or substituted or unsubstituted aryl or heteroaryl; R³ is optionally present, and when present is an oxygen that forms a carbonyl, or a substituted or unsubstituted C₁-C₆ alkyl;

R⁴ is NH, carbonyl, or branched or unbranched C₁-C₆ alkyl;

R⁵ is optionally present, and when present is a substituted or unsubstituted C₁-C₆ alkyl, halogen, —CF₃, or —OCF₃;

[symbol] is a single or double bond;

X is C, CH, O, or N;

N—Ar is selected from pyridazine, pyrazine, and pyrimidine;

Y is a bond, NH, or branched or linear C₁-C₆ substituted or unsubstituted alkyl; and

Z is H or a substituted or unsubstituted aryl, biaryl, heteroaryl, cycloalkyl, heterocycle, or alkyl, wherein said substituent is selected from hydroxyl, —CF₃, —OCF₃, halogen, nitrile, aryl, C₁-C₆ alkoxy, amide, amino, alkyl, aminocarboxamide, substituted or unsubstituted carboxamide, or a C₁-C₆ alkyl ester.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs.

As used herein, the term “halogen” means fluoro, chloro, bromo, or iodo.

The term “alkyl” means an aliphatic hydrocarbon group which may be straight or branched having about 1 to about 6 carbon atoms in the chain (or the number of carbons designated by “Cₙ-Cₙ”, where n is the numerical range of carbon atoms). Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl.

The term “alkoxy” means groups of from 1 to 6 carbon atoms of a straight, branched, or cyclic configuration and combinations thereof attached to the parent structure through an oxygen. Examples include methoxy, ethoxy, propoxy, isopropoxy, butoxy, cyclopropyloxy, cyclohexyloxy, and the like. Alkoxy also includes methylenedioxy and ethylenedioxy in which each oxygen atom is bonded to the atom, chain, or ring from which the methylenedioxy or ethylenedioxy group is pendant so as to form a ring. Thus, for example, phenyl substituted by alkoxy may be. for example,

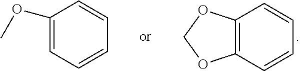

The term “cycloalkyl” means a non-aromatic, saturated or unsaturated, mono- or multi-cyclic ring system of about 3 to about 7 carbon atoms, or of about 5 to about 7 carbon atoms, and which may include at least one double bond. Exemplary cycloalkyl groups include, without limitation, cyclopropyl, cyclobutyl, cyclopentyl, cyclohexyl, cycloheptyl, cyclopropenyl, cyclobutenyl, cyclopentenyl, cyclophenyl, anti-bicyclopropane, and syn-tricyclopropane.

The term “aryl” means an aromatic monocyclic or multi-cyclic (polycyclic) ring system of 6 to about 19 carbon atoms, or of 6 to about 10 carbon atoms, and includes arylalkyl groups. The ring system of the aryl group may be optionally substituted. Representative aryl groups of the present invention include, but are not limited to, groups such as phenyl, naphthyl, azulenyl, phenanthrenyl, anthracenyl, fluorenyl, pyrenyl, triphenylenyl, chrysenyl, and naphthacenyl.

As used herein the term “biaryl” includes not only such traditional biaryl groups as biphenyl, but fused variants thereof, naphthyl-containing and heteroatom-containing variants thereof, and benzhydryl variants thereof.

The term “heteroaryl” means an aromatic monocyclic or multi-cyclic ring system of about 5 to about 19 ring atoms, or about 5 to about 10 ring atoms, in which one or more of the atoms in the ring system is/are element(s) other than carbon, for example, nitrogen, oxygen, or sulfur. In the case of multi-cyclic ring system, only one of the rings needs to be aromatic for the ring system to be defined as “heteroaryl.” Preferred heteroaryls contain about 5 to 6 ring atoms. The prefix aza, oxa, thia, or thio before heteroaryl means that at least a nitrogen, oxygen, or sulfur atom, respectively, is present as a ring atom. A nitrogen, carbon, or sulfur atom in the heteroaryl ring may be optionally oxidized; the nitrogen may optionally be quaternized. Representative heteroaryls include pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, pthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-a]pyridinyl, [1,2,4]triazolo[1,5-a]pyridinyl, thieno[2,3-b]furanyl, thieno[2,3-b]pyridinyl, thieno[3,2-b]pyridinyl, furo[2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8-tetrahydroimidazo[1,2-a]pyrazinyl, 6,7-dihydro-4H-pyrazolo[5,1-c][1,4]oxazinyl, 2-oxo-2,3-dihydrobenzo[d]oxazolyl, 3,3-dimethyl-2-oxoindolinyl, 2-oxo-2,3-dihydro-1H-pyrrolo[2,3-b]pyridinyl, benzo[c][1,2,5]oxadiazolyl, benzo[c][1,2,5]thiadiazolyl, 3,4-dihydro-2H-benzo[b][1,4]oxazinyl, 5,6,7,8-tetrahydro-[1,2,4]triazolo[4,3-a]pyrazinyl, [1,2,4]triazolo[4,3-a]pyrazinyl, 3-oxo-[1,2,4]triazolo[4,3-a]pyridin-2(3H)-yl, and the like.

As used herein, “heterocycle” refers to a stable 3- to 18-membered ring (radical) of carbon atoms and from one to five heteroatoms selected from nitrogen, oxygen, and sulfur. The heterocycle may be a monocyclic or a polycyclic ring system, which may include fused, bridged, or spiro ring systems; and the nitrogen, carbon, or sulfur atoms in the heterocycle may be optionally oxidized; the nitrogen atom may be optionally quaternized; and the ring may be partially or fully saturated. Examples of such heterocycles include, without limitation, azepinyl, azocanyl, pyranyl dioxanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, and thiamorpholinyl sulfone.

Further heterocycles and heteroaryls are described in Katritzky et al., eds., Comprehensive Heterocyclic Chemistry: The Structure, Reactions, Synthesis and Use of Heterocyclic Compounds, Vol. 1-8, Pergamon Press, N.Y. (1984), which is hereby incorporated by reference in its entirety.

The terms “carboxamide” or “amide” as used herein refer to C(O)NRₐR_{b} wherein Rₐ and R_{b} are each independently hydrogen, alkyl or any other suitable substituent.

The term “aminocarboxamide” means NH₂XC(O)NRₐR_{b} where X is phenyl or heterocycle and Rₐ and R_{b} are each independently hydrogen, alkyl, or any other chemically suitable substituent.

The phrases “substituted or unsubstituted” and “optionally substituted” mean a group may (but does not necessarily) have a substituent at each substitutable atom of the group (including more than one substituent on a single atom), and the identity of each substituent is independent of the others.

The term “substituted” means that one or more hydrogen on a designated atom is replaced with a selection from the indicated group, provided that the designated atom's normal valency is not exceeded. “Unsubstituted” atoms bear all of the hydrogen atoms dictated by their valency. When a substituent is oxo (i.e., ═O), then 2 hydrogens on the atom are replaced. Combinations of substituents and/or variables are permissible only if such combinations result in stable compounds. By “stable compound” it is meant a compound that is sufficiently robust to survive isolation to a useful degree of purity from a reaction mixture and formulation into an efficacious therapeutic agent.

By “compound(s) of the invention” and equivalent expressions, it is meant compounds herein described, which expression includes the prodrugs, the pharmaceutically acceptable salts, the oxides, and the solvates, e.g. hydrates, where the context so permits.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. The present invention is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. All tautomeric forms are also intended to be included.

As would be understood by a person of ordinary skill in the art, the recitation of “a compound” is intended to include salts, solvates, oxides, and inclusion complexes of that compound as well as any stereoisomeric form, or a mixture of any such forms of that compound in any ratio. Thus, in accordance with some embodiments of the invention, a compound as described herein, including in the contexts of pharmaceutical compositions, methods of treatment, and compounds per se, is provided as the salt form.

The term “solvate” refers to a compound in the solid state, where molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

Inclusion complexes are described in Remington, The Science and Practice of Pharmacy, 19th Ed. 1:176-177 (1995), which is hereby incorporated by reference in its entirety. The most commonly employed inclusion complexes are those with cyclodextrins, and all cyclodextrin complexes, natural and synthetic, are specifically encompassed by the present invention.

The term “pharmaceutically acceptable salt” refers to salts prepared from pharmaceutically acceptable non-toxic acids or bases including inorganic acids and bases and organic acids and bases.

The term “pharmaceutically acceptable” means it is, within the scope of sound medical judgment, suitable for use in contact with the cells of humans and lower animals without undue toxicity, irritation, allergic response and the like, and are commensurate with a reasonable benefit/risk ratio.

In one embodiment of the compound of formula (I),
R^{l} and R² are H;
R³ is an oxygen that forms a carbonyl;
R⁴ is NH;
[symbol] is a single bond; and
X is N.

In accordance with this embodiment, Z may be an unsubstituted phenyl ring or a phenyl ring substituted with a hydroxyl, —OCF₃, halogen, a nitrile, a benzene ring, C₁-C₆ alkoxy, or —CONH₂. Compounds of this embodiment include, without limitation:

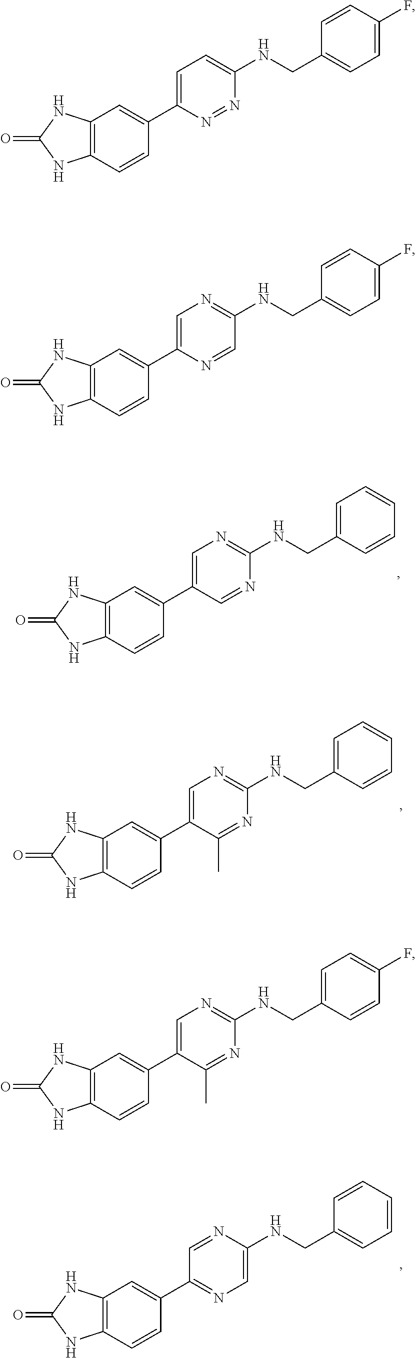 [SMILES: O=c1[nH]c2ccc(-c3ccc(NCc4ccc(F)cc4)nn3)cc2[nH]1 ; O=c1[nH]c2ccc(-c3cnc(NCc4ccc(F)cc4)cn3)cc2[nH]1 ; O=c1[nH]c2ccc(-c3cnc(NCc4ccccc4)nc3)cc2[nH]1 ; Cc1nc(NCc2ccccc2)ncc1-c1ccc2[nH]c(=O)[nH]c2c1 ; Cc1nc(NCc2ccc(F)cc2)ncc1-c1ccc2[nH]c(=O)[nH]c2c1 ; O=c1[nH]c2ccc(-c3cnc(NCc4ccccc4)cn3)cc2[nH]1]

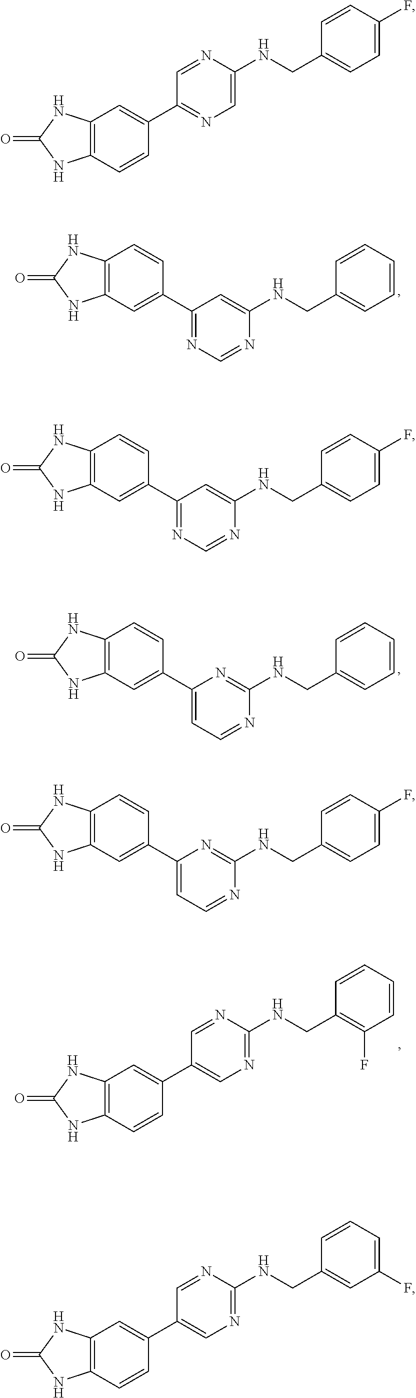

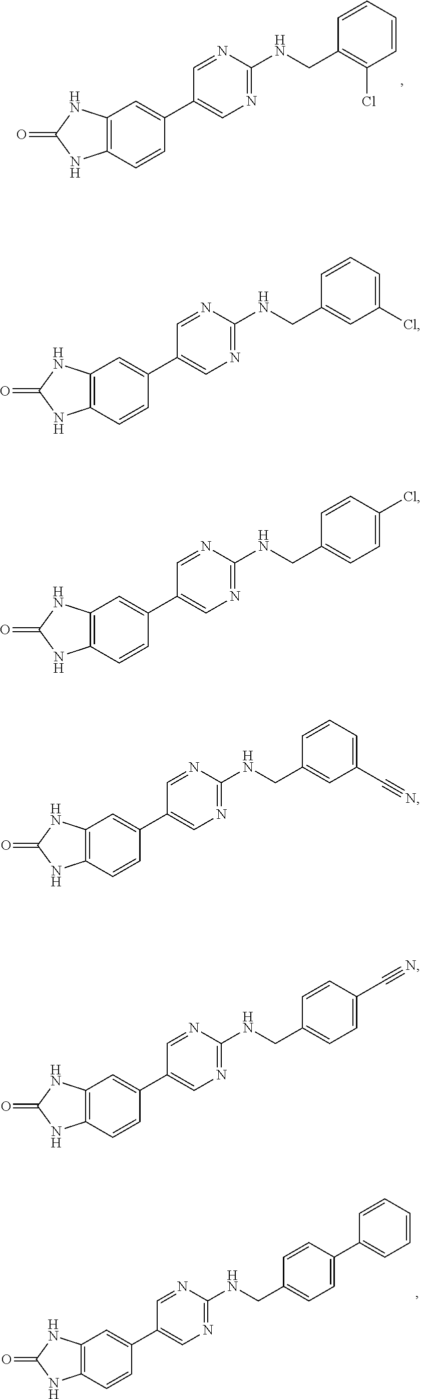 [SMILES: O=c1[nH]c2ccc(-c3cnc(NCc4ccccc4Cl)nc3)cc2[nH]1 ; O=c1[nH]c2ccc(-c3cnc(NCc4cccc(Cl)c4)nc3)cc2[nH]1 ; O=c1[nH]c2ccc(-c3cnc(NCc4ccc(Cl)cc4)nc3)cc2[nH]1 ; N#Cc1cccc(CNc2ncc(-c3ccc4[nH]c(=O)[nH]c4c3)cn2)c1 ; N#Cc1ccc(CNc2ncc(-c3ccc4[nH]c(=O)[nH]c4c3)cn2)cc1 ; O=c1[nH]c2ccc(-c3cnc(NCc4ccc(-c5ccccc5)cc4)nc3)cc2[nH]1]

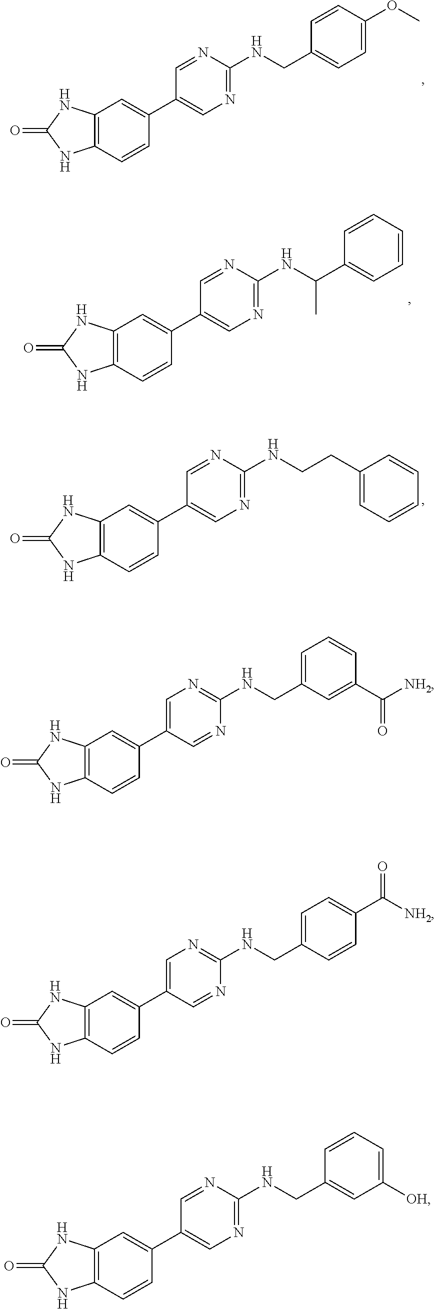 [SMILES: COc1ccc(CNc2ncc(-c3ccc4[nH]c(=O)[nH]c4c3)cn2)cc1 ; CC(Nc1ncc(-c2ccc3[nH]c(=O)[nH]c3c2)cn1)c1ccccc1 ; O=c1[nH]c2ccc(-c3cnc(NCCc4ccccc4)nc3)cc2[nH]1 ; NC(=O)c1cccc(CNc2ncc(-c3ccc4[nH]c(=O)[nH]c4c3)cn2)c1 ; NC(=O)c1ccc(CNc2ncc(-c3ccc4[nH]c(=O)[nH]c4c3)cn2)cc1 ; O=c1[nH]c2ccc(-c3cnc(NCc4cccc(O)c4)nc3)cc2[nH]1]

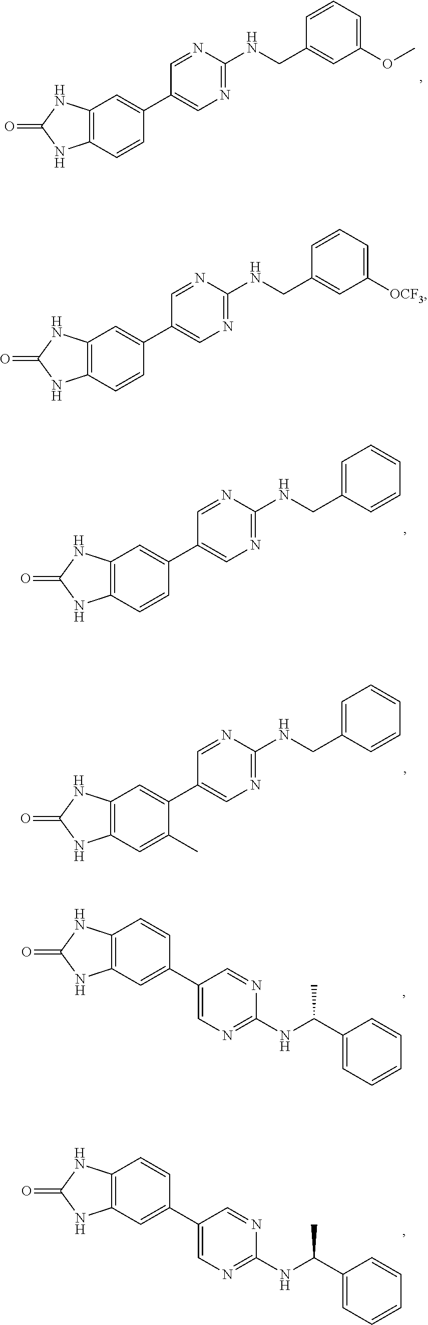 [SMILES: COc1cccc(CNc2ncc(-c3ccc4[nH]c(=O)[nH]c4c3)cn2)c1 ; O=c1[nH]c2ccc(-c3cnc(NCc4cccc(OC(F)(F)F)c4)nc3)cc2[nH]1 ; O=c1[nH]c2ccc(-c3cnc(NCc4ccccc4)nc3)cc2[nH]1 ; Cc1cc2[nH]c(=O)[nH]c2cc1-c1cnc(NCc2ccccc2)nc1 ; C[C@@H](Nc1ncc(-c2ccc3[nH]c(=O)[nH]c3c2)cn1)c1ccccc1 ; C[C@H](Nc1ncc(-c2ccc3[nH]c(=O)[nH]c3c2)cn1)c1ccccc1]

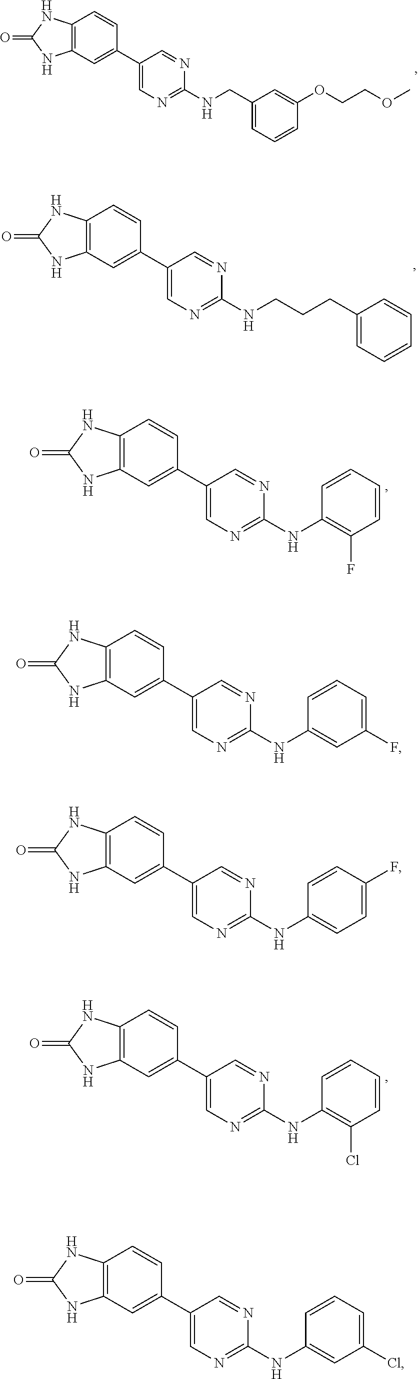

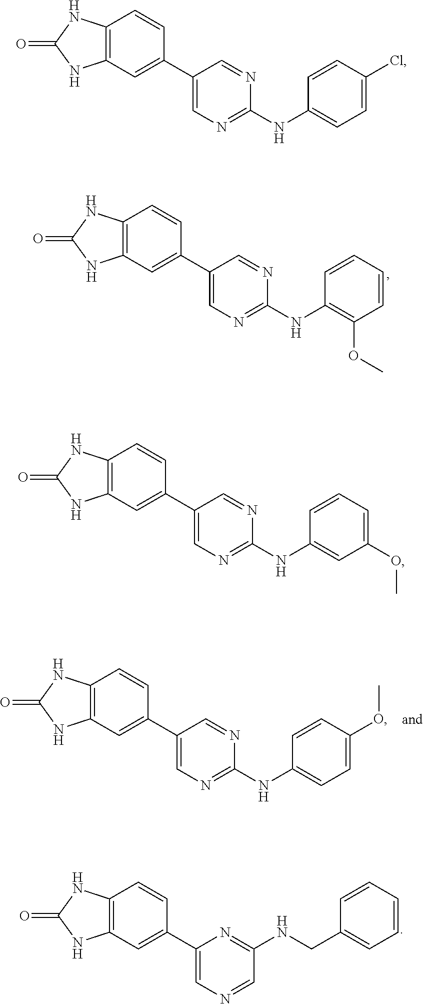 [SMILES: O=c1[nH]c2ccc(-c3cnc(Nc4ccc(Cl)cc4)nc3)cc2[nH]1 ; COc1ccccc1Nc1ncc(-c2ccc3[nH]c(=O)[nH]c3c2)cn1 ; COc1cccc(Nc2ncc(-c3ccc4[nH]c(=O)[nH]c4c3)cn2)c1 ; COc1ccc(Nc2ncc(-c3ccc4[nH]c(=O)[nH]c4c3)cn2)cc1 ; "and" ; O=c1[nH]c2ccc(-c3cncc(NCc4ccccc4)n3)cc2[nH]1]

In a further embodiment, Z may be selected from pyridinyl and naphthalene.

Compounds of this embodiment include, without limitation:

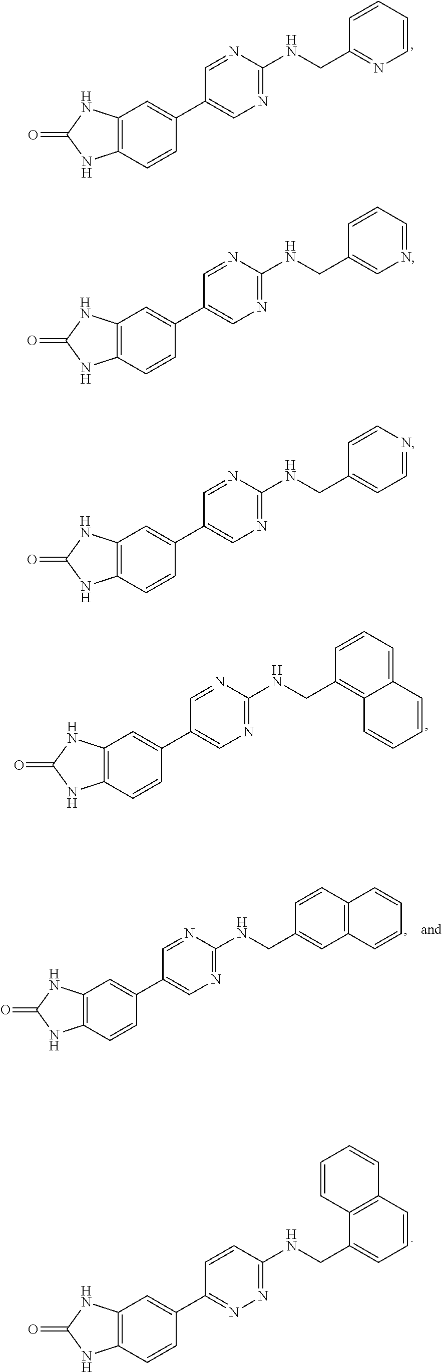 [SMILES: O=c1[nH]c2ccc(-c3cnc(NCc4ccccn4)nc3)cc2[nH]1 ; O=c1[nH]c2ccc(-c3cnc(NCc4cccnc4)nc3)cc2[nH]1 ; O=c1[nH]c2ccc(-c3cnc(NCc4ccncc4)nc3)cc2[nH]1 ; O=c1[nH]c2ccc(-c3cnc(NCc4cccc5ccccc45)nc3)cc2[nH]1 ; O=c1[nH]c2ccc(-c3cnc(NCc4ccc5ccccc5c4)nc3)cc2[nH]1 ; O=c1[nH]c2ccc(-c3ccc(NCc4cccc5ccccc45)nn3)cc2[nH]1]

In another embodiment of the compound of formula (I),
R² is H;
R⁴ is NH;
[symbol] is a double bond;
Y is CH₂; and
X is C or N.

In accordance with this embodiment, the compound may be selected from

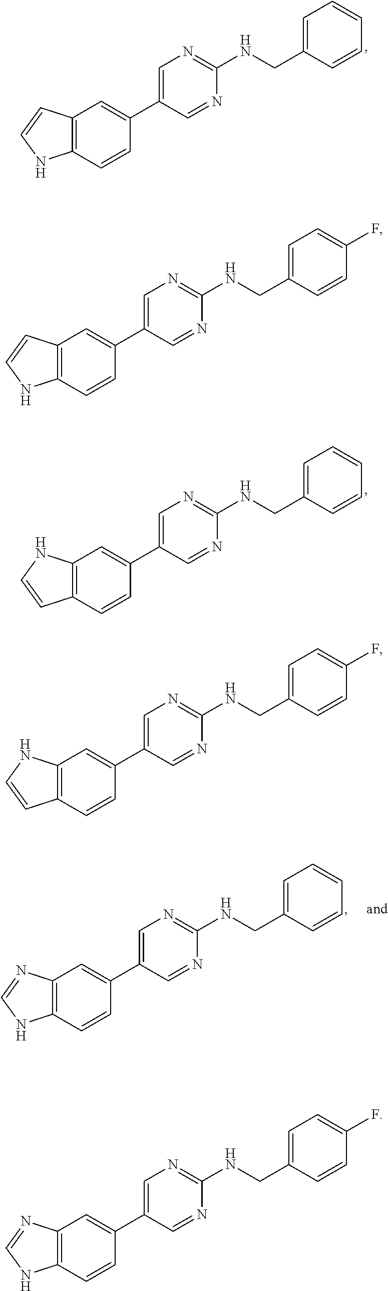 [SMILES: c1ccc(CNc2ncc(-c3ccc4[nH]ccc4c3)cn2)cc1 ; Fc1ccc(CNc2ncc(-c3ccc4[nH]ccc4c3)cn2)cc1 ; c1ccc(CNc2ncc(-c3ccc4cc[nH]c4c3)cn2)cc1 ; Fc1ccc(CNc2ncc(-c3ccc4cc[nH]c4c3)cn2)cc1 ; c1ccc(CNc2ncc(-c3ccc4[nH]cnc4c3)cn2)cc1 ; Fc1ccc(CNc2ncc(-c3ccc4[nH]cnc4c3)cn2)cc1]

In yet another embodiment of the compound of formula (I),
R¹ and R² are H;
R³ is an oxygen that forms a carbonyl;
R⁵ is NH;
[symbol] is a single bond; and
X is CH.

In accordance with this embodiment, the compound may be selected from

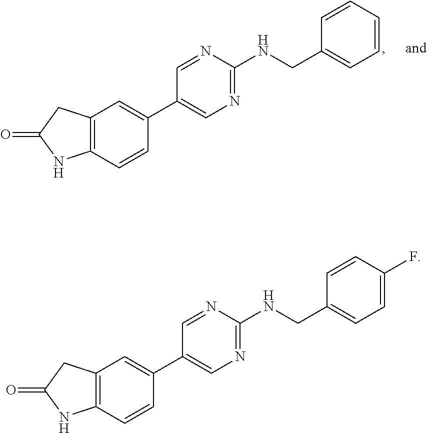 [SMILES: O=C1Cc2cc(-c3cnc(NCc4ccccc4)nc3)ccc2N1 ; O=C1Cc2cc(-c3cnc(NCc4ccc(F)cc4)nc3)ccc2N1]

In a further embodiment of the compound of formula (I),
R¹ is CH₃;
R² is H;
R³ is an oxygen that forms a carbonyl;
R⁴ is NH;
Y is CH₂; and
X is N.

In accordance with this embodiment, the compound may be selected from

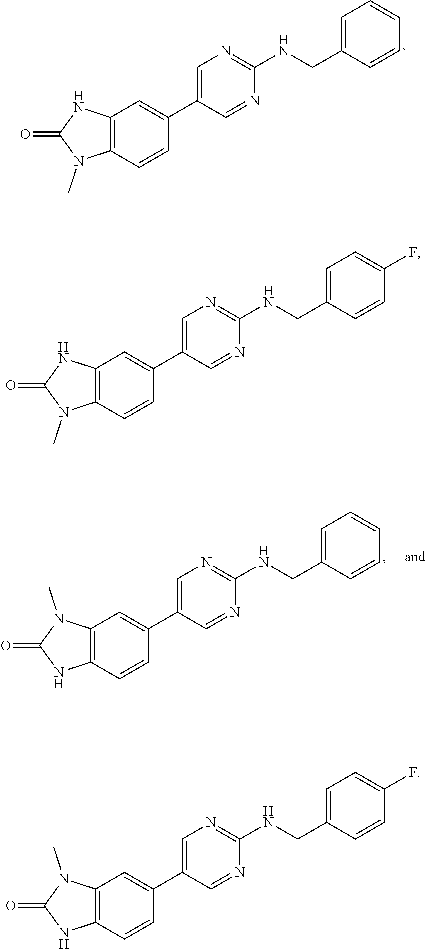 [SMILES: Cn1c(=O)[nH]c2cc(-c3cnc(NCc4ccccc4)nc3)ccc21 ; Cn1c(=O)[nH]c2cc(-c3cnc(NCc4ccc(F)cc4)nc3)ccc21 ; Cn1c(=O)[nH]c2ccc(-c3cnc(NCc4ccccc4)nc3)cc21 ; Cn1c(=O)[nH]c2ccc(-c3cnc(NCc4ccc(F)cc4)nc3)cc21]

In another embodiment of the compound of formula (I),
R¹ is H;
R² is H;
R³ is an oxygen that forms a carbonyl;
R⁴ is NH;
Y is CH₂;
X is N; and

Z is a heteroaryl.

In accordance with this embodiment, the compound may be selected from

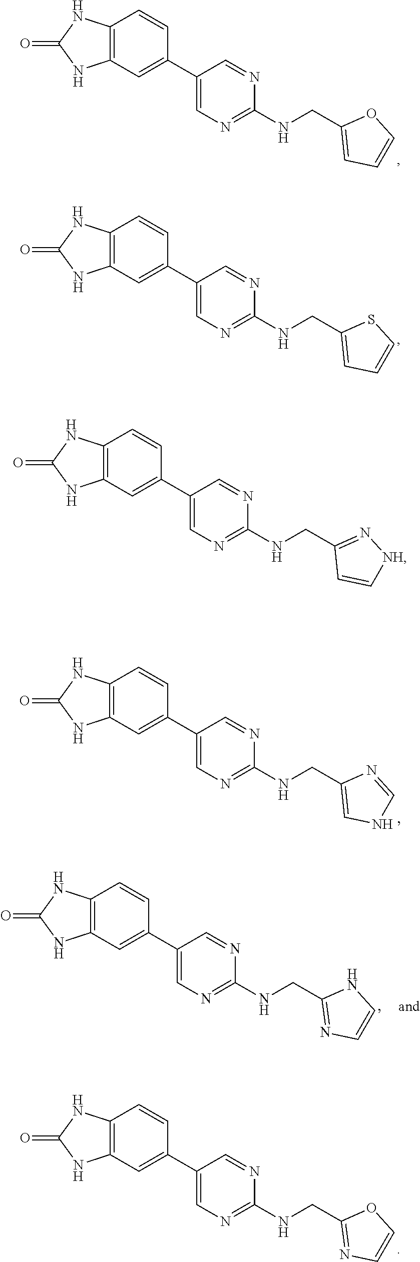 [SMILES: O=c1[nH]c2ccc(-c3cnc(NCc4ccco4)nc3)cc2[nH]1 ; O=c1[nH]c2ccc(-c3cnc(NCc4cccs4)nc3)cc2[nH]1 ; O=c1[nH]c2ccc(-c3cnc(NCc4cc[nH]n4)nc3)cc2[nH]1 ; O=c1[nH]c2ccc(-c3cnc(NCc4c[nH]cn4)nc3)cc2[nH]1 ; O=c1[nH]c2ccc(-c3cnc(NCc4ncc[nH]4)nc3)cc2[nH]1 ; O=c1[nH]c2ccc(-c3cnc(NCc4ncco4)nc3)cc2[nH]1]

In a further embodiment of the compound of formula (I),
R¹ is H;
R² is a C₁-C₆ alkyl, cycloalkyl, aryl, or heteroaryl;
R³ is an oxygen that forms a carbonyl;
R⁴ is NH;
Y is CH₂;
X is N; and
Z is a phenyl ring.

In accordance with this embodiment, the compound may be selected from

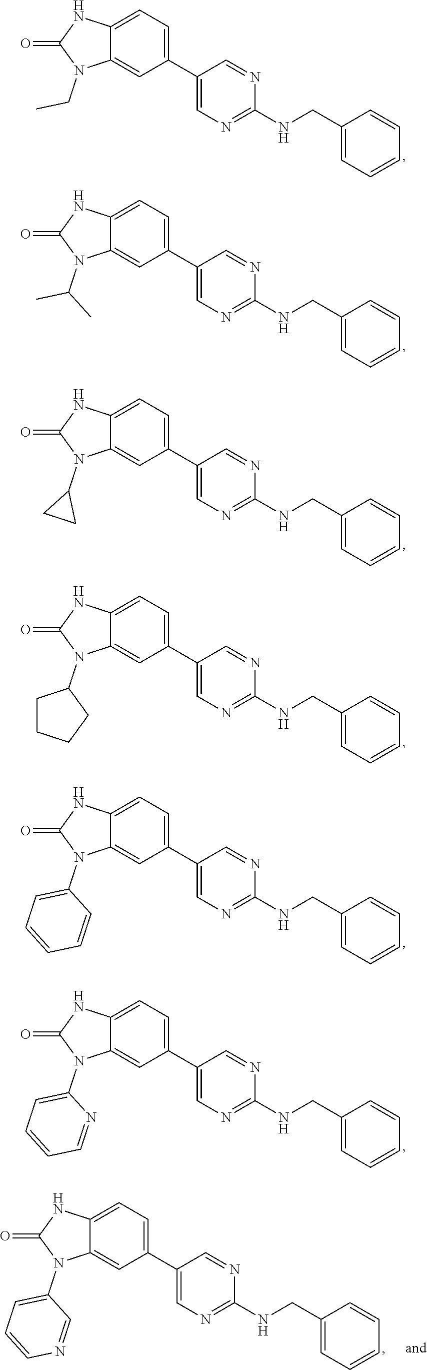

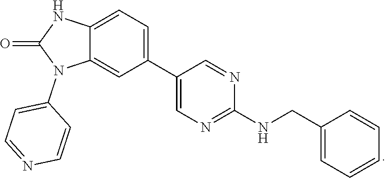 [SMILES: O=c1[nH]c2ccc(-c3cnc(NCc4ccccc4)nc3)cc2n1-c1ccncc1]

In accordance with one embodiment, Z may be an unsubstituted phenyl ring or a phenyl ring substituted with a halogen, a nitrile, a benzene ring, C₁-C₆ alkoxy, or —CONH₂. Z may be selected from pyridinyl, cyclohexane, naphthalene, and morpholine. Y may be selected from a bond, CH₂, CH(CH₃), CH₂CH₂, CH₂CH(CH₃), and CH(CH₃)CH₂.

Another aspect of the present invention relates to a method of inhibiting activity of a kinase in a cell. This method involves contacting the cell with a compound of formula (I) under conditions effective to inhibit activity of the kinase in the cell.

In one embodiment, the kinase is a dual-specificity tyrosine phosphorylation-regulated kinase (“DYRK”). The kinase may be a dual-specificity tyrosine phosphorylation-regulated kinase 1A (“DYRK1A”).

The cell may be a mammalian cell. Mammalian cells include cells from, for example, mice, hamsters, rats, cows, sheep, pigs, goats, and horses, monkeys, dogs (e.g., Canis familiaris), cats, rabbits, guinea pigs, and primates, including humans. For example, the cell may be a human cell.

In one embodiment, the cell is a pancreatic beta cell. If needed, methods for determining whether a cell has a pancreatic beta cell phenotype are known in the art and include, without limitation, incubating the cell with glucose and testing whether insulin expression in the cell is increased or induced. Other methods include testing whether beta cell specific transcription factors are expressed, the detection of beta cell specific gene products with the help of RNA quantitative PCR, the transplantation of a candidate cell in diabetic mice, and subsequent testing of the physiologic response following said transplantation as well analyzing the cells with electron microscopy.

In another embodiment, the cell is a cancer cell.

In yet another embodiment, the cell is a neural cell.

Methods of the present invention may be carried out ex vivo or in vivo. When carried out ex vivo, a population of cells may be, according to one embodiment, provided by obtaining cells from a pancreas and culturing the cells in a liquid medium suitable for the in vitro or ex vivo culture of mammalian cells, in particular human cells. For example, and without limitation, a suitable and non-limiting culture medium may be based on a commercially available medium such as RPMI1640 from Invitrogen.

A further aspect of the present invention relates to a method of increasing cell proliferation in a population of pancreatic beta cells. This method involves contacting a population of pancreatic beta cells with a compound of formula (I) under conditions effective to increase cell proliferation in the population of pancreatic beta cells.

In one embodiment, contacting is carried out with a composition (i.e., a single composition) comprising the compound.

The method may further involve contacting the population of pancreatic beta cells with a transforming growth factor beta (TGFβ) superfamily signaling pathway inhibitor. In accordance with this embodiment, the method may be carried out with a composition comprising the compound and the TGFβ superfamily signaling pathway inhibitor. In another embodiment, the compound of formula (I) and the TGFβ superfamily signaling pathway inhibitor separately contact a population of pancreatic beta cells simultaneously or in sequence.

TGFβ superfamily signaling pathway inhibitors include small molecules and other (e.g., neutralizing monoclonal antibodies, synthetic/recombinant peptide inhibitors, and siRNA) inhibitors of the BMP family of receptors, activing and inhibin receptors, GDF11 receptors and related receptors.

TGFβ superfamily signaling pathway inhibitors are also known in the art and include, without limitation, SB431542, SB505124, A-83-01, Decorin, soluble TGF-β receptor,

Ierdelimumab, metelimumab, AP-12009, Follistatin, FLRG, GAST-1, GDF8 propeptide, MYO-029, Noggin, chordin, Cer/Dan, ectodin, and Sclerostin (see Tsuchida et al., “Inhibitors of the TGF-beta Superfamily and their Clinical Applications,” Mini Rev. Med. Chem. 6(11):1255-61 (2006), which is hereby incorporated by reference in its entirety.

Other inhibitors of TGF-β signaling include, without limitation, 2-(3-(6-Methylpyridin-2-yl)-1H-pyrazol-4-yl)-1,5 napththyridine; [3-(Pyridin-2-yl)-4-(4-quinoyl)]-1H-pyrazole; 3-(6-Methylpyridin-2-yl)-4-(4-quinolyl)-1-phenylthiocarbamoyl-1H-pyrazole; SB-431542; SM16; SB-505124; and 2-(3-(6-Methylpyridin-2-yl)-1H-pyrazol-4-yl)-1,5 napththyridine (ALK5 Inhibitor II) (see U.S. Pat. No. 8,298,825, which is hereby incorporated by reference in its entirety).

Inhibitors of TGF-β signaling are described in Callahan et al., J. Med. Chem. 45:999-1001 (2002); Sawyer et al., J. Med. Chem. 46:3953-3956 (2003); Gellibert et al., J. Med. Chem. 47:4494-4506 (2004); Tojo et al., Cancer Sci. 96:791-800 (2005); Valdimarsdottir et al., APMIS 113:773-389 (2005); Petersen et al., Kidney International 73:705-715 (2008); Yingling et al., Nature Rev. Drug Disc. 3:1011-1022 (2004); Byfield et al., Mol. Pharmacol. 65:744-752 (2004); Dumont et al., Cancer Cell 3:531-536 (2003); PCT Publication No. WO 2002/094833; PCT Publication No. WO 2004/026865; PCT Publication No. WO 2004/067530; PCT Publication No. WO 2009/032667; PCT Publication No. WO 2004/013135; PCT Publication No. WO 2003/097639; PCT Publication No. WO 2007/048857; PCT Publication No. WO 2007/018818; PCT Publication No. WO 2006/018967; PCT Publication No. WO 2005/039570; PCT Publication No. WO 2000/031135; PCT Publication No. WO 1999/058128; U.S. Pat. Nos. 6,509,318; 6,090,383; 6,419,928; 9,927,738; 7,223,766; 6,476,031; 6,419,928; 7,030,125; 6,943,191; U.S. Patent Application Publication No. 2005/0245520; U.S. Patent Application Publication No. 2004/0147574; U.S. Patent Application Publication No. 2007/0066632; U.S. Patent Application Publication No. 2003/0028905; U.S. Patent Application Publication No. 2005/0032835; U.S. Patent Application Publication No. 2008/0108656; U.S. Patent Application Publication No. 2004/015781; U.S. Patent Application Publication No. 2004/0204431; U.S. Patent Application Publication No. 2006/0003929; U.S. Patent Application Publication No. 2007/0155722; U.S. Patent Application Publication No. 2004/0138188 and U.S. Patent Application Publication No. 2009/0036382, which are hereby incorporated by reference in their entirety.

Exemplary inhibitors of TGF-β signaling include, but are not limited to, AP-12009 (TGF-β Receptor type II antisense oligonucleotide), Lerdelimumab (CAT 152, antibody against TGF-β Receptor type II) GC-1008 (antibody to all isoforms of human TGF-β), ID11 (antibody to all isoforms of murine TGF-β), soluble TGF-β, soluble TGF-β Receptor type II, dihydropyrroloimidazole analogs (e.g., SKF-104365), triarylimidazole analogs (e.g., SB-202620 (4-(4-(4-fluorophenyl)-5-(pyridin-4-yl)-1H-imidazol-2-yl)benzoic acid) and SB-203580 (4-(4-Fluorophenyl)-2-(4-methylsulfinyl phenyl)-5-(4-pyridyl)-1H-imidazole)), RL-0061425, 1,5-naphthyridine aminothiazole and pyrazole derivatives (e.g., 4-(6-methyl-pyridin-2-yl)-5-(1,5-naphthyridin-2-yl)-1,3-thiazole-2-amine and 2-[3-(6-methyl-pyridin-2-yl)-1H-pyrazole-4-yl]-1,5-naphthyridine), SB-431542 (4-(5-Benzol[1,3]dioxol-5-yl-4-pyridin-2-yl-1H-imidazol-2-yl)-benzamide), GW788388 (4-(4-(3-(pyridin-2-yl)-1H-pyrazol-4-yl)pyridin-2-yl)-N-(tetrahydro-2H-pyran-4-yl)benzamide), A-83-01 (3-(6-Methyl-2-pyridinyl)-N-phenyl-4-(4-quinolinyl)-1H-pyrazole-1-carbothioamide), Decorin, Lefty 1, Lefty 2, Follistatin, Noggin, Chordin, Cerberus, Gremlin, Inhibin, BIO (6-bromo-indirubin-3′-oxime), Smad proteins (e.g., Smad6, Smad7), and Cystatin C.

Inhibitors of TGF-β signaling also include molecules which inhibit TGF-β Receptor type I. Inhibitors of TGF-β Receptor type I include, but are not limited to, soluble TGF-β Receptor type I; AP-11014 (TGF-β Receptor type I antisense oligonucleotide); Metelimumab (CAT 152, TGF-β Receptor type I antibody); LY550410; LY580276 (3-(4-fluorophenyl)-5,6-dihydro-2-(6-methylpyridin-2-yl)-4H-pyrrolo[1,2-b]pyrazole); LY364947 (4-[3-(2-Pyridinyl)-1H-pyrazol-4-yl]-quinoline); LY2109761; LY573636 (N-((5-bromo-2-thienyl)sulfonyl)-2,4-dichlorobenzamide); SB-505124 (2-(5-Benzo[1,3]dioxo1-5-yl-2-tert-butyl-3H-imidazol-4-yl)-6-methylpyridine); SD-208 (2-(5-Chloro-2-fluorophenyl)-4-[(4-pyridyl)amino]pteridine); SD-093; KI2689; SM16; FKBP12 protein; and 3-(4-(2-(6-methylpyridin-2-yl)H-imidazo[1,2-a]pyridin-3-yl)quinolin-7-yloxy)-N,N-dimethylpropan-1-amine.

Inhibitors of TGF-β Receptor type I are described in Byfield and Roberts, Trends Cell Biol. 14:107-111 (2004); Sawyer et al., Bioorg. Med. Chem. Lett. 14:3581-3584 (2004); Sawyer et al., J. Med. Chem. 46:3953-3956 (2003); Byfield et al., Mol. Pharmacol. 65:744-752 (2004); Gellibert et al., J. Med. Chem. 47:4494-4506 (2004); Yingling et al., Nature Rev. Drug Disc. 3:1011-1022 (2004); Dumont et al., Cancer Cell 3:531-536 (2003); Tojo et al., Cancer Sci. 96:791-800 (2005); PCT Publication No. WO 2004/026871; PCT Publication No. WO 2004/021989; PCT Publication No. WO 2004/026307; PCT Publication No. WO 2000/012497; U.S. Pat. Nos. 5,731,424; 5,731,144; 7,151,169; U.S. Patent Application Publication No. 2004/00038856 and U.S. Patent Application Publication No.

2005/0245508, all of which are herein incorporated in their entirety.

In one embodiment, the TGFβ superfamily signaling pathway inhibitor includes compounds that interfere with TGFβ superfamily ligands, receptors, and/or downstream signaling molecules (e.g., SMADs) or nuclear targets (e.g., chromatin modifying complexes and transcription factors).

In one embodiment, the TGFβ superfamily signaling pathway inhibitor may be antisera that neutralize, e.g., TGFβ ligand.

In another embodiment, the TGFβ superfamily signaling pathway inhibitor is selected from the group consisting of an inhibitor of TGFP/TGFβ receptor binding, activin or inhibin/activin receptor binding, and bone morphogenetic protein (BMP)/BMP receptor binding.

The TGFβ superfamily signaling pathway inhibitor may be an inhibitor of TGFP/TGFβ receptor binding selected from the group consisting of LY364947 and GW788388.

The TGFβ superfamily signaling pathway inhibitor may be an inhibitor of activin or inhibin/activin receptor binding selected from the group consisting of SB431542 and Alk5 inhibitor II. Additional exemplary inhibitors of activin or inhibin/activin receptor binding may be selected from the group consisting of SB-505124, BYM388, follistatin, follistatin-related protein (FSRP), follistatin domains (i.e., Fs2, Fs12, Fs123), A-83-01, Cripto, GW788388, BAMBI, and Sotatercept (see Byfield et al., “SB-505124 is a Selective Inhibitor of Transforming Growth Factor-Beta Type I Receptors ALK4, ALKS, and ALK7,” Mol. Pharmacol. 65(3):744-52 (2004); Lach-Trifilieffa et al., “An Antibody Blocking Activin Type II Receptors Induces Strong Skeletal Muscle Hypertrophy and Protects from Atrophy,” Mol. Cell. Biol. 34(4):606-18 (2014); Zhang et al., “Inhibition of Activin Signaling Induces Pancreatic Epithelial Cell Expansion and Diminishes Terminal Differentiation of Pancreatic f3-Cells,” Diabetes 53(8):2024-33 (2004); Harrington et al., “Structural Basis for the Inhibition of Activin Signalling by Follistatin,” EMBO J. 25(5):1035-45 (2006); Tojo et al., “The ALK-5 Inhibitor A-83-01 Inhibits Smad Signaling and Epithelial-to-Mesenchymal Transition by Transforming Growth Factor-Beta,” Cancer Sci. 96(11):790-800 (2005); Yan et al., “Human BAMBI Cooperates with Smad7 to Inhibit Transforming Growth Factor-Beta Signaling,” J. Biol. Chem. 284(44):30097-104 (2009); Tan et al., “Targeted Inhibition of Activin Receptor-Like Kinase 5 Signaling Attenuates Cardiac Dysfunction Following Myocardial Infarction,” Am. J. Physiol. Heart Circ. Physiol. 298(5):H1415-25 (2010); and Gokoffski et al., “Activin and GDF11 Collaborate in Feedback Control of Neuroepithelial Stem Cell Proliferation and Fate,” Develop. 138(19):4131-42 (2011), which are hereby incorporated by reference in their entirety).

The TGFβ superfamily signaling pathway inhibitor may be an inhibitor of BMP/BMP receptor binding. An exemplary inhibitor of BMP/BMP receptor binding is LDN193189. Additional exemplary BMP inhibitors may be selected from the group consisting of noggin, sclerostin, chordin, CTGF, follistatin, gremlin, inhibin, DMH1, DMH2, Dorsomorphin, K02288, LDN212854, DM 3189, BMP-3, and BAMBI (see WO 2014018691 Al and Mohedas et al., “Development of an ALK2-Biased BMP Type I Receptor Kinase Inhibitor,” ACS Chem. Biol. 8(6):1291-302 (2013); Yan et al., “Human BAMBI Cooperates with Smad7 to Inhibit Transforming Growth Factor-Beta Signaling,” J. Biol. Chem. 284(44):30097-104 (2009), which are hereby incorporated by reference in their entirety).

The TGFβ superfamily signaling pathway inhibitor may be a SMAD signaling pathway inhibitor. Exemplary SMAD signaling pathway inhibitors may be selected from the group including, without limitation, SMAD3 siRNA, SMAD 2/3 siRNA, PD169316, SB203580, SB202474, specific inhibitor of Smad3 (SIS3), HSc025, and SB525334 (see Qureshi et al., “Smad Signaling Pathway is a Pivotal Component of Tissue Inhibitor of Metalloproteinases-3 Regulation by Transforming Growth Factor Beta in Human Chondrocytes,” BBA Mol. Cell Res. 1783(9):1605-12 (2008); Hasegawa et al., “A Novel Inhibitor of Smad-Dependent Transcriptional Activation Suppresses Tissue Fibrosis in Mouse Models of Systemic Sclerosis, Arthritis Rheum. 60(11):3465-75 (2009); and Ramdas et al., “Canonical Transforming Growth Factor-β Signaling Regulates Disintegrin Metalloprotease Expression in Experimental Renal Fibrosis via miR-29,” Am. J. Pathol. 183(6):1885-96 (2013), which are hereby incorporated by reference in their entirety).

Additional exemplary SMAD signaling pathway inhibitors include, without limitation, miR-100, LDN 193189, SMAD-binding peptide aptamers (e.g., Trx-FoxH1, Trx-Le1, Trx-CBP, Trx-SARA), pirfenidone, and LDN193189 (see Fu et al., “MicroRNA-100 Inhibits Bone Morphogenetic Protein-Induced Osteoblast Differentiation by Targeting Smad,” Eur. Rev. Med. Pharmacol. Sci. 20(18):3911-19 (2016); Boergermann et al., “Dorsomorphin and LDN-193189 Inhibit BMP-Mediated Smad, p38 and Akt signalling in C2C12 Cells,” Int. J. Biochem. Cell Biol. 42(11):1802-7 (2010); Cui et al., “Selective Inhibition of TGF-Responsive Genes by Smad-Interacting Peptide Aptamers from FoxH1, Lef1 and CBP,” Oncogene 24:3864-74 (2005); Zhao et al., “Inhibition of Transforming Growth Factor-Beta1-Induced Signaling and Epithelial-to-Mesenchymal Transition by the Smad-Binding Peptide Aptamer Trx-SARA,” Mol. Biol. Cell 17:3819-31 (2006); Li et al., “Oral Pirfenidone Protects Against Fibrosis by Inhibiting Fibroblast Proliferation and TGF-β Signaling in a Murine Colitis Model,” Biochem. Pharmacol. 117:57-67 (2016); and Cook et al., “BNIP Signaling Balances Murine Myeloid Potential Through SMAD-Independent p38MAPK and NOTCH Pathways,” Blood 124(3):393-402 (2014), which are hereby incorporated by reference in their entirety).

The TGFβ superfamily signaling pathway inhibitor may be an inhibitor of the trithorax complex. Exemplary trithorax complex inhibitors include, without limitation, WDR5-0103, MI-1, MI-2, MI-2-2, MLS001171971-01, ML227, MCP-1, RBBS siRNA, and MLL1 siRNA (see Senisterra et al., “Small-Molecule Inhibition of MLL Activity by Disruption of its Interaction with WDRS,” Biochem. J. 449(1):151-9 (2013); Cierpicki et al., “Challenges and Opportunities in Targeting the Menin-MLL Interaction,” Future Med. Chem. 6(4):447-62 (2014); Lee et al., “Roles of DPY30 in the Proliferation and Motility of Gastric Cancer Cells,” PLOS One 10(7):e0131863 (2015); and Zhou et al., “Combined Modulation of Polycomb and Trithorax Genes Rejuvenates β Cell Replication,” J. Clin. Invest. 123(11):4849-4858 (2013), which are hereby incorporated by reference in their entirety).

The TGFβ superfamily signaling pathway inhibitor may be an inhibitor of the polycomb repressive complex 2 (“PRC2”). Exemplary PRC2 inhibitors include GSK926, EPZ005687, GSK126, GSK343, Ell, UNC1999, EPZ6438, Constellation Compound 3, EZH2 siRNA, and 3-deazaneplanocin A (see Verma et al., “Identification of Potent, Selective, Cell-Active Inhibitors of the Histone Lysine Methyltransferase EZH2,” ACS Med. Chem. Lett. 3:1091-6 (2012); Xu et al., “Targeting EZH2 and PRC2 Dependence as Novel Anticancer Therapy,” Exp. Hematol. 43:698-712 (2015); Knutson et al., “A Selective Inhibitor of EZH2 Blocks H3K27 Methylation and Kills Mutant Lymphoma Cells,” Nat. Chem. Biol. 8:890-6 (2012); Qi et al., “Selective Inhibition of Ezh2 by a Small Molecule Inhibitor Blocks Tumor Cells Proliferation,” Proc. Natl Acad. Sci. USA 109:21360-65 (2012); McCabe et al., “EZH2 Inhibition as a Therapeutic Strategy for Lymphoma with EZH2-Activating Mutations,” Nature 492:108-12 (2012); Nasveschuk et al., “Discovery and Optimization of Tetramethylpiperidinyl Benzamides as Inhibitors of EZH2,” ACS Med. Chem. Lett. 5:378-83 (2014); Brooun et al., “Polycomb Repressive Complex 2 Structure with Inhibitor Reveals a Mechanism of Activation and Drug Resistance,” Nature Comm. 7:11384 (2016); Fiskus et al., “Histone Deacetylase Inhibitors Deplete Enhancer of Zeste 2 and Associated Polycomb Repressive Complex 2 Proteins in Human Acute Leukemia Cells,” Mol. Cancer Ther. 5(12):3096-104 (2006); and Fiskus et al., “Combined Epigenetic Therapy with the Histone Methyltransferase EZH2 Inhibitor 3-Deazaneplanocin A and the Histone Deacetylase Inhibitor Panobinostat Against Human AML Cells,” Blood 114(13):2733-43 (2009), which are hereby incorporated by reference in their entirety.)

The method may further involve contacting the population of pancreatic beta cells with a glucagon-like peptide-1 receptor (GLP1R) agonist and/or a Dipeptidyl Peptidase IV (“DDP4”) inhibitor. In accordance with this embodiment, the method may be carried out with a composition comprising a compound according to formula (I) and the glucagon-like peptide-1 receptor (GLP1R) agonist and/or the DDP4 inhibitor, and, optionally, the TGFβ superfamily signaling pathway inhibitor. In another embodiment, the compound of formula (I), the GLP1R agonist and/or the DDP4 inhibitor, and, optionally, the TGFβ superfamily signaling pathway inhibitor each contact the population of pancreatic beta cells simultaneously or in sequence.

Glucagon-like peptide-1 receptor agonists mimic the effects of the incretin hormone GLP-1, which is released from the intestine in response to food intake. Their effects include increasing insulin secretion, decreasing glucagon release, increasing satiety, and slowing gastric emptying. An alternate approach to enhancing GLP1 concentrations in blood is prevention of its degradation by the enzyme DPP4. The GLP1 receptor agonists and the DDP4 inhibitors are among the most widely used drugs for the treatment of Type 2 diabetes (Campbell et al., “Pharmacology, Physiology and Mechanisms of Incretin Hormone Action,” Cell Metab. 17:819-37 (2013); Guo X-H., “The Value of Short- and Long-Acting Glucagon-Like Peptide Agonists in the Management of Type 2 Diabetes Mellitus: Experience with Exenatide,” Curr. Med. Res. Opinion 32(1):61-76 (2016); Deacon et al., “Dipeptidyl Peptidase-4 Inhibitors for the Treatment of Type 2 Diabetes: Comparison, Efficacy and Safety,” Expert Opinion on Pharmacotherapy 14:2047-58 (2013); Lovshin, “Glucagon-Like Peptide-1 Receptor Agonists: A Class Update for Treating Type 2 Diabetes,” Can. J. Diabetes 41:524-35 (2017); and Yang et al., “Lixisenatide Accelerates Restoration of Normoglycemia and Improves Human Beta Cell Function and Survival in Diabetic Immunodeficient NOD-scid IL2rg(null) RIP-DTR Mice Engrafted With Human Islets,” Diabetes Metab. Syndr. Obes. 8:387-98 (2015)).

Suitable GLP1R agonists include, e.g. and without limitation, exenatide, liraglutide, exenatide LAR, taspoglutide, lixisenatide, albiglutide, dulaglutide, and semaglutide. Exenatide and Exenatide LAR are synthetic exendin-4 analogues obtained from the saliva of the Heloderma suspectum (lizard). Liraglutide is an acylated analogue of GLP-1 that self-associates into a heptameric structure that delays absorption from the subcutaneous injection site. Taspoglutide shares 3% homology with the native GLP-1 and is fully resistant to DPP-4 degradation. Lixisenatide is a human GLP1R agonist. Albiglutide is a long-acting GLP-1 mimetic, resistant to DPP-4 degradation. Dulaglutide is a long-acting GLP1 analogue. Semaglutide is a GLP1R agonist approved for the use of T2D. Clinically available GLP1R agonists include, e.g., exenatide, liraglutide, albiglutide, dulaglutide, lixisenatide, semaglutide.

In some embodiments of the methods and compositions of the present invention, the GLP1R agonist is selected from the group consisting of GLP1(7-36), extendin-4, liraglutide, lixisenatide, semaglutide, and combinations thereof.

Additional suitable GLP1 agonists include, without limitation, disubstituted-7-aryl-5,5-bis(trifluoromethyl)-5,8-dihydropyrimido[4,5-d]pyrimidine-2,4(1H,3H)-dione compounds and derivatives thereof, e.g., 7-(4-Chlorophenyl)-1,3-dimethyl-5,5-bis(trifluoromethyl)-5,8-dihydropyrimido[4,5-d]pyrimidine-2,4(1H,3H)-dione (see, e.g., Nance et al., “Discovery of a Novel Series of Orally Bioavailable and CNS Penetrant Glucagon-like Peptide-1 Receptor (GLP-1R) Noncompetitive Antagonists Based on a 1,3-Di sub stituted-7-aryl-5,5-bis(trifluoromethyl)-5,8-dihydropyrimido[4,5-d]pyrimidine-2,4(1H,3H)-dione Core,” J. Med. Chem. 60:1611-1616 (2017), which is hereby incorporated by reference in its entirety).

Further suitable GLP1 agonists include positive allosteric modulators (“PAMS”) of GLP 1R, e.g., (S)-2-cyclopentyl-N-((1-isopropylpyrrolidin-2-yl)methyl)-10-methyl-1-oxo-1,2-dihydropyrazino-[1,2-a]indole-4-carboxamide; (R)-2-cyclopentyl-N-((1-isopropylpyrrolidin-2-yl)methyl)-10-methyl-1-oxo-1,2-dihydropyrazino[1,2-a]indole-4-carboxamide; 2-cyclopentyl-N-(((S)-1-isopropylpyrrolidin-2-yl)methyl)-10-methyl -1-oxo-1,2,3,4-tetrahydropyrazino[1,2-a]indole-4-carboxamide; N-(((S)-1-isopropylpyrrolidin-2-yl)methyl)-10-methyl-1-oxo-2-((S)-tetrahydrofuran-3-yl)-1,2-dihydropyrazino[1,2-a]indole-4-carboxamide; N-(((R)-1-isopropylpyrrolidin-2-yl)methyl)-10-methyl-1-oxo-2-((S)-tetrahydrofuran-3-yl)-1,2-dihydropyrazino[1,2-a]indole-4-carboxamide; (S)-2-cyclopentyl-8-fluoro-N-((1-isopropylpyrrolidin-2-yl)methyl)-10-methyl-1-oxo-1,2-dihydropyrazino[1,2-a]indole-4-carboxamide; (R)-2-cyclopentyl-8-fluoro-N-((1-isopropylpyrrolidin-2-yl)methyl)-10-methyl-1-oxo-1,2-dihydropyrazino[1,2-a]indole-4-carboxamide; (R)-2-cyclopentyl-N-(((S)-1-isopropylpyrrolidin-2-yl)methyl)-10-methyl-1-oxo-1,2,3,4-tetrahydropyrazino[1,2-a]indole-4-carboxamide; (S)-2-cyclopentyl-N-(((S)-1-isopropylpyrrolidin-2-yl)methyl)-10-methyl-1-oxo-1,2,3,4-tetrahydropyrazino[1,2-a]indole-4-carboxamide; (S)-10-chloro-2-cyclopentyl-N-((1-isopropylpyrrolidin-2-yl)methyl)-1-oxo-1,2-dihydropyrazino[1,2-a]indole-4-carboxamide; (R)-10-chloro-2-cyclopentyl-N-((1-isopropylpyrrolidin-2-yl)methyl)-1-oxo-1,2-dihydropyrazino[1,2-a]indole-4-carboxamide; (S)-10-bromo-2-cyclopentyl-N-((1-isopropylpyrrolidin-2-yl)methyl)-1-oxo-1,2-dihydropyrazino[1,2-a]indole-4-carboxamide; (R)-10-bromo-2-cyclopentyl-N-((1-isopropylpyrrolidin-2-yl)methyl)-1-oxo-1,2-dihydropyrazino[1,2-a]indole-4-carboxamide; (R)-N-sopropylpyrrolidin-2-yl)methyl)-10-methyl-1-oxo-2-phenyl-1,2-dihydropyrazino[1,2-a]indole-4-carboxamide; (S)-10-cyano-2-cyclopentyl-N-((1-isopropylpyrrolidin-2-yl)methyl)-1-oxo-1,2-dihydropyrazino[1,2-a]indole-4-carboxamide; (S)-2-cyclopentyl-N-((1-isopropylpyrrolidin-2-yl)methyl)-1-oxo-10-vinyl-1,2-dihydropyrazino[1,2-a]indole-4-carboxamide; (S)-N-((1-isopropylpyrroli din-2-yl)methyl)-10-methyl -2-(1-methyl-1H-pyrazol-4-yl)-1-oxo-1,2-dihydropyrazino[1,2-a]indole-4-carboxamide; (R)-N-((1-isopropylpyrrolidin-2-yl)methyl)-10-methyl-2-(1-methyl-1H-pyrazol-4-yl)-1-oxo-1,2-dihydropyrazino[1,2-a]indole-4-carboxamide; (S)-N-((1-isopropylpyrrolidin-2-yl)methyl)-10-methyl -1-oxo-2-(pyridin-3-yl)-1,2-dihydropyrazino[1,2-a]indole-4-carboxamide; (R)-N-((1-isopropylpyrrolidin-2-yl)methyl)-10-methyl-1-oxo-2-(pyridin-3-yl)-1,2-dihydropyrazino[1,2-a]indole-4-carboxamide; N-(azetidin-2-ylmethyl)-2-cyclopentyl-10-methyl-1-oxo-1,2-dihydropyrazino [1,2-a]indole-4-carboxamide; and 2-cyclopentyl-N-((1-isopropylazetidin-2-yl)methyl)-10-methyl-1-oxo-1,2-dihydropyrazino[1,2-a]indole-4-carboxamide; or pharmaceutically acceptable salts thereof (see PCT Publication No. WO 2017/117556, which is hereby incorporated by reference in its entirety).

Suitable DDP4 inhibitors include, without limitation, sitagliptin, vildagliptin, saxagliptin, alogliptin, teneligliptin, and anagliptin.

According to one embodiment, “pancreatic beta cells” are primary human pancreatic beta cells.

In one embodiment of carrying out this and other methods of the present invention, contacting does not induce beta cell death or DNA damage. Moreover, contacting may induce beta cell differentiation and increase glucose-stimulated insulin secretion.

In another embodiment, the method is carried out to enhance cell survival. For example, the method may be carried out to enhance cell survival of a treated population of cells relative to an untreated population of cells. Alternatively, the method may be carried out to decrease cell death or apoptosis of a treated population of cells relative to an untreated population of cells.

A further aspect of the present invention relates to a composition comprising a compound of formula (I) described herein and a carrier.

The composition may further comprise a transforming growth factor beta (TGF(3) superfamily signaling pathway inhibitor.

In another embodiment, the composition may further comprise a glucagon-like peptide-1 receptor (GLP1R) agonist or a Dipeptidyl Peptidase IV (DDP4) inhibitor.

The carrier may be a pharmaceutically-acceptable carrier.

While it may be possible for compounds of formula (I) to be administered as the raw chemical, they may also be administered as a pharmaceutical composition. In accordance with an embodiment of the present invention, there is provided a pharmaceutical composition comprising a compound of formula (I) or a pharmaceutically acceptable salt or solvate thereof, together with one or more pharmaceutically carriers thereof and optionally one or more other therapeutic ingredients.

The carrier(s) must be “acceptable” in the sense of being compatible with the other ingredients of the formulation and not deleterious to the recipient thereof. Furthermore, notwithstanding the statements herein regarding the term “compound” including salts thereof as well, so that independent claims reciting “a compound” will be understood as referring to salts thereof as well, if in an independent claim reference is made to a compound or a pharmaceutically acceptable salt thereof, it will be understood that claims which depend from that independent claim which refer to such a compound also include pharmaceutically acceptable salts of the compound, even if explicit reference is not made to the salts in the dependent claim.

Formulations include those suitable for oral, parenteral (including subcutaneous, intradermal, intramuscular, intravenous, and intraarticular), rectal and topical (including dermal, buccal, sublingual, and intraocular) administration. The most suitable route may depend upon the condition and disorder of the recipient. The formulations may conveniently be presented in unit dosage form and may be prepared by any of the methods well known in the art of pharmacy. Such methods include the step of bringing into association a compound of formula (I) or a pharmaceutically acceptable salt or solvate thereof (“active ingredient”) with the carrier, which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets, or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary, or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

The pharmaceutical compositions may include a “pharmaceutically acceptable inert carrier,” and this expression is intended to include one or more inert excipients, which include, for example and without limitation, starches, polyols, granulating agents, microcrystalline cellulose, diluents, lubricants, binders, disintegrating agents, and the like. If desired, tablet dosages of the disclosed compositions may be coated by standard aqueous or nonaqueous techniques. “Pharmaceutically acceptable carrier” also encompasses controlled release means.

Pharmaceutical compositions may also optionally include other therapeutic ingredients, anti-caking agents, preservatives, sweetening agents, colorants, flavors, desiccants, plasticizers, dyes, and the like. Any such optional ingredient must be compatible with the compound of formula (I) to insure the stability of the formulation. The composition may contain other additives as needed including, for example, lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to, binders, fillers, disintegrants, lubricants, anti-microbial agents, and coating agents.

Dose ranges for adult humans vary, but may generally be from about 0.005 mg to 10 g/day orally. Tablets or other forms of presentation provided in discrete units may conveniently contain an amount of compound of formula (I) which is effective at such dosage or as a multiple of the same, for instance, units containing 5 mg to 500 mg, or around 10 mg to 200 mg. The precise amount of compound administered to a patient will be the responsibility of the attendant physician. However, the dose employed will depend on a number of factors, including the age and sex of the patient, the precise disorder being treated, and its severity.

A dosage unit (e.g., an oral dosage unit) can include from, for example, 1 to 30 mg, 1 to 40 mg, 1 to 100 mg, 1 to 300 mg, 1 to 500 mg, 2 to 500 mg, 3 to 100 mg, 5 to 20 mg, 5 to 100 mg (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg) of a compound described herein.

Additional information about pharmaceutical compositions and their formulation is described in Remington: The Science and Practice of Pharmacy, 20^{th} Edition, 2000, which is hereby incorporated by reference in its entirety.

The agents can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), or by other routes. The agents can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g., PCT Publication No. WO 97/11682, which is hereby incorporated by reference in its entirety) via a liposomal formulation (see, e.g., EP Patent No. 736299, PCT Publication No. WO 99/59550, and PCT Publication No. WO 97/13500, which is hereby incorporated by reference in its entirety), via formulations described in PCT Publication No. WO 03/094886 (which is hereby incorporated by reference in its entirety) or in some other form. The agents can also be administered transdermally (i.e., via reservoir-type or matrix-type patches, microneedles, thermal poration, hypodermic needles, iontophoresis, electroporation, ultrasound, or other forms of sonophoresis, jet injection, or a combination of any of the preceding methods (Prausnitz et al. Nature Reviews Drug Discovery 3:115 (2004), which is hereby incorporated by reference in its entirety). The agents can be administered locally.

The agents can be administered in the form a suppository or by other vaginal or rectal means. The agents can be administered in a transmembrane formulation as described in PCT Publication No. WO 90/07923, which is hereby incorporated by reference in its entirety. The agents can be administered non-invasively via the dehydrated particles described in U.S. Pat. No. 6,485,706, which is hereby incorporated by reference in its entirety. The agents can be administered in an enteric-coated drug formulation as described in PCT Publication No. WO 02/49621, which is hereby incorporated by reference in its entirety. The agents can be administered intranasaly using the formulation described in U.S. Pat. No. 5,179,079, which is hereby incorporated by reference in its entirety. Formulations suitable for parenteral injection are described in PCT Publication No. WO 00/62759, which is hereby incorporated by reference in its entirety. The agents can be administered using the casein formulation described in U.S. Patent Application Publication No. 2003/0206939 and PCT Publication No. WO 00/06108, which are hereby incorporated by reference in their entirety. The agents can be administered using the particulate formulations described in U.S. Patent Application Publication No.

20020034536, which is hereby incorporated by reference in its entirety.

The agents, alone or in combination with other suitable components, can be administered by pulmonary route utilizing several techniques including, but not limited to, intratracheal instillation (delivery of solution into the lungs by syringe), intratracheal delivery of liposomes, insufflation (administration of powder formulation by syringe or any other similar device into the lungs), and aerosol inhalation. Aerosols (e.g., jet or ultrasonic nebulizers, metered-dose inhalers (“MDIs”), and dry-Powder inhalers (“DPIs”)) can also be used in intranasal applications. Aerosol formulations are stable dispersions or suspensions of solid material and liquid droplets in a gaseous medium and can be placed into pressurized acceptable propellants, such as hydrofluoroalkanes (HFAs, i.e., HFA-134a and HFA-227, or a mixture thereof), dichlorodifluoromethane (or other chlorofluorocarbon propellants such as a mixture of Propellants 11, 12, and/or 114), propane, nitrogen, and the like. Pulmonary formulations may include permeation enhancers such as fatty acids, and saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion.

Pulmonary formulations may also include surfactants which include, but are not limited to, bile salts and those described in U.S. Pat. No. 6,524,557 and references therein, which are hereby incorporated by reference in their entirety. The surfactants described in U.S. Pat. No. 6,524,557, e.g., a C₈-C₁₆ fatty acid salt, a bile salt, a phospholipid, or alkyl saccharide are advantageous in that some of them also reportedly enhance absorption of the compound in the formulation.

Also suitable in the invention are dry powder formulations comprising a therapeutically effective amount of active compound blended with an appropriate carrier and adapted for use in connection with a dry-powder inhaler. Absorption enhancers that can be added to dry powder formulations include those described in U.S. Pat. No. 6,632,456, which is hereby incorporated by reference in its entirety. PCT Publication No. WO 02/080884, which is hereby incorporated by reference in its entirety, describes new methods for the surface modification of powders. Aerosol formulations may include those described in U.S. Pat. Nos. 5,230,884 and 5,292,499; PCT Publication Nos. WO 017/8694 and 01/78696; and U.S. Patent Application Publication No. 2003/019437, 2003/0165436; and PCT Publication No. WO 96/40089 (which includes vegetable oil), which are hereby incorporated by reference in their entirety. Sustained release formulations suitable for inhalation are described in U.S. Patent Application Publication Nos. 2001/0036481, 2003/0232019, and 2004/0018243 as well as in PCT Publication Nos. WO 01/13891, 02/067902, 03/072080, and 03/079885, which are hereby incorporated by reference in their entirety.

Pulmonary formulations containing microparticles are described in PCT Publication No. WO 03/015750, U.S. Patent Application Publication No. 2003/0008013, and PCT Publication No. WO 00/00176, which are hereby incorporated by reference in their entirety. Pulmonary formulations containing stable glassy state powder are described in U.S. Patent Application Publication No. 2002/0141945 and U.S. Pat. No. 6,309,671, which are hereby incorporated by reference in their entirety. Other aerosol formulations are described in EP Patent No. 1338272, PCT Publication No. WO 90/09781, U.S. Pat. Nos. 5,348,730 and 6,436,367, PCT Publication No. WO 91/04011, and U.S. Pat. Nos. 6,294,153 and 6,290,987, which are hereby incorporated by reference in their entirety, which describe a liposomal based formulation that can be administered via aerosol or other means.

Powder formulations for inhalation are described in U.S. Patent Application Publication No. 2003/0053960 and PCT Publication No. WO 01/60341, which are hereby incorporated by reference in their entirety. The agents can be administered intranasally as described in U.S. Patent Application Publication No. 2001/0038824, which is hereby incorporated by reference in its entirety.

Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy.

Other devices for generating aerosols employ compressed gases, usually hydrofluorocarbons and chlorofluorocarbons, which are mixed with the medicament and any necessary excipients in a pressurized container. These devices are likewise described in standard textbooks such as Sprowls and Remington.

The agent can be incorporated into a liposome to improve half-life. The agent can also be conjugated to polyethylene glycol (“PEG”) chains. Methods for pegylation and additional formulations containing PEG-conjugates (i.e., PEG-based hydrogels, PEG modified liposomes) can be found in Harris and Chess, Nature Reviews Drug Discovery 2:214-221, which is hereby incorporated by reference in its entirety, and the references therein. The agent can be administered via a nanocochleate or cochleate delivery vehicle (BioDelivery Sciences International). The agents can be delivered transmucosally (i.e., across a mucosal surface such as the vagina, eye, or nose) using formulations such as that described in U.S. Pat. No. 5,204,108, which is hereby incorporated by reference in its entirety. The agents can be formulated in microcapsules as described in PCT Publication No. WO 88/01165, which is hereby incorporated by reference in its entirety. The agent can be administered intra-orally using the formulations described in U.S. Patent Application Publication No. 2002/0055496, PCT Publication No. WO 00/47203, and U.S. Pat. No. 6,495,120, which are hereby incorporated by reference in their entirety. The agent can be delivered using nanoemulsion formulations described in PCT Publication No. WO 01/91728, which is hereby incorporated by reference in its entirety.

Another aspect of the present invention relates to a method of treating a subject for a condition associated with an insufficient level of insulin secretion. This method involves administering to a subject in need of treatment for a condition associated with an insufficient level of insulin secretion a compound or composition of the present invention.

In one embodiment, the treatment methods of the present invention are carried out under conditions effective to increase pancreatic beta cell mass in the subject to treat the subject for an insufficient level of insulin secretion.

In one embodiment, the compound or composition may be administered with or coincident with a TGFβ superfamily signaling pathway inhibitor.

In another embodiment, the compound or composition may be administered with or coincident with a glucagon-like peptide-1 receptor (GLP1R) agonist or a Dipeptidyl Peptidase IV (DDP4) inhibitor. Suitable glucagon-like peptide-1 receptor (GLP1R) agonists or a Dipeptidyl Peptidase IV (DDP4) inhibitors are described in detail above. In accordance with this embodiment, the administering is carried out under conditions effective to cause a synergistic increase in pancreatic beta cell mass in the subject to treat the subject for an insufficient level of insulin secretion.

As used herein, a condition associated with an insufficient level of insulin secretion means a condition where a subject produces a lower plasma level of insulin than is required to maintain normal glucose levels in the blood such that the subject with the condition associated with insufficient insulin secretion becomes hyperglycemic. In such a condition, the pancreatic beta cells of the afflicted subject secrete an insufficient level of insulin to maintain the presence of a normal concentration of glucose in the blood (i.e., normoglycemica).

According to one embodiment, one of the conditions associated with an insufficient level of insulin secretion is insulin resistance. Insulin resistance is a condition in which a subject's cells become less sensitive to the glucose-lowering effects of insulin. Insulin resistance in muscle and fat cells reduces glucose uptake (and, therefore, local storage of glucose as glycogen and triglycerides), whereas insulin resistance in liver cells results in reduced glycogen synthesis and storage and a failure to suppress glucose production and release into the blood. Insulin resistance normally refers to reduced glucose-lowering effects of insulin. However, other functions of insulin can also be affected. For example, insulin resistance in fat cells reduces the normal effects of insulin on lipids and results in reduced uptake of circulating lipids and increased hydrolysis of stored triglycerides. Increased mobilization of stored lipids in these cells elevates free fatty acids in the blood plasma. Elevated blood fatty-acid concentrations, reduced muscle glucose uptake, and increased liver glucose production all contribute to elevated blood glucose levels. If insulin resistance exists, more insulin needs to be secreted by the pancreas. If this compensatory increase does not occur, blood glucose concentrations increase and type II diabetes occurs.

According to another embodiment, one of the conditions associated with an insufficient level of insulin secretion is diabetes. Diabetes can be divided into two broad types of diseases: type I (T1D) and type II (T2D). The term “diabetes” also refers herein to a group of metabolic diseases in which patients have high blood glucose levels, including type I diabetes (T1D), type II diabetes (T2D), gestational diabetes, congenital diabetes, maturity onset diabetes (MODY), cystic fibrosis-related diabetes, hemochromatosis-related diabetes, drug-induced diabetes (e.g., steroid diabetes), and several forms of monogenic diabetes.

Thus, in one embodiment, the subject has been diagnosed as having one or more of type I diabetes (T1D), type II diabetes (T2D), gestational diabetes, congenital diabetes, maturity onset diabetes (MODY), cystic fibrosis-related diabetes, hemochromatosis-related diabetes, drug-induced diabetes, or monogenic diabetes.

According to another embodiment, a condition associated with an insufficient level of insulin secretion is metabolic syndrome. Metabolic syndrome is generally used to define a constellation of abnormalities that is associated with increased risk for the development of type II diabetes and atherosclerotic vascular disease. Related conditions and symptoms include, but are not limited to, fasting hyperglycemia (diabetes mellitus type II or impaired fasting glucose, impaired glucose tolerance, or insulin resistance), high blood pressure; central obesity (also known as visceral, male-pattern or apple-shaped adiposity), meaning overweight with fat deposits mainly around the waist; decreased HDL cholesterol; and elevated triglycerides.

In one embodiment, the subject has been diagnosed as having metabolic syndrome or insulin resistance.

Other conditions that may be associated with an insufficient level of insulin secretion include, without limitation, hyperuricemia, fatty liver (especially in concurrent obesity) progressing to non-alcoholic fatty liver disease, polycystic ovarian syndrome (in women), and acanthosis nigricans.

Related disorders may also be treated pursuant to the treatment methods of the present invention including, without limitation, any disease associated with a blood or plasma glucose level outside the normal range, preferably hyperglycemia. Consequently, the term “related disorders” includes impaired glucose tolerance (IGT), impaired fasting glucose (IFG), insulin resistance, metabolic syndrome, postprandial hyperglycemia, and overweight/obesity. Such related disorders can also be characterized by an abnormal blood and/or plasma insulin level.

The methods described herein may be carried out to treat a subject with conditions associated with beta cell failure or deficiency. Such conditions include, without limitation, type I diabetes (T1D), type II diabetes (T2D), gestational diabetes, congenital diabetes, maturity onset diabetes (MODY), cystic fibrosis-related diabetes, hemochromatosis-related diabetes, drug-induced diabetes, or monogenic diabetes. Drug induced diabetes relates to a condition that is caused through the use of drugs that are toxic to beta cells (e.g., steroids, antidepressants, second generation antipsychotics, and immunosuppressive. Exemplary immunosuppressive drugs include, but are not limited to, members of the cortisone family (e.g., prednisone and dexamethasome), rapamycin/sirolimus, everolimus, and calciuneurin inhibitors (e.g., FK-506/tacrolimus).

Additional conditions associated with beta cell deficiency include, without limitation, pancreatectomy, partial pancreatectomy, pancreas transplantation, and pancreatic islet transplantation.

The methods described herein may be carried out to treat a subject at risk of developing Type II Diabetes. For example, a patient at risk of developing Type II Diabetes has pre-diabetes/metabolic syndrome. The patient at risk of developing Type II Diabetes may have been treated with a psychoactive drug, including but not limited to a selective serotonin reuptake inhibitors (“SSRI”) for depression, obsessive compulsive disorder (“OCD”), etc.

In carrying out treatment methods, a compound of Formula (I) or composition containing such compound and a TGFβ superfamily signaling pathway inhibitor are administered under conditions effective to increase pancreatic beta cell mass in the subject to treat the subject for a condition associated with an insufficient level of insulin secretion.

A compound or composition described herein and/or TGFβ superfamily signaling pathway inhibitor may be administered to increase pancreatic beta cell mass in the subject, which will result in an increased level of insulin secretion in the subject.

The compound and/or composition and TGFβ superfamily signaling pathway inhibitor may be formulated as separate pharmaceutical compositions or a single pharmaceutical composition comprising both the compound of formula (I) and TGFβ superfamily signaling pathway inhibitor. Such pharmaceutical composition(s) may comprise a therapeutically effective amount of the compound of formula (I) and/or TGFβ superfamily signaling pathway inhibitor.

Thus, a combination or combinatorial therapy or treatment of a compound of formula (I) and TGFβ superfamily signaling pathway inhibitor may be administered. The terms “combination” or “combinatorial therapy” or “combinatory treatment” mean a treatment where at least two compounds are co-administered to a subject to cause a biological effect, in this case a synergistic effect. In a combinatorial therapy, the at least two drugs may be administered together or separately, at the same time or sequentially. Simultaneous administration is not required, as long as the drugs produce a synergistic effect in the subject to improve the subject's conditions. Also, the at least two drugs may be administered through different routes and protocols. As a result, although they may be formulated together, the drugs of a combination may also be formulated separately.

A further aspect relates to a method of treating a subject for a neurological disorder. This method involves administering to a subject in need of treatment for a neurological disorder a compound of formula (I) under conditions effective to treat the subject for the condition.

The subject may have diabetes and/or has been diagnosed as having one or more of Down's Syndrome and a neurodegenerative disease.

In carrying out the treatment methods, administering of compounds to a subject may involve administering pharmaceutical compositions containing the compound(s) (i.e., a DYRK1A inhibitor of formula (I) and TGF(3 superfamily signaling pathway inhibitor) in therapeutically effective amounts, which means an amount of compound effective in treating the stated conditions and/or disorders in the subject. Such amounts generally vary according to a number of factors well within the purview of ordinarily skilled artisans. These include, without limitation: the particular subject, as well as its age, weight, height, general physical condition, and medical history, the particular compound used, as well as the carrier in which it is formulated and the route of administration selected for it; the length or duration of treatment; and the nature and severity of the condition being treated.

Administering typically involves administering pharmaceutically acceptable dosage forms, which means dosage forms of compounds described herein and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition, which is hereby incorporated by reference in its entirety.

In carrying out treatment methods, the drug (i.e., a compound of formula (I) and, optionally, a TGFβ superfamily signaling pathway inhibitor) may be contained, in any appropriate amount, in any suitable carrier substance. The drug may be present in an amount of up to 99% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols.

Pharmaceutical compositions may be formulated to release the active drug substantially immediately upon administration or at any predetermined time or time period after administration.

Controlled release formulations include (i) formulations that create a substantially constant concentration of the drug(s) within the body over an extended period of time; (ii) formulations that after a predetermined lag time create a substantially constant concentration of the drug(s) within the body over an extended period of time; (iii) formulations that sustain drug(s) action during a predetermined time period by maintaining a relatively, constant, effective drug level in the body with concomitant minimization of undesirable side effects associated with fluctuations in the plasma level of the active drug substance; (iv) formulations that localize drug(s) action by, e.g., spatial placement of a controlled release composition adjacent to or in the diseased tissue or organ; and (v) formulations that target drug(s) action by using carriers or chemical derivatives to deliver the drug to a particular target cell type.

Administration of drugs in the form of a controlled release formulation is especially preferred in cases in which the drug has (i) a narrow therapeutic index (i.e., the difference between the plasma concentration leading to harmful side effects or toxic reactions and the plasma concentration leading to a therapeutic effect is small; in general, the therapeutic index (“TI”) is defined as the ratio of median lethal dose (LD₅₀) to median effective dose (ED₅₀)); (ii) a narrow absorption window in the gastro-intestinal tract; or (iii) a very short biological half-life so that frequent dosing during a day is required in order to sustain the plasma level at a therapeutic level.

Any of a number of strategies can be pursued to obtain controlled release in which the rate of release outweighs the rate of metabolism of the drug in question. Controlled release may be obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner (single or multiple unit tablet or capsule compositions, oil solutions, suspensions, emulsions, microcapsules, microspheres, nanoparticles, patches, and liposomes).

Thus, administering may be carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes. Compounds may be administered alone or with suitable pharmaceutical carriers, and can be in solid or liquid form, such as tablets, capsules, powders, solutions, suspensions, or emulsions.

The subject may be a mammalian subject. In one embodiment, the subject is a human subject. Suitable human subjects include, without limitation, children, adults, and elderly subjects having a beta-cell and/or insulin deficiency.

The subject may be bovine, ovine, porcine, feline, equine, murine, canine, lapine, etc.

The administering step may increase the number of proliferating pancreatic beta cells in the subject by at least about 5%, 6%, 7%, or more.

Administering may increase glucose-stimulated insulin secretion in pancreatic beta cells of the subject.

The designation of a compound is meant to designate the compound per se, as well as any pharmaceutically acceptable salt, hydrate, isomer, racemate, ester, or ether thereof. The designation of a compound is meant to designate the compound as specifically designated per se, as well as any pharmaceutically acceptable salt thereof.

Within the context of the present disclosure, by “treating” it is meant preventive or curative treatment.

Treatment may designate, in particular, the correction, decrease in the rate of change, or reduction of an impaired glucose homeostasis. The level of glucose in blood fluctuates throughout the day. Glucose levels are usually lower in the morning, before the first meal of the day and rise after meals for some hours. Consequently, the term treatment includes the control of blood glucose level by increasing or decreasing blood glucose level depending on the condition of the subject and the daytime in order to reach normal glucose levels. The term treatment more particularly includes a temporary or persistent reduction of blood glucose level in a subject having diabetes or a related disorder. The term “treatment” or “treating” also designates an improvement in insulin release (e.g., by pancreatic beta cells).

As used herein, the phrase “control of blood glucose level” refers to the normalization or the regulation of the blood or plasma glucose level in a subject having abnormal levels (i.e., levels that are below or above a known reference, median, or average value for a corresponding subject with a normal glucose homeostasis).

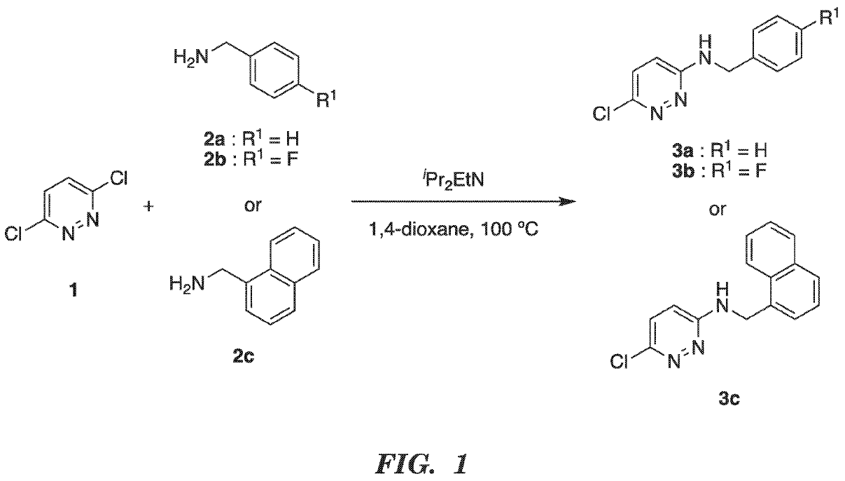

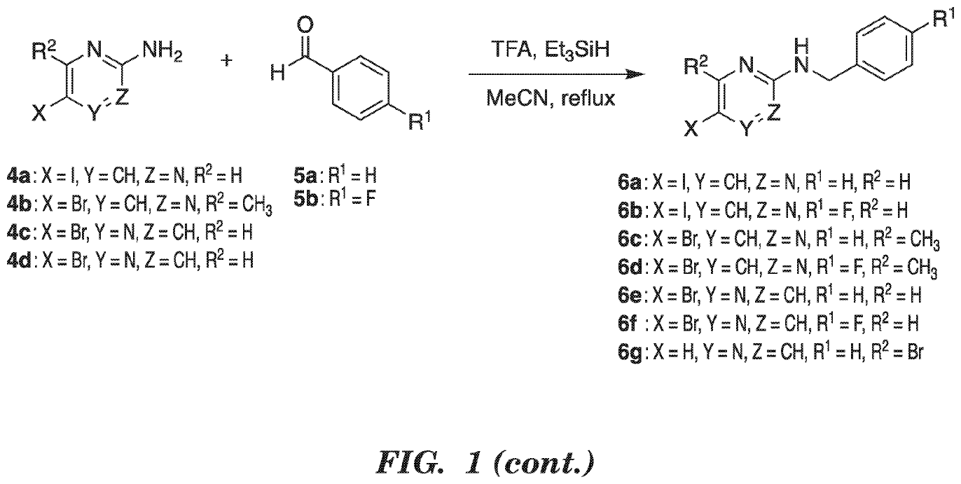

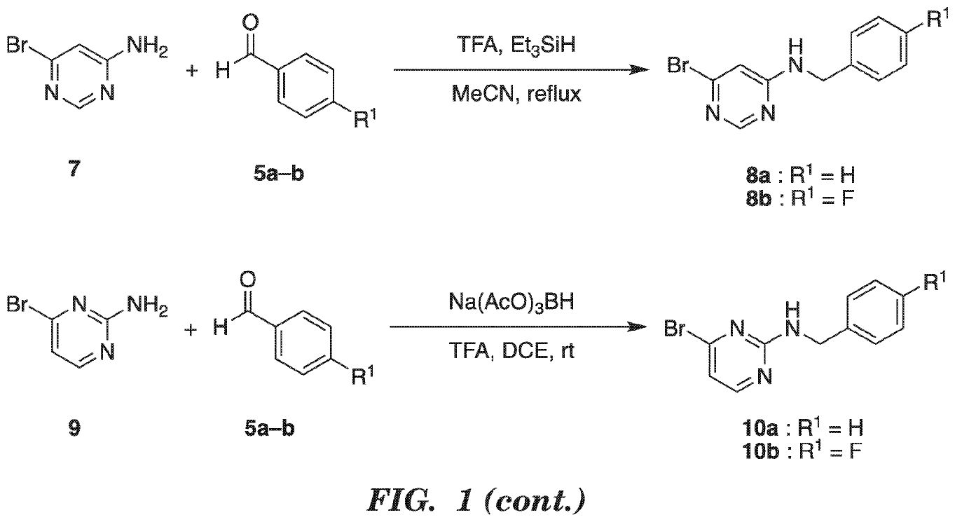

Compounds referred to herein in the examples are referenced by names and also numbers (e.g., la). Structures corresponding to the numbers are identified in FIGS. 1-9. For example, compound (la) is shown in FIG. 1.

### EXAMPLES

### Example 1—Synthesis of Kinase Selective Pyridine-Based DYRK1A Inhibitors

General Experimental Conditions

All reactions involving air-sensitive reagents were carried out with magnetic stirring and in oven-dried glassware with rubber septa under argon unless otherwise stated. All commercially available chemicals and reagent grade solvents were used without further purification unless otherwise specified. Thin-layer chromatography (TLC) was performed on Baker-flex® silica gel plates (IB2-F) using UV-light (254 and 365 nm) detection or visualizing agents (ninhydrin or phosphomolybdic acid stain) and flash chromatography was carried out on silica gel (230-400 mesh) using Teledyne Isco CombiFlash® Rf. NMR spectra were acquired at room temperature using a Bruker DRX-600 spectrometer at 600 MHz for ¹H and 150 MHz for ¹³C. Chemical shifts (δ) are given in parts per million (ppm) with reference to solvent signals [¹H-NMR: CDCl₃ (7.26 ppm), CD₃OD (3.30 ppm), DMSO-d₆ (2.49 ppm); ¹³C-NMR: CDCl₃ (77.0 ppm), CD₃OD (49.0 ppm), DMSO-d₆ (39.5 ppm)]. Signal patterns are reported as s (singlet), d (doublet), t (triplet), q (quartet), quin (quintet), sex (sextet), sep (septet), m (multiplet), br (broad), dd (doublet of doublets), dt (doublet of triplets), td (triplet of doublets), and tt (triplet of triplets). Coupling constants (d) are given in Hz. LCMS analysis was conducted on an Agilent Technologies G1969A high-resolution API-TOF mass spectrometer attached to an Agilent Technologies 1200 HPLC system. Samples were ionized by electrospray ionization (ESI) in positive mode and reported as m/z (relative intensity) for the molecular ion [M].

### Example 2—General Procedure for the Preparation of N-Benzyl-6-chloropyridazin-3-amine—Method A

### N-Benzyl-6-chloropyridazin-3-amine (3a)

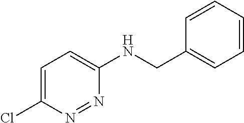 [SMILES: Clc1ccc(NCc2ccccc2)nn1]

To a solution of 3,6-dichloropyridazine (1) (200.0 mg, 1.34 mmol) in anhydrous 1,4-dioxane (2 mL) was added benzylamine (0.15 mL, 1.34 mmol) and N,N-diisopropylethylamine (1 mL) under nitrogen. The resulting mixture was stirred at 100° C. for 20 h. After being quenched with H₂O (5 mL), the aqueous layer was extracted with EtOAc (2×20 mL). The combined organic extracts were washed with brine, dried over anhydrous Na₂SO₄, filtered, and concentrated. The residue was purified by column chromatography on silica gel (EtOAc/hexane, 30:70) to afford 3a (66.3 mg, 22%) as a white solid; ¹H NMR (CDCl₃, 600 MHz) 7.36 (4 H, d, J=2.4 Hz), 7.30 (1 H, s), 7.15 (1 H, d, J=8.5 Hz), 6.63 (1 H, d, J=8.5 Hz), 5.25 (1 H, s), 4.60 (2 H, d, J=6.1 Hz); HRMS (ESI-TOF) m/z: [M+H]⁺ calculated for C₁₁H₁₁ClN₃ 220.0636; found 220.0689.

### 6-Chloro-N-(4-fluorobenzyl)pyridazin-3-amine (3b)

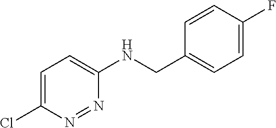 [SMILES: Fc1ccc(CNc2ccc(Cl)nn2)cc1]

The above compound (3b) was prepared from 1 and 2b following Method A and purified by column chromatography on silica gel (EtOAc/hexane, 30:70 to 45:55) to give 3b (10%) as a white solid; ¹H NMR (CDCl₃, 600 MHz) 7.30 (2 H, t, J=6.1 Hz), 7.14 (1 H, d, J=9.8 Hz), 7.00 (2 H, t, J=8.5 Hz), 6.63 (1 H, d, J=9.8 Hz), 4.53 (2 H, s); HRMS (ESI-TOF) m/z: [M+H]⁺ calculated for C₁₁H₁₀ClFN₃ 238.0542; found 238.0544.

### 6-Chloro-N-(naphthalen-1-ylmethyl)pyridazin-3-amine (3c)

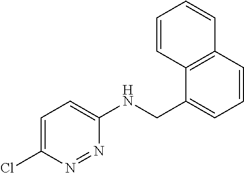 [SMILES: Clc1ccc(NCc2cccc3ccccc23)nn1]

The above compound (3c) was prepared from 1 and 2c following Method A and purified by column chromatography on silica gel (EtOAc/hexane, 50:50) to give 3c (22%) as a light brown solid; ¹H NMR (CDCl₃, 600 MHz) 8.05-8.04 (1 H, d), 7.93-7.91 (1 H, d), 7.86-7.85 (1 H, d), 7.58-7.53 (3 H, m), 7.47-7.45 (1 H, t), 7.19-7.17 (1 H, d), 6.67-6.65 (1 H, d), 5.09-5.08 (2 H, d); HRMS (ESI-TOF) m/z: [M+H]⁺ calculated for C₁₅H₁₃ClN₃ 270.0793; found 270.0796.

### Example 3—General Procedure for the Preparation of N-Benzylhalopyrimidin-Amine—Method B

### N-Benzyl-5-iodopyrimidin-2-amine (6a)

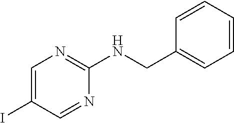 [SMILES: Ic1cnc(NCc2ccccc2)nc1]

To a solution of 5-iodopyrimidin-2-amine (4a) (250.0 mg, 1.13 mmol) in anhydrous acetonitrile (8 mL) was added benzaldehyde (5a) (0.12 mL, 1.13 mmol), triethylsilane (0.96 mL, 5.99 mmol) and trifluoroacetic acid (0.48 mL, 6.33 mmol) under nitrogen. The resulting mixture was refluxed for 17 h. After being quenched with sat. NaHCO₃ (5 mL), water and EtOAc were added. The layers were separated. The combined organic phases were washed with brine, dried over anhydrous Na₂SO₄, filtered, and concentrated. The residue was purified by column chromatography on silica gel (EtOAc/hexane, 10:90) to afford 6a (119.4 mg, 80%) as a white solid; ¹H NMR (CDCl₃, 600 MHz) 8.25 (2 H, s), 7.36-7.32 (4 H, m), 7.31-7.28 (1 H, m), 6.08 (1 H, br), 4.58 (2 H, d, J=6.1 Hz); HRMS (ESI-TOF) m/z: [M+H]⁺ calculated for C₁₁H₁₁IN₃ 311.9992; found 311.9981.

### N-(4-Fluorobenzyl)-5-iodopyrimidin-2-amine (6b)

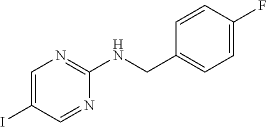 [SMILES: Fc1ccc(CNc2ncc(I)cn2)cc1]

The above compound (6b) was prepared from 4a and 5b following Method B and purified by column chromatography on silica gel (EtOAc/hexane, 10:90 to 15:85) to give 6b (91%) as a pale yellow solid; ¹H NMR (CDCl₃, 600 MHz) 8.36 (2 H, s), 7.30 (2 H, dd, J=8.5, 6.1 Hz), 7.02 (2 H, t, J=8.5 Hz), 5.67 (1 H, br), 4.56 (2 H, d, J=6.1 Hz); HRMS (ESI-TOF) m/z: [M+H]⁺ calculated for C₁₁H₁₀FIN₃ 329.9898; found 329.9903.

### N-Benzyl-5-bromo-4-methylpyrimidin-2-amine (6c)

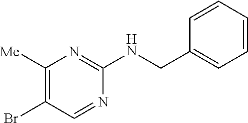 [SMILES: Cc1nc(NCc2ccccc2)ncc1Br]

The above compound (6c) was prepared from 4b and 5a following Method B and purified by column chromatography on silica gel (EtOAc/hexane, 10:90) to give 6c (90%) as a yellow solid; ¹H NMR (CDCl₃, 600 MHz) 8.20 (1 H, s), 7.34-7.33 (4 H, m), 7.28-7.26 (1 H, m), 5.58 (1 H, s), 4.61 (2 H, d, J=6.1 Hz), 2.44 (3 H, s); HRMS (ESI-TOF) m/z: [M+H]⁺ calculated for C₁₂H₁₃⁷⁹BrN₃ and C₁₂H₁₃⁸¹BrN₃ 278.0287, 280.0267; found 278.0282, 280.0262.

### 5-Bromo-N-(4-fluorobenzyl)-4-methylpyrimidin-2-amine (6d)

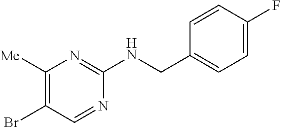 [SMILES: Cc1nc(NCc2ccc(F)cc2)ncc1Br]

The above compound (6d) was prepared from 4b and 5b following Method B and purified by column chromatography on silica gel (EtOAc/hexane, 10:90) to give 6d (90%) as a yellow solid; ¹H NMR (CDCl₃, 600 MHz) 8.23 (1 H, s), 7.30 (2 H, t, J=8.5 Hz), 7.01 (2 H, t, J=8.5 Hz), 5.62 (1 H, br), 4.57 (2 H, d, J=4.9 Hz), 2.45 (3 H, s); HRMS (ESI-TOF) m/z: [M+H]⁺ calculated for C₁₂H₁₂⁷⁹BrFN₃ and C₁₂H₁₂⁸¹BrFN₃ 296.0193, 298.0173; found 296.0194, 298.0175.

### N-Benzyl-5-bromopyrazin-2-amine (6e)

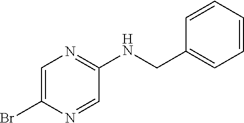 [SMILES: Brc1cnc(NCc2ccccc2)cn1]

The above compound (6e) was prepared from 4c and 5a following Method B and purified by column chromatography on silica gel (EtOAc/hexane, 20:80) to give 6e (83%) as a yellow solid; ¹H NMR (CDCl₃, 600 MHz) 8.12 (1 H, s), 7.70 (1 H, s), 7.40-7.33 (5 H, m), 5.07 (1 H, br), 4.55 (2 H, s); HRMS (ESI-TOF) m/z: [M+H]⁺ calculated for C₁₁H₁₁⁷⁹BrN₃ and C₁₁H₁₁⁸¹BrN₃ 264.0131, 266.0110; found 264.0123, 266.0120.

### 5-Bromo-N-(4-fluorobenzyl)pyrazin-2-amine (6f)

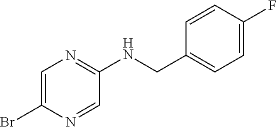 [SMILES: Fc1ccc(CNc2cnc(Br)cn2)cc1]

The above compound (6f) was prepared from 4c and 5b following Method B and purified by column chromatography on silica gel (EtOAc/hexane, 20:80) to give 6f (87%) as a yellow solid; ¹H NMR (CDCl₃, 600 MHz) 8.10 (1 H, s), 7.67 (1 H, s), 7.30 (2 H, t, J=6.1 Hz), 7.03 (2 H, t, J=8.5 Hz), 5.00 (1 H, br), 4.50 (2 H, s); HRMS (ESI-TOF) m/z: [M+H]⁺ calculated for C₁₁H₁₀⁷⁹BrFN₃ and C₁₁H₁₀⁸¹BrFN₃ 282.0037, 284.0016; found 282.0056, 284.0035.

### N-Benzyl-6-bromopyrazin-2-amine (6g)

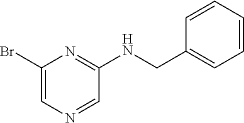 [SMILES: Brc1cncc(NCc2ccccc2)n1]

The above compound (6g) was prepared from 4d and 5a following Method B and purified by column chromatography on silica gel (EtOAc/hexane, 10:90 to 20:80) to give 6g (52%) as a white solid; ¹H NMR (CDCl₃, 600 MHz) 7.92 (1 H, s), 7.78 (1 H, s), 7.38-7.31 (5 H, m), 5.01 (1 H, br), 4.55 (2 H, d, J=6.1 Hz); HRMS (ESI-TOF) m/z: [M+H]⁺ calculated for C₁₁H₁₁⁷⁹BrN₃ and C₁₁H₁₁⁸¹BrN₃ 264.0131, 266.0111; found 264.0125, 266.0105.

### N-Benzyl-6-bromopyrimidin-4-amine (8a)

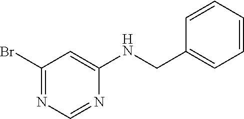 [SMILES: Brc1cc(NCc2ccccc2)ncn1]

The above compound (8a) was prepared from 7 and 5a following Method B and purified by column chromatography on silica gel (EtOAc/hexane, 20:80) to give 8a (71%) as a white solid; ¹H NMR (CDCl₃, 600 MHz) 8.26 (1 H, s), 7.37-7.30 (5 H, m), 6.55 (1 H, s), 4.51 (2 H, s); HRMS (ESI-TOF) m/z: [M+H]⁺ calculated for C₁₁H₁₁⁷⁹BrN₃ and C₁₁H₁₁⁸¹BrN₃ 264.0131, 266.0110; found 264.0136, 266.0116.

### 6-Bromo-N-(4-fluorobenzyl)pyrimidin-4-amine (8b)

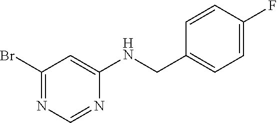 [SMILES: Fc1ccc(CNc2cc(Br)ncn2)cc1]

The above compound was prepared from 7 and 5b following Method B and purified by column chromatography on silica gel (EtOAc/hexane, 20:80) to give 8b (65%) as a white solid; ¹H NMR (CDCl₃, 600 MHz) 8.22 (1 H, s), 7.28-7.26 (2 H, m), 7.04 (2 H, t, J=8.5 Hz), 6.53 (1 H, s), 4.49 (2 H, s); HRMS (ESI-TOF) m/z: [M+H]⁺ calculated for C₁₁H₁₀⁷⁹BrFN₃ and C₁₁H₁₀⁸¹BrFN₃ 282.0037, 284.0016; found 282.0033, 284.0010.

### Example 4—General Procedure for the Preparation of N-Benzyl-4-bromopyrimidin-2-amine—Method C

### N-Benzyl-4-bromopyrimidin-2-amine (10a)

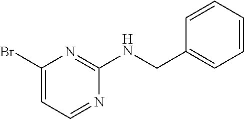 [SMILES: Brc1ccnc(NCc2ccccc2)n1]

To a solution of 4-bromopyrimidin-2-amine (9) (100.0 mg, 0.58 mmol) and sodium triacetoxyborohydride (365.6 mg, 1.72 mmol) in anhydrous 1,2-dichloroethane (10 mL) was added benzaldehyde (5a) (0.12 mL, 1.15 mmol) and trifluoroacetic acid (0.13 mL, 1.72 mmol), respectively, under nitrogen. The resulting mixture was stirred at room temperature for 1 h. A sodium triacetoxyborohydride (365.6 mg, 1.72 mmol) was successively added and the reaction was stirred for 20 h. To the mixture was added sodium triacetoxyborohydride (365.6 mg, 1.72 mmol) and 5a (0.12 mL, 1.15 mmol) and stirred at room temperature for another 20 h. After being quenched with sat. NaHCO₃ (5 mL), water and EtOAc were added. The layers were separated. The combined organic phases were washed with brine, dried over anhydrous Na₂SO₄, filtered, and concentrated. The residue was purified by column chromatography on silica gel (EtOAc/hexane, 15:85 to 20:80) to afford 10a (110.0 mg, 72%) as a white solid; ¹H NMR (CDCl₃, 600 MHz) 8.00 (1 H, br), 7.35-7.34 (4 H, m), 7.31-7.27 (1 H, m), 6.75 (1 H, d, J=4.9 Hz), 4.64 (2 H, d, J=6.1 Hz); HRMS (ESI-TOF) m/z: [M+H]⁺ calculated for C₁₁H₁₁⁷⁹BrN₃ and C₁₁H₁₁⁸¹BrN₃ 264.0131, 266.0110; found 264.0128, 266.0110.

### 4-Bromo-N-(4-fluorobenzyl)pyrimidin-2-amine (10b)

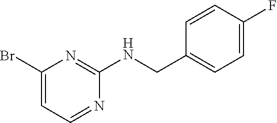 [SMILES: Fc1ccc(CNc2nccc(Br)n2)cc1]

The above compound (10b) was prepared from 9 and 5b following Method C and purified by column chromatography on silica gel (EtOAc/hexane, 20:80) to give 10b (65%) as a clear oil; ¹H NMR (CDCl₃, 600 MHz) 8.00 (1 H, br), 7.31-7.29 (2 H, m), 7.02 (2 H, t, J=8.5 Hz), 6.76 (1 H, d, J=4.9 Hz), 4.66 (1 H, s), 4.59 (2 H, d, J=6.1 Hz); HRMS (ESI-TOF) m/z: [M+H]⁺ calculated for C₁₁H₁₀⁷⁹BrFN₃ and C₁₁H₁₀⁸¹BrFN₃ 282.0037, 284.0016; found 282.0030, 284.0008.

The synthetic schemes for intermediates 3a-c, 6a-g, 8 a-b, and 10a-b, discussed supra, are shown in FIG. 1.

### Di-tert-butyl 5-bromo-2-oxo-1H-benzoldfimidazole-1,3(2H)-dicarboxylate (12)

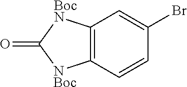 [SMILES: CC(C)(C)OC(=O)n1c(=O)n(C(=O)OC(C)(C)C)c2cc(Br)ccc21]

To a solution of 5-bromo-1H-benzo[d]imidazol-2(3H)-one (11) (0.5 g, 2.36 mmol) in anhydrous THF (10 mL) was added (Boc)₂O (2.1 g, 9.44 mmol) and DMAP (0.3 g, 2.44 mmol). The resulting mixture was stirred at room temperature for 12 h. After being quenched with H₂O (5 mL), the aqueous layer was extracted with EtOAc (2×20 mL). The combined organic extracts were washed with brine, dried over anhydrous Na₂SO₄, filtered, and concentrated. The residue was purified by column chromatography on silica gel (EtOAc/hexane, 25:75) to afford 12 (0.95 g, 98%) as a white solid; ¹H NMR (CDCl₃, 600 MHz) 8.10 (1 H, s), 7.76 (1 H, d), 7.37 (1 H, d), 1.68 (9 H, s), 1.67 (9 H, s).

### 5-(4,4,5,5-Tetramethyl-1,3,2-dioxaborolan-2-yl)-1H-benzo[d]imidazol-2(3H)-one trifluoroacetate salt (13a)

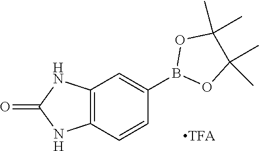 [SMILES: CC1(C)OB(c2ccc3[nH]c(=O)[nH]c3c2)OC1(C)C]

To a solution of di-tert-butyl-5-bromo-2-oxo-1H-benzo[d]imidazole-1,3(2H)-dicarboxylate (12) (0.9 g, 2.18 mmol), bis(pinacolato)diboron (1.1 g, 4.36 mmol) and Pd(dppf)Cl₂ (90 mg, 0.11 mmol) in 1,4-dioxane (15 mL) was added KOAc (0.65 g, 6.54 mmol) under nitrogen. The resulting mixture was stirred at 80° C. for 12 h. The reaction was allowed to cool to room temperature and filtered through a celite pad. EtOAc and water were added. The layers were separated. The combined organic phases were washed with brine, dried over anhydrous Na₂SO₄, filtered, and concentrated. The residue was purified by column chromatography on silica gel (EtOAc/hexane, 10:90) to afford intermediate di-tert-butyl 2-oxo-5-(4,4,5,5-tetramethyl-1,3,2-dioxaborolan-2-yl)-1H-benzo[d]imidazole-1,3(2H)-dicarboxylate as a white solid. To a solution of intermediate in CH₂Cl₂ (10 mL) was added TFA (2.5 mL). The reaction mixture was stirred at room temperature for 12 h. The solvent was removed in vacuo and the product (13a) was used in next step without further purification; ¹H NMR (DMSO-d₆, 600 MHz) 10.73 (1 H, s), 10.60 (1 H, s), 7.24 (1 H, d, J=8.5 Hz), 7.13 (1 H, s), 6.88 (1 H, d, J=7.3 Hz), 1.23 (12 H, s). HRMS (ESI-TOF) m/z: [M+H]⁺ calculated for C₁₃H₁₈BN₂O₃ 261.1405; found 261.1404.

### Example 5—General Procedure for the Preparation of Phenylboronic Acid Pinacol ester—Method D

### 5-(4,4,5,5-Tetramethyl-1,3,2-dioxaborolan-2-yl)-1H-indole (15a)

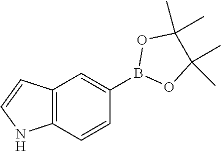 [SMILES: CC1(C)OB(c2ccc3[nH]ccc3c2)OC1(C)C]

To a mixture of 14a (150.0 mg, 0.76 mmol), bis(pinacolato)diboron (291.4 mg, 1.15 mmol), KOAc (225.2 mg, 2.30 mmol), and PdCl₂(dppf).CH₂Cl₂ (65.5 mg, 0.07 mmol) was added anhydrous 1,4-dioxane (5 mL) under nitrogen. The reaction was put into a preheated oil bath (80° C.), and stirred for 16 h. After being quenched by the addition of water, the aqueous layer was extracted with EtOAc (2×15 mL). The combined organic extracts were washed with brine, dried over anhydrous Na₂SO₄, filtered, and concentrated. The residue was purified by column chromatography on silica gel (EtOAc/hexane, 15:85) to afford 15a (149.4 mg, 80%) as a yellow solid; ¹H NMR (CDCl₃, 600 MHz) 8.28 (1 H, s), 8.21 (1 H, s), 7.66 (1 H, d, J=7.3 Hz), 7.38 (1 H, d, J=8.5 Hz), 7.18 (1 H, s), 6.57 (1 H, s), 1.38 (12 H, s); HRMS (ESI-TOF) m/z: [M+H]⁺ calculated for C₁₄H₁₉BNO₂ 244.1503; found 244.1504.

### 6-(4,4,5,5-tetramethyl-1,3,2-dioxaborolan-2-yl)-1H-indole (15b)

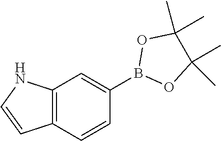 [SMILES: CC1(C)OB(c2ccc3cc[nH]c3c2)OC1(C)C]

The above compound (15b) was prepared from 14b following Method D and purified by column chromatography on silica gel (EtOAc/hexane, 15:85) to give 15b (72%) as a white solid; ¹H NMR (CDCl₃, 600 MHz) 8.30 (1 H, s), 7.93 (1 H, s), 7.68 (1 H, d, J=7.3 Hz), 7.56 (1 H, d, J=7.3 Hz), 6.58 (1 H, s), 1.40 (12 H, s); HRMS (ESI-TOF) m/z: [M+H]⁺ calculated for C₁₄H₁₉BNO₂ 244.1503; found 244.1571.

### 1-Methyl-5-(4,4,5,5-tetramethyl-1,3,2-dioxaborolan-2-yl)-1H-benzo[d]imidazol-2 (3H)-one (15c)

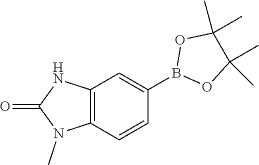 [SMILES: Cn1c(=O)[nH]c2cc(B3OC(C)(C)C(C)(C)O3)ccc21]

The above compound (15c) was prepared from 14c following Method D and purified by column chromatography on silica gel (EtOAc/hexane, 40:60) to give 15c (64%) as a light brown solid; ¹H NMR (CDCl₃, 600 MHz) 10.21 (1 H, s), 7.58-7.57 (2 H, m), 6.98 (1 H, d, J=8.5 Hz), 3.44 (3 H, s), 1.35 (12 H, s); HRMS (ESI-TOF) m/z: [M+H]⁺ calculated for C₁₄H₂₀BN₂O₃ 275.1561; found 275.1552.

### 1-Methyl-6-(4,4,5,5-tetramethyl-1,3,2-dioxaborolan-2-yl)-1H-benzo[d]imidazol-2 (3H)-one (15d)

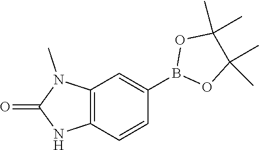 [SMILES: Cn1c(=O)[nH]c2ccc(B3OC(C)(C)C(C)(C)O3)cc21]

The above compound was prepared from 14d following Method D and purified by column chromatography on silica gel (EtOAc/hexane, 40:60) to give 15d (75%) as a white solid; ¹H NMR (CDCl₃, 600 MHz) 10.79 (1 H, s), 7.60 (1 H, d, J=7.3 Hz), 7.45 (1 H, s), 7.17 (1 H, d, J=7.3 Hz), 3.48 (3 H, s), 1.38 (12 H, s); HRMS (ESI-TOF) m/z: [M+H]⁺ calculated for C₁₄H₂₀BN₂O₃ 275.1561; found 275.1561.

### 5-(4,4,5,5-Tetramethyl-1, 3,2-dioxaborolan-2-yl)indolin-2-one (15e)

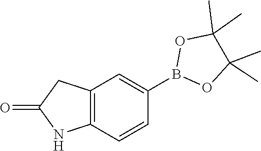 [SMILES: CC1(C)OB(c2ccc3c(c2)CC(=O)N3)OC1(C)C]

The above compound was prepared from 14e following Method D and purified by column chromatography on silica gel (EtOAc/hexane, 50:50) to give 15e (94%) as a brown solid; ¹H NMR (CDCl₃, 600 MHz) 9.18 (1 H, s), 7.71 (1 H, d, J=8.5 Hz), 7.68 (1 H, s), 6.92 (1 H, d, J=7.3 Hz), 3.54 (2 H, s), 1.35 (12 H, s); HRMS (ESI-TOF) m/z: [M+H]⁺ calculated for C₁₄H₁₉BNO₃ 260.1453; found 260.1461.

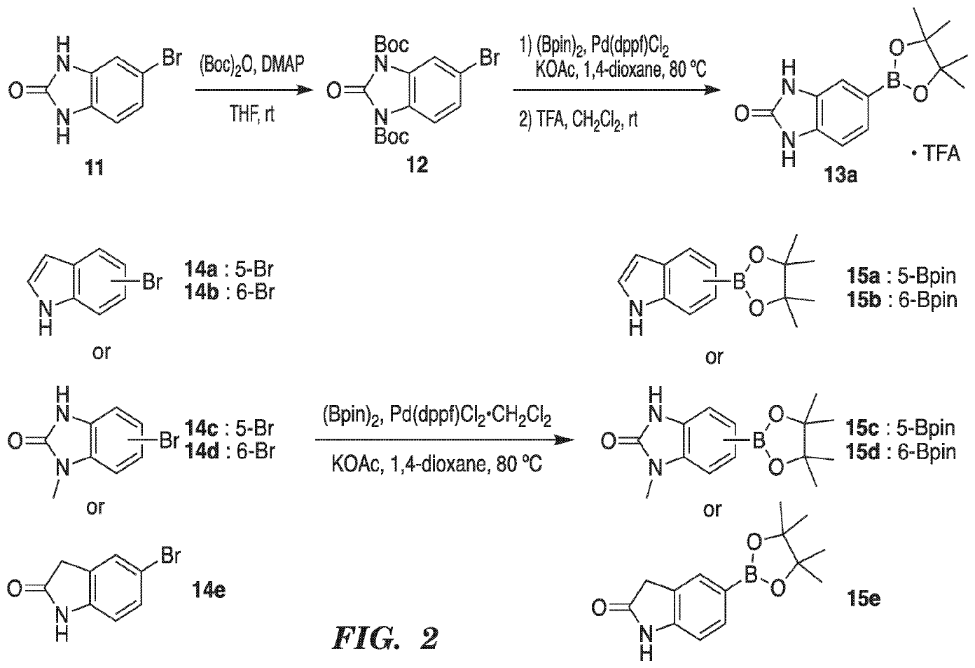

The synthetic schemes for intermediates 12, 13a, and 15a-e, discussed supra, are shown in FIG. 2.

### Example 6—General Procedure for the Preparation of Benzimidazolonyl/Indolyl Benzyl Heterocyclic Amines—Method E

### 5-(6-((4-Fluorobenzyl)amino)pyridazin-3-yl)-1H-benzo[d]imidazol-2 (3H)-one (16a)

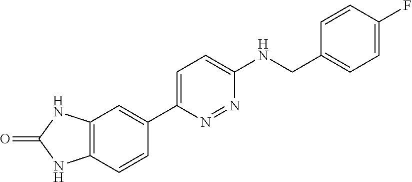 [SMILES: O=c1[nH]c2ccc(-c3ccc(NCc4ccc(F)cc4)nn3)cc2[nH]1]

To a mixture of 13a (37.6 mg, 0.10 mmol), 3b (21.7 mg, 0.09 mmol), and Pd(PPh₃)₄ (10.5 mg, 0.009 mmol) was added anhydrous acetonitrile (1 mL) and anhydrous DMF (1 mL) under nitrogen. The reaction was stirred at room temperature for 10 min then 1 M Na₂CO₃ (0.27 mL, 0.27 mmol) was added. The mixture was put into a preheated oil bath (90° C.), and stirred for 16 h. After being quenched by the addition of water, the aqueous layer was extracted with EtOAc (2×10 mL). The combined organic extracts were washed with brine, dried over anhydrous Na₂SO₄, filtered, and concentrated. The residue was purified by column chromatography on silica gel (MeOH/CH₂Cl₂, 10:90) to afford 16a (5.4 mg, 18%) as a pale yellow solid; ¹H NMR (DMSO-d₆, 600 MHz) 10.72 (1 H, s), 10.69 (1 H, s), 7.74 (1 H, d, J=9.8 Hz), 7.54 (1 H, s), 7.50 (1 H, d, J=9.8 Hz), 7.42-7.38 (3 H, m), 7.14(2 H, t, J=8.5 Hz), 6.96 (1 H, d, J=7.3 Hz), 6.90 (1 H, d, J=9.8 Hz), 4.58 (2 H, d, J=6.1 Hz); HRMS (ESI-TOF) m/z: [M+H]⁺ calculated for C₁₈H₁₅FN₅O 336.1255; found 336.1265.

### 5-(2-((4-Fluorobenzyl)amino)pyrimidin-5-yl)-1H-benzo[d]imidazol-2(3H)-one (16b)

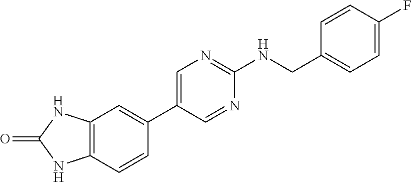 [SMILES: O=c1[nH]c2ccc(-c3cnc(NCc4ccc(F)cc4)nc3)cc2[nH]1]

The above compound (16b) was prepared from 13a and 6b following Method E and purified by column chromatography on silica gel (MeOH/CH₂Cl₂, 5:95 to 10:90) to give 16b (37%) as a pale yellow solid; ¹H NMR (DMSO-d₆, 600 MHz) 10.69-10.64 (2 H, m), 8.52 (2 H, s), 7.80 (1 H, s), 7.35 (2 H, s), 7.12-6.94 (5 H, m), 4.50 (2 H, s); HRMS (ESI-TOF) m/z: [M+H]⁺ calculated for C₁₈H₁₅FN₅O 336.1255; found 336.1253.

### 5-(2-(Benzylamino)pyrimidin-5-yl)-1H-benzo[d]yl)-2(3H)-one (16c)

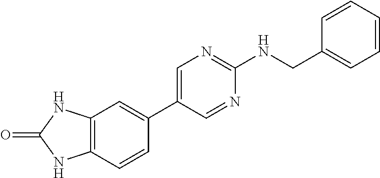 [SMILES: O=c1[nH]c2ccc(-c3cnc(NCc4ccccc4)nc3)cc2[nH]1]

The above compound was prepared from 13a and 6a following Method E and purified by column chromatography on silica gel (MeOH/CH₂Cl₂, 5:95 to 10:90) to give 16c (23%) as a light brown solid; ¹H NMR (DMSO-d₆, 600 MHz) 10.68 (1 H, s), 10.63 (1 H, s), 8.51 (2 H, s), 7.78 (1 H, t, J=6.1 Hz), 7.33-7.28 (4 H, m), 7.21-7.19 (1 H, m), 7.12 (1 H, d, J=9.8 Hz), 7.07 (1 H, s), 6.95 (1 H, d, J=7.3 Hz), 4.53 (2 H, d, J=6.1 Hz); HRMS (ESI-TOF) m/z: [M+H]⁺ calculated for C₁₈H₁₆N₅O 318.1349; found 318.1342.

### Example 7—General Procedure for the Preparation of Benzimidazolonyl/indolyl Benzyl Heterocyclic Amines—Method F

### 5-(2-(Benzylamino)pyrimidin-5-yl)-1-methyl-1H-benzo[d]imidazol-2 (3H)-one (17a)

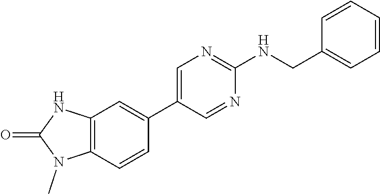 [SMILES: Cn1c(=O)[nH]c2cc(-c3cnc(NCc4ccccc4)nc3)ccc21]

To a mixture of 15c (37.7 mg, 0.14 mmol), 6a (42.8 mg, 0.14 mmol), and Pd(PPh₃)₄ (16.2 mg, 0.01 mmol) in a microwave reaction vial was added anhydrous 1,4-dioxane (1 mL) and 1 M Na₂CO₃ (0.28 mL, 0.28 mmol) under nitrogen. The reaction mixture was irradiated under microwave conditions for 10 min at 160° C. After being quenched by the addition of water, the aqueous layer was extracted with EtOAc (2×10 mL). The combined organic extracts were washed with brine, dried over anhydrous Na₂SO₄, filtered, and concentrated. The residue was purified by column chromatography on silica gel (MeOH/CH₂Cl₂, 2.5:97.5 to 5:95) to afford 17a (7.0 mg, 15%) as a pale yellow solid; ¹1⁻1 NMR (DMSO-d₆, 600 MHz) 10.93 (1 H, s), 8.54 (2 H, s), 7.81 (1 H, t, J=6.1 Hz), 7.32-7.28 (5 H, m), 7.24-7.20 (2 H, m), 7.13 (1 H, s), 4.54 (2 H, d, J=6.1 Hz), 3.28 (3 H, s); HRMS (ESI-TOF) m/z: [M+H]⁺ calculated for C₁₉H₁₈N₅O 332.1506; found 332.1527.

### 5-(2-((4-Fuorobenzyl)amino)pyrimidin-5-yl)-1-methyl-1H-benzo[d]imidazol-2(3H)-one (17b)

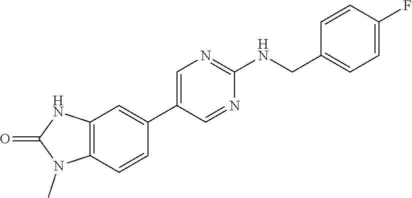 [SMILES: Cn1c(=O)[nH]c2cc(-c3cnc(NCc4ccc(F)cc4)nc3)ccc21]

The above compound (17b) was prepared from 15c and 6b following Method F and purified by column chromatography on silica gel (MeOH/CH₂Cl₂, 2.5:97.5 to 5:95) to give 17b (32%) as a pale yellow solid; ¹H NMR (DMSO-d₆, 600 MHz) 10.93 (1 H, s), 8.55 (2 H, s), 7.81 (1 H, t, J=6.1 Hz), 7.35 (2 H, t, J=6.1 Hz), 7.23 (1 H, d, J=7.3 Hz), 7.13-7.12 (4 H, m), 4.51 (2 H, d, J=6.1 Hz), 3.28 (3 H, s); HRMS (ESI-TOF) m/z: [M +1-1]⁺ calculated for C₁₉H₁₇FN₅O 350.1412; found 350.1432.

### 6-(2-(Benzylamino)pyrimidin-5-yl)-1-methyl-1H-benzo[d]imidazol-2(3H)-one (17c)

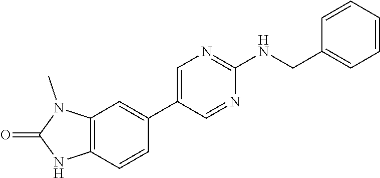 [SMILES: Cn1c(=O)[nH]c2ccc(-c3cnc(NCc4ccccc4)nc3)cc21]

The above compound (17c) was prepared from 15d and 6a following Method F and purified by column chromatography on silica gel (MeOH/CH₂Cl₂, 2.5:97.5 to 5:95) to give 17c (55%) as a white solid; ¹H NMR (DMSO-d₆, 600 MHz) 10.86 (1 H, s), 8.60 (2 H, s), 7.84 (1 H, t, J=6.1 Hz), 7.36-7.34 (3 H, m), 7.20 (1 H, d, J=8.5 Hz), 7.12 (2 H, t, J=8.5 Hz), 6.99 (1 H, d, J=8.5 Hz), 4.50 (2 H, d, J=6.1 Hz), 3.30 (3 H, s); HRMS (ESI-TOF) m/z: [M+H]⁺ calculated for C₁₉H₁₈N₅O 332.1506; found 332.1496.

### 6-(2-((4-Fluorobenzyl)amino)pyrimidin-5-yl)-1-methyl-1H-benzo[d]imidazol-2 (3H)-one (17d)

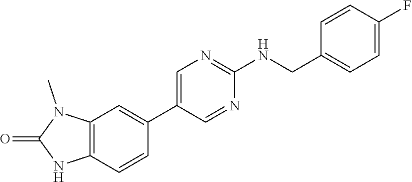 [SMILES: Cn1c(=O)[nH]c2ccc(-c3cnc(NCc4ccc(F)cc4)nc3)cc21]

The above compound (17d) was prepared from 15d and 6b following Method F and purified by column chromatography on silica gel (MeOH/CH₂Cl₂, 2.5:97.5 to 5:95) to give 17d (43%) as a white solid; ¹H NMR (DMSO-d₆, 600 MHz) 10.86 (1 H, s), 8.60 (2 H, s), 7.83 (1 H, t, J=6.1 Hz), 7.36-7.34 (3 H, m), 7.20 (1 H, d, J=7.3 Hz), 7.12 (2 H, t, J=8.5 Hz), 6.99 (1 H, d, J=8.5 Hz), 4.50 (2 H, d, J=4.9 Hz), 3.30 (3 H, s); HRMS (ESI-TOF) m/z: [M+H]⁺ calculated for C₁₉H₁₇FN₅O 350.1412; found 350.1421.

### N-Benzyl-5-(1H-indol-5-yl)pyrimidin-2-amine (17e)

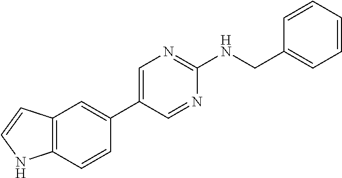 [SMILES: c1ccc(CNc2ncc(-c3ccc4[nH]ccc4c3)cn2)cc1]

The above compound (17e) was prepared from 15a and 6a following Method F and purified by column chromatography on silica gel (EtOAc/hexane, 40:60) to give 17e (70%) as a pale yellow solid; ¹H NMR (DMSO-d₆, 600 MHz) 8.58 (2 H, s), 7.72 (2 H, s), 7.43 (1 H, d, J=6.1 Hz), 7.35-7.30 (5 H, m), 7.20 (1 H, s), 6.43 (1 H, s), 4.54 (2 H, s); HRMS (ESI-TOF) m/z: [M+H]⁺ calculated for C₁₉H₁₇N₄ 301.1448; found 301.1442.

### N-(4-Fluorobenzyl)-5-(1H-indol-5-yl)pyrimidin-2-amine (17f)

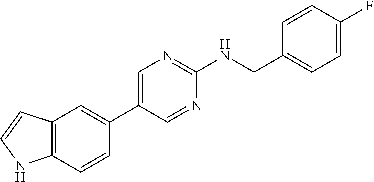 [SMILES: Fc1ccc(CNc2ncc(-c3ccc4[nH]ccc4c3)cn2)cc1]

The above compound (17f) was prepared from 15a and 6b following Method F and purified by column chromatography on silica gel (EtOAc/hexane, 30:70 to 60:40) to give 17f (77%) as a white solid; ¹H NMR (DMSO-d₆, 600 MHz) 8.59 (2 H, s), 7.73 (2 H, s), 7.44-7.29 (5 H, m), 7.12 (2 H, d, J=9.8 Hz), 6.44 (1 H, s), 4.52 (2 H, s); HRMS (ESI-TOF) m/z: [M+H]⁺ calculated for C₁₉H₁₆FN₄ 319.1354; found 319.1367.

### N-Benzyl-5-(1H-indol-6-yl)pyrimidin-2-amine (17g)

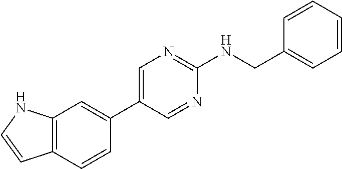 [SMILES: c1ccc(CNc2ncc(-c3ccc4cc[nH]c4c3)cn2)cc1]

The above compound (17f) was prepared from 15b and 6a following Method F and purified by column chromatography on silica gel (EtOAc/hexane, 40:60 to 50:50) to give 17g (32%) as a white solid; ¹H NMR (CD₃OD, 600 MHz) 8.56 (2 H, s), 7.60 (1 H, d, J=8.5), 7.52 (1 H, s), 7.36 (2 H, m), 7.30 (2 H, t, J=8.5 Hz), 7.25 (1 H, d, J=3.7 Hz), 7.22 (1 H, t, J=7.3 Hz), 7.18 (1 H, d, J=8.5 Hz), 6.44 (1 H, d, J=3.7 Hz), 4.64 (2 H, s); HRMS (ESI-TOF) m/z: [M+H]⁺ calculated for C₁₉H₁₇N₄ 301.1448; found 301.1426.

### N-(4-Fluorobenzyl)-5-(1H-indol-6-yl)pyrimidin-2-amine (17h)

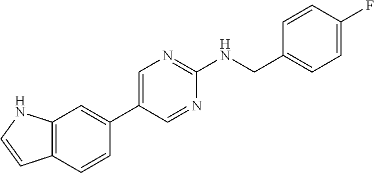 [SMILES: Fc1ccc(CNc2ncc(-c3ccc4cc[nH]c4c3)cn2)cc1]

The above compound (17h) was prepared from 15b and 6b following Method F and purified by column chromatography on silica gel (EtOAc/hexane, 30:70 to 60:40) to give 17h (72%) as a pale yellow solid; ¹H NMR (CD₃OD, 600 MHz) 8.56 (2 H, s), 7.60 (1 H, d, J=8.5), 7.52 (1 H, s), 7.38 (2 H, dd, J=7.9, 6.1 Hz), 7.25 (1 H, d, J=3.7 Hz), 7.17 (1 H, d, J=7.3 Hz), 7.03 (2 H, t, J=8.5 Hz), 6.44 (1 H, d, J=3.7 Hz), 4.61 (2 H, s); HRMS (ESI-TOF) m/z: [M+H]⁺ calculated for C₁₉H₁₆FN₄ 319.1354; found 319.1342.

### 5-(1H-Benzo[d]imidazol-5-yl)-N-benzylpyrimidin-2-amine (171)

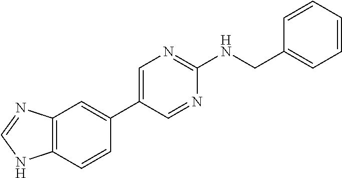 [SMILES: c1ccc(CNc2ncc(-c3ccc4[nH]cnc4c3)cn2)cc1]

The above compound (17i) was prepared from 15e and 6a following Method F and purified by column chromatography on silica gel (MeOH/CH₂Cl₂, 5:95v) to give 17i (71%) as a pale yellow solid; ¹H NMR (CD₃OD, 600 MHz) 8.58 (2 H, s), 8.19 (1 H, s), 7.74 (1 H, s), 7.68 (1 H, d, J=7.3), 7.45 (1 H, d, J=8.5 Hz), 7.36 (2 H, d, J=7.3 Hz), 7.30 (2 H, t, J=7.3 Hz), 7.22 (1 H, t, J=7.3 Hz), 4.64 (2 H, s); HRMS (ESI-TOF) m/z: [M+H]⁺ calculated for C₁₈H₁₆N₅ 302.1400; found 302.1390.

### 5-(1H-Benzo[d]imidazol-5-yl)-N-(4-fluorobenzyl)pyrimidin-2-amine (17j)

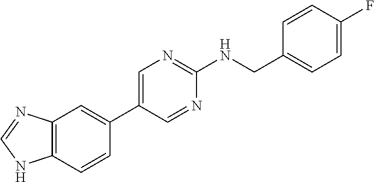 [SMILES: Fc1ccc(CNc2ncc(-c3ccc4[nH]cnc4c3)cn2)cc1]

The above compound (17j) was prepared from 15e and 6b following Method F and purified by column chromatography on silica gel (MeOH/CH₂Cl₂, 5:95 to 10:90) to give 17j (56%) as a white solid; ¹H NMR (CD₃OD, 600 MHz) 8.58 (2 H, s), 8.19 (1 H, s), 7.72 (2 H, br), 7.45 (1 H, d, J=8.5 Hz), 7.38 (2 H, dd, J=6.1, 5.5 Hz), 7.03 (2 H, t, J=9.8 Hz), 4.61 (2 H, s); HRMS (ESI-TOF) m/z: [M+H]⁺ calculated for C₁₈H₁₅FN₅ 320.1306; found 320.1292.

### 5-(2-(Benzylamino)-4-methylpyrimidin-5yl)-1H-benzo[d]imidazol-2 (3H)-one (18a)

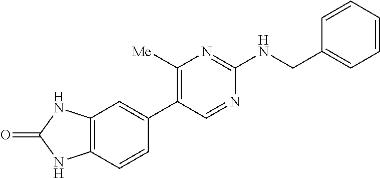 [SMILES: Cc1nc(NCc2ccccc2)ncc1-c1ccc2[nH]c(=O)[nH]c2c1]

The above compound (18a) was prepared from 13b and 6c following Method F and purified by column chromatography on silica gel (MeOH/CH₂Cl₂, 5:95 to 10:90) to give 18a (36%) as a yellow solid; ¹H NMR (DMSO-d₆, 600 MHz) 10.63 (2 H, s), 8.03 (1 H, s), 7.64 (1 H, br), 7.33-7.28 (4 H, m), 7.20 (1 H, t, J=6.1 Hz), 6.94 (1 H, d, J=7.3 Hz), 6.85 (1 H, d, J=6.1 Hz), 6.82 (1 H, s), 4.52 (2 H, d, J=6.1 Hz), 2.23 (3 H, s); HRMS (ESI-TOF) m/z: [M+H]⁺ calculated for C₁₉H₁₈N₅O 332.1506; found 332.1509.

### 5-(2-((4-Fluorobenzyl)amino)-4-methylpyrimidin-5-yl)-1H-benzo[d]imidazol-2(3H)-one (18b)

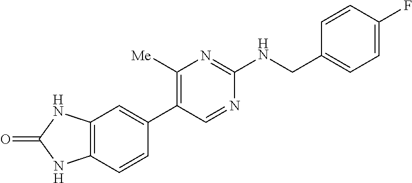 [SMILES: Cc1nc(NCc2ccc(F)cc2)ncc1-c1ccc2[nH]c(=O)[nH]c2c1]

The above compound (18b) was prepared from 13b and 6d following Method F and purified by column chromatography on silica gel (MeOH/CH₂Cl₂, 5:95 to 10:90) to give 18b (33%) as a yellow solid; ¹H NMR (DMSO-d₆, 600 MHz) 10.64 (2 H, s), 8.03 (1 H, s), 7.66 (1 H, s), 7.35 (2 H, t, J=7.3 Hz), 7.12 (1 H, t, J=8.5 Hz), 6.94 (1 H, d, J=8.5 Hz), 6.85 (1 H, d, J=8.5 Hz), 6.82 (1 H, s), 4.48 (2 H, d, J=6.1 Hz), 2.23 (3 H, s); HRMS (ESI-TOF) m/z: [M+H]⁺ calculated for C₁₉H₁₇FN₅O 350.1412; found 350.1409.

### 5-(5-(Benzylamino)pyrazin-2-yl)-1H-benzo[d]imidazol-2(3H)-one (18c)

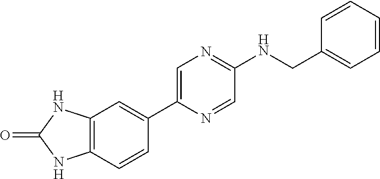 [SMILES: O=c1[nH]c2ccc(-c3cnc(NCc4ccccc4)cn3)cc2[nH]1]

The above compound (18c) was prepared from 13b and 6e following Method F and purified by column chromatography on silica gel (MeOH/CH₂Cl₂, 5:95 to 10:90) to give 18c (26%) as a brown solid; ¹H NMR (CD₃OD, 600 MHz) 8.35 (1 H, s), 7.96 (1 H, s), 7.51-7.50 (2 H, m), 7.36 (2 H, d, J=7.3 Hz), 7.31 (2 H, t, J=7.3 Hz), 7.23 (1 H, t, J=8.5 Hz), 7.08 (1 H, d, J=8.5 Hz), 4.58 (2 H, s); HRMS (ESI-TOF) m/z: [M+H]⁺ calculated for C₁₈H₁₆N₅O 318.1349; found 318.1349.

### 5-(5-((4-Fluorobenzyl)amino)pyrazin-2-yl)-1H-benzo[d]imidazol-2 (3H)-one (18d)

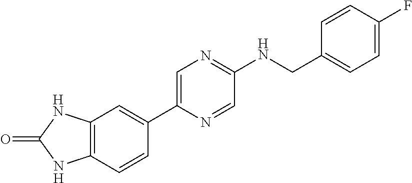 [SMILES: O=c1[nH]c2ccc(-c3cnc(NCc4ccc(F)cc4)cn3)cc2[nH]1]

The above compound (18d) was prepared from 13b and 6f following Method F and purified by column chromatography on silica gel (MeOH/CH₂Cl₂, 5:95 to 10:90) to give 18d (31%) as a brown solid; ¹H NMR (CD₃OD, 600 MHz) 8.36 (1 H, s), 7.96 (1 H, s), 7.51-7.50 (2 H, m), 7.38 (2 H, dd, J=7.9, 6.1 Hz), 7.08 (1 H, d, J=8.5 Hz), 7.04 (2 H, t, J=8.5 Hz), 4.56 (2 H, s); HRMS (ESI-TOF) m/z: [M+H]⁺ calculated for C₁₈H₁₅FN₅O 336.1255; found 336.1259.

### 5-(6-(Benzylamino)pyrimidin-4-yl)-1H-benzo[d]imidazol-2(3H)-one (18e)

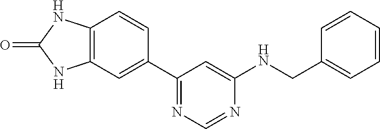 [SMILES: O=c1[nH]c2ccc(-c3cc(NCc4ccccc4)ncn3)cc2[nH]1]

The above compound (18e) was prepared from 13b and 8a following Method F and purified by column chromatography on silica gel (MeOH/CH₂Cl₂, 5:95 to 10:90) to give 18e (44%) as a yellow solid; ¹H NMR (CD₃OD, 600 MHz) 8.43 (1 H, s), 7.59 (2 H, s), 7.36-7.31 (4 H, m), 7.24 (1 H, t, J=7.3 Hz), 7.10 (1 H, d, J=8.5 Hz), 6.84 (1 H, s), 4.62(2 H, s); HRMS (ESI-TOF) m/z: [M+H]⁺ calculated for C₁₈H₁₆N₅O 318.1349; found 318.1347.

### 5-(6-((4-Fluorobenzyl)amino)pyrimidin-4-yl)-1H-benzo[d]imidazol-2(3H)-one (18f)

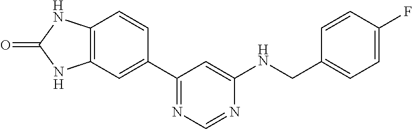 [SMILES: O=c1[nH]c2ccc(-c3cc(NCc4ccc(F)cc4)ncn3)cc2[nH]1]

The above compound (18f) was prepared from 13b and 8b following Method F and purified by column chromatography on silica gel (MeOH/CH₂Cl₂, 5:95 to 10:90) to give 18f (37%) as a yellow solid; ¹H NMR (DMSO-d₆, 600 MHz) 10.81 (1 H, s), 10.77 (1 H, s), 8.44 (1 H, s), 7.83 (1 H, t, J=6.1 Hz), 7.62-7.57 (2 H, m), 7.37 (2 H, s), 7.14 (2 H, t, J=8.5 Hz), 6.99 (1 H, d, J=8.5 Hz), 6.90 (1 H, s), 4.55 (2 H, s); HRMS (ESI-TOF) m/z: [M+H]⁺ calculated for C₁₈H₁₅FN₅O 336.1255; found 336.1283.

### 5-(2-(Benzylamino)pyrimidin-4-yl)-1H-benzo[d]imidazol-2(3H)-one (181)

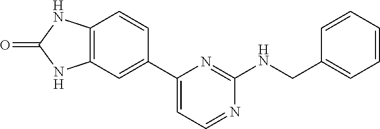 [SMILES: O=c1[nH]c2ccc(-c3ccnc(NCc4ccccc4)n3)cc2[nH]1]

The above compound (18i) was prepared from 13b and 10a following Method F and purified by column chromatography on silica gel (MeOH/CH₂Cl₂, 5:95) to give 18i (34%) as a yellow solid; ¹H NMR (CD₃OD, 600 MHz) 8.23 (1 H, d, J=4.8 Hz), 7.82 (1 H, s), 7.79 (1 H, d, J=8.5 Hz), 7.39 (2 H, d, J=7.3 Hz), 7.30 (2 H, t, J=8.5 Hz), 7.20 (1 H, t, J=7.3 Hz), 7.10-7.08 (2 H, m), 4.67 (2 H, s); HRMS (ESI-TOF) m/z: [M+H]⁺ calculated for C₁₈H₁₆N₅O 318.1349; found 318.1349.

### 5-(2-((4-Fluorobenzyl)amino)pyrimidin-4-yl)-1H-benzo[d]imidazol-2(3H)-one (18j)

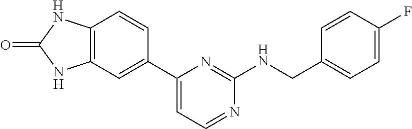 [SMILES: O=c1[nH]c2ccc(-c3ccnc(NCc4ccc(F)cc4)n3)cc2[nH]1]

The above compound (18j) was prepared from 13b and 10b following Method F and purified by column chromatography on silica gel (MeOH/CH₂Cl₂, 5:95) to give 18j (36%) as a yellow solid; ¹H NMR (CD₃OD, 600 MHz) 8.23(1 H, d, J=4.9 Hz), 7.82 (1 H, s), 7.79 (1 H, d, J=9.8 Hz), 7.41 (2 H, dd, J=8.2, 4.9 Hz), 7.11-7.08 (2 H, m), 7.02(2 H, t, J=9.8 Hz), 4.64 (2 H, s); HRMS (ESI-TOF) m/z: [M+H]⁺ calculated for C₁₈H₁₅FN₅O 336.1255; found 336.1284.

### 5-(2-(Benzylamino)pyrimidin-5-yl)indolin-2-one (18k)

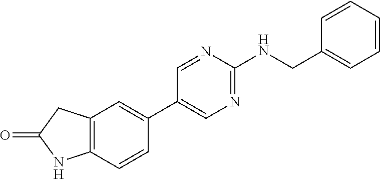 [SMILES: O=C1Cc2cc(-c3cnc(NCc4ccccc4)nc3)ccc2N1]

The above compound (18k) was prepared from 14e and 6a following Method F and purified by column chromatography on silica gel (EtOAc/hexane, 30:70 to 80:20) to give 18k (39%) as a yellow solid; ¹H NMR (DMSO-d₆, 600 MHz) 10.41 (1 H, s), 8.52 (2 H, s), 7.79 (1 H, t, J=7.3 Hz), 7.45 (1 H, s), 7.38 (1 H, d, J=7.3 Hz), 7.32-7.28 (5 H, m), 7.20 (1 H, t, J=7.3 Hz), 6.84 (1 H, d, J=7.3 Hz), 4.53 (2 H, d, J=6.1 Hz), 3.49 (2 H, s); HRMS (ESI-TOF) m/z: [M+H]⁺ calculated for C₁₉H₁₇N₄O 317.1397; found 317.1382.

### 5-(2-((4-Fluorobenzyl)amino)pyrimidin-5-yl)indolin-2-one (181)

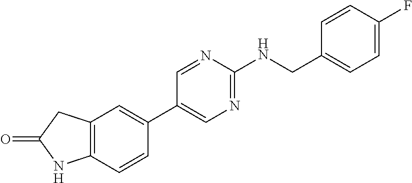 [SMILES: O=C1Cc2cc(-c3cnc(NCc4ccc(F)cc4)nc3)ccc2N1]

The above compound (18l) was prepared from 14e and 6b following Method F and purified by column chromatography on silica gel (MeOH/CH₂Cl₂, 2.5:97.5 to 5:95) to give 18l (28%) as a white solid; ¹H NMR (DMSO-d₆, 600 MHz) 10.41 (1 H, s), 8.52 (2 H, s), 7.81(1 H, t, J=7.3 Hz), 7.45 (1 H, s), 7.39 (1 H, d, J=8.5 Hz), 7.34(2 H, t, J=6.1 Hz), 7.11 (2 H, t, J=7.3 Hz), 6.84 (1 H, d, J=8.5 Hz), 4.50 (2 H, d, J=6.1 Hz), 3.50 (2 H, s); HRMS (ESI-TOF) m/z: [M+H]⁺ calculated for C₁₉H₁₆FN₄O 335.1303; found 335.1304.

### 5-(6-(Benzylamino)pyrazin-2-yl)-1H-benzo[d]imidazol-2(3H)-one (18m)

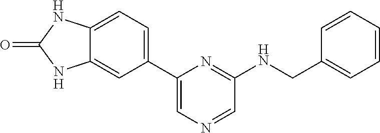 [SMILES: O=c1[nH]c2ccc(-c3cncc(NCc4ccccc4)n3)cc2[nH]1]

The above compound (18m) was prepared from 13b and 6g following Method I and purified by column chromatography on silica gel (MeOH/CH₂Cl₂, 5:95) to give 18l (58%) as a light brown solid; ¹H NMR (DMSO-d₆, 600 MHz) 10.74 (1 H, s), 10.71 (1 H, s), 8.19 (1 H, s), 7.84 (1 H, s), 7.62 (2 H, t, J=6.1 Hz), 7.58 (1 H, s), 7.39 (2 H, d, J=7.3 Hz), 7.33 (2 H, t, J=7.3 Hz), 7.23 (1 H, t, J=7.3 Hz), 6.96 (1 H, d, J=8.5 Hz), 4.57 (2 H, d, J=6.1 Hz); HRMS (ESI-TOF) m/z: [M+H]⁺ calculated for C₁₈H₁₆N₅O 318.1350; found 318.1303.

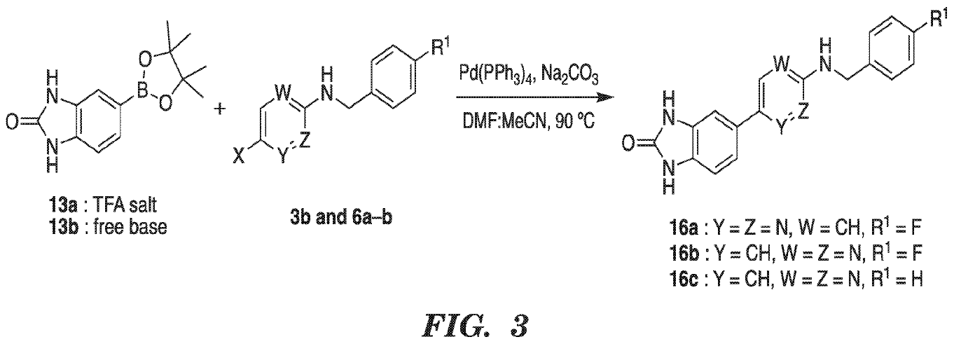

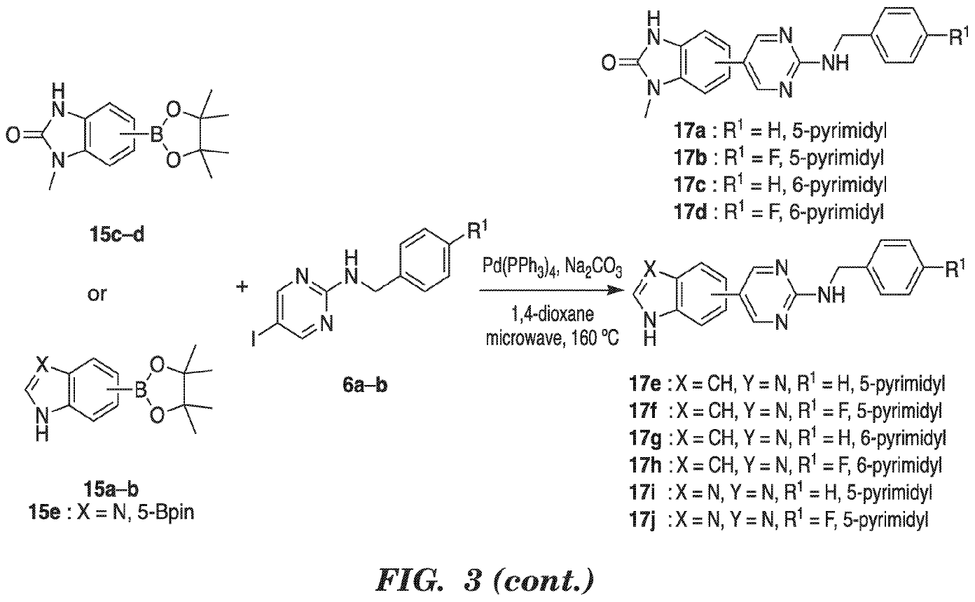

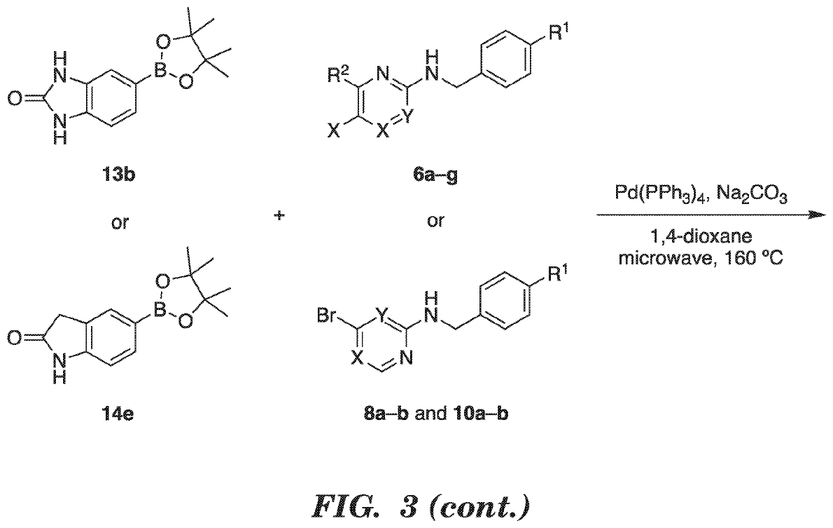

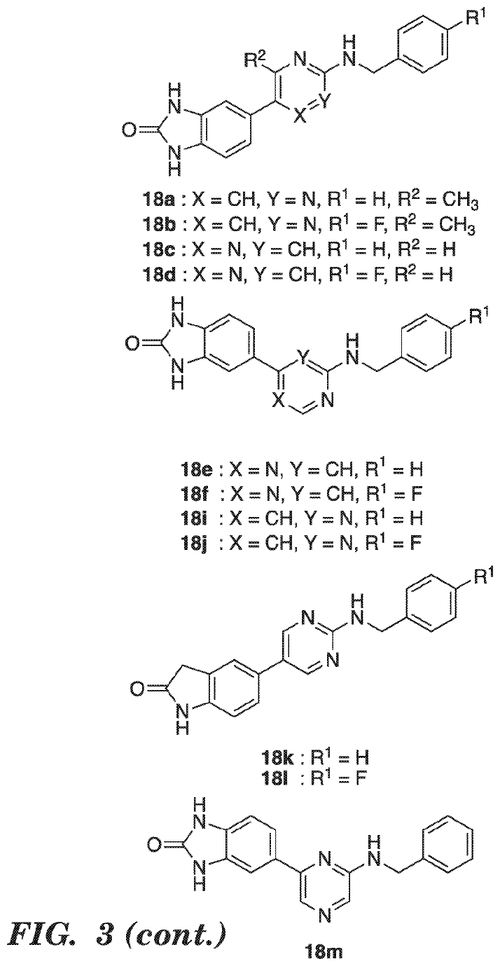

The synthetic schemes for compounds 16a-c, 17a-j, and 18a-m, discussed supra, are shown in FIG. 3.

### 5-Iodo-N-(pyridin-2-ylmethyl)pyrimidin-2-amine (20a)

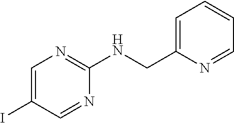 [SMILES: Ic1cnc(NCc2ccccn2)nc1]

The above compound (20a) was prepared from 4a and 19a following Method B and purified by column chromatography on silica gel (EtOAc/hexane, 40:60 to 50:50) to give 20a (56%) as a yellow solid; ¹H NMR (CDCl₃, 600 MHz) 8.56 (1 H, d, J=4.9 Hz), 8.40 (2 H, s), 7.67 (1 H, t, J=7.3 Hz), 7.31 (1 H, d, J=7.3 Hz), 7.21 (1 H, t, J=7.3 Hz), 6.38 (1 H, br), 4.71 (2 H, d, J=6.1 Hz); HRMS (ESI-TOF) m/z: [M+H]⁺ calculated for C₁₀H₁₀lN₄ 312.9945; found 312.9950.

### 5-Iodo-N-(pyridin-3-ylmethyl)pyrimidin-2-amine (20b)

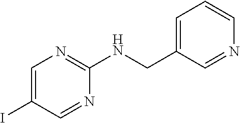 [SMILES: Ic1cnc(NCc2cccnc2)nc1]

The above compound (20b) was prepared from 4a and 19b following Method B and purified by column chromatography on silica gel (MeOH/CH₂Cl₂, 5:95) to give 20b (91%) as a yellow solid; ¹H NMR (CDCl₃, 600 MHz) 8.61 (1 H, s), 8.53 (1 H, s), 8.35 (2 H, s), 7.67 (1 H, d, J=7.3 Hz), 7.27 (1 H, s), 5.85 (1 H, br), 4.61 (2 H, d, J=6.1 Hz); HRMS (ESI-TOF) m/z: [M+H]⁺ calculated for C₁₀H₁₀IN₄ 312.9945; found 312.9939.

### 5-Iodo-N-(pyridin-4-ylmethyl)pyrimidin-2-amine (20c)

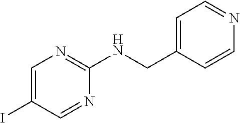 [SMILES: Ic1cnc(NCc2ccncc2)nc1]

The above compound (20c) was prepared from 4a and 19c following Method B and purified by column chromatography on silica gel (MeOH/CH₂Cl₂, 5:95) to give 20c (65%) as a brown solid; ¹H NMR (CDCl₃, 600 MHz) 8.54 (2 H, d, J=4.9 Hz), 8.37 (2 H, s), 7.24 (2 H, d, J=4.9 Hz), 5.86 (1 H, br), 4.63 (2 H, d, J=6.1 Hz); HRMS (ESI-TOF) m/z: [M+H]⁺ calculated for C₁₀H₁₀IN₄ 312.9945; found 312.9943.

### N-(2-Fluorobenzyl)-5-iodopyrimidin-2-amine (22a)

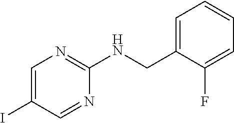 [SMILES: Fc1ccccc1CNc1ncc(I)cn1]

The above compound (22a) was prepared from 4a and 21a following Method B and purified by column chromatography on silica gel (EtOAc/hexane, 10:90) to give 22a (90%) as a white solid; ¹H NMR (CDCl₃, 600 MHz) 8.36 (2 H, s), 7.36 (1 H, t, J=7.3 Hz), 7.28-7.24 (1 H, m), 7.10-7.04 (2 H, m), 5.74 (1 H, br), 4.65 (2 H, d, J=4.9 Hz); HRMS (ESI-TOF) m/z: [M+H]⁺ calculated for C₁₁H₁₀FIN₃ 329.9898; found 329.9903.

### N-(3-Fluorobenzyl)-5-iodopyrimidin-2-amine (22b)

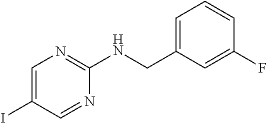 [SMILES: Fc1cccc(CNc2ncc(I)cn2)c1]

The above compound (22b) was prepared from 4a and 21b following Method B and purified by column chromatography on silica gel (EtOAc/hexane, 10:90 to 20:80) to give 22b (73%) as a white solid; ¹H NMR (CDCl₃, 600 MHz) 8.38 (2 H, s), 7.29 (1 H, q, J=6.1 Hz), 7.10 (1 H, d, J=7.3 Hz), 7.03 (1 H, d, J=9.8 Hz), 6.96 (1 H, td, J=8.5, 2.4 Hz), 5.62 (1 H, br), 4.60 (2 H, d, J=6.1 Hz); HRMS (ESI-TOF) m/z: [M+H]⁺ calculated for C₁₁H₁₀FIN₃ 329.9898; found 329.9891.

### N-(2-Chlorobenzyl)-5-iodopyrimidin-2-amine (22c)

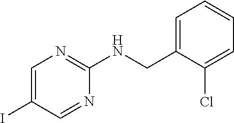 [SMILES: Clc1ccccc1CNc1ncc(I)cn1]

The above compound (22c) was prepared from 4a and 21c following Method B and purified by column chromatography on silica gel (EtOAc/hexane, 10:90) to give 22c (88%) as a white solid; ¹H NMR (CDCl₃, 600 MHz) 8.34 (2 H, s), 7.40-7.37 (2 H, m), 7.23-7.22 (2 H, m), 7.03 (1 H, d, J=9.8 Hz), 5.94 (1 H, br), 4.68 (2 H, d, J=4.9 Hz); HRMS (ESI-TOF) m/z: [M+H]⁺ calculated for C₁₁H₁₀ClIN₃ 345.9602; found 345.9594.

### N-(3-Chlorobenzyl)-5-iodopyrimidin-2-amine (22d)

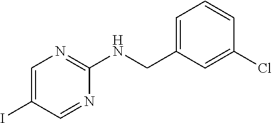 [SMILES: Clc1cccc(CNc2ncc(I)cn2)c1]

The above compound (22d) was prepared from 4a and 21d following Method B and purified by column chromatography on silica gel (EtOAc/hexane, 10:90 to 20:80) to give 22d (89%) as a white solid; ¹H NMR (CDCl₃, 600 MHz) 8.38 (2 H, s), 7.38-7.25 (3 H, m), 7.20 (1 H, d, J=6.1 Hz), 5.70 (1 H, br), 4.58 (2 H, d, J=6.1 Hz); HRMS (ESI-TOF) m/z: [M+H]⁺ calculated for C₁₁H₁₀ClIN₃ 345.9602; found 345.9619.

### N-(4-Chlorobenzyl)-5-iodopyrimidin-2-amine (22e)

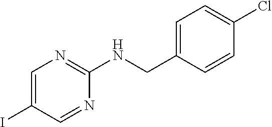 [SMILES: Clc1ccc(CNc2ncc(I)cn2)cc1]

The above compound (22e) was prepared from 4a and 21e following Method B and purified by column chromatography on silica gel (EtOAc/hexane, 10:90) to give 22e (86%) as a white solid; ¹H NMR (CDCl₃, 600 MHz) 8.36 (2 H, s), 7.30 (2 H, d, J=8.5 Hz), 7.26 (2 H, d, J=8.5 Hz), 5.71 (1 H, br), 4.56 (2 H, d, J=6.1 Hz); HRMS (ESI-TOF) m/z: [M+H]⁺ calculated for C₁₁H₁₀ClIN₃ 345.9602; found 345.9657.

### 3-(((5-Iodopyrimidin-2-yl)amino)methyl)benzonitrile (22f)

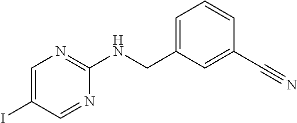 [SMILES: N#Cc1cccc(CNc2ncc(I)cn2)c1]

The above compound (22f) was prepared from 4a and 21f following Method B and purified by column chromatography on silica gel (EtOAc/hexane, 30:70 to 60:40) to give 22f (90%) as a white solid; ¹H NMR (CDCl₃, 600 MHz) 8.38 (2 H, s), 7.62-7.43 (4 H, m), 5.75 (1 H, br), 4.63 (2 H, s); HRMS (ESI-TOF) m/z: [M+H]⁺ calculated for C₁₂H₁₀IN₄ 336.9945; found 336.9944.

### 4-(((5-Iodopyrimidin-2-yl)amino)methyl)benzonitrile (22g)

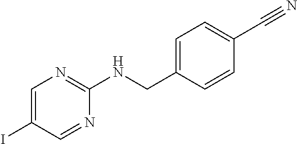 [SMILES: N#Cc1ccc(CNc2ncc(I)cn2)cc1]

The above compound (22g) was prepared from 4a and 21g following Method B and purified by column chromatography on silica gel (EtOAc/hexane, 30:70 to 60:40) to give 22g (42%) as a white solid; ¹H NMR (CDCl₃, 600 MHz) 8.40 (2 H, s), 7.62 (2 H, d, J=7.3 Hz), 7.43 (2 H, d, J=8.5 Hz), 5.78 (1 H, br), 4.68 (2 H, d, J=6.1 Hz); HRMS (ESI-TOF) m/z: [M+H]⁺ calculated for C₁₂H₁₀IN₄ 336.9945; found 336.9924.

### N-([1,1′-Biphenyl]-4-ylmethyl)-5-iodopyrimidin-2-amine (22h)

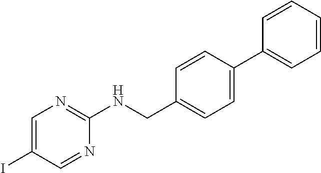 [SMILES: Ic1cnc(NCc2ccc(-c3ccccc3)cc2)nc1]

The above compound (22h) was prepared from 4a and 21h following Method B and purified by column chromatography on silica gel (EtOAc/hexane, 30:70 to 50:50) to give 22h (89%) as a yellow solid; ¹H NMR (CDCl₃, 600 MHz) 8.38 (2 H, s), 7.59-7.56 (4 H, m), 7.44 (2 H, t, J=7.3 Hz), 7.40 (2 H, d, J=8.5 Hz), 7.34 (1 H, t, J=7.3 Hz), 5.68 (1 H, br), 4.64 (2 H, d, J=6.1 Hz); HRMS (ESI-TOF) m/z: [M+H]⁺ calculated for C₁₇H₁₅IN₃ 388.0305; found 388.0299.

### 5-Iodo-N-(4-methoxybenzyl)pyrimidin-2-amine (22i)

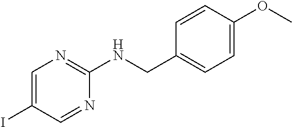 [SMILES: COc1ccc(CNc2ncc(I)cn2)cc1]

The above compound (22i) was prepared from 4a and 21i following Method B and purified by column chromatography on silica gel (EtOAc/hexane, 30:70) to give 22i (83%) as a white solid; ¹H NMR (CDCl₃, 600 MHz) 8.33 (2 H, br), 7.26 (2 H, d, J=7.3 Hz), 6.88 (2 H, d, J=8.5 Hz), 5.83 (1 H, br), 4.50 (2 H, d, J=6.1 Hz), 3.81 (3 H, s); HRMS (ESI-TOF) m/z: [M+H]⁺ calculated for C₁₂H₁₃IN₃O 342.0098; found 342.0271.

### 3-(((5-Iodopyrimidin-2-yl)amino)methyl)phenol (22j)

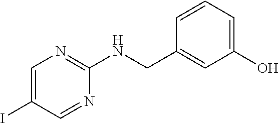 [SMILES: Oc1cccc(CNc2ncc(I)cn2)c1]

The above compound (22j) was prepared from 4a and 21j following Method B and purified by column chromatography on silica gel (EtOAc/hexane, 20:80 to 30:70) to give 22j (90%) as a white solid; ¹H NMR (CDCl₃, 600 MHz) 8.38 (2 H, s), 7.19 (1 H, t, J=7.3 Hz), 6.86 (1 H, d, J=7.3 Hz), 6.76-6.73 (2 H, m), 5.54 (1 H, br), 4.55 (2 H, d, J=6.1 Hz); HRMS (ESI-TOF) m/z: [M+H]⁺ calculated for C₁₁H₁₁IN₃O 327.9941; found 328.0179.

### 5-Iodo-N-(3-methoxybenzyl)pyrimidin-2-amine (22k)

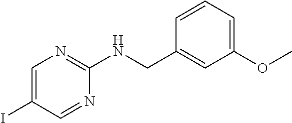 [SMILES: COc1cccc(CNc2ncc(I)cn2)c1]

The above compound (22k) was prepared from 4a and 21k following Method B and purified by column chromatography on silica gel (EtOAc/hexane, 30:70 to 40:60) to give 22k (70%) as a white solid; ¹H NMR (CDCl₃, 600 MHz) 8.39 (2 H, s), 7.25 (1 H, d, J=7.3 Hz), 6.91 (1 H, d, J=7.3 Hz), 6.87 (1 H, s), 6.82 (1 H, d, J=6.1 Hz), 5.47 (1 H, br), 4.57 (2 H, d, J=6.1 Hz), 3.80 (3 H, s); HRMS (ESI-TOF) m/z: [M+H]⁺ calculated for C₁₂H₁₃IN₃O 342.0098; found 342.0322.

### 5-Iodo-N-(3-(trifluoromethoxy)benzyl)pyrimidin-2-amine (22l)

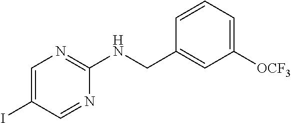 [SMILES: FC(F)(F)Oc1cccc(CNc2ncc(I)cn2)c1]

The above compound (221) was prepared from 4a and 21l following Method B and purified by column chromatography on silica gel (EtOAc/hexane, 30:70 to 40:60) to give 22l (98%) as a white solid; ¹H NMR (CDCl₃, 600 MHz) 8.30 (2 H, br), 7.35 (1 H, t, J=7.3 Hz), 7.26 (1 H, d, J=6.1 Hz), 7.18 (1 H, s), 7.13 (1 H, d, J=7.3 Hz), 6.05 (1 H, br), 4.61 (2 H, d, J=6.1 Hz); HRMS (ESI-TOF) m/z: [M+H]⁺ calculated for C₁₂H₁₀F₃IN₃O 395.9815; found 395.9847.

### 5-Iodo-N-(3-phenylpropyl)pyrimidin-2-amine (22m)

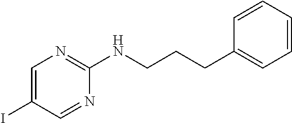 [SMILES: Ic1cnc(NCCCc2ccccc2)nc1]

The above compound (22m) was prepared from 4a and 21m following Method B and purified by column chromatography on silica gel (EtOAc/hexane, 20:80) to give 22m (44%) as a white solid; ¹H NMR (CDCl₃, 500 MHz) 8.36 (2 H, s), 7.30-7.25 (2 H, m), 7.20-7.18 (3 H, m), 5.11 (1 H, br), 3.41 (2 H, q, J=6.1 Hz), 2.70 (2 H, t, J=7.1 Hz), 1.94 (2 H, quin, J=7.6 Hz); HRMS (ESI-TOF) m/z: [M+H]⁺ calculated for C₁₃H₁₅IN₃ 340.0306; found 340.0302.

### 5-Iodo-N-(naphthalen-1-ylmethyl)pyrimidin-2-amine (24a)

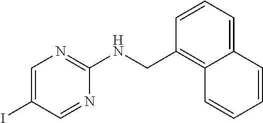 [SMILES: Ic1cnc(NCc2cccc3ccccc23)nc1]

The above compound (24a) was prepared from 4a and 23a following Method B and purified by column chromatography on silica gel (EtOAc/hexane, 20:80) to give 24a (94%) as a white solid; ¹H NMR (CDCl₃, 600 MHz) 8.31 (2 H, br), 8.01 (1 H, t, J=4.9 Hz), 7.90 (1 H, t, J=4.9 Hz), 7.84 (1 H, d, J=8.54 Hz), 7.52-7.50 (2 H, m), 7.49 (1 H, d, J=7.3 Hz), 7.44 (1 H, t, J=7.3 Hz), 6.08 (1 H, br), 4.98 (2 H, d, J=6.1 Hz); HRMS (ESI-TOF) m/z: [M+H]⁺ calculated for C₁₅H₁₃IN₃ 362.0149; found 362.0124.

### 5-Iodo-N-(naphthalen-2-ylmethyl)pyrimidin-2-amine (24b)

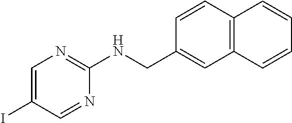 [SMILES: Ic1cnc(NCc2ccc3ccccc3c2)nc1]

The above compound (24b) was prepared from 4a and 23b following Method B and purified by column chromatography on silica gel (EtOAc/hexane, 20:80 to 40:60) to give 24b (76%) as a white solid; ¹H NMR (CDCl₃, 600 MHz) 8.36 (2 H, br), 7.83-7.77 (4 H, m), 7.48-7.44 (3 H, m), 5.76 (1 H, br), 4.76 (2 H, d, J=6.1 Hz); HRMS (ESI-TOF) m/z: [M+H]⁺ calculated for C₁₅H₁₃IN₃ 362.0149; found 362.0131.

### 5-Iodo-N-phenylpyrimidin-2-amine (26)

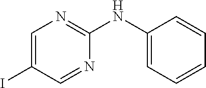 [SMILES: Ic1cnc(Nc2ccccc2)nc1]

To a solution of 5-iodopyrimidin-2-amine (4a) (200.0 mg, 0.90 mmol) and sodium tert-butoxide (87.0 mg, 0.90 mmol), XantPhos (26.2 mg, 0.045 mmol) and Pd₂(dba)₃ (41.4 mg, 0.045 mmol) in anhydrous toluene (2 mL) was added iodobenzene (25) (0.10 mL, 0.90 mmol). The resulting mixture was purged with nitrogen for 10 min then stirred at 95° C. for 20 h. After being quenched by the addition of water, the aqueous layer was extracted with EtOAc (2×20 mL). The combined organic extracts were washed with brine, dried over anhydrous Na₂SO₄, filtered, and concentrated. The residue was purified by column chromatography on silica gel (EtOAc/hexane, 15:85 to 30:70) to afford 26 (49.2 mg, 18%) as a white solid; ¹H NMR (CDCl₃, 600 MHz) 8.53 (2 H, s), 7.56 (2 H, d, J=8.5 Hz), 7.35 (2 H, t, J=8.5 Hz), 7.19 (1 H, br), 7.08 (1 H, t, J=7.3 Hz); HRMS (ESI-TOF) m/z: [M+H]⁺ calculated for C₁₀H₉IN₃ 297.9836; found 297.9839.

### Example 8—General Procedure for the Preparation of 5-iodophenethylpyrimidin-2-amine—Method G

### 5-Iodo-N-(1-phenylethyl)pyrimidin-2-amine (28a)

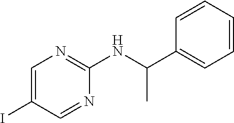 [SMILES: CC(Nc1ncc(I)cn1)c1ccccc1]

To a solution 4a (100.0 mg, 0.45 mmol) in anhydrous DMF (3 mL) was added 60% NaH (23.5 mg, 0.59 mmol) at 0° C. under nitrogen and stirred for 20 min. 27a (100.4 mg, 0.542 mmol) was added. The reaction was stirred at 0° C. and slowly increased to room temperature for 2 h. After being quenched by the addition of water, the aqueous layer was extracted with EtOAc (2×20 mL). The combined organic extracts were washed with brine, dried over anhydrous Na₂SO₄, filtered, and concentrated. The residue was purified by column chromatography on silica gel (EtOAc/hexane, 10:90 to 15:85) to afford 28a (108.5 mg, 74%) as a white solid; ¹H NMR (CDCl₃, 600 MHz) 8.35 (2 H, s), 7.36-7.32 (4 H, m), 7.26-7.24 (1 H, m), 5.42 (1 H, br), 5.10 (1 H, quit, J=7.3 Hz), 1.55 (3 H, d, J=6.1 Hz); HRMS (ESI-TOF) m/z: [M+H]⁺ calculated for C₁₂H₁₃IN₃ 326.0149; found 326.0695.

### 5-Iodo-N-phenethylpyrimidin-2-amine (28b)

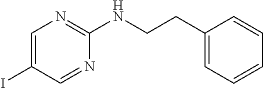 [SMILES: Ic1cnc(NCCc2ccccc2)nc1]

The above compound (28b) was prepared from 4a and 26b following Method G. The reaction was stirred at 60° C. for 16 h. The residue was purified by column chromatography on silica gel (EtOAc/hexane, 20:80 to 30:70) to afford 28b (8%) as a white solid; ¹H NMR (CDCl₃, 600 MHz) 8.36 (2 H, s), 7.31 (2 H, t, J=7.3 Hz), 7.24-7.21 (3 H, m), 5.22 (1 H, br), 3.65 (2 H, q, J=7.3 Hz), 2.90 (2 H, t, J=7.3 Hz); HRMS (ESI-TOF) m/z: [M+H]⁺ calculated for C₁₂H₁₃IN₃ 326.0149; found 326.0129.

### N-(5-Iodopyrimidin-2-yl)benzamide (28c)

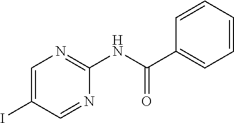 [SMILES: O=C(Nc1ncc(I)cn1)c1ccccc1]

The above compound (28c) was prepared from 4a and 26c following Method G. The reaction was stirred at room temperature for 24 h. The residue was purified by column chromatography on silica gel (EtOAc/hexane, 20:80 to 30:70) to afford 28c (14%) as a yellow solid; ¹H NMR (CDCl₃, 600 MHz) 8.36 (2 H, s), 7.31 (2 H, t, J=7.3 Hz), 7.24-7.21 (3 H, m), 5.22 (1 H, br), 3.65 (2 H, q, J=7.3 Hz), 2.90 (2 H, t, J=7.3 Hz); HRMS (ESI-TOF) m/z: [M+H]⁺ calculated for C₁₁H₉IN₃O 325.9785; found 325.9786.

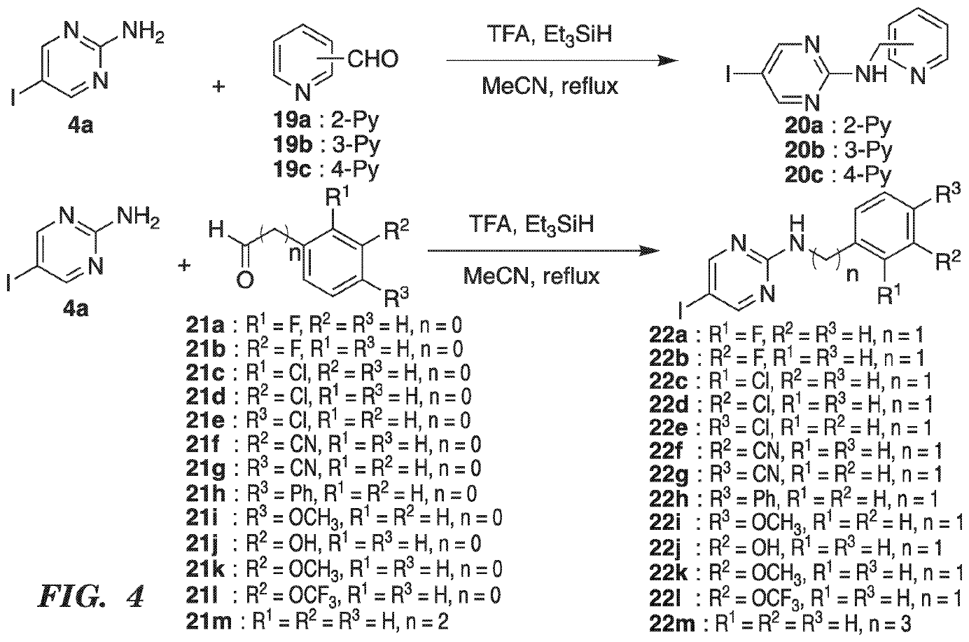

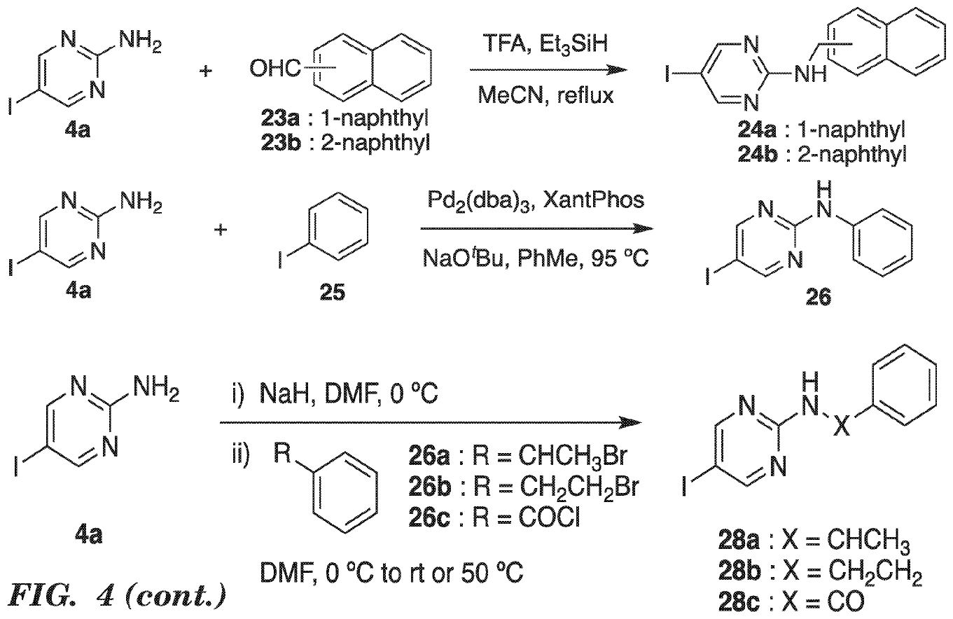

The synthetic schemes for intermediates 20a-c, 22a-m, 24a-b, 26, and 28a-c, discussed supra, are shown in FIG. 4.

### 5-(2-((2-Fluorobenzyl)amino)pyrimidin-5-yl)-1H-benzo[d]imidazol-2(3H)-one (29a)

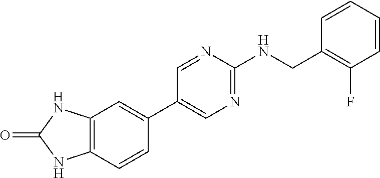 [SMILES: O=c1[nH]c2ccc(-c3cnc(NCc4ccccc4F)nc3)cc2[nH]1]

The above compound (29a) was prepared from 13b and 22a following Method F and purified by column chromatography on silica gel (MeOH/CH₂Cl₂, 5:95 to 10:90) to give 29a (15%) as a yellow solid; ¹H NMR (CD₃OD, 600 MHz) 8.51 (2 H, s), 7.39 (1 H, t, J=7.3 Hz), 7.25 (1 6H, q, J=7.3 Hz), 7.21-7.19 (2 H, m), 7.11-7.05 (3 H, m), 4.68 (2 H, s); HRMS (ESI-TOF) m/z: [M+H]⁺ calculated for C₁₈H₁₅FN₅O 336.1255; found 336.1259.

### 5-(2-((3-Fluorobenzyl)amino)pyrimidin-5-yl)-1H-benzo[d]imidazol-2(3H)-one (29b)

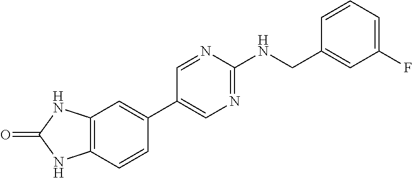 [SMILES: O=c1[nH]c2ccc(-c3cnc(NCc4cccc(F)c4)nc3)cc2[nH]1]

The above compound (29b) was prepared from 13b and 22b following Method F and purified by column chromatography on silica gel (MeOH/CH₂Cl₂, 5:95 to 10:90) to give 29b (21%) as a yellow solid; ¹H NMR (CD₃OD, 600 MHz) 8.51 (2 H, s), 7.30 (1 H, q, J=7.3 Hz), 7.21-7.19 (3 H, m), 7.11-7.07 (2 H, m), 6.93 (1 H, td, J=8.5, 2.4 Hz), 4.63 (2 H, s); HRMS (ESI-TOF) m/z: [M+H]⁺ calculated for C₁₈H₁₅FN₅O 336.1255; found 336.1250.

### 5-(2-((2-Chlorobenzyl)amino)pyrimidin-5-yl)-1H-benzo[d]imidazol-2(3H)-one (29c)

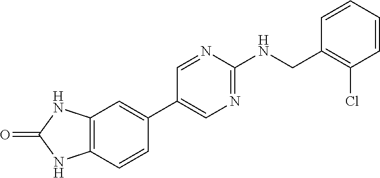 [SMILES: O=c1[nH]c2ccc(-c3cnc(NCc4ccccc4Cl)nc3)cc2[nH]1]

The above compound (29c) was prepared from 13b and 22c following Method F and purified by column chromatography on silica gel (MeOH/CH₂Cl₂, 5:95) to give 29c (15%) as a yellow solid; ¹H NMR (CD₃OD, 600 MHz) 8.51 (2 H, s), 7.40-7.39 (2 H, m), 7.25-7.22 (2 H, m), 7.21-7.19 (2 H, m), 7.10 (1 H, d, J=8.5 Hz), 4.71 (2 H, s); HRMS (ESI-TOF) m/z: [M+H]⁺ calculated for C₁₈H₁₅ClN₅O 352.0960; found 352.0969.

### 5-(2-((3-Chlorobenzyl)amino)pyrimidin-5-yl)-1H-benzo[d]imidazol-2(3H)-one (29d)

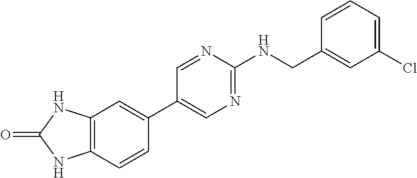 [SMILES: O=c1[nH]c2ccc(-c3cnc(NCc4cccc(Cl)c4)nc3)cc2[nH]1]

The above compound (29d) was prepared from 13b and 22d following Method F and purified by column chromatography on silica gel (MeOH/CH₂Cl₂, 5:95) to give 29d (18%) as a yellow solid; ¹H NMR (CD₃OD, 600 MHz) 8.51 (2 H, s), 7.36 (1 H, s), 7.28 (2 H, d, J=6.1 Hz), 7.22-7.19 (3 H, m), 7.10 (1 H, d, J=8.5 Hz), 4.62 (2 H, s); HRMS (ESI-TOF) m/z: [M+H]⁺ calculated for C₁₈H₁₅ClN₅O 352.0960; found 352.0940.

### 5-(2-((4-Chlorobenzyl)amino)pyrimidin-5-yl)-1H-benzo[d]imidazol-2(3H)-one (29e)

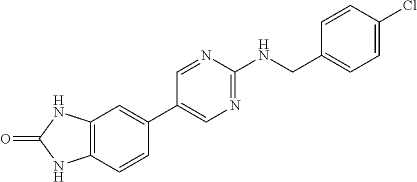 [SMILES: O=c1[nH]c2ccc(-c3cnc(NCc4ccc(Cl)cc4)nc3)cc2[nH]1]

The above compound (29e) was prepared from 13b and 22e following Method F and purified by column chromatography on silica gel (MeOH/CH₂Cl₂, 5:95) to give 29e (21%) as a yellow solid; ¹H NMR (CD₃OD, 600 MHz) 8.50 (2 H, s), 7.34 (2 H, d, J=8.5 Hz), 7.29 (2 H, d, J=8.5 Hz), 7.21-7.18 (2 H, m), 7.10 (1 H, d, J=7.3 Hz), 4.60 (2 H, s); HRMS (ESI-TOF) m/z: [M+H]⁺ calculated for C₁₈H₁₅ClN₅O 352.0960; found 352.0963.

### 3-(((5-(2-Oxo-2,3-dihydro-1H-benzo[d]imidazol-5-yl)pyrimidin-2-yl)amino)methyl)benzonitrile (29f)

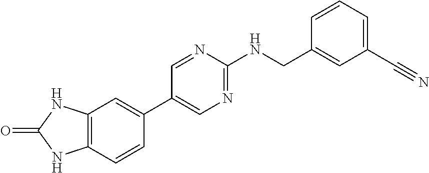 [SMILES: N#Cc1cccc(CNc2ncc(-c3ccc4[nH]c(=O)[nH]c4c3)cn2)c1]

The above compound (29f) was prepared from 13b and 22f following Method F and purified by column chromatography on silica gel (MeOH/CH₂Cl₂, 5:95) to give 29f (20%) as a yellow solid; ¹H NMR (DMSO-d₆, 600 MHz) 10.69 (1 H, s), 10.64 (1 H, s), 8.53 (2 H, s), 7.87 (1 H, t, J=6.1 Hz), 7.74 (1 H, s), 7.69 (1 H, d, J=8.5 Hz), 7.66 (1 H, d, J=8.5 Hz), 7.52 (1 H, t, J=7.3 Hz), 7.13 (1 H, t, J=9.8 Hz), 7.08 (1 H, s), 6.95 (1 H, d, J=8.5 Hz), 4.57 (2 H, d, J=7.3 Hz); HRMS (ESI-TOF) m/z: [M+H]⁺ calculated for C₁₉H₁₅N₆O 343.1302; found 343.1291.

### 4-(((5-(2-Oxo-2,3-dihydro-1H-benzo[d]imidazol-5-yl)pyrimidin-2-yl)amino)methyl)benzonnrile (29g)

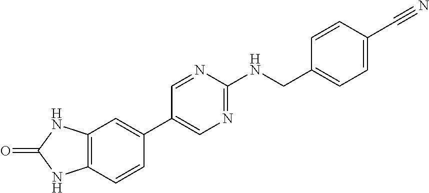 [SMILES: N#Cc1ccc(CNc2ncc(-c3ccc4[nH]c(=O)[nH]c4c3)cn2)cc1]

The above compound (29g) was prepared from 13b and 22g following Method F and purified by column chromatography on silica gel (MeOH/CH₂Cl₂, 5:95 to 10:90) to give 29g (24%) as a yellow solid; ¹H NMR (DMSO-d₆, 600 MHz) 10.69 (1 H, s), 10.64 (1 H, s), 8.52 (2 H, s), 7.90 (1 H, t, J=6.1 Hz), 7.77 (2 H, d, J=8.5 Hz), 7.49 (2 H, d, J=7.3 Hz), 7.12 (1 H, d, J=6.1 Hz), 7.07 (1 H, s), 6.95 (1 H, d, J=8.5 Hz), 4.60 (2 H, d, J=6.1 Hz); HRMS (ESI-TOF) m/z: [M+H]⁺ calculated for C₁₉H₁₅N₆O 343.1302; found 343.1300.

### 5-(2-(([1,1′-Biphenyl]-4-ylmethyl)amino)pyrimidin-5-yl)-1H-benzo[d]imidazol-2(3H)-one (29h)

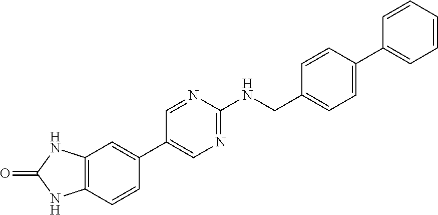 [SMILES: O=c1[nH]c2ccc(-c3cnc(NCc4ccc(-c5ccccc5)cc4)nc3)cc2[nH]1]

The above compound (29h) was prepared from 13b and 22h following Method F and purified by column chromatography on silica gel (MeOH/CH₂Cl₂, 5:95 to 10:90) to give 29h (18%) as a yellow solid; ¹H NMR (DMSO-d₆, 600 MHz) 10.69 (1 H, s), 10.64 (1 H, s), 8.53 (2 H, s), 7.84 (1 H, t, J=6.1 Hz), 7.63-7.59 (4 H, m), 7.45-7.40 (4 H, m), 7.33 (1 H, d, J=7.3 Hz), 7.13 (1 H, d, J=7.3 Hz), 7.08 (1 H, s), 6.95 (1 H, d, J=8.5 Hz), 4.57 (2 H, d, J=4.9 Hz); HRMS (ESI-TOF) m/z: [M+H]⁺ calculated for C₂₄H₂₀N₅O 394.1662; found 394.1663.

### 5-(2-((4-Methoxybenzyl)amino)pyrimidin-5-yl)-1H-benzo[d]imidazol-2 (3H)-one (29i)

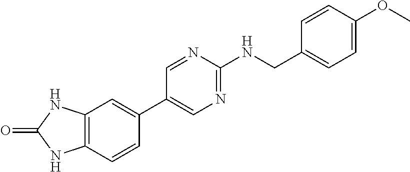 [SMILES: COc1ccc(CNc2ncc(-c3ccc4[nH]c(=O)[nH]c4c3)cn2)cc1]

The above compound (29i) was prepared from 13b and 22i following Method F and purified by column chromatography on silica gel (MeOH/CH₂Cl₂, 5:95 to 10:90) to give 29i (12%) as a yellow solid; ¹H NMR (DMSO-d₆, 600 MHz) 10.71 (1 H, s), 10.65 (1 H, s), 8.51 (2 H, s), 7.74 (1 H, t, J=6.1 Hz), 7.24 (2 H, d, J=7.3 Hz), 7.12 (1 H, d, J=7.3 Hz), 7.07 (1 H, s), 6.95 (1 H, d, J=7.3 Hz), 6.85 (2 H, d, J=8.5 Hz), 4.45 (2 H, d, J=6.1 Hz), 3.70 (3 H, s); HRMS (ESI-TOF) m/z: [M+H]⁺ calculated for C₁₉H₁₈N₅O₂ 348.1455; found 348.1619.

### 5-(2-((1-Phenylethyl)amino)pyrimidin-5-yl)-1H-benzo[d]imidazol-2(3H)-one (29j)

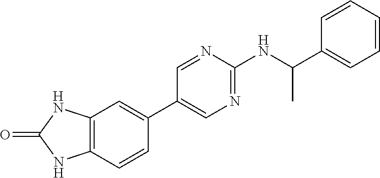 [SMILES: CC(Nc1ncc(-c2ccc3[nH]c(=O)[nH]c3c2)cn1)c1ccccc1]

The above compound (29j) was prepared from 13b and 28a following Method F and purified by column chromatography on silica gel (MeOH/CH₂Cl₂, 5:95) to give 29j (32%) as a pale yellow solid; ¹H NMR (CD₃OD, 600 MHz) 8.44 (2 H, s), 7.38 (2 H, d, J=7.3 Hz), 7.28 (2 H, t, J=7.3 Hz), 7.19-7.15 (3 H, m), 7.07 (1 H, d, J=8.5 Hz), 5.15 (1 H, q, J=7.3 Hz), 1.53 (3 H, d, J=6.1 Hz); HRMS (ESI-TOF) m/z: [M+H]⁺ calculated for C₁₉H₁₈N₅O 332.1506; found 332.1976.

### 5-(2-(Phenethylamino)pyrimidin-5-yl)-1H-benzo[d]imidazol-2(3H)-one (29k)

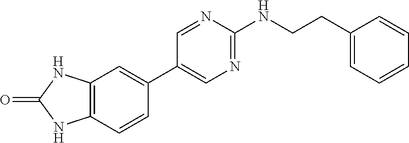 [SMILES: O=c1[nH]c2ccc(-c3cnc(NCCc4ccccc4)nc3)cc2[nH]1]

The above compound (29k) was prepared from 13b and 28b following Method F and purified by column chromatography on silica gel (MeOH/CH₂Cl₂, 5:95) to give 29k (17%) as a yellow solid; ¹H NMR (DMSO-d₆, 600 MHz) 10.71 (1 H, s), 10.66 (1 H, s), 8.52 (2 H, s), 7.30-7.27(3 H, m), 7.24 (2 H, d, J=7.3 Hz), 7.20-7.18 (1 H, m), 7.14(1 H, d, J=8.5 Hz), 7.08 (1 H, s), 6.96 (1 H, d, J=7.9 Hz), 3.51 (2 H, q, J=8.5 Hz), 2.85 (2 H, t, J=8.5 Hz); EIRMS (ESI-TOF) m/z: [M+H]⁺ calculated for C₁₉H₁₈N₅O 332.1506; found 332.1551.

### 5-(2-((3-Phenylpropyl)amino)pyrimidin-5-yl)-1H-benzo[d]imidazol-2(3H)-one (291)

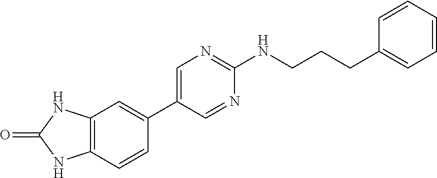 [SMILES: O=c1[nH]c2ccc(-c3cnc(NCCCc4ccccc4)nc3)cc2[nH]1]

The above compound (291) was prepared from 13b and 22m following Method I and purified by column chromatography on silica gel (MeOH/CH₂Cl₂, 5:95) to give 291 (34%) as a white solid; ¹H NMR (DMSO-d₆, 600 MHz) 10.66 (1 H, s), 10.61 (1 H, s), 8.50 (2 H, s), 7.28-7.24 (3 H, m), 7.22 (2 H, d, J=7.6 Hz), 7.16 (1 H, t, J=7.65 Hz), 7.12 (1 H, d, J=8.1 Hz), 7.07 (1 H, s), 6.96 (1 H, d, J=8.0 Hz), 3.33-3.29 (2 H, m), 2.64 (2 H, t, J=7.6 Hz), 1.84 (2 H, quin, J=7.6 Hz); HRMS (ESI-TOF) m/z: [M+H]⁺ calculated for C₂₀H₂₀N₅O 346.1663; found 346.1684.

### 5-(2-((Pyridin-2-ylmethyl)amino)pyrimidin-5-yl)-1H-benzo[d]yl)-2 (3H)-one (30a)

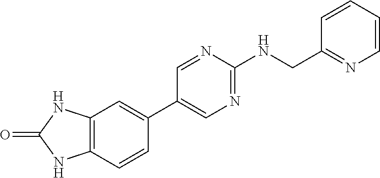 [SMILES: O=c1[nH]c2ccc(-c3cnc(NCc4ccccn4)nc3)cc2[nH]1]

The above compound (30a) was prepared from 13b and 20a following Method F and purified by column chromatography on silica gel (MeOH/CH₂Cl₂, 10:90) to give 30a (7%) as a brown solid; ¹H NMR (DMSO-d₆, 600 MHz) 10.70 (1 H, s), 10.65 (1 H, s), 8.55 (2 H, s), 7.88-7.84 (2 H, m), 7.43-7.36 (3 H, m), 7.13 (1 H, d, J=7.3 Hz), 7.08 (1 H, s), 6.96 (1 H, d, J=8.5 Hz), 4.66 (2 H, d, J=2.4 Hz); HRMS (ESI-TOF) m/z: [M+H]⁺ calculated for C₁₇H₁₅N₆O 319.1302; found 319.1311.

### 5-(2-((Pyridin-3-ylmethyl)amino)pyrimidin-5-yl)-1H-benzo[d]imidazol-2(3H)-one (30b)

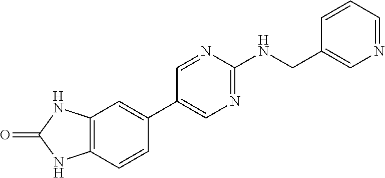 [SMILES: O=c1[nH]c2ccc(-c3cnc(NCc4cccnc4)nc3)cc2[nH]1]

The above compound (30b) was prepared from 13b and 20b following Method F and purified by column chromatography on silica gel (MeOH/CH₂Cl₂, 5:95 to 10:90) to give 30b (16%) as a pale yellow solid; ¹H NMR (DMSO-d₆, 600 MHz) 10.71 (1 H, s), 10.66 (1 H, s), 8.55 (2 H, s), 8.25 (1 H, d, J=6.7 Hz), 7.95 (1 H, t, J=6.7 Hz), 7.79 (1 H, t, J=7.3 Hz), 7.18 (1 H, s), 7.13 (1 H, d, J=8.5 Hz), 7.09-7.08 (2 H, m), 6.96 (1 H, d, J=7.3 Hz), 4.66 (2 H, d, J=6.1 Hz); HRMS (ESI-TOF) m/z: [M+H]⁺ calculated for C₁₇H₁₅N₆O 319.1302; found 319.1284.

### 5-(2-((Pyridin-4-ylmethyl)amino)pyrimidin-5-yl)-1H-benzo[d]imidazol-2 (3H)-one (30c)

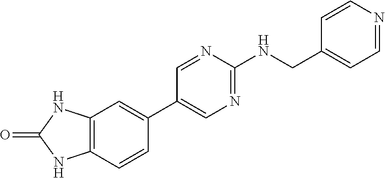 [SMILES: O=c1[nH]c2ccc(-c3cnc(NCc4ccncc4)nc3)cc2[nH]1]

The above compound (30c) was prepared from 13b and 20c following Method F and purified by column chromatography on silica gel (MeOH/CH₂Cl₂, 10:90) to give 30c (16%) as a yellow solid; ¹H NMR (CD₃OD, 600 MHz) 8.51-8.47 (3 H, m), 7.53 (2 H, d, J=6.1 Hz), 7.50 (1 H, d, J=6.1 Hz), 7.20-7.18 (2 H, m), 7.10 (1 H, d, J=8.5 Hz), 4.73 (2 H, s); HRMS (ESI-TOF) m/z: [M+H]⁺ calculated for C₁₇H₁₅N₆O 319.1302; found 319.1297.

### 5-(2-((Naphthalen-1-ylmethyl)amino)pyrimidin-5-yl)-1H-benzo[d]imidazol-2 (3H)-one (30d)

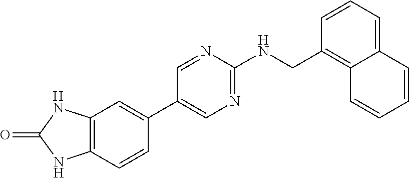 [SMILES: O=c1[nH]c2ccc(-c3cnc(NCc4cccc5ccccc45)nc3)cc2[nH]1]

The above compound (30d) was prepared from 13b and 24a following Method F and purified by column chromatography on silica gel (MeOH/CH₂Cl₂, 10:90) to give 30d (21%) as a yellow solid; ¹H NMR (DMSO-d₆, 600 MHz) 10.69 (1 H, s), 10.64 (1 H, s), 8.54 (2 H, s), 8.17 (1 H, d, J=8.5 Hz), 7.94 (1 H, d, J=7.3 Hz), 7.85 (1 H, t, J=6.1 Hz), 7.81 (1 H, d, J=7.3 Hz), 7.57-7.52 (2 H, m), 7.47-7.43 (2 H, m), 7.13 (1 H, d, J=6.1 Hz), 7.08 (1 H, s), 6.95 (1 H, d, J=8.5 Hz), 5.00 (2 H, d, J=7.3 Hz); HRMS (ESI-TOF) m/z: [M+H]⁺ calculated for C₂₂H₁₈N₅O 368.1506; found 368.1695.

### 5-(2-((Naphthalen-2-ylmethyl)amino)pyrimidin-5-yl)-1H-benzo[d]imidazol-2(3H)-one (30e)

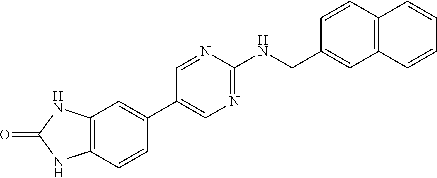 [SMILES: O=c1[nH]c2ccc(-c3cnc(NCc4ccc5ccccc5c4)nc3)cc2[nH]1]

The above compound (30e) was prepared from 13b and 24b following Method F and purified by column chromatography on silica gel (MeOH/CH₂Cl₂, 2.5:97.5 to 5:95) to give 30e (13%) as a yellow solid; ¹H NMR (DMSO-d₆, 600 MHz) 10.68 (1 H, s), 10.63 (1 H, s), 8.53 (2 H, s), 7.91 (1 H, t, J=7.3 Hz), 7.86-7.84 (3 H, m), 7.79 (1 H, s), 7.50 (1 H, d, J=7.3 Hz), 7.48-7.43 (2 H, m), 7.13 (1 H, d, J=8.5 Hz), 7.07 (1 H, s), 6.95 (1 H, d, J=7.3 Hz), 4.70 (2 H, d, J=6.1 Hz); HRMS (ESI-TOF) m/z: [M+H]⁺ calculated for C₂₂H₁₈N₅O 368.1506; found 368.1486.

### 5-(6-((Naphthalen-1-ylmethyl)amino)pyridazin-3-yl)-1H-benzo[d]imidazol-2 (3H)-one (31)

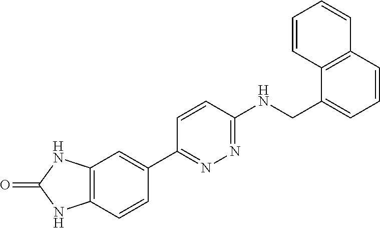 [SMILES: O=c1[nH]c2ccc(-c3ccc(NCc4cccc5ccccc45)nn3)cc2[nH]1]

To a solution 3c (40 mg, 0.148 mmol), 13b (42 mg, 0.148 mmol) and Pd(dppf)Cl₂ (12 mg, 0.015 mmol) in DMF (2 mL) and H₂O (0.5 mL) was added K₃PO₄ (94 mg, 0.444 mmol). The mixture was stirred at 90° C. under nitrogen for 12 h. After cooled to room temperature, the mixture was concentrated and purified by column chromatography on silica gel (MeOH/CH₂Cl₂, 20:80) to give 31 (10 mg, 18%) as a dark yellow solid; ¹H NMR (DMSO-d₆, 600 MHz) 10.98 (1 H, s), 10.95 (1 H, s), 8.35-8.33 (1 H, d), 8.14-8.13 (1 H, d), 8.03-8.02 (1 H, d), 7.99-7.97 (1 H, d), 7.65-7.53 (7 H, m), 7.11-7.09 (1 H, d), 5.09 (2 H, d,); HRMS (ESI-TOF) m/z: [M+H]⁺ calculated for C₂₂H₁₈N₅O 368.1506; found 368.1502.

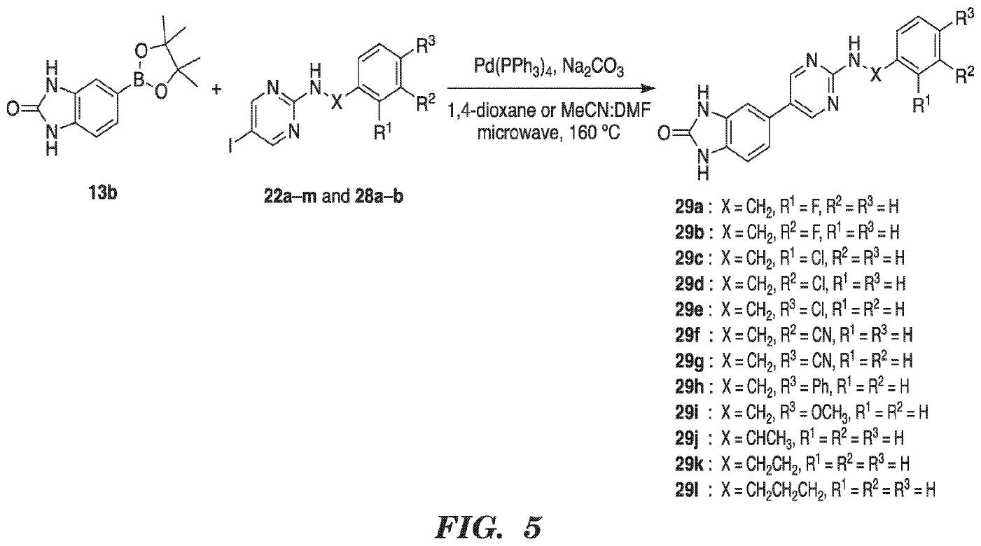

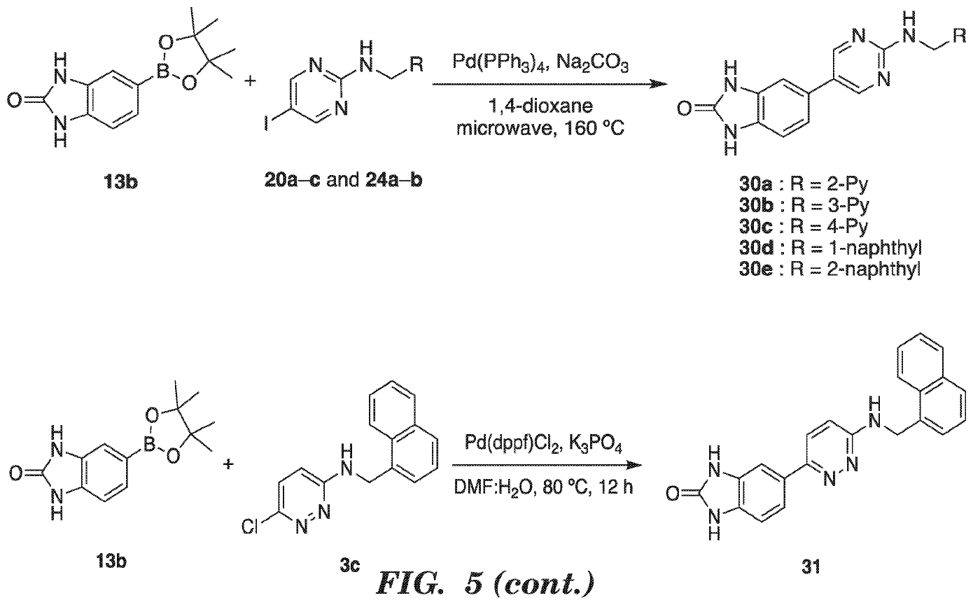

The synthetic schemes for compounds 29a-1, 30a-e, and 31, discussed supra, are shown in FIG. 5.

### Example 9—General Procedure for the Preparation of (((5-(2-Oxo-2,3-dihydro-1H-benzo[d]imidazol-5-yl)pyrimidin-2-yl)amino)methyl)benzamide—Method H

### 3-(((5-(2-Oxo-2,3-dihydro-1H-benzo[d]imidazol-5-yl)pyrimidin-2-yl)amino)methyl)benzamide (32a)

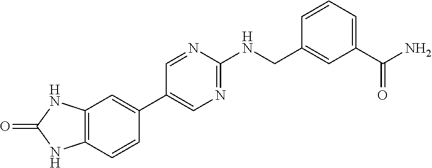 [SMILES: NC(=O)c1cccc(CNc2ncc(-c3ccc4[nH]c(=O)[nH]c4c3)cn2)c1]

To a mixture of 29f (4.2 mg, 0.012 mmol) and K₂CO₃ (0.5 mg, 0.004 mmol) in anhydrous DMF (0.2 mL) was added 50% hydrogen peroxide (0.01 mL) at 0° C. The reaction was stirred at 0° C. to room temperature for 16 h. The reaction was filtered and solvent was removed in vacuo. The crude product was washed with ether and hexane to afford 32a (3.2 mg, 74%) as a pale yellow solid; ¹H NMR (DMSO-d₆, 600 MHz) 10.69 (1 H, s), 10.64 (1 H, s), 8.52 (2 H, s), 7.93 (1 H, s), 7.83 (1 H, d, J=7.3 Hz), 7.71 (1 H, d, J=7.3 Hz), 7.46 (1 H, d, J=7.3 Hz), 7.38-7.35 (2 H, m), 7.32 (1 H, s), 7.13 (1 H, d, J=8.5 Hz), 7.08 (1 H, s), 6.95 (1 H, d, J=7.3 Hz); HRMS (ESI-TOF) m/z: [M+H]⁺ calculated for C₁₉H₁₇N₆O₂ 361.1408; found 361.1404.

### 4-(((5-(2-Oxo-2,3-dihydro-1H-benzo[d]imidazo1-5-yl)pyrimidin-2-yl)amino)methyl)benzamide (32b)

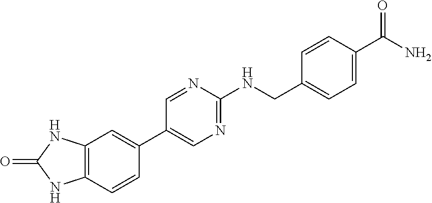 [SMILES: NC(=O)c1ccc(CNc2ncc(-c3ccc4[nH]c(=O)[nH]c4c3)cn2)cc1]

The above compound (32b) was prepared from 29g following Method H and give 32b (94%) as a yellow solid; ¹H NMR (DMSO-d₆, 600 MHz) 8.50 (2 H, s), 7.89 (1 H, s), 7.80-7.79 (3 H, m), 7.36 (2 H, d, J=8.5 Hz), 7.28 (1 H, s), 7.05-7.02 (2 H, m), 6.89 (1 H, d, J=8.5 Hz), 4.57 (2 H, d, J=6.1 Hz); HRMS (ESI-TOF) m/z: [M+H]⁺ calculated for C₁₉H₁₇N₆O₂ 361.1408; found 361.1380.

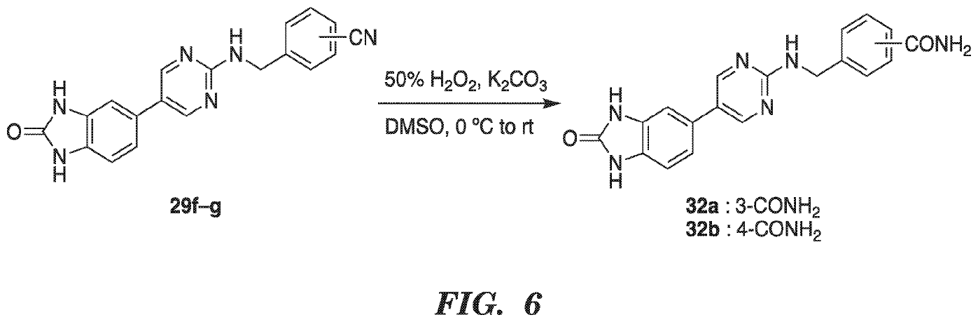

The synthetic schemes for compounds 32a-b, discussed supra, are shown in FIG. 6.

### 5-Bromo-N-phenylpyrimidine-2-carboxamide (34)

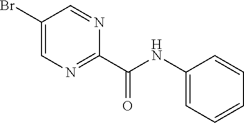 [SMILES: O=C(Nc1ccccc1)c1ncc(Br)cn1]

To a mixture of 33 (100.0 mg, 0.49 mmol), HATU (225.0 mg, 0.59 mmol), and aniline (0.045 mL, 0.49 mmol) in anhydrous DMF (1 mL) was added N,N-diisopropylethylamine (0.17 mL, 0.98 mmol) under nitrogen. The reaction mixture was stirred at 80° C. for 16 h. After being quenched by the addition of water, the aqueous layer was extracted with EtOAc (2×15 mL). The combined organic extracts were washed with brine, dried over anhydrous Na₂SO₄, filtered, and concentrated. The residue was purified by column chromatography on silica gel (EtOAc/hexane, 50:50) to give 34 (92.2 mg, 67%) as a yellow solid; ¹H NMR (CDCl₃, 600 MHz) 9.77 (1 H, s), 8.98 (2 H, s), 7.78 (2 H, d, J=8.5 Hz), 7.41 (2 H, t, J=7.3 Hz), 7.19 (1 H, t, J=7.3 Hz); HRMS (ESI-TOF) m/z: [M+H]⁺ calculated for C₁₁H₉⁷⁹BrN₃O and C₁₁H₉⁸¹BrN₃O 277.9924, 279.9904; found 277.9920, 279.9899.

### N-((5-Bromopyrimidin-2-yl)methyl)aniline (36)

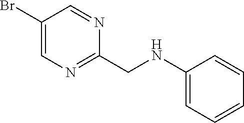 [SMILES: Brc1cnc(CNc2ccccc2)nc1]

To a mixture of 35 (130.0 mg, 0.52 mmol) and K₂CO₃ (143.0 mg, 1.03 mmol) in anhydrous acetonitrile (1 mL) was added aniline (0.05 mL, 0.52 mmol) under nitrogen. The reaction mixture was stirred at 80° C. for 4 h. After being quenched by the addition of water, the aqueous layer was extracted with EtOAc (2×15 mL). The combined organic extracts were washed with brine, dried over anhydrous Na₂SO₄, filtered, and concentrated. The residue was purified by column chromatography on silica gel (EtOAc/hexane, 20:80 to 30:70) to give 36 (81.3 mg, 60%) as a yellow solid; ¹H NMR (CDCl₃, 600 MHz) 8.78 (2 H, s), 7.20 (2 H, t, J=7.3 Hz), 6.76-6.71 (3 H, m), 4.94 (1 H, br), 4.54 (2 H, s); HRMS (ESI-TOF) m/z: [M+H]⁺ calculated for C₁₁H₁₁⁷⁹BrN₃ and C₁₁H₁₁⁸¹BrN₃ 264.0131, 266.0111; found 264.0117, 266.0096.

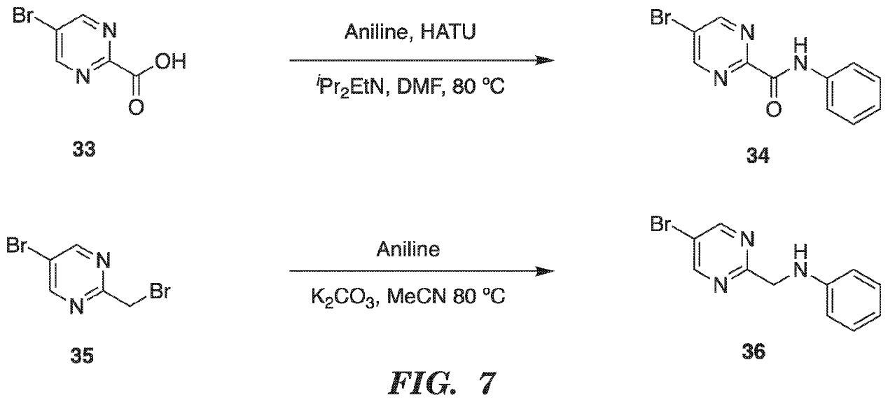

The synthetic schemes for intermediate compounds 34 and 36, discussed supra, are shown in FIG. 7.

### Example 10—General Procedure for the Preparation of Benzimidazolonyl Benzyl Heterocyclic Amines—Method I

### 5-(2-((3-Hydroxybenzyl)amino)pyrimidin-5-yl)-1H-benzo[d]imidazol-2(3H)-one (37a)

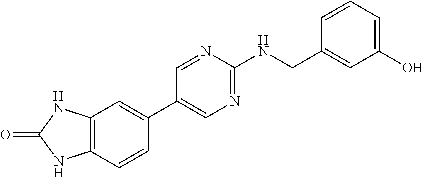 [SMILES: O=c1[nH]c2ccc(-c3cnc(NCc4cccc(O)c4)nc3)cc2[nH]1]

To a mixture of 13b (32.0 mg, 0.12 mmol), 22j (40.0 mg, 0.12 mmol), and Pd(PPh₃)₄ (14.0 mg, 0.01 mmol) in a microwave reaction vial was added anhydrous DMF (1 mL), was added anhydrous acetonitrile (1 mL) and 1 M Na₂CO₃ (0.24 mL, 0.24 mmol) under nitrogen. The reaction mixture was irradiated under microwave conditions for 15 min at 160° C. After being quenched by the addition of water, the aqueous layer was extracted with EtOAc (2×10 mL). The combined organic extracts were washed with brine, dried over anhydrous Na₂SO₄, filtered, and concentrated. The residue was purified by column chromatography on silica gel (MeOH/CH₂Cl₂, 10:90 to 15:85) to give 37a (13.6 mg, 33%) as a brown solid; ¹H NMR (DMSO-d₆, 600 MHz) 10.70 (1 H, s), 10.64 (1 H, s), 9.26 (1 H, s), 8.51 (2 H, s), 7.74 (1 H, t, J=7.3 Hz), 7.13 (1 H, d, J=7.3 Hz), 7.08-7.06 (2 H, m), 6.95 (1 H, d, J=7.3 Hz), 6.73-6.71 (2 H, m), 6.58 (1 H, d, J=7.3 Hz), 4.45 (2 H, d, J=6.1 Hz); HRMS (ESI-TOF) m/z: [M+H]⁺ calculated for C₁₈H₁₆N₅O₂ 334.1299; found 334.1312.

### 5-(2-((3-Methoxybenzyl)amino)pyrimidin-5-yl)-1H-benzo[d]imidazol-2(3H)-one (37b)

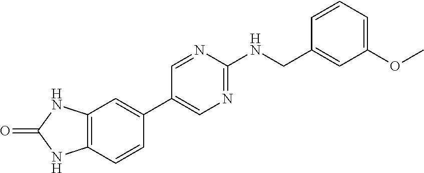 [SMILES: COc1cccc(CNc2ncc(-c3ccc4[nH]c(=O)[nH]c4c3)cn2)c1]

The above compound (37b) was prepared from 13b and 22k following Method I and purified by column chromatography on silica gel (MeOH/CH₂Cl₂, 5:95 to 10:90) to give 37b (22%) as a yellow solid; ¹H NMR (DMSO-d₆, 600 MHz) 10.70 (1 H, s), 10.65 (1 H, s), 8.52 (2 H, s), 7.78 (1 H, t, J=6.1 Hz), 7.20 (1 H, t, J=7.3 Hz), 7.13 (1 H, d, J=7.3 Hz), 7.07 (1 H, s), 6.95 (1 H, d, J=8.5 Hz), 6.88 (2 H, s), 6.77 (1 H, d, J=8.5 Hz), 4.50 (2 H, d, J=7.3 Hz), 3.71 (3 H, s); HRMS (ESI-TOF) m/z: [M+H]⁺ calculated for C₁₉H₁₈N₅O₂ 348.1455; found 348.1694.

### 5-(2-((3-(Trifluoromethoxy)benzyl)amino)pyrimidin-5-yl)-1H-benzo[d]imidazol-2 (3H)-one (37c)

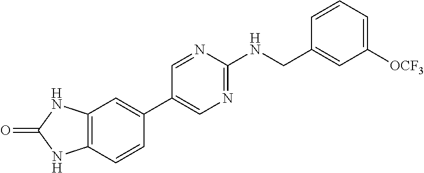 [SMILES: O=c1[nH]c2ccc(-c3cnc(NCc4cccc(OC(F)(F)F)c4)nc3)cc2[nH]1]

The above compound (37c) was prepared from 13b and 221 following Method I and purified by column chromatography on silica gel (MeOH/CH₂Cl₂, 5:95) to give 37c (32%) as a yellow solid; ¹H NMR (DMSO-d₆, 600 MHz) 10.70 (1 H, s), 10.65 (1 H, s), 8.53 (2 H, s), 7.87 (1 H, t, J=7.3 Hz), 7.44 (1 H, t, J=8.5 Hz), 7.35 (1 H, d, J=7.3 Hz), 7.28 (1 H, s), 7.20 (1 H, d, J=7.3 Hz), 7.13 (1 H, d, J=7.3 Hz), 7.08 (1 H, s), 6.95 (1 H, d, J=7.3 Hz), 4.57 (2 H, d, J=4.9 Hz); HRMS (ESI-TOF) m/z: [M+H]⁺ calculated for C₁₉H₁₅F₃N₅O₂ 402.1172; found 402.1136.

### 5-(2-Aminopyrimidin-5-yl)-1H-benzo[d]imidazol-2 (3H)-one (38a)

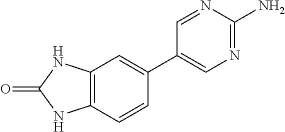 [SMILES: Nc1ncc(-c2ccc3[nH]c(=O)[nH]c3c2)cn1]

The above compound (38a) was prepared from 13b and 28c following Method I and purified by column chromatography on silica gel (MeOH/CH₂Cl₂, 10:90) to give 38a (17%) as a yellow solid; ¹H NMR (DMSO-d₆, 600 MHz) 10.71 (1 H, s), 10.66 (1 H, s), 8.46 (2 H, s), 7.13 (1 H, d, J=8.5 Hz), 7.07 (1 H, s), 6.95 (1 H, d, J=7.3 Hz), 6.67 (2 H, s); HRMS (ESI-TOF) m/z: [M+H]⁺ calculated for C₁₁H₁₀N₅O 228.0880; found 228.0888.

### 5-(2-(Phenylamino)pyrimidin-5-yl)-1H-benzo[d]imidazol-2(3H)-one (38b)

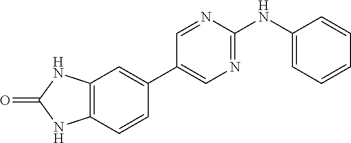 [SMILES: O=c1[nH]c2ccc(-c3cnc(Nc4ccccc4)nc3)cc2[nH]1]

The above compound (38b) was prepared from 13b and 26 following Method I and purified by column chromatography on silica gel (MeOH/CH₂Cl₂, 5:95) to give 38b (35%) as a yellow solid; ¹H NMR (DMSO-d₆, 600 MHz) 10.77 (1 H, s), 10.71 (1 H, s), 9.71 (1 H, s), 8.74 (2 H, s), 7.78 (2 H, d, J=8.5 Hz), 7.28 (2 H, t, J=8.5 Hz), 7.24 (1 H, d, J=7.3 Hz), 7.18 (1 H, s), 7.00 (1 H, d, J=8.5 Hz), 6.94 (1 H, t, J=8.5 Hz), 6.67 (2 H, s); HRMS (ESI-TOF) m/z: [M+H]⁺ calculated for C₁₇H₁₄N₅O 304.1193; found 304.1169.

### 5-(2-Oxo-2,3-dihydro-1H-benzo[d]imidazol-5-yl)-N-phenylpyrimidine-2-carboxamide (38c)

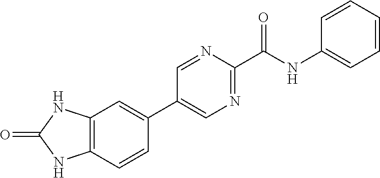 [SMILES: O=C(Nc1ccccc1)c1ncc(-c2ccc3[nH]c(=O)[nH]c3c2)cn1]

The above compound (38c) was prepared from 13b and 34 following Method I and purified by column chromatography on silica gel (MeOH/CH₂Cl₂, 10:90 to 15:85) to give 38c (11%) as a yellow solid; ¹H NMR (DMSO-d₆, 600 MHz) 10.93 (1 H, s), 10.91 (1 H, s), 10.78 (1 H, s), 9.28 (2 H, s), 7.90 (2 H, d, J=8.5 Hz), 7.48 (1 H, d, J=7.3 Hz), 7.41 (1 H, s), 7.37 (2 H, t, J=7.3 Hz), 7.14-7.10 (2 H, m); HRMS (ESI-TOF) m/z: [M+H]⁺ calculated for C₁₈H₁₄N₅O₂ 332.1142; found 332.1129.

### 5-(2-((Phenylamino)methyl)pyrimidin-5-yl)-1H-benzo[d]imidazol-2(3H)-one (38d)

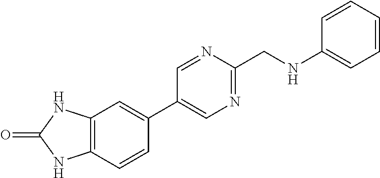 [SMILES: O=c1[nH]c2ccc(-c3cnc(CNc4ccccc4)nc3)cc2[nH]1]

The above compound (38d) was prepared from 13b and 36 following Method I and purified by column chromatography on silica gel (MeOH/CH₂Cl₂, 10:90) to give 38d (33%) as a yellow solid; ¹H NMR (DMSO-d₆, 600 MHz) 10.85 (1 H, s), 10.80 (1 H, s), 9.02 (2 H, s), 7.33 (1 H, d, J=8.5 Hz), 7.27 (1 H, s), 7.05-7.03 (3 H, m), 6.64 (2 H, d, J=8.5 Hz), 6.51 (1 H, t, J=7.3 Hz), 6.25 (1 H, t, J=2.4 Hz), 4.46 (2 H, d, J=6.1 Hz); HRMS (ESI-TOF) m/z: [M+H]⁺ calculated for C₁₈H₁₆N₅O 318.1349; found 318.1327.

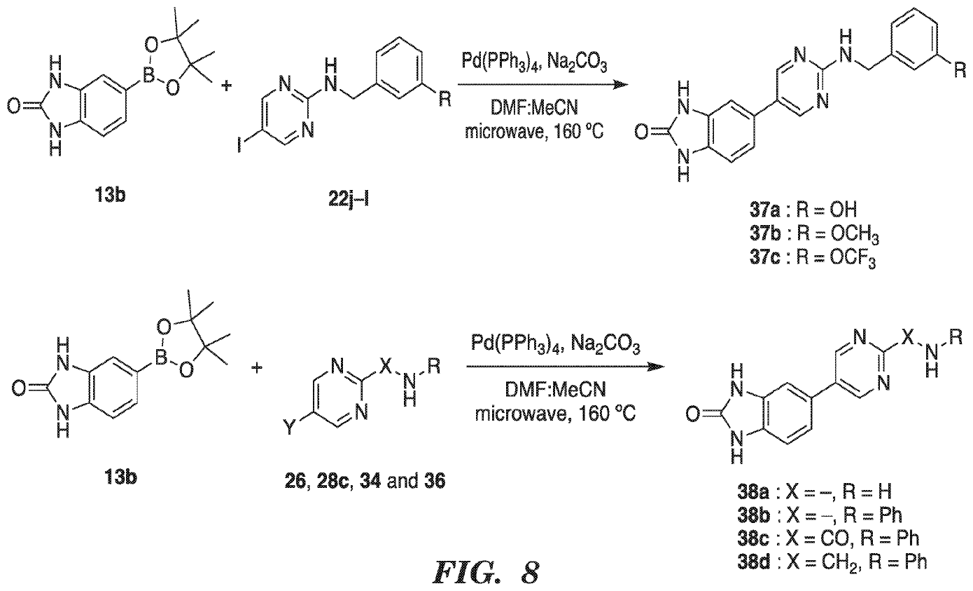

The synthetic schemes for compounds 37a-c and 38a-d, discussed supra, are shown in FIG. 8.

### 3-Bromo-2-methyl-6-nitroandine (40)

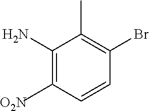

To a solution of 39 (500 mg, 3.29 mmol) in acetic acid (25 mL) was added NBS (585 mg, 3.29 mmol). The reaction mixture was refluxed for 2 h. After being quenched by the addition of water, the aqueous layer was extracted with EtOAc (2×20 mL). The combined organic extracts were washed with brine, dried over anhydrous Na₂SO₄, filtered, and concentrated. The residue was purified by column chromatography on silica gel (EtOAc/hexane, 10:90 to 15:85) to afford 40 (661.1 mg, 87%) as an orange solid; ¹H NMR (CDCl₃, 600 MHz) 7.41(1 H, d, J=8.5 Hz), 6.56 (1 H, d, J=8.5 Hz), 4.77 (2 H, br), 2.44 (3 H, s); HRMS (ESI-TOF) m/z: [M+H]⁺ calculated for C₇H₈⁷⁹BrN₂O₂ and C₇H₈⁸¹BrN₂O₂ 230.9764, 232.9744; found 230.9727, 232.9710.

### 4-Bromo-3-methylbenzene-1,2-diamine (41a)

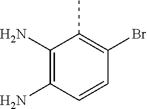

To a mixture of 40 (600 mg, 2.60 mmol), iron (725 mg, 13.0 mmol) and ammonium chloride (736 mg, 13.8 mmol) were added ethanol (20 mL) and water (4 mL). The reaction mixture was refluxed for 3 h. After cooled down, the reaction was filtered through a Celite pad and solvent was removed in vacuo. EtOAc and water were added. The aqueous layer was extracted with EtOAc (2×20 mL). The combined organic extracts were washed with brine, dried over anhydrous Na₂SO₄, filtered, and concentrated. The residue was purified by column chromatography on silica gel (EtOAc/hexane, 50:50) to afford 41a (449.2 mg, 86%) as a brown oil; ¹H NMR (CDCl₃, 600 MHz) 6.90 (1 H, d, J=8.5 Hz), 6.50 (1 H, d, J=8.5 Hz), 3.54 (2 H, br), 3.29 (2 H, br), 2.30 (3 H, s); HRMS (ESI-TOF) m/z: [M+H]⁺ calculated for C₇H₁₀⁷⁹BrN₂ and C₇H₁₀⁸¹BrN₂ 201.0022, 203.0002; found 201.0112, 203.009.

### Example 11—General Procedure for the Preparation of Benzimidazolone—Method J

### 5-Bromo-4-methyl-1H-benzo[d]imidazol-2(3H)-one (42a)

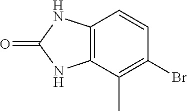

To a solution of 41a (200 mg, 1.00 mmol) in anhydrous dichloromethane (10 mL) was added triethylamine (0.36 mL, 2.59 mmol). The reaction was purged with nitrogen and stirred in an ice-bath. Triphosgene (148 mg, 0.497 mmol) was added under nitrogen at 0° C. The reaction mixture was stirred at 0° C. for 40 min. After being quenched by the addition of saturated sodium bicarbonate, the aqueous layer was extracted with EtOAc (2×15 mL). The combined organic extracts were washed with brine, dried over anhydrous Na₂SO₄, filtered, and concentrated. The crude product was washed with hexane and ether to afford 42a (168.2 mg, 74%) as a light brown solid; ¹H NMR (DMSO-d₆, 600 MHz) 10.93 (1 H, s), 10.74 (1 H, s), 7.12 (1 H, d, J=8.5 Hz), 6.70 (1 H, d, J=8.5 Hz), 2.28 (3 H, s); HRMS (ESI-TOF) m/z: [M+H]⁺ calculated for C₈H₈⁷⁹BrN₂O and C₈H₈⁸¹BrN₂O 226.9815, 228.9715; found 226.9801, 228.9781.

### 5-Bromo-6-methyl-1H-benzo[d]imidazol-2(3H)-one (42b)

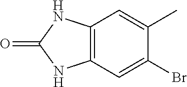

The above compound (42b) was prepared from 41b following Method J to give 42b (76%) as a light brown solid; ¹H NMR (DMSO-d₆, 600 MHz) 10.86 (1 H, s), 10.70 (1 H, s), 10.63 (1 H, s), 7.05 (1 H, s), 6.89 (1 H, s), 2.28 (3 H, s); HRMS (ESI-TOF) m/z: [M+H]⁺ calculated for C₈H₈⁷⁹BrN₂O and C₈H₈⁸¹BrN₂O 226.9815, 228.9715; found 226.9911, 228.9890.

### 4-Methyl-5-(4,4,5,5-tetramethyl-1,3,2-dioxaborolan-2-yl)-1H-benzo[d]yl)-2(3H)-one (43a)

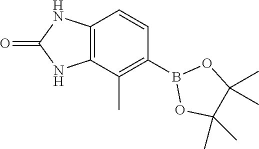 [SMILES: Cc1c(B2OC(C)(C)C(C)(C)O2)ccc2[nH]c(=O)[nH]c12]

The above compound (43a) was prepared from 42a following Method D. A mixture of 1,4-dioxane and DMF (4:1) was used as a solvent and the mixture was stirred at 80° C. for 67 h . The reaction was purified by column chromatography on silica gel (MeOH/CH₂Cl₂, 2.5:97.5 to 5:95) to give 43a (36%) as a light brown solid; ¹H NMR (CDCl₃, 600 MHz) 10.42 (1 H, s), 10.34 (1 H, s), 7.60 (1 H, d, J=7.3 Hz), 6.99 (1 H, d, J=8.5 Hz), 2.64 (3 H, s), 1.36 (12 H, s); HRMS (ESI-TOF) m/z: [M+H]⁺ calculated for C₁₄H₂₀BN₂O₃ 275.1561; found 275.1570.

### 5-Methyl-6-(4,4,5,5-tetramethyl-1,3,2-dioxaborolan-2-yl)-1H-benzo[d]imidazol-2(3H)-one (43b)

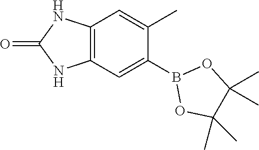 [SMILES: Cc1cc2[nH]c(=O)[nH]c2cc1B1OC(C)(C)C(C)(C)O1]

The above compound (43b) was prepared from 42b following Method D. A mixture of 1,4-dioxane and DMF (4:1) was used as a solvent and the mixture was stirred at 80° C. for 67 h . The reaction was purified by column chromatography on silica gel (MeOH/CH₂Cl₂, 2.5:97.5 to 5:95) to give 43b (30%) as a brown solid; ¹H NMR (CDCl₃, 600 MHz) 9.80 (1 H, s), 9.17 (1 H, s), 7.51 (1 H, s), 6.90 (1 H, s), 2.55 (3 H, s), 1.33 (12 H, s); HRMS (ESI-TOF) m/z: [M+H]⁺ calculated for C₁₄H₂₀BN₂O₃ 275.1561; found 275.1572.

### 5-(2-(Benzylamino)pyrimidin-5-yl)-4-methyl-1H-benzo[d]imidazol-2(3H)-one (44a)

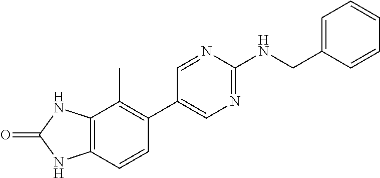 [SMILES: Cc1c(-c2cnc(NCc3ccccc3)nc2)ccc2[nH]c(=O)[nH]c12]

The above compound (44a) was prepared from 43a and 6a following Method I and purified by column chromatography on silica gel (MeOH/CH₂Cl₂, 5:95) to give 44a (34%) as a pale yellow solid; ¹H NMR (DMSO-d₆, 600 MHz) 10.75 (1 H, s), 10.62 (1 H, s), 8.24 (2 H, s), 7.78 (1 H, t, J=4.9 Hz), 7.33-7.29 (4 H, m), 7.21 (1 H, t, J=7.3 Hz), 6.79 (2 H, q, J=7.3 Hz), 4.53 (2 H, d, J=6.1 Hz), 2.17 (3 H, s); HRMS (ESI-TOF) m/z: [M+H]⁺ calculated for C₁₉H₁₈N₅O 332.1506; found 332.1496.

### 5-(2-(Benzylamino)pyrimidin-5-yl)-6-methyl-1H-benzo[d]yl)-2 (3H)-one (44b)

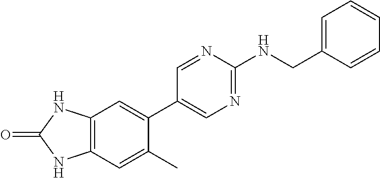 [SMILES: Cc1cc2[nH]c(=O)[nH]c2cc1-c1cnc(NCc2ccccc2)nc1]

The above compound (44b) was prepared from 43b and 6a following Method I and purified by column chromatography on silica gel (MeOH/CH₂Cl₂, 5:95 to 10:90) to give 44b (29%) as a light gray solid; ¹H NMR (DMSO-d₆, 600 MHz) 10.56-10.54 (2 H, m), 8.24 (2 H, s), 7.78 (1 H, s), 7.32-7.21 (5 H, m), 6.82 (1 H, s), 6.69 (1 H, s), 4.53 (2 H, s), 2.18 (3 H, s); HRMS (ESI-TOF) m/z: [M+H]⁺ calculated for C₁₉H₁₈N₅O 332.1506; found 332.1501.

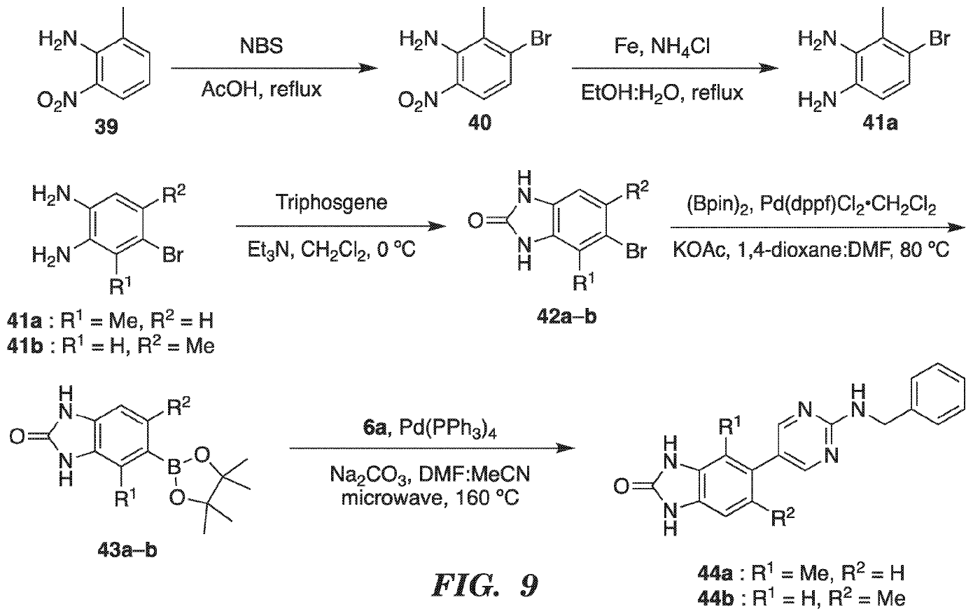

The synthetic schemes for compounds 44a-b, discussed supra, are shown in FIG. 9.

### Example 12—General Procedure for the Preparation of 2-Phenyl-5-pyrimidine-2-amines—Method K

### N-(2-Fluorophenyl)-5-iodopyrimidin-2-amine (47a)

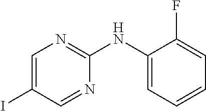 [SMILES: Fc1ccccc1Nc1ncc(I)cn1]

To a mixture of 2-chloro-5-iodopyrimidine 45 (100.0 mg, 0.42 mmol) and 2-fluoroaniline 46a (69.3 mg, 0.62 mmol) in 1,4-dioxane (2 mL) was acetic acid (0.4 mL). The reaction was stirred at 110° C. for 24 h. After being quenched by the addition of water, the aqueous layer was extracted with EtOAc (2×10 mL). The combined organic extracts were washed with brine, dried over anhydrous Na₂SO₄, filtered, and concentrated. The residue was purified by column chromatography on silica gel (EtOAc/hexane, 10:90) to afford 47a (42.4 mg, 32%) as a white solid; ¹H NMR (CDCl₃, 500 MHz) 8.57 (2 H, s), 8.35 (1 H, t, J=8.6 Hz), 7.30 (1 H, br), 7.16-7.09 (2 H, m), 7.02-6.98 (1 H, m); HRMS (ESI-TOF) m/z: [M+H]⁺ calculated for C₁₀H₈FIN₃ 315.9742; found 315.9747.

### N-(3-Fluorophenyl)-5-iodopyrimidin-2-amine (47b)

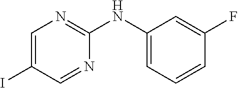 [SMILES: Fc1cccc(Nc2ncc(I)cn2)c1]

The above compound (47b) was prepared from 45 and 46b following Method K and purified by column chromatography on silica gel (EtOAc/hexane, 20:80) to give 47b (93%) as a pale yellow solid; ¹H NMR (CDCl₃, 500 MHz) 8.56 (2 H, s), 7.64 (1 H, dt, J=11.7, 2.5 Hz), 7.29-7.24 (1 H, m), 7.16-7.14 (2 H, m), 6.75 (1 H, td, J=8.1, 1.5 Hz); HRMS (ESI-TOF) m/z: [M+H]⁺ calculated for C₁₀H₈FIN₃ 315.9742; found 315.9762.

### N-(4-Fluorophenyl)-5-iodopyrimidin-2-amine (47c)

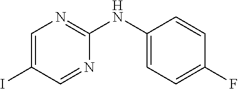 [SMILES: Fc1ccc(Nc2ncc(I)cn2)cc1]

The above compound (47c) was prepared from 45 and 46c following Method K and purified by column chromatography on silica gel (EtOAc/hexane, 20:80) to give 47c (86%) as a pale yellow solid; ¹H NMR (CDCl₃, 500 MHz) 8.51 (2 H, s), 7.51-7.48 (2 H, m), 7.18 (1 H, br), 7.04 (2 H, t, J=8.1 Hz); HRMS (ESI-TOF) m/z: [M+H]⁺ calculated for C₁₀H₈FIN₃ 315.9742; found 315.9756.

### N-(2-Chlorophenyl)-5-iodopyrimidin-2-amine (47d)

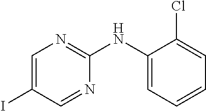 [SMILES: Clc1ccccc1Nc1ncc(I)cn1]

The above compound (47d) was prepared from 45 and 46d following Method K and purified by column chromatography on silica gel (EtOAc/hexane, 5:95) to give 47d (11%) as a light purple solid; ¹H NMR (CDCl₃, 500 MHz) 8.57 (2 H, s), 8.42 (1 H, d, J=8.1 Hz), 7.60 (1 H, br), 7.40 (1 H, d, J=7.6 Hz), 7.29 (1 H, t, J=8.4 Hz), 6.99 (1 H, t, J=7.1 Hz); HRMS (ESI-TOF) m/z: [M+H]⁺ calculated for C₁₀H₈ClIN₃ 331.9446; found 331.9439.

### N-(3-Chlorophenyl)-5-iodopyrimidin-2-amine (47e)

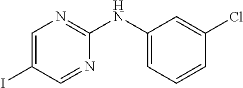 [SMILES: Clc1cccc(Nc2ncc(I)cn2)c1]

The above compound (47e) was prepared from 45 and 46e following Method K and purified by column chromatography on silica gel (EtOAc/hexane, 10:90) to give 47e (85%) as a yellow solid; ¹H NMR (CDCl₃, 500 MHz) 8.56 (2 H, s), 7.78 (1 H, t, J=2.0 Hz), 7.35 (1 H, d, J=7.6 Hz), 7.24 (1 H, t, J=8.1 Hz), 7.11 (1 H, br), 7.03 (1 H, d, J=7.1 Hz); HRMS (ESI-TOF) m/z: [M+H]⁺ calculated for C₁₀H₈ClIN₃ 331.9446; found 331.9448.

### N-(4-Chlorophenyl)-5-iodopyrimidin-2-amine (47f)

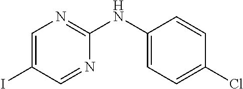 [SMILES: Clc1ccc(Nc2ncc(I)cn2)cc1]

The above compound (47f) was prepared from 45 and 46f following Method K and purified by column chromatography on silica gel (EtOAc/hexane, 20:80 to 40:60) to give 47f (86%) as a light brown solid; ¹H NMR (CDCl₃, 500 MHz) 8.53 (2 H, s), 7.52 (2 H, d, J=8.6 Hz), 7.30 (2 H, d, J=8.6 Hz), 7.10 (1 H, br); HRMS (ESI-TOF) m/z: [M+H]⁺ calculated for C₁₀H₈ClIN₃ 331.9446; found 331.9455.

### 5-Iodo-N-(2-methoxyphenyl)pyrimidin-2-amine (47g)

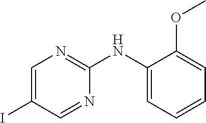 [SMILES: COc1ccccc1Nc1ncc(I)cn1]

The above compound (47g) was prepared from 45 and 46g following Method K and purified by column chromatography on silica gel (EtOAc/hexane, 5:95) to give 47g (60%) as a white solid; ¹H NMR (CDCl₃, 500 MHz) 8.54 (2 H, s), 8.39-8.37 (1 H, m), 7.76 (1 H, br), 7.06-6.99 (2 H, m), 6.91-6.89 (1 H, m), 3.90 (3 H, s); HRMS (ESI-TOF) m/z: [M+H]⁺ calculated for C₁₁H₁₁IN₃O 327.9942; found 328.0039.

### 5-Iodo-N-(3-methoxyphenyl)pyrimidin-2-amine (47h)

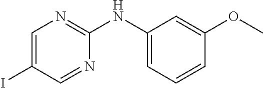 [SMILES: COc1cccc(Nc2ncc(I)cn2)c1]

The above compound (47h) was prepared from 45 and 46h following Method K and purified by column chromatography on silica gel (EtOAc/hexane, 15:85) to give 47h (77%) as a white solid; ¹H NMR (CDCl₃, 500 MHz) 8.53 (2 H, s), 7.34 (1 H, t, J=2.0 Hz), 7.24 (1 H, t, J=8.1 Hz), 7.16 (1 H, br), 7.05 (1 H, d, J=8.1 Hz), 6.62 (1 H, dd, J=8.1, 2.5 Hz), 3.83 (3 H, s); HRMS (ESI-TOF) m/z: [M+H]⁺ calculated for C₁₁H₁₁IN₃O 327.9942; found 327.9940.

### 5-Iodo-N-(4-methoxyphenyl)pyrimidin-2-amine (47i)

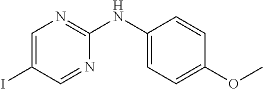 [SMILES: COc1ccc(Nc2ncc(I)cn2)cc1]

The above compound (47i) was prepared from 45 and 46h following Method K and purified by column chromatography on silica gel (EtOAc/hexane, 15:85) to give 47h (65%) as a pale yellow solid; ¹H NMR (CDCl₃, 500 MHz) 8.47 (2 H, s), 7.42 (2 H, d, J=9.2 Hz), 6.98 (1 H, br), 6.90 (2 H, d, J=9.2 Hz), 3.81 (3 H, s); HRMS (ESI-TOF) m/z: [M+H]⁺ calculated for C₁₁H₁₁IN₃O 327.9942; found 328.0026.

### 5-(2-((2-Fluorophenyl)amino)pyrimidin-5-yl)-1H-benzo[d]imidazol-2(3H)-one (48a)

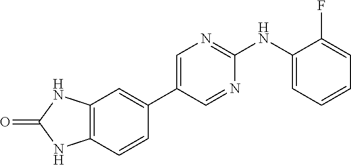 [SMILES: O=c1[nH]c2ccc(-c3cnc(Nc4ccccc4F)nc3)cc2[nH]1]

The above compound (48a) was prepared from 13b and 47a following Method I and purified by column chromatography on silica gel (MeOH/CH₂Cl₂, 5:95 to 10:90) to give 48a (26%) as a brown solid; ¹H NMR (DMSO-d₆, 500 MHz) 10.72 (1 H, s), 10.66 (1 H, s), 9.12 (1 H, s), 8.67 (2 H, s), 7.77 (1 H, t, J=8.1 Hz), 7.23-7.13 (5 H, m), 6.99(1 H, d, J=8.1 Hz); HRMS (ESI-TOF) m/z: [M+H]⁺ calculated for C₁₇H₁₃FN₅O 322.1099; found 322.1085.

### 5-(2-((3-Fluorophenyl)amino)pyrimidin-5-yl)-1H-benzo[d]imidazol-2(3H)-one (48b)

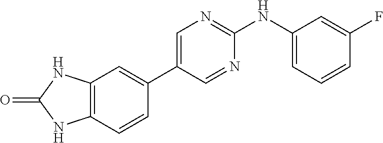 [SMILES: O=c1[nH]c2ccc(-c3cnc(Nc4cccc(F)c4)nc3)cc2[nH]1]

The above compound (48b) was prepared from 13b and 47b following Method I and purified by column chromatography on silica gel (MeOH/CH₂Cl₂, 5:95 to 10:90) to give 48b (32%) as a yellow solid; ¹H NMR (DMSO-d₆, 500 MHz) 10.75 (1 H, s), 10.68 (1 H, s), 9.94 (1 H, s), 8.78 (2 H, s), 7.85 (1 H, d, J=13.2 Hz), 7.50 (1 H, d, J=8.6 Hz), 7.30 (1 H, q, J=7.6 Hz), 7.25 (1 H, d, J=8.1 Hz), 7.19 (1 H, s), 7.00 (1 H, d, J=8.1 Hz), 6.74 (1 H, td, J=8.1, 2.0 Hz); HRMS (ESI-TOF) m/z: [M+H]⁺ calculated for C₁₇H₁₃FN₅O 322.1099; found 322.1075.

### 5-(2-((4-Fluorophenyl)amino)pyrimidin-5-yl)-1H-benzo[d]imidazol-2(3H)-one (48c)

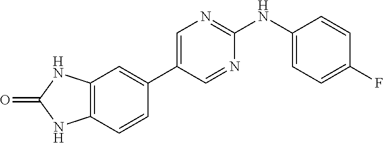 [SMILES: O=c1[nH]c2ccc(-c3cnc(Nc4ccc(F)cc4)nc3)cc2[nH]1]

The above compound (48c) was prepared from 13b and 47c following Method I and purified by column chromatography on silica gel (MeOH/CH₂Cl₂, 5:95 to 10:90) to give 48c (23%) as a yellow solid; ¹H NMR (DMSO-d₆, 500 MHz) 10.73 (1 H, s), 10.67 (1 H, s), 9.71 (1 H, s), 8.72 (2 H, s), 7.79-7.76 (2 H, m), 7.22 (1 H, d, J=7.6 Hz), 7.17(1 H, s), 7.13 (2 H, t, J=9.2 Hz), 7.00 (1 H, d, J=8.1 Hz); HRMS (ESI-TOF) m/z: [M+H]⁺ calculated for C₁₇H₁₃FN₅O 322.1099; found 322.1103.

### 5-(2-((2-Chlorophenyl)amino)pyrimidin-5-yl)-1H-benzo[d]imidazol-2 (3H)-one (48d)

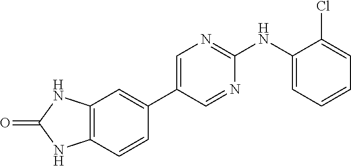 [SMILES: O=c1[nH]c2ccc(-c3cnc(Nc4ccccc4Cl)nc3)cc2[nH]1]

The above compound (48d) was prepared from 13b and 47d following Method I and purified by column chromatography on silica gel (MeOH/CH₂Cl₂, 5:95) to give 48d (39%) as a light brown solid; ¹H NMR (DMSO-d₆, 500 MHz) 10.72 (1 H, s), 10.67 (1 H, s), 8.81 (1 H, s), 8.68 (2 H, s), 7.87 (1 H, d, J=5.6 Hz), 7.49 (1 H, d, J=6.1 Hz), 7.34 (1 H, s), 7.21-7.15 (3 H, m), 6.99 (1 H, d, J=5.1 Hz); HRMS (ESI-TOF) m/z: [M+H]⁺ calculated for C₁₇H₁₃ClN₅O 338.0804; found 338.0761.

### 5-(2-((3-Chlorophenyl)amino)pyrimidin-5-yl)-1H-benzo[d]imidazol-2 (3H)-one (48e)

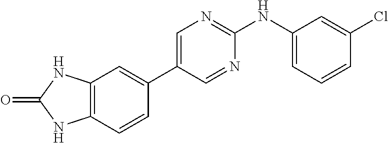 [SMILES: O=c1[nH]c2ccc(-c3cnc(Nc4cccc(Cl)c4)nc3)cc2[nH]1]

The above compound (48e) was prepared from 13b and 47e following Method I and purified by column chromatography on silica gel (MeOH/CH₂Cl₂, 5:95) to give 48e (27%) as a yellow solid; ¹H NMR (DMSO-d₆, 500 MHz) 10.75 (1 H, s), 10.68 (1 H, s), 9.92 (1 H, s), 8.79 (2 H, s), 8.05 (1 H, s),7.67 (1 H, d, J=8.1 Hz), 7.31 (1 H, t, J=8.1 Hz), 7.25 (1 H, d, J=8.6 Hz), 7.20 (1 H, s), 7.00 (1 H, d, J=7.1 Hz), 6.97 (1 H, d, 7.6 Hz); HRMS (ESI-TOF) m/z: [M+H]⁺ calculated for C₁₇H₁₃ClN₅O 338.0804; found 338.0797.

### 5-(2-((4-Chlorophenyl)amino)pyrimidin-5yl)-1H-benzo[d]imidazol-2 (3H)-one (48f)

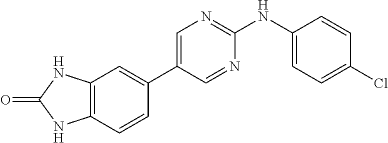 [SMILES: O=c1[nH]c2ccc(-c3cnc(Nc4ccc(Cl)cc4)nc3)cc2[nH]1]

The above compound (48f) was prepared from 13b and 47f following Method I and purified by column chromatography on silica gel (MeOH/CH₂Cl₂, 5:95 to 10:90) to give 48f (27%) as a yellow solid; ¹H NMR (DMSO-d₆, 500 MHz) 10.74 (1 H, s), 10.68 (1 H, s), 9.85 (1 H, s), 8.75 (2 H, s), 7.83 (2 H, d, J=9.2 Hz), 7.33 (2 H, d, J=8.6 Hz), 7.24 (1 H, dd, J=7.6, 1.5 Hz), 7.18 (1 H, d, J=1.0 Hz), 7.00 (1 H, d, J=7.6 Hz); HRMS (ESI-TOF) m/z: [M+H]⁺ calculated for C₁₇H₁₃ClN₅O 338.0804; found 338.0808.

### 5-(2-((2-Methoxyphenyl)amino)pyrimidin-5-yl)-1H-benzo[d]imidazol-2(3H)-one (48g)

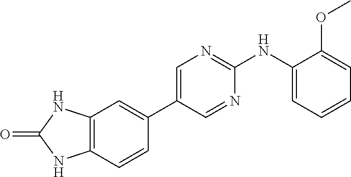 [SMILES: COc1ccccc1Nc1ncc(-c2ccc3[nH]c(=O)[nH]c3c2)cn1]

The above compound (48g) was prepared from 13b and 47g following Method I and purified by column chromatography on silica gel (MeOH/CH₂Cl₂, 5:95) to give 48g (28%) as a yellow solid; ¹H NMR (DMSO-d₆, 500 MHz) 10.72 (1 H, s), 10.67 (1 H, s), 8.71 (2 H, s), 8.17 (1 H, d, J=8.6 Hz), 8.13 (1 H, s), 7.22 (1 H, d, J=7.6 Hz), 7.16 (1 H, s), 7.06-6.99(3 H, m), 6.95 (1 H, t, J=8.6 Hz), 3.86 (3 H, s); HRMS (ESI-TOF) m/z: [M+H]⁺ calculated for C₁₈H₁₆N₅O₂ 334.1299; found 334.1316.

### 5-(2-((3-Methoxyphenyl)amino)pyrimidin-5-yl)-1H-benzo[d]imidazol-2(3H)-one (48h)

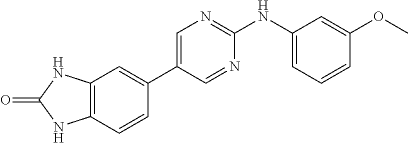 [SMILES: COc1cccc(Nc2ncc(-c3ccc4[nH]c(=O)[nH]c4c3)cn2)c1]

The above compound (48h) was prepared from 13b and 47h following Method I and purified by column chromatography on silica gel (MeOH/CH₂Cl₂, 5:95 to 10:90) to give 48h (31%) as a yellow solid; ¹H NMR (DMSO-d₆, 500 MHz) 10.73 (1 H, s), 10.67 (1 H, s), 9.66 (1 H, s), 8.74 (2 H, s), 7.51 (1 H, t, J=2.0 Hz), 7.35 (1 H, d, J=9.7 Hz), 7.23 (1 H, dd, J=7.9, 2.0 Hz), 7.19-7.16 (2 H, m), 7.00 (1 H, d, J=7.6 Hz), 6.53 (1 H, dd, J=8.1, 2.5 Hz), 3.74 (3 H, s); HRMS (ESI-TOF) m/z: [M+H]⁺ calculated for C₁₈H₁₆N₅O₂ 334.1299; found 334.1297.

### 5-(2-((4-Methoxyphenyl)amino)pyrimidin-5-yl)-1H-benzo[d]imidazol-2(3H)-one (48i)

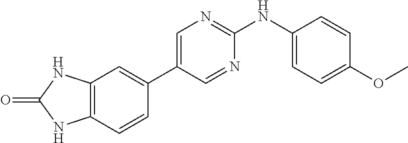 [SMILES: COc1ccc(Nc2ncc(-c3ccc4[nH]c(=O)[nH]c4c3)cn2)cc1]

The above compound (48i) was prepared from 13b and 47i following Method I and purified by column chromatography on silica gel (MeOH/CH₂Cl₂, 5:95 to 10:90) to give 48i (28%) as a brown solid; ¹H NMR (DMSO-d₆, 500 MHz) 10.71 (1 H, s), 10.65 (1 H, s), 9.46 (1 H, s), 8.67 (2 H, s), 7.64 (2 H, d, J=8.6 Hz), 7.20 (1 H, d, J=8.1 Hz), 7.15 (1 H, s), 6.99 (1 H, d, J=7.6 Hz), 6.88 (2 H, d, J=8.6 Hz), 3.72 (3 H, s); HRMS (ESI-TOF) m/z: [M+H]⁺ calculated for C₁₈H₁₆N₅O₂ 334.1299; found 334.1393.

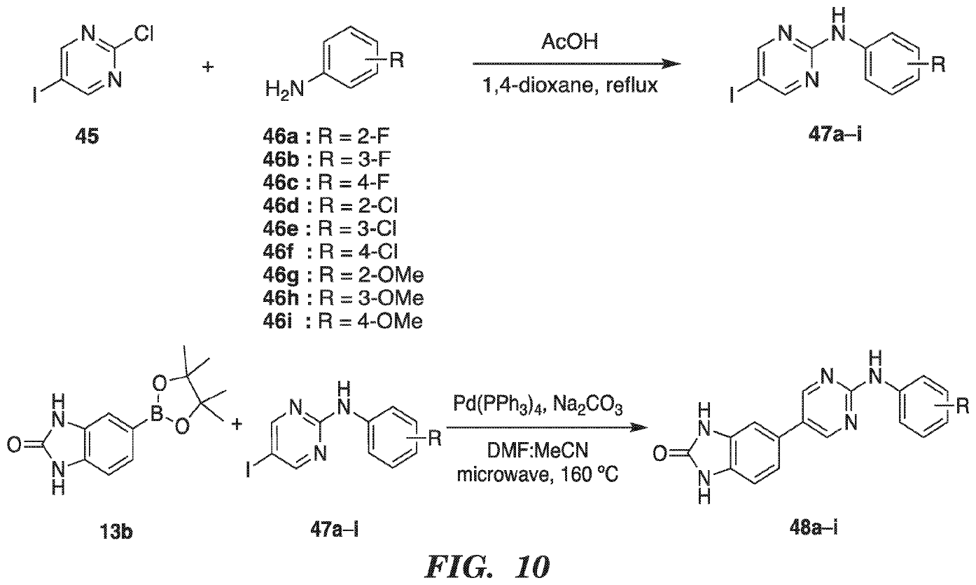

The synthetic schemes for compounds 48a-i, discussed supra, are shown in FIG. 10.

### N-(Furan-2-ylmethyl)-5-iodopyrimidin-2-amine (50a)

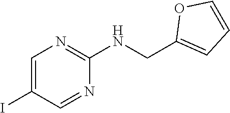 [SMILES: Ic1cnc(NCc2ccco2)nc1]

The above compound (50a) was prepared from 4a and 49a following Method B and purified by column chromatography on silica gel (EtOAc/hexane, 20:80) to give 50a (66%) as a white solid; ¹H NMR (CDCl₃, 500 MHz) 8.40 (2 H, s), 7.36 (1 H, s), 6.32 (1 H, s), 6.24 (1 H, s), 5.54 (1 H, br), 4.58 (2 H, d, J=6.1 Hz); HRMS (ESI-TOF) m/z: [M+H]⁺ calculated for C₉H₉IN₃O 301.9785; found 301.9799

### 5-Iodo-N-(thiophen-2-ylmethyl)pyrimidin-2-amine (50b)

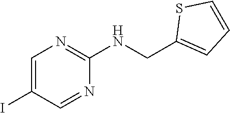 [SMILES: Ic1cnc(NCc2cccs2)nc1]

The above compound (50b) was prepared from 4a and 49b following Method B and purified by column chromatography on silica gel (EtOAc/hexane, 20:80) to give 50b (80%) as a pale yellow solid; ¹H NMR (CDCl₃, 500 MHz) 8.42 (2 H, s), 7.21 (1 H, d, J=4.1 Hz), 7.01 (1 H, d, J=3.0 Hz), 6.95 (1 H, dd, J=4.8, 3.0 Hz), 5.46 (1 H, br), 4.76 (2 H, d, J=5.6 Hz); HRMS (ESI-TOF) m/z: [M+H]⁺ calculated for C₉H₉IN₃S 317.9557; found 317.9669

### N-((1H-Imidazol-2-yl)methyl)-5-iodopyrimidin-2-amine (50c)

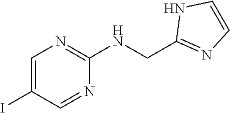 [SMILES: Ic1cnc(NCc2ncc[nH]2)nc1]

The above compound (50c) was prepared from 4a and 49c following Method B and purified by column chromatography on silica gel (MeOH/CH₂Cl₂, 5:95 to 10:90) to give 50c (66%) as a yellow solid; ¹H NMR (CD₃OD, 500 MHz) 8.43 (2 H, s), 6.94 (2 H, s), 4.58 (2 H, s), 4.54 (1 H, br); HRMS (ESI-TOF) m/z: [M+H]⁺ calculated for C₈H₉IN₅ 301.9898; found 301.9889

### N-((1H-Imidazol-4-yl)methyl)-5-iodopyrimidin-2-amine (50d)

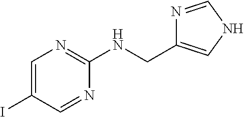 [SMILES: Ic1cnc(NCc2c[nH]cn2)nc1]

The above compound (50d) was prepared from 4a and 49d following Method B and purified by column chromatography on silica gel (MeOH/CH₂Cl₂, 10:90 to 15:85) to give 50d (82%) as a pale yellow solid; ¹H NMR (DMSO-d₆, 500 MHz) 11.79 (1 H, br), 8.40 (2 H, s), 7.54-7.51 (2 H, m), 6.84 (1 H, s), 4.34 (2 H, d, J=5.6 Hz); HRMS (ESI-TOF) m/z: [M+H]⁺ calculated for C₈H₉IN₅ 301.9898; found 301.9888

### N-((1H-Pyrazol-3-Amethyl)-5-iodopyrimidin-2-amine (50e)

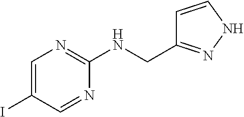 [SMILES: Ic1cnc(NCc2cc[nH]n2)nc1]

The above compound (50e) was prepared from 4a and 49e following Method B and purified by column chromatography on silica gel (MeOH/CH₂Cl₂, 5:95) to give 50e (71%) as a white solid; ¹H NMR (CDCl₃, 500 MHz) 8.43 (2 H, s), 7.51 (1 H, d, J=2.0 Hz), 6.25 (1 H, d, J=2.0 Hz), 5.73 (1 H, br), 4.62 (2 H, d, J=5.6 Hz); HRMS (ESI-TOF) m/z: [M+H]⁺ calculated for C₈H₉IN₅ 301.9898; found 301.9920

### 5-Iodo-N-(oxazol-2-ylmethyl)pyrimidin-2-amine (50f)

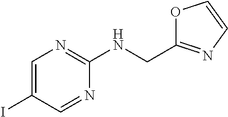 [SMILES: Ic1cnc(NCc2ncco2)nc1]

The above compound (50f) was prepared from 4a and 49f following Method B and purified by column chromatography on silica gel (EtOAc/hexane, 50:50 to 80:20) to give 50f (80%) as a white solid; ¹H NMR (CDCl₃, 500 MHz) 8.43 (2 H, s), 7.61 (1 H, s), 7.07 (1 H, s), 5.72 (1 H, br), 4.73 (2 H, d, J=6.1 Hz); HRMS (ESI-TOF) m/z: [M+H]⁺ calculated for C₈H₈IN₄O 302.9738; found 302.9808.

### 5-(2-((Furan-2-ylmethyl)amino)pyrimidin-5-yl)-1H-benzo[d]imidazol-2(3H)-one (51a)

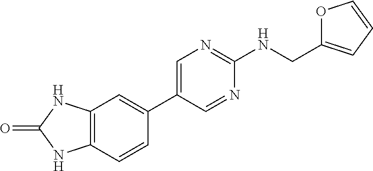 [SMILES: O=c1[nH]c2ccc(-c3cnc(NCc4ccco4)nc3)cc2[nH]1]

The above compound (51a) was prepared from 13b and 50a following Method I and purified by column chromatography on silica gel (MeOH/CH₂Cl₂, 5:95) to give 51a (22%) as a yellow solid; ¹H NMR (DMSO-d₆, 600 MHz) 10.66 (1 H, s), 10.62 (1 H, s), 8.54 (2 H, s), 7.62 (1 H, t, J=6.1 Hz), 7.53 (1 H, d, J=1.0 Hz), 7.13 (1 H, dd, J=8.1, 1.5 Hz), 7.08(1 H, s), 6.96 (1 H, d, J=7.6 Hz), 6.35 (1 H, dd, J=3.0, 1.5 Hz), 6.21 (1 H, d, J=2.5 Hz), 4.51 (2 H, d, J=6.1 Hz); HRMS (ESI-TOF) m/z: [M+H]⁺ calculated for C₁₆H₁₄N₅O₂ 308.1142; found 308.1149.

### 5-(2-((Thiophen-2-ylmethyl)amino)pyrimidin-5-yl)-1H-benzo[d]imidazol-2(3H)-one (51b)

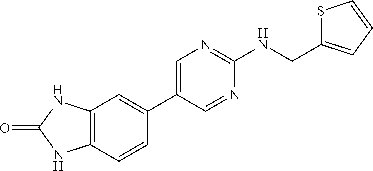 [SMILES: O=c1[nH]c2ccc(-c3cnc(NCc4cccs4)nc3)cc2[nH]1]

The above compound (51b) was prepared from 13b and 50b following Method I and purified by column chromatography on silica gel (MeOH/CH₂Cl₂, 5:95) to give 51b (29%) as a yellow solid; ¹H NMR (DMSO-d₆, 600 MHz) 10.67 (1 H, s), 10.62 (1 H, s), 8.55 (2 H, s), 7.79 (1 H, t, J=6.1 Hz), 7.32 (1 H, d, J=4.6 Hz), 7.14 (1 H, dd, J=7.9, 1.5 Hz), 7.09 (1 H, s), 6.99 (1 H, d, J=3.0 Hz), 6.97-6.93 (2 H, m), 4.68 (2 H, d, J=6.1 Hz); HRMS (ESI-TOF) m/z: [M+H]⁺ calculated for C₁₆H₁₄N₅OS 324.0914; found 324.0910.

### 5-(2-(((1H-Imidazol-2-yl)methyl)amino)pyrimidin-5-yl)-1H-benzo[d]imidazol-2(3H)-one (51c)

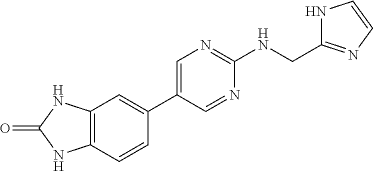 [SMILES: O=c1[nH]c2ccc(-c3cnc(NCc4ncc[nH]4)nc3)cc2[nH]1]

The above compound (51c) was prepared from 13b and 50c following Method I and purified by column chromatography on silica gel (MeOH/CH₂Cl₂, 10:90 to 20:80) to give 51c (34%) as a gray solid; ¹H NMR (DMSO-d₆, 600 MHz) 11.67 (1 H, br), 10.67 (1 H, s), 10.62 (1 H, s), 8.54 (2 H, s), 7.47 (1 H, t, J=5.6 Hz), 7.14 (1 H, dd, J=8.1, 1.5 Hz), 7.08 (1 H, s), 6.97-6.96 (2 H, m), 6.77 (1 H, br), 4.52 (2 H, d, J=6.1 Hz); HRMS (ESI-TOF) m/z: [M+H]⁺ calculated for C₁₅H₁₄N₇O 308.1254; found 308.1248.

### 5-(2-(((1H-Imidazol-4-yl)methyl)amino)pyrimidin-5-yl)-1H-benzo[d]imidazol-2(3H)-one (51d)

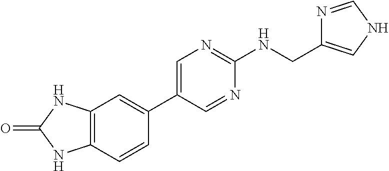 [SMILES: O=c1[nH]c2ccc(-c3cnc(NCc4c[nH]cn4)nc3)cc2[nH]1]

The above compound (51d) was prepared from 13b and 50d following Method I and purified by column chromatography on silica gel (MeOH/CH₂Cl₂, 10:90 to 20:80) to give 51d (7%) as a brown solid; ¹H NMR (DMSO-d₆, 600 MHz) 10.66 (1 H, s), 10.62 (1 H, s), 8.53 (2 H, s), 8.39 (1 H, s), 7.52 (1 H, s), 7.31 (1 H, br), 7.14 (1 H, dd, J=7.4, 1.5 Hz), 7.08 (1 H, s), 6.96 (1 H, d, J=8.1 Hz), 6.87 (1 H, br), 4.43 (2 H, d, J=5.6 Hz); HRMS (ESI-TOF) m/z: [M+H]⁺ calculated for C₁₅H₁₄N₇O 308.1254; found 308.1251.

### 5-(2-(((1H-Pyrazol-3-yl)methyl)amino)pyrimidin-5-yl)-1H-benzo[d]imidazol-2(3H)-one (51e)

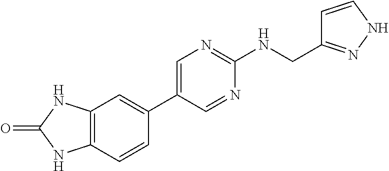 [SMILES: O=c1[nH]c2ccc(-c3cnc(NCc4cc[nH]n4)nc3)cc2[nH]1]

The above compound (51e) was prepared from 13b and 50e following Method I and purified by column chromatography on silica gel (MeOH/CH₂Cl₂, 5:95) to give 51e (10%) as a light brown solid; ¹H NMR (DMSO-d₆, 600 MHz) 10.67 (1 H, s), 10.62 (1 H, s), 8.53 (2 H, s), 7.59 (1 H br), 7.44 (1 H, br), 7.14 (1 H, dd, J=7.9, 1.5 Hz), 7.08 (1 H, s), 6.96 (1 H, d, J=8.1 Hz), 6.14 (1 H, br), 4.51 (2 H, s); HRMS (ESI-TOF) m/z: [M+H]⁺ calculated for C₁₅H₁₄N₇O 308.1254; found 308.1359.

### 5-(2-((Oxazol-2-ylmethyl)amino)pyrimidin-5-yl)-1H-benzo[d]imidazol-2(3H)-one (51f)

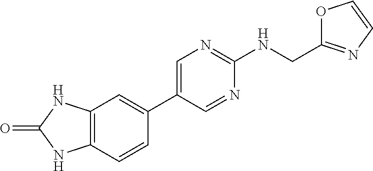 [SMILES: O=c1[nH]c2ccc(-c3cnc(NCc4ncco4)nc3)cc2[nH]1]

The above compound (51f) was prepared from 13b and 50f following Method I and purified by column chromatography on silica gel (MeOH/CH₂Cl₂, 5:95 to 10:90) to give 51f (15%) as a light purple solid; ¹H NMR (DMSO-d₆, 500 MHz) 10.67 (1 H, s), 10.62 (1 H, s), 8.54 (2 H, s), 7.99 (1 H, s), 7.78 (1 H, t, J=5.6 Hz), 7.11 (1 H, s), 7.08 (1 H, s), 6.96(1 H, d, J=8.1 Hz), 4.62 (2 H, d, J=6.1 Hz); HRMS (ESI-TOF) m/z: [M+H]⁺ calculated for C₁₅H₁₃N₆O₂ 309.1095; found 309.1190.

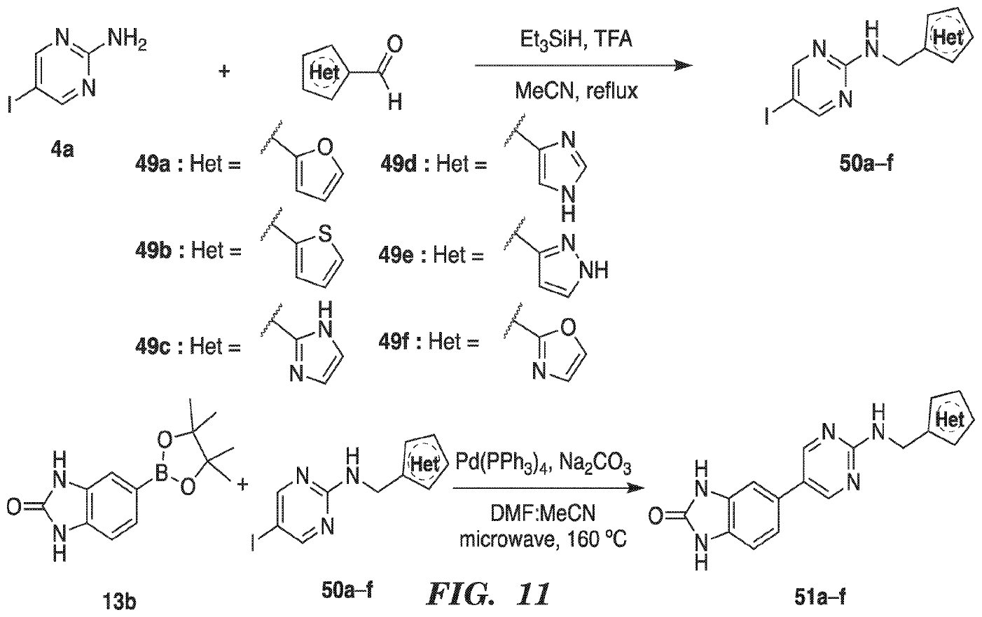

The synthetic schemes for compounds 51a-f, discussed supra, are shown in FIG. 11.

### Example 13—General Procedure for the Preparation of 5-Bromo-2-nitroaniline—Method L

### 5-Bromo-N-ethyl-2-naroaniline (53a)

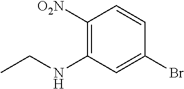

To a solution of 4-bromo-2-fluoro-1-nitrobenzene (52) (500 mg, 2.27 mmol) and potassium carbonate (628 mg, 4.55 mmol) in anhydrous DMF (5 mL) was added 2 M ethanamine in THF (2.3 mL, 4.55 mmol). The resulting mixture was stirred at 80° C. for 22 h. After being quenched with water (10 mL), the aqueous layer was extracted with EtOAc (2×30 mL). The combined organic extracts were washed with brine, dried over anhydrous Na₂SO₄, filtered, and concentrated. The residue was purified by column chromatography on silica gel (EtOAc/hexane, 5: 95 to 10:90) to afford 53a (520.0 mg, 93%) as a yellow solid; ¹H NMR (CDCl₃, 500 MHz) 8.03 (1 H, d, J=9.2 Hz), 7.97 (1 H, br), 7.01 (1 H, d, J=1.5 Hz), 6.75 (1 H, dd, J=9.2, 1.5 Hz), 3.36-3.30 (2 H, m), 1.38 (3 H, t, J=7.6 Hz); HRMS (ESI-TOF) m/z: [M+H]⁺ calculated for C₈H₁₀⁷⁹Br N₂O₂ and C₈H₁₀⁸¹Br N₂O₂ 244.9921, 246.9900; found 244.9920, 246.9898.

### 5-Bromo-N-isopropyl-2-nitroaniline (53b)

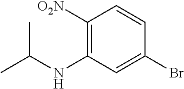

The above compound (53b) was prepared from 52 and isopropylamine following Method L and purified by column chromatography on silica gel (EtOAc/hexane, 5:95 to 10:90) to give 53b (99%) as an orange solid; ¹H NMR (CDCl₃, 500 MHz) 8.03-8.00 (2 H, m), 7.02 (1 H, d, J=1.5 Hz), 6.72 (1 H, dd, J=8.9, 2.0 Hz), 3.84-3.74 (1 H, m), 1.34 (6 H, d, J=6.6 Hz); HRMS (ESI-TOF) m/z: [M+H]⁺ calculated for C₉H₁₂⁷⁹Br N₂O₂ and C₉H₁₂⁸¹Br N₂O₂ 259.0077, 261.0057; found 259.0077, 261.0059.

### 5-Bromo-N-cyclopropyl-2-nitroaniline (53c)

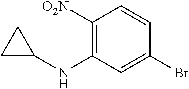 [SMILES: O=[N+]([O-])c1ccc(Br)cc1NC1CC1]

The above compound (53c) was prepared from 52 and cyclopropylamine following Method L and purified by column chromatography on silica gel (EtOAc/hexane, 5:95 to 10:90) to give 53c (99%) as a yellow solid; ¹H NMR (CDCl₃, 500 MHz) 8.07 (1 H, br), 8.01 (1 H, d, J=9.2 Hz), 7.48 (1 H, d, J=2.0 Hz), 6.81 (1 H, dd, J=9.2, 2.0 Hz), 2.58-2.55 (1 H, m), 0.97-0.93 (2 H, m), 0.69-0.66 (2 H, m); HRMS (ESI-TOF) m/z: [M+H]⁺ calculated for C₉H₁₀⁷⁹BrN₂O₂ and C₉H₁₀⁸¹BrN₂O₂ 256.9921, 258.9900; found 256.9917, 258.9902.

### 5-Bromo-N-cyclopentyl-2-nitroaniline (53d)

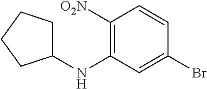 [SMILES: O=[N+]([O-])c1ccc(Br)cc1NC1CCCC1]

The above compound (53d) was prepared from 52 and cyclopentylamine following Method L and purified by column chromatography on silica gel (EtOAc/hexane, 5:95 to 10:90) to give 53d (97%) as a yellow solid; ¹H NMR (CDCl₃, 600 MHz) 8.11 (1 H, br), 8.02 (1 H, d, J=9.8 Hz), 7.05 (1 H, s), 6.73 (1 H, dd, J=8.5, 2.4 Hz), 3.94-3.89 (1 H, m), 2.14-2.09 (2 H, m), 1.83-1.78 (2 H, m), 1.73-1.66 (2 H, m), 1.65-1.61 (2 H, m).

### 5-Bromo-2-nitro-N-phenylaniline (53e)

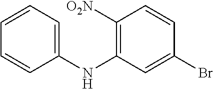 [SMILES: O=[N+]([O-])c1ccc(Br)cc1Nc1ccccc1]

The above compound (53e) was prepared from 52 and aniline following Method L. The reaction was stirred at 80° C. for 16 h and purified by column chromatography on silica gel (EtOAc/hexane, 5:95 to 10:90) to give 57e (70%) as an orange solid; ¹H NMR (CDCl₃, 500 MHz) 9.51 (1 H, br), 8.07 (1 H, d, J=9.2 Hz), 7.46 (2 H, t, J=7.6 Hz), 7.33-7.27 (4 H, m), 6.88 (1 H, dd, J=8.9, 2.0 Hz); HRMS (ESI-TOF) m/z: [M+H]⁺ calculated for C₁₂H₁₀⁷⁹BrN₂O₂ and C₁₂H₁₀⁸¹BrN₂O₂ 292.9921, 294.9900; found 292.9924, 294.9898

### Example 14—General Procedure for the Preparation of 5-Bromo-2-nitroaniline—Method M

### N-(5-Bromo-2-nitrophenyl)pyridin-2-amine (57f)

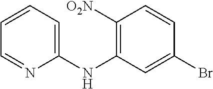 [SMILES: O=[N+]([O-])c1ccc(Br)cc1Nc1ccccn1]

To a solution of pyridine-2-amine (257.0 mg, 2.73 mmol) in anhydrous THF (5 mL) was added potassium tert-butoxide (408 mg, 3.64 mmol) at 0° C. The resulting mixture was purged with nitrogen and stirred at 0° C. for 1 h. 4-Bromo-2-fluoro-1-nitrobenzene (52) (400 mg, 1.82 mmol) was added and the reaction was stirred at 0° C. for 1 h. After being quenched with water (10 mL), the aqueous layer was extracted with EtOAc (2×30 mL). The combined organic extracts were washed with brine, dried over anhydrous Na₂SO₄, filtered, and concentrated. The residue was purified by column chromatography on silica gel (EtOAc/hexane, 5:95 to 10:90) to afford 53f (418.3 mg, 78%) as an orange solid; ¹H NMR (CDCl₃, 500 MHz) 10.28 (1 H, br), 9.19 (1 H, d, J=2.0 Hz), 8.40 (1 H, d, J=5.1 Hz), 8.10 (1 H, d, J=9.2 Hz), 7.67(1 H, td, J=8.9, 2.0 Hz), 7.07 (1 H, dd, J=8.9, 2.0 Hz), 7.01 (1 H, dd, J=7.1, 5.1 hz), 6.95 (1 H, d, J=8.1 Hz); HRMS (ESI-TOF) m/z: [M+H]⁺ calculated for C₁₁H₉⁷⁹BrN₃O₂ and C₁₁H₉⁸¹BrN₃O₂ 293.9873, 295.9853; found 293.9863, 295.9883.

### N-(5-Bromo-2-nitrophenyl)pyridin-3-amine (53g)

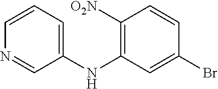 [SMILES: O=[N+]([O-])c1ccc(Br)cc1Nc1cccnc1]

The above compound (53g) was prepared from 52 and pyridine-3-amine following Method M and purified by column chromatography on silica gel (EtOAc/hexane, 40:60) to give 53g (63%) as an orange solid; ¹H NMR (CDCl₃, 500 MHz) 9.44 (1 H, br), 8.62 (1 H, d, J=2.5 Hz), 8.55 (1 H, dd, J=4.8, 1.5 Hz), 8.10 (1 H, d, J=9.2 Hz), 7.63 (1 H, dd, J=8.1, 2.0 Hz), 7.41 (1 H, dd, J=8.4, 4.6 Hz), 6.97 (1 H, dd, J=9.2, 2.0 Hz); HRMS (ESI-TOF) m/z: [M+H]⁺ calculated for C₁₁H₉⁷⁹BrN₃O₂ and C₁₁H₉⁸¹BrN₃O₂ 293.9873, 295.9853; found 293.9889, 295.9868.

### N-(5-Bromo-2-nitrophenyl)pyridin-4-amine (53h)

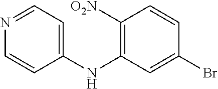 [SMILES: O=[N+]([O-])c1ccc(Br)cc1Nc1ccncc1]

The above compound (53h) was prepared from 52 and pyridine-4-amine following Method M and purified by column chromatography on silica gel (MeOH/CH₂Cl₂, 5:95) to give 53h (60%) as a brown solid; ¹H NMR (CDCl₃, 500 MHz) 9.37 (1 H, br), 8.57 (2 H, d, J=5.1 Hz), 8.09 (2 H, d, J=9.2 Hz), 7.70 (1 H, d, J=1.5 Hz), 7.14 (2 H, dd, J=4.6, 1.5 Hz), 7.10 (1 H, dd, J=8.9, 1.5 Hz); HRMS (ESI-TOF) m/z: [M+H]⁺ calculated for C₁₁H₉⁷⁹BrN₃O₂ and C₁₁H₉⁸¹BrN₃O₂ 293.9873, 295.9853; found 293.9853, 295.9832.

### Example 15—General Procedure for the Preparation of 5-bromo-N¹-alkylbenzene-1,2-diamine—Method N

### 5-Bromo-N¹-ethylbenzene-1,2-diamine (54a)

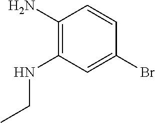

To a solution of 53a (438 mg, 1.79 mmol) in ethanol (10 mL) and water (2 mL) was added ammonium chloride (507 mg, 9.47 mmol) and iron (499 mg, 8.94 mmol). The resulting mixture was refluxed for 24 h. The reaction was filtered through a Celite pad then solvent was removed under reduced pressure. The crude product was extracted with EtOAc (2×40 mL) and water (15 mL). The combined organic extracts were washed with brine, dried over anhydrous Na₂SO₄, filtered, and concentrated. The residue was purified by column chromatography on silica gel (EtOAc/hexane, 15:85 to 20:80) to afford 54a (343.1 mg, 89%) as a brown liquid; ¹H NMR (CDCl₃, 500 MHz) 6.76-6.73 (2 H, m), 6.57 (1 H, d, J=8.1 Hz), 3.27 (3 H, br), 3.12 (2 H, q, J=7.1 Hz), 1.30 (1 H, t, J=7.1 Hz); HRMS (ESI-TOF) m/z: [M+H]⁺ calculated for C₈H₁₂⁷⁹BrN₂ and C₈H₁₂⁸¹BrN₂ 215.0179, 217.0158; found 215.0232, 217.0207.

### 5-Bromo-N¹-isopropylbenzene-1,2-diamine (54b)

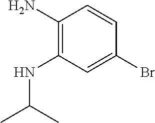

The above compound (54b) was prepared from 53b following Method N and purified by column chromatography on silica gel (EtOAc/hexane, 30:70) to give 54b (90%) as a light brown liquid; ¹H NMR (CDCl₃, 500 MHz) 6.74-6.72 (2 H, m), 6.56 (1 H, d, J=8.6 Hz), 3.59-3.52 (1 H, m), 3.27 (3 H, br), 1.23 (6 H, d, J=6.1 Hz); HRMS (ESI-TOF) m/z: [M+H]⁺ calculated for C₉H₁₄⁷⁹BrN₂ and C₉H₁₄⁸¹BrN₂ 229.0355, 231.0315; found 229.0472, 231.0448

### Example 16—General Procedure for the Preparation of 5-bromo-N¹-alkylbenzene-1,2-diamine—Method O

### 5-Bromo-N¹-cyclopentylbenzene-1,2-diamine (54d)

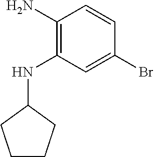 [SMILES: Nc1ccc(Br)cc1NC1CCCC1]

To a solution of 53c (174 mg, 0.61 mmol) in ethanol (10 mL) and water (6 mL) was added sodium dithionite (850 mg, 4.88 mmol). The resulting mixture was room temperature for 4 h. The reaction was filtered through a Celite pad then solvent was removed under reduced pressure. The crude product was extracted with EtOAc (2×40 mL) and water (15 mL). The combined organic extracts were washed with brine, dried over anhydrous Na₂SO₄, filtered, and concentrated. The residue was purified by column chromatography on silica gel (EtOAc/hexane, 15:85) to afford 54d (106.5 mg, 68%) as a brown liquid; ¹H NMR (CDCl₃, 600 MHz) 6.75-6.73 (2 H, m), 6.56 (1 H, d, J=8.5 Hz), 3.73 (1 H, quin, J=6.1 Hz), 3.27 (3 H, br), 2.06-2.03 (2 H, m), 1.75-1.71 (2 H, m), 1.67-1.62 (2 H, m), 1.52-1.47 (2 H, m); HRMS (ESI-TOF) m/z: [M+H]⁺ calculated for C₁₁H₁₆⁷⁹BrN₂ and C₁₁H₁₆⁸¹BrN₂ 255.0492, 257.0471; found 255.0546, 257.0525.

### 5-Bromo-N¹-phenylbenzene-1,2-diamine (54e)

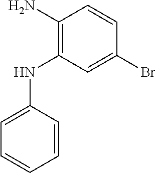 [SMILES: Nc1ccc(Br)cc1Nc1ccccc1]

The above compound (54e) was prepared from 53e following Method N and purified by column chromatography on silica gel (EtOAc/hexane, 20:80 to 30:70) to give 54e (88%) as a light purple solid; ¹H NMR (CDCl₃, 500 MHz) 7.26-7.22 (3 H, m), 7.08 (1 H, dd, J=8.4, 2.5 Hz), 6.88 (1 H, t, J=7.1 Hz), 6.78 (2 H, d, J=7.6 Hz), 6.68 (1 H, d, J=8.1 Hz), 5.16 (1 H, br), 3.73 (2 H, br); HRMS (ESI-TOF) m/z: [M+H]⁺ calculated for C₁₂F1₁₂⁷⁹BrN₂ and C₁₂H₁₂⁸¹BrN₂ 263.0179, 265.0158; found 263.0329, 265.0306.

### 5-Bromo-N¹-(pyridin-2-yl)benzene-1,2-diamine (54f)

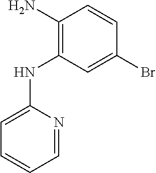 [SMILES: Nc1ccc(Br)cc1Nc1ccccn1]

The above compound (54f) was prepared from 53f following Method O and purified by column chromatography on silica gel (EtOAc/hexane, 20:80 to 40:60) to give 54f (45%) as a brown liquid; ¹H NMR (CDCl₃, 500 MHz) 8.17 (1 H, d, J=5.1 Hz), 7.47 (1 H, td, J=7.6, 2.0 Hz), 7.36 (1 H, d, J=2.5 Hz), 7.16 (1 H, dd, J=8.6, 2.0 Hz), 6.74-6.92 (2 H, m), 6.47 (1 H, d, J=8.1 Hz), 6.06 (1 H, br), 3.85 (2 H, br); HRMS (ESI-TOF) m/z: [M+H]⁺ calculated for C₁₁H₁₁⁷⁹BrN₃ and C₁₁H₁₁⁸¹BrN₃ 264.0131, 266.0111; found 264.0148, 266.0128.

### 5-Bromo-N¹-(pyridin-3-yl)benzene-1,2-diamine (54g)

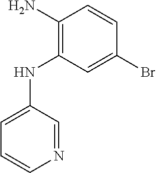 [SMILES: Nc1ccc(Br)cc1Nc1cccnc1]

The above compound (54g) was prepared from 53g following Method O and purified by column chromatography on silica gel (MeOH/CH₂Cl₂, 5:95) to give 54g (42%) as a yellow solid; ¹H NMR (CDCl₃, 500 MHz) 8.20 (1 H, d, J=2.5 Hz), 8.13 (1 H, dd, J=4.6, 1.5 Hz), 7.23 (1 H, d, J=2.0 Hz), 7.15-7.12 (2 H, m), 7.03-7.01 (1 H, m), 6.70 (1 H, d, J=8.6 Hz), 5.20 (1 H, br), 3.75 (2 H, br); HRMS (ESI-TOF) m/z: [M+H]⁺ calculated for C₁₁H₁₁⁷⁹BrN₃ and C₁₁H₁₁⁸¹BrN₃ 264.0131, 266.0111; found 264.0131, 266.0110.

### Example 17—General Procedure for the Preparation of 6-bromo-1-alkyl-1H-benzo[d]imidazol-2(3H)-one—Method P

### 6-Bromo-1-ethyl-1H-benzo[d]imidazol-2(3H)-one (55a)

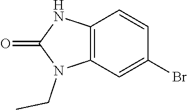

To a solution of 54a (103.0 mg, 0.48 mmol) in anhydrous dichloromethane (4 mL) was added triphosgene (71.0 mg, 0.24 mmol) and triethylamine (0.17 mL, 1.24 mmol) at 0° C. The resulting mixture was purged with nitrogen then stirred at 0° C. for 1 h. After being quenched with water (5 mL), the aqueous layer was extracted with EtOAc (2×20 mL). The combined organic extracts were washed with brine, dried over anhydrous Na₂SO₄, filtered, and concentrated. The residue was purified by column chromatography on silica gel (EtOAc/hexane, 20:80 to 40:60) to afford 55a (95.8 mg, 83%) as an off-solid solid; ¹H NMR (DMSO-d₆, 500 MHz) 10.95 (1 H, s), 7.11 (1 H, dd, J=8.1, 2.0 Hz), 6.91 (1 H, d, J=8.1 Hz), 3.80 (2 H, q, J=7.1 Hz), 1.16 (3 H, t, J=7.1 Hz); HRMS (ESI-TOF) m/z: [M+H]⁺ calculated for C₉H₁₀⁷⁹BrN₂O and C₉H₁₀⁸¹BrN₂O 240.9972, 242.9951; found 240.9973 242.9953.

### 6-Bromo-1-isopropyl-1H-benzo[d]imidazol-2 (3H)-one (55b)

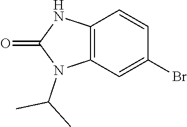

The above compound (55b) was prepared from 54b following Method P and purified by column chromatography on silica gel (EtOAc/hexane, 20:80 to 40:60) to give 55b (54%) as a pale pink solid; ¹H NMR (CDCl₃, 500 MHz) 8.07 (1 H, s), 7.17 (1 H, dd, J=8.1, 1.0 Hz), 6.91 (1 H, d, J=8.1 Hz), 4.70-4.65 (1 H, m), 1.53 (6 H, d, J=2.5 Hz); HRMS (ESI-TOF) m/z: [M+H]⁺ calculated for C₁₀H₁₂⁷⁹BrN₂O and C₁₀H₁₂⁸¹BrN₂O 255.0128, 257.0180; found 255.0128, 257.0106.

### 6-Bromo-1-cyclopropyl-1H-benzo[d]imidazol-2(3H)-one (55c)

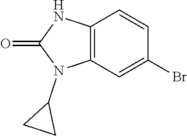 [SMILES: O=c1[nH]c2ccc(Br)cc2n1C1CC1]

The above compound (55c) was prepared from 53c following Method N and P. The crude product from first step was used in next step without further purification. The crude product was purified by column chromatography on silica gel (EtOAc/hexane, 30:70 to 60:40) to give 55c (44% over 2 steps) as a light brown solid; ¹H NMR (DMSO-d₆, 500 MHz) 10.86 (1 H, s), 7.26 (1 H, d, J=1.5 Hz), 7.13 (1 H, dd, J=8.1, 1.5 Hz), 6.88 (1 H, d, J=8.1 Hz), 2.84-2.79 (1 H, m), 1.01-0.98 (2 H, m), 0.86-0.83 (2 H, m); HRMS (ESI-TOF) m/z: [M+H]⁺ calculated for C₁₀H₁₀⁷⁹BrN₂O and C₁₀H₁₀⁸¹BrN₂O 252.9972, 254.9951; found 252.9983, 254.9963.

### 6-Bromo-1-cyclopentyl-1H-benzo[d]imidazol-2(3H)-one (55d)

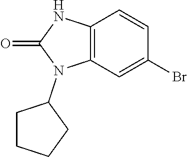 [SMILES: O=c1[nH]c2ccc(Br)cc2n1C1CCCC1]

The above compound (55d) was prepared from 54d following Method P and purified by column chromatography on silica gel (EtOAc/hexane, 20:80 to 40:60) to give 55d (53%) as a white solid; ¹H NMR (CDCl₃, 600 MHz) 10.59 (1 H, s), 7.18-7.17 (2 H, m), 7.00 (1 H, d, J=8.5 Hz), 4.84 (1 H, quin, J=8.5 Hz), 2.11-1.98 (6 H, m), 1.79-1.75 (2 H, m); HRMS (ESI-TOF) m/z: [M+H]⁺ calculated for C₁₂H₁₄⁷⁹BrN₂O and C₁₂H₁₄⁸¹BrN₂O 281.0285, 283.0264; found 281.0293, 283.0274.

### 6-Bromo-1-phenyl-1H-benzo[d]imidazol-2 (3H)-one (55e)

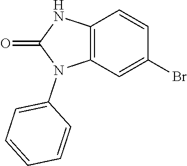 [SMILES: O=c1[nH]c2ccc(Br)cc2n1-c1ccccc1]

The above compound (55e) was prepared from 54e following Method P. The reaction was stirred at 0° C. to room temperature for 16 h and purified by column chromatography on silica gel (EtOAc/hexane, 20:80 to 30:70) to give 59e (78%) as an off-white solid; ¹H NMR (CDCl₃, 500 MHz) 9.17 (1 H, s), 7.59-7.56 (2 H, m), 7.53-7.51 (2 H, m), 7.46 (1 H, t, J=7.6 Hz), 7.23 (1 H, dd, J=8.4, 2.0 Hz), 7.17 (1 H, d, J=1.5 Hz), 7.00(1 H, d, J=8.6 Hz); HRMS (ESI-TOF) m/z: [M+H]⁺ calculated for C₁₃H₁₀⁷⁹BrN₂O and C₁₃H₁₀⁸¹BrN₂O 288.9972, 290.9951; found 288.9962, 290.9944.

### 6-Bromo-1-(pyridin-2-yl)-1H-benzo[d]yl)-2 (3H)-one (55f)

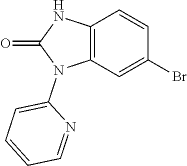 [SMILES: O=c1[nH]c2ccc(Br)cc2n1-c1ccccn1]

The above compound (55f) was prepared from 54f following Method P. The reaction was stirred at 0° C. to room temperature for 1.5 h and purified by column chromatography on silica gel (EtOAc/hexane, 35:65 to 70:30) to give 55f (44%) as a white solid; ¹H NMR (CDCl₃, 500 MHz) 8.60-8.59 (1 H, m), 8.27 (1 H, d, J=2.0 Hz), 8.12 (2 H, d, J=8.6 Hz), 7.88 (1 H, td, J=7.4, 2.0 Hz), 7.29-7.26 (1 H, m), 6.96 (1 H, d, J=8.6 Hz); HRMS (ESI-TOF) m/z: [M+H]⁺ calculated for C₁₂H₉⁷⁹BrN₃O and C₁₂H₉⁸¹BrN₃O 289.9924, 291.9904; found 289.9796, 291.9775.

### 6-Bromo-1-(pyridin-3-yl)-1H-benzo[d]imidazol-2 (3H)-one (55g)

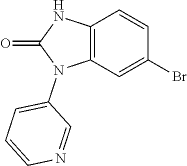 [SMILES: O=c1[nH]c2ccc(Br)cc2n1-c1cccnc1]

The above compound (55g) was prepared from 54g following Method P and purified by column chromatography on silica gel (MeOH/CH₂Cl₂, 5:95) to give 55g (59%) as a brown solid; ¹H NMR (CD₃OD, 500 MHz) 8.78 (1 H, s), 8.64 (1 H, s), 8.06-8.04 (1 H, m), 7.66 (1 H, dd, J=7.9, 4.6 Hz), 7.29 (1 H, dd, J=8.4, 2.0 Hz), 7.18 (1 H, d, J=1.5 Hz), 7.07 (1 H, d, J=8.6 Hz); HRMS (ESI-TOF) m/z: [M+H]⁺ calculated for C₁₂H₉⁷⁹BrN₃O and C₁₂H₉⁸¹BrN₃O 289.9924, 291.9904; found 289.9923, 291.9903.

### 6-Bromo-1-(pyridin-4-yl)-1H-benzo[d]imidazol-2(3H)-one (55h)

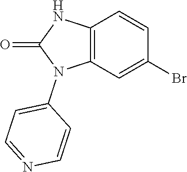 [SMILES: O=c1[nH]c2ccc(Br)cc2n1-c1ccncc1]

The above compound (55h) was prepared from 53h following Method O and P. The crude product from first step was used in next step without further purification. The crude product was purified by column chromatography on silica gel (MeOH/CH₂Cl₂, 5:95) to give 55h (20% over 2 steps) as a pale yellow solid; ¹H NMR (DMSO-d₆, 500 MHz) 11.50 (1 H, s), 8.73 (2 H, d, J=6.1 Hz), 7.66 (2 H, d, J=6.1 Hz), 7.38 (1 H, d, J=1.5 Hz), 7.28(1 H, dd, J=8.1, 2.0 Hz), 7.05 (1 H, d, J=8.1 Hz); HRMS (ESI-TOF) m/z: [M+H]⁺ calculated for C₁₂H₉⁷⁹BrN₃O and C₁₂H₉⁸¹BrN₃O 289.9924, 291.9904; found 289.9938, 291.9920.

### 1-Ethyl-6-(4,4,5,5-tetramethyl-1,3,2-dioxaborolan-2-yl)-1H-benzo[d]yl)-2 (3H)-one (56a)

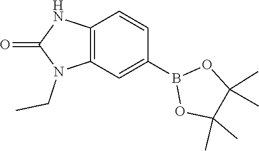 [SMILES: CCn1c(=O)[nH]c2ccc(B3OC(C)(C)C(C)(C)O3)cc21]

The above compound (56a) was prepared from 55a following Method D and purified by column chromatography on silica gel (MeOH/CH₂Cl₂, 2.5:97.5 to 5:95) to give 56a (86%) as a light pink solid; ¹H NMR (CDCl₃, 500 MHz) 10.36 (1 H, s), 7.58 (1 H, d, J=7.6 Hz), 7.44 (1 H, s), 7.14 (1 H, d, J=7.6 Hz), 3.98 (2 H, q, J=7.6 Hz), 1.46-1.29 (15 H, m);

HRMS (ESI-TOF) m/z: [M+H]⁺ calculated for C₁₅H₂₂BN₂O₃ 289.1718; found 289.1728.

### 1-Isopropyl-6-(4,4,5,5-tetramethyl-1,3,2-dioxaborolan-2-yl)-1H-benzo[d]imidazol-2(3H)-one (56b)

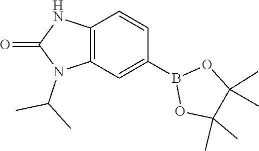 [SMILES: CC(C)n1c(=O)[nH]c2ccc(B3OC(C)(C)C(C)(C)O3)cc21]

The above compound (56b) was prepared from 55b following Method D and purified by column chromatography on silica gel (EtOAc/hexane, 30:70 to 40:60) to give 56b (99%) as a light brown solid; ¹H NMR (CDCl₃, 500 MHz) 10.40 (1 H, s), 7.56-7.54 (2 H, m), 7.12 (1 H, d, J=7.6 Hz), 4.77-4.69 (1 H, m), 1.58 (6 H, d, J=7.1 Hz), 1.35 (12 H, s); HRMS (ESI-TOF) m/z: [M+H]⁺ calculated for C₁₆H₂₄BN₂O₃ 303.1875; found 303.1886.

### 1-Cyclopropyl-6-(4,4,5,5-tetramethyl-1, 3, 2-dioxaborolan-2-yl)-1H-benzo[d]imidazol-2 (3H)-one (56c)

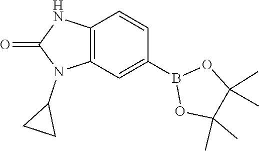 [SMILES: CC1(C)OB(c2ccc3[nH]c(=O)n(C4CC4)c3c2)OC1(C)C]

The above compound (56c) was prepared from 55c following Method D and purified by column chromatography on silica gel (EtOAc/hexane, 50:50 to 60:40) to give 56c (88%) as a yellow solid; ¹H NMR (CDCl₃, 500 MHz) 8.86 (1 H, s), 7.61 (1 H, s), 7.57 (1 H, d, J=8.1 Hz), 7.06 (1 H, d, J=7.6 Hz), 2.90-2.86 (1 H, m), 1.36 (12 H, s), 1.17-1.13 (2 H, m), 1.08-1.05 (2 H, m); HRMS (ESI-TOF) m/z: [M+H]⁺ calculated for C₁₆H₂₂BN₂O₃ 301.1718; found 301.1777.

### 1-Cyclopentyl-6-(4,4,5,5-tetramethyl-1,3,2-dioxaborolan-2-yl)-1H-benzo[d]imidazol-2(3H)-one (56d)

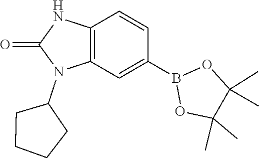 [SMILES: CC1(C)OB(c2ccc3[nH]c(=O)n(C4CCCC4)c3c2)OC1(C)C]

The above compound (56d) was prepared from 55d following Method D and purified by column chromatography on silica gel (EtOAc/hexane, 50:50 to 60:40) to give 56d (99%) as a white solid; ¹H NMR (CDCl₃, 500 MHz) 9.52 (1 H, s), 7.55 (1 H, d, J=7.6 Hz), 7.47 (1 H, s), 7.09 (1 H, d, J=8.1 Hz), 4.80 (1 H, quin, J=8.6 Hz), 2.24-2.20 (2 H, m), 2.04-1.99 (4 H, m), 1.74-1.72 (2 H, m), 1.36 (12 H, s); HRMS (ESI-TOF) m/z: [M+H]⁺ calculated for C₁₈H₂₆BN₂O₃ 329.2031; found 329.2052.

### 1-Phenyl-6-(4,4,5,5-tetramethyl-1,3,2-dioxaborolan-2-yl)-1H-benzo[d]imidazol-2(3H)-one (56e)

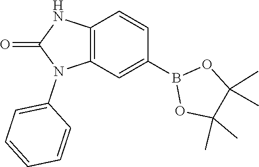 [SMILES: CC1(C)OB(c2ccc3[nH]c(=O)n(-c4ccccc4)c3c2)OC1(C)C]

The above compound (56e) was prepared from 55e following Method D and purified by column chromatography on silica gel (EtOAc/hexane, 30:70 to 40:60) to give 56e (84%) as a light brown solid; ¹H NMR (CDCl₃, 500 MHz) 9.38 (1 H, s), 7.61 (1 H, d, J=8.6 Hz), 7.57-7.53 (5 H, m), 7.44 (1 H, s), 7.14 (1 H, d, J=7.6 Hz), 1.32 (12 H, s); HRMS (ESI-TOF) m/z: [M+H]⁺ calculated for C₁₉H₂₂BN₂O₃ 337.1718; found 337.1723.

### 1-(Pyridin-2-yl)-6-(4,4,5,5-tetramethyl-1,3,2-dioxaborolan-2-yl)-1H-benzo[d]imidazol-2(3H)-one (56f)

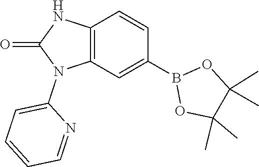 [SMILES: CC1(C)OB(c2ccc3[nH]c(=O)n(-c4ccccn4)c3c2)OC1(C)C]

The above compound (56f) was prepared from 55f following Method D and purified by column chromatography on silica gel (EtOAc/hexane, 30:70 to 35:75) to give 56f (57%) as a white solid; ¹H NMR (CDCl₃, 500 MHz) 9.06 (1 H, s), 8.66 (1 H, d, J=5.1 Hz), 8.24 (1 H, s), 7.99 (1 H, d, J=8.1 Hz), 7.88 (1 H, td, J=7.4, 2.0 Hz), 7.64 (1 H, d, J=8.1 Hz), 7.29-7.27 (1 H, m), 7.12 (1 H, d, J=7.6 Hz), 1.34 (12 H, s); HRMS (ESI-TOF) m/z: [M+H]⁺ calculated for C₁₈H₂₁BN₃O₃ 338.1671; found 338.1678.

### 1-(Pyridin-4-yl)-6-(4,4,5,5-tetramethyl-1,3,2-dioxaborolan-2-yl)-1H-benzo[d]imidazol-2(3H)-one (56h)

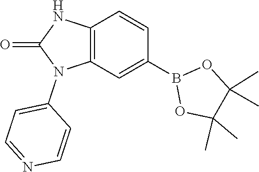 [SMILES: CC1(C)OB(c2ccc3[nH]c(=O)n(-c4ccncc4)c3c2)OC1(C)C]

The above compound (56h) was prepared from 55h following Method D and purified by column chromatography on silica gel (EtOAc/hexane, 30:70 to 35:75) to give 56h (90%) as a yellow solid; ¹H NMR (CDCl₃, 500 MHz) 8.81 (2 H, d, J=6.1 Hz), 8.46 (1 H, s), 7.67-7.64 (3 H, m), 7.14 (1 H, d, J=7.6 Hz), 1.34 (12 H, s); HRMS (ESI-TOF) m/z: [M+H]⁺ calculated for C₁₈H₂₁BN₃O₃ 338.1671; found 338.1898.

### 6-(2-(Benzylamino)pyrimidin-5-yl)-1-ethyl-1H-benzo[d]imidazol-2(3H)-one (57a)

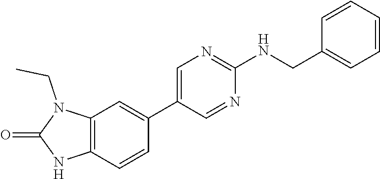 [SMILES: CCn1c(=O)[nH]c2ccc(-c3cnc(NCc4ccccc4)nc3)cc21]

The above compound (57a) was prepared from 56a and 6a following Method I and purified by column chromatography on silica gel (MeOH/CH₂Cl₂, 5:95) to give 57a (51%) as a light brown solid; ¹H NMR (DMSO-d₆, 500 MHz) 10.82 (1 H, s), 8.60 (2 H, s), 7.79 (1 H, t, J=6.6 Hz), 7.40 (1 H, s), 7.33-7.28 (4 H, m), 7.20 (2 H, t, J=8.6 Hz), 7.00 (1 H, d, J=7.6 Hz), 4.54 (2 H, d, J=6.1 Hz), 3.85 (2 H, q, J=7.1 Hz), 1.20 (3 H, t, J=7.1 Hz); HRMS (ESI-TOF) m/z: [M+H]⁺ calculated for C₂₀H₂₀N₅O 346.1663; found 346.1979.

### 6-(2-(Benzylamino)pyrimidin-5-yl)-1-isopropyl-1H-benzo[d]imidazol-2(3H)-one (57b)

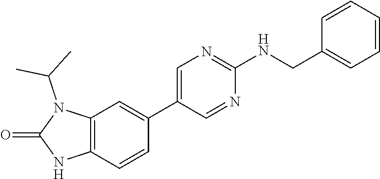 [SMILES: CC(C)n1c(=O)[nH]c2ccc(-c3cnc(NCc4ccccc4)nc3)cc21]

The above compound (57b) was prepared from 56b and 6a following Method I and purified by column chromatography on silica gel (MeOH/CH₂Cl₂, 2.5:97.5 to 5:95) to give 57b (43%) as a white solid; ¹H NMR (DMSO-d₆, 500 MHz) 10.80 (1 H, s), 8.58 (2 H, s), 7.78 (1 H, t, J=6.6 Hz), 7.42 (1 H, s), 7.33-7.28 (4 H, m), 7.22-7.16 (2 H, m), 6.99 (1 H, d, J=7.6 Hz), 4.62-4.53 (3 H, m), 1.45 (6 H, d, J=6.6 Hz); HRMS (ESI-TOF) m/z: [M+H]⁺ calculated for C₂₁H₂₂N₅O 360.1819; found 360.1953.

### 6-(2-(Benzylamino)pyrimidin-5-yl)-1-cyclopropyl-1H-benzo[d]imidazol-2(3H)-one (57c)

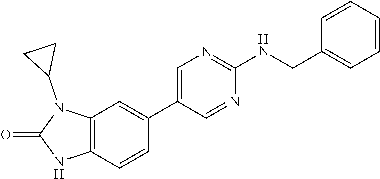 [SMILES: O=c1[nH]c2ccc(-c3cnc(NCc4ccccc4)nc3)cc2n1C1CC1]

The above compound (57c) was prepared from 56c and 6a following Method I and purified by column chromatography on silica gel (MeOH/CH₂Cl₂, 2.5:97.5 to 5:95) to give 57c (49%) as a white solid; ¹H NMR (DMSO-d₆, 500 MHz) 10.73 (1 H, s), 8.56 (2 H, s), 7.79 (1 H, t, J=6.6 Hz), 7.33-7.28 (5 H, m), 7.21-7.17 (2 H, m), 6.97 (1 H, d, J=8.1 Hz), 4.54 (2 H, d, J=6.1 Hz), 2.84-2.82 (1 H, m), 1.03-0.99 (2 H, m), 0.89-0.86 (2 H, m); HRMS (ESI-TOF) m/z: [M+H]⁺ calculated for C₂₁H₂₂N₅O 358.1663; found 358.1667.

### 6-(2-(Benzylamino)pyrimidin-5-yl)-1-cyclopentyl-1H-benzo[d]imidazol-2(3H)-one (57d)

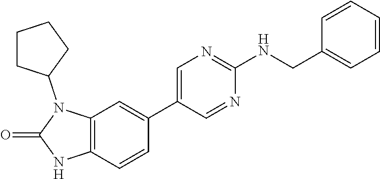 [SMILES: O=c1[nH]c2ccc(-c3cnc(NCc4ccccc4)nc3)cc2n1C1CCCC1]

The above compound (57d) was prepared from 56d and 6a following Method I and purified by column chromatography on silica gel (EtOAc/hexane, 25:75 to 50:50) to give 57d (43%) as a pale yellow solid; ¹H NMR (DMSO-d₆, 500 MHz) 10.84 (1 H, s), 8.57 (2 H, s), 7.79 (1 H, t, J=6.1 Hz), 7.33-7.28 (5 H, m), 7.22-7.16 (2 H, m), 7.00(1 H, d, J=8.1 Hz), 4.72 (1 H, quin, J=8.6 Hz), 4.54 (2 H, d, J=6.1 Hz), 2.13-2.09 (2 H, m), 1.89-1.84 (4 H, m), 1.64-1.62 (2 H, m); HRMS (ESI-TOF) m/z: [M+H]⁺ calculated for C₂₃H₂₄N₅O 386.1976; found 386.2100.

### 6-(2-(Benzylamino)pyrimidin-5-yl)-1phenyl-1H-benzo[d]imidazol-2 (3H)-one (57e)

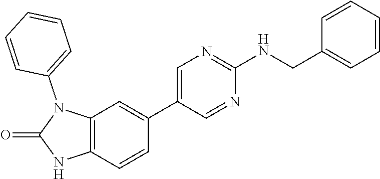 [SMILES: O=c1[nH]c2ccc(-c3cnc(NCc4ccccc4)nc3)cc2n1-c1ccccc1]

The above compound (57e) was prepared from 56e and 6a following Method I and purified by column chromatography on silica gel (MeOH/CH₂Cl₂, 2.5:97.5 to 5:95) to give 57e (43%) as a light purple solid; ¹H NMR (DMSO-d₆, 500 MHz) 11.18 (1 H, s), 8.50 (2 H, s), 7.78 (1 H, t, J=5.6 Hz), 7.60-7.53 (4 H, m), 7.42 (1 H, t, J=7.1 Hz), 7.32-7.26 (5 H, m), 7.19 (1 H, t, J=6.6 Hz), 7.13-7.11 (2 H, m), 4.51 (2 H, d, J=6.1 Hz); HRMS (ESI-TOF) m/z: [M+H]⁺ calculated for C₂₄H₂₀N₅O 394.1662; found 394.1666.

### 6-(2-(Benzylamino)pyrimidin-5-yl)-1-(pyridin-2-yl)-1H-benzo[d]imidazol-2(3H)-one (57f)

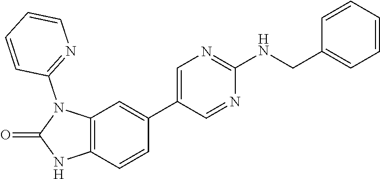 [SMILES: O=c1[nH]c2ccc(-c3cnc(NCc4ccccc4)nc3)cc2n1-c1ccccn1]

The above compound (570 was prepared from 56f and 6a following Method I and purified by column chromatography on silica gel (EtOAc/hexane₂, 25:75 to 50:50) to give 57f (47%) as a white solid; ¹H NMR (DMSO-d₆, 500 MHz) 11.33 (1 H, s), 8.61 (1 H, dd, J=4.8, 1.0 Hz), 8.52 (2 H, s), 8.08-8.04 (2 H, m), 7.98 (1 H, td, J=7.4, 2.0 Hz), 7.82-7.80 (1 H, t, J=6.6 Hz), 7.33-7.28 (6 H, m), 7.20 (1 H, t, J=6.6 Hz), 7.12 (1 H, d, J=8.1 Hz), 4.54 (2 H, d, J=6.6 Hz); HRMS (ESI-TOF) m/z: [M+H]⁺ calculated for C₂₃H₁₉N₆O 395.1615; found 395.1601.

### 6-(2-(Benzylamino)pyrimidin-5-yl)-1-(pyridin-3-yl)-1H-benzo[d]imidazol-2(3H)-one (57g)

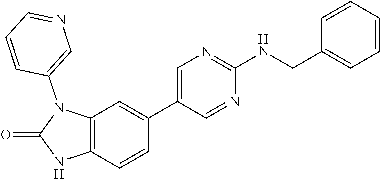 [SMILES: O=c1[nH]c2ccc(-c3cnc(NCc4ccccc4)nc3)cc2n1-c1cccnc1]

The above compound (57g) was prepared from 56g and 6a following Method I and purified by column chromatography on silica gel (MeOH/CH₂Cl₂, 2.5:97.5 to 5:95) to give 57g (47%) as a gray solid; ¹H NMR (DMSO-d₆, 500 MHz) 11.30 (1 H, s), 8.83 (1 H, d, J=2.0 Hz), 8.61 (1 H, dd, J=4.6, 1.0 Hz), 8.54 (2 H, s), 8.06 (1 H, d, J=8.1 Hz), 7.79 (1 H, t, J=6.6 Hz), 7.59 (1 H, dd, J=7.9, 5.1 Hz), 7.31-7.26 (5 H, m), 7.22-7.19 (2 H, m), 7.13 (1 H, d, J=8.6 Hz), 4.52 (2 H, d, J=6.6 Hz); HRMS (ESI-TOF) m/z: [M+H]⁺ calculated for C₂₃H₁₉N₆O 395.1615; found 395.1613.

### 6-(2-(Benzylamino)pyrimidin-5-yl)-1-(pyridin-4-yl)-1H-benzo[d]imidazol-2(3H)-one (57h)

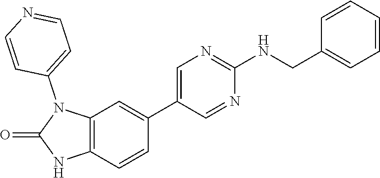 [SMILES: O=c1[nH]c2ccc(-c3cnc(NCc4ccccc4)nc3)cc2n1-c1ccncc1]

The above compound (57h) was prepared from 56h and 6a following Method I and purified by column chromatography on silica gel (MeOH/CH₂Cl₂, 2.5:97.5 to 5:95) to give 57h (17%) as a white solid; ¹H NMR (DMSO-d₆, 500 MHz) 11.38 (1 H, s), 8.71 (2 H, d, J=6.1 Hz), 8.58 (2 H, s), 7.81 (1 H, t, J=6.6 Hz), 7.74 (2 H, d, J=6.1 Hz), 7.43 (1 H, s), 7.34-7.27 (5 H, m), 7.20 (1 H, t, J=6.6 Hz), 7.14 (1 H, d, J=8.1 Hz), 4.53 (2 H, d, J=6.1 Hz); HRMS (ESI-TOF) m/z: [M+H]⁺ calculated for C₂₃H₁₉N₆O 395.1615; found 395.1882.

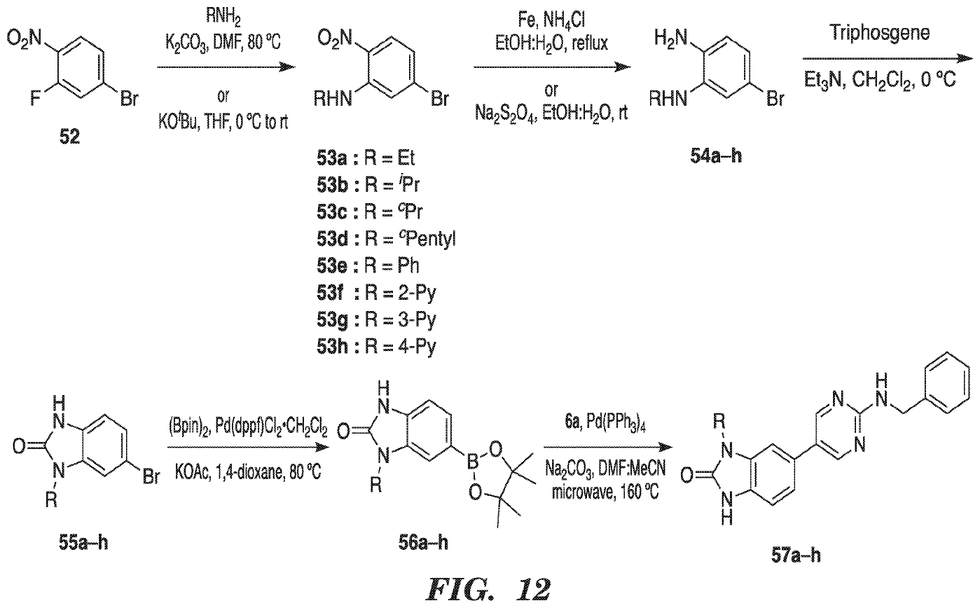

The synthetic schemes for compounds 57a-h, discussed supra, are shown in FIG. 12.

### Example 18—General Procedure for the Preparation of 5-Iodo-N-(1-phenylethyl)pyrimidin-2-amine—Method Q

### (R)-5-Iodo-N-(1-phenylethyl)pyrimidin-2-amine (59a)

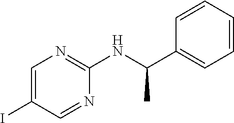 [SMILES: C[C@@H](Nc1ncc(I)cn1)c1ccccc1]

To a solution of 2-chloro-5-iodopyrimidine 58 (250.0 mg, 1.04 mmol) in NMP (1 mL) was added (R)-(+)-a-methylbenzylamine (0.17 ml, 1.35 mmol). The reaction mixture was irradiated in microwave at 160° C. for 90 min. The solvent was removed in vacuo then purified by column chromatography on silica gel (EtOAc/hexane, 10:90) to give 59a (98%) as a white solid; ¹H NMR (CDCl₃, 600 MHz) 8.35 (2 H, s), 7.36-7.32 (4 H, m), 7.28-7.24 (1 H, m), 5.43 (1 H, d, J=8.5 Hz), 5.10 (1 H, quin, J=7.3 Hz), 1.55 (3 H, d, J=7.3 Hz); HRMS (ESI-TOF) m/z: [M+H]⁺ calculated for C₁₂H₁₃IN₃ 326.0149; found 326.0379.

### (S)-5-Iodo-N-(1-phenylethyl)pyrimidin-2-amine (59b)

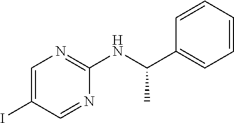 [SMILES: C[C@H](Nc1ncc(I)cn1)c1ccccc1]

The above compound (59b) was prepared from 58 and (S)-(-)-α-methylbenzylamine following Method I and purified by column chromatography on silica gel (EtOAc/hexane, 210:90) to give 59b (98%) as a white solid; ¹H NMR (CDCl₃, 600 MHz) 8.34 (2 H, s), 7.36-7.32 (4 H, m), 7.26-7.24 (1 H, m), 5.45 (1 H, d, J=6.1 Hz), 5.10 (1 H, quin, J=7.3 Hz), 1.55 (3 H, d, J=6.1 Hz); HRMS (ESI-TOF) m/z: [M+H]⁺ calculated for C₁₂H₁₃1N₃ 326.0149; found 326.0126.

### (R)-5-(2-((1-Phenylethyl)amino)pyrimidin-5-yl)-1H-benzo[d]imidazol-2(3H)-one (60a)

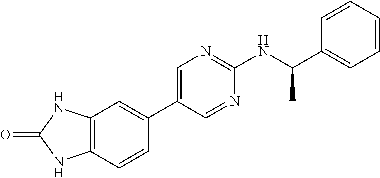 [SMILES: C[C@@H](Nc1ncc(-c2ccc3[nH]c(=O)[nH]c3c2)cn1)c1ccccc1]

The above compound (60a) was prepared from 13b and 59a following Method I and purified by column chromatography on silica gel (MeOH/CH₂Cl₂, 5:95) to give 60a (26%) as a yellow solid; ¹H NMR (DMSO-d₆, 500 MHz) 10.66 (1 H, s), 10.60 (1 H, s), 8.48 (2 H, s), 7.73 (1 H, d, J=8.1 Hz), 7.39 (2 H, d, J=7.1 Hz), 7.28 (2 H, t, J=7.1Hz), 7.17 (1 H, t, J=7.1 Hz), 7.1 (1 H, d, J=8.1 Hz), 7.05 (1 H, s), 6.94 (1 H, d, J=8.1 Hz), 5.13 (1 H, quin, J=7.6 Hz), 1.44 (3 H, d, J=7.1 Hz); HRMS (ESI-TOF) m/z: [M+H]⁺ calculated for C₁₉H₁₈N₅O 332.1506; found 332.1533.

### (S)-5-(2-((1-Phenylethyl)amino)pyrimidin-5-yl)-1H-benzo[d]imidazol-2(3H)-one (60b)

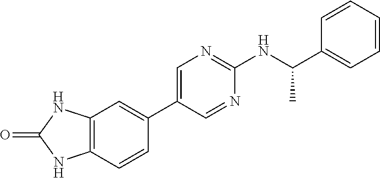 [SMILES: C[C@H](Nc1ncc(-c2ccc3[nH]c(=O)[nH]c3c2)cn1)c1ccccc1]

The above compound (60b) was prepared from 13b and 59b following Method I and purified by column chromatography on silica gel (MeOH/CH₂Cl₂, 5:95) to give 60b (18%) as a yellow solid; ¹H NMR (DMSO-d₆, 500 MHz) 10.66 (1 H, s), 10.60 (1 H, s), 8.48 (2 H, s), 7.73 (1 H, d, J=8.6 Hz), 7.39 (2 H, d, J=7.6 Hz), 7.29 (2 H, t, J=7.6 Hz), 7.17 (1 H, t, J=7.1 Hz), 7.10 (1 H, d, J=8.1 Hz), 7.05 (1 H, s), 6.94 (1 H, d, J=8.1 Hz), 5.13 (1 H, quin, J=7.6 Hz), 1.44 (3 H, d, J=6.6 Hz); HRMS (ESI-TOF) m/z: [M+H]⁺ calculated for C₁₉H₁₈N₅O 332.1506; found 332.1500.

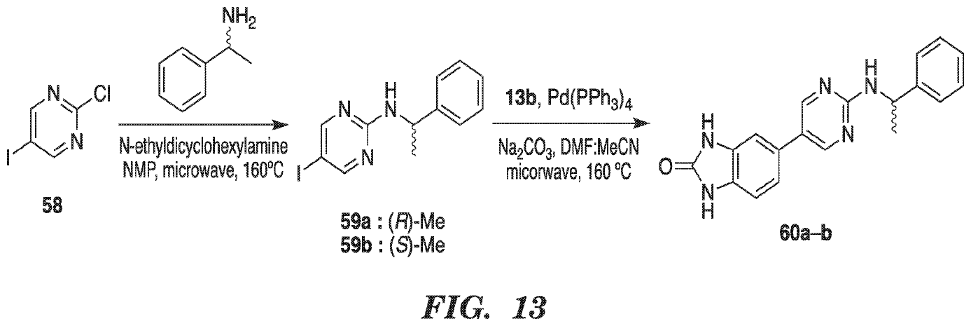

The synthetic schemes for compounds 60a-b, discussed supra, are shown in FIG. 13.

### 5-Iodo-N-(3-(2-methoxyethoxy)benzyl)pyrimidin-2-amine (61)

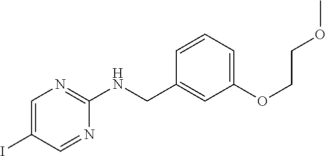 [SMILES: COCCOc1cccc(CNc2ncc(I)cn2)c1]

To a solution of 22j (60.0 mg, 0.18 mmol) in anhydrous DMF (2 mL) was added 1-chlor-2-methoxyethan (0.02 ml, 0.18 mmol), potassium carbonate (51 mg, 0.37 mmol) and sodium iodide (0.3 mg, 0.002 mmol). The reaction mixture was irradiated in microwave at 160° C. for 30 min. After being quenched with water (5 mL), the aqueous layer was extracted with EtOAc (2×10 mL). The combined organic extracts were washed with brine, dried over anhydrous Na₂SO₄, filtered, and concentrated. The residue was purified by column chromatography on silica gel (EtOAc/hexane, 20:80 to 30:70) to give 61a (99%) as a white solid; ¹H NMR (CDCl₃, 600 MHz) 8.27 (2 H, br), 7.25 (1 H, t, J=8.5 Hz), 6.92-6.91 (2 H, m), 6.85 (1 H, dd, J=7.3, 2.4 Hz), 6.02 (1 H, s), 4.54 (2 H, d, J=4.9 Hz), 4.11 (2 H, dd, J=4.9, 3.7 Hz), 3.74 (2 H, dd, J=4.9, 3.7 Hz), 3.44 (3 H, s); HRMS (ESI-TOF) m/z: [M+H]⁺ calculated for C₁₄H₁₇IN₃O₂386.0360; found 386.0260.

### 5-(2-((3-(2-Methoxyethoxy)benzyl)amino)pyrimidin-5-yl)-1H-benzo[d]imidazol-2(3H)-one (62)

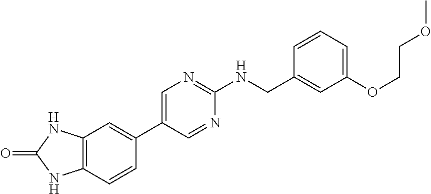 [SMILES: COCCOc1cccc(CNc2ncc(-c3ccc4[nH]c(=O)[nH]c4c3)cn2)c1]

The above compound (62) was prepared from 13b and 61 following Method I and purified by column chromatography on silica gel (MeOH/CH₂Cl₂, 5:95 to 10:90) to give 62 (27%) as a pale yellow solid; ¹H NMR (DMSO-d₆, 500 MHz) 10.66 (1 H, s), 10.61 (1 H, s), 8.52 (2 H, s), 7.73 (1 H, t, J=5.6 Hz), 7.19 (1 H, t, J=7.6 Hz), 7.13 (1 H, d, J=8.1 Hz), 7.08 (1 H, s), 6.95 (1 H, d, J=8.1 Hz), 6.88 (2 H, s), 6.77 (1 H, d, J=7.1 Hz), 4.50 (2 H, d, J=5.6 Hz), 4.04 (2 H, s), 3.62 (2 H, s), 3.28 (3 H, s); HRMS (ESI-TOF) m/z: [M+H]⁺ calculated for C₂₁H₂₂N₅O₃ 392.1717; found 392.1706.

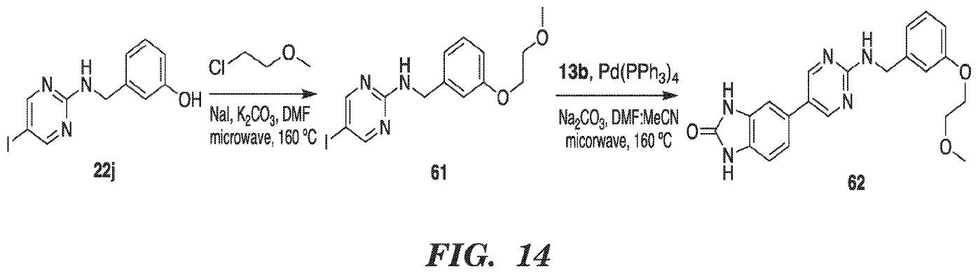

The synthetic scheme for compound 62, discussed supra, is shown in FIG. 14.

### Example 19—DYRK1A Binding Assays/IC50 Assay

Compounds were tested for DYRK1A binding activity at the commercial kinase profiling service DiscoverX. DiscoverX uses proprietary KINOMEscan® Assay (Fabian et al., “A Small Molecule-kinase Interaction Map for Clinical Kinase Inhibitors,” Nat. Biotechnol. 23(3):329-336 (2005), which is hereby incorporated by reference in its entirety). Compounds were screened for DYRK1A activity at a single concentration of 3 μM in duplicates. Similarly, the dissociation constant K_{d} of the hit compounds from the initial screening was determined at DiscoverX using their proprietary KINOMEscan® Assay. K_{d} values are determined using eleven serial three fold dilutions with the highest concentration of 60 μM. The results of the binding assay are displayed in Table 1.

Some compounds were also tested for IC50 (nM) activity using the DYRK1/DYRK1A assay from Reaction Biology Corp., the results of which are also displayed in Table 1.

TABLE 1
DYRK1A Binding Activity

| Compound | STRUCTURE | Kd (nM) | IC50 (nM) |
|---|---|---|---|
| 16a | 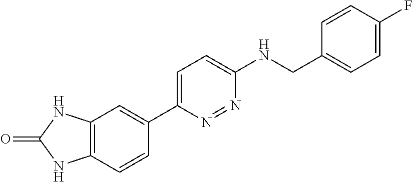 [SMILES: O=c1[nH]c2ccc(-c3ccc(NCc4ccc(F)cc4)nn3)cc2[nH]1] | 7000 | — |
| 16b | 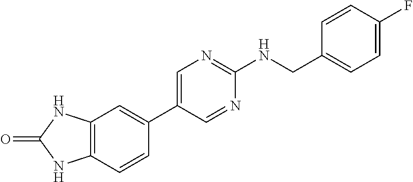 [SMILES: O=c1[nH]c2ccc(-c3cnc(NCc4ccc(F)cc4)nc3)cc2[nH]1] | 73, 82, 140 | — |
| 16c | 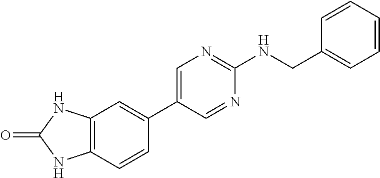 [SMILES: O=c1[nH]c2ccc(-c3cnc(NCc4ccccc4)nc3)cc2[nH]1] | 50, 95, 80, 60 | — |
| 17c | 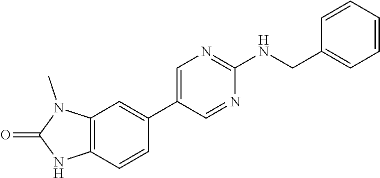 [SMILES: Cn1c(=O)[nH]c2ccc(-c3cnc(NCc4ccccc4)nc3)cc21] | 59 | — |
| 17d | 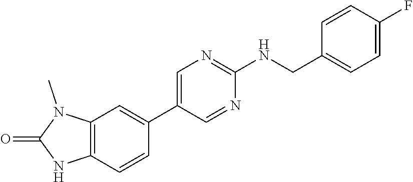 [SMILES: Cn1c(=O)[nH]c2ccc(-c3cnc(NCc4ccc(F)cc4)nc3)cc21] | 90 | — |
| 17i | 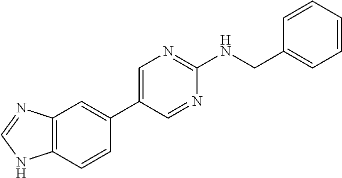 [SMILES: c1ccc(CNc2ncc(-c3ccc4[nH]cnc4c3)cn2)cc1] | 2000 | — |
| 17j | 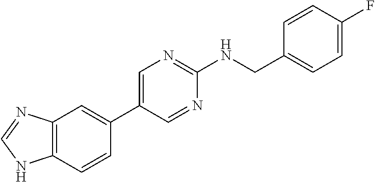 [SMILES: Fc1ccc(CNc2ncc(-c3ccc4[nH]cnc4c3)cn2)cc1] | 2300 | — |
| 18c | 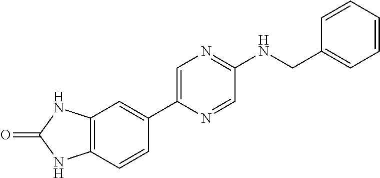 [SMILES: O=c1[nH]c2ccc(-c3cnc(NCc4ccccc4)cn3)cc2[nH]1] | 170 | — |
| 18d | 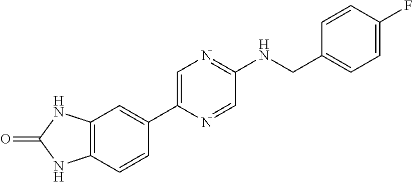 [SMILES: O=c1[nH]c2ccc(-c3cnc(NCc4ccc(F)cc4)cn3)cc2[nH]1] | 260 | — |
| 18i | 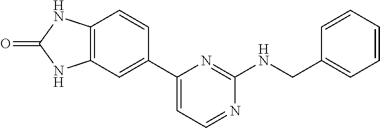 [SMILES: O=c1[nH]c2ccc(-c3ccnc(NCc4ccccc4)n3)cc2[nH]1] | TBD | — |
| 18j | 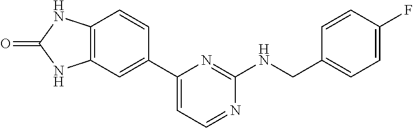 [SMILES: O=c1[nH]c2ccc(-c3ccnc(NCc4ccc(F)cc4)n3)cc2[nH]1] | TBD | — |
| 18k | 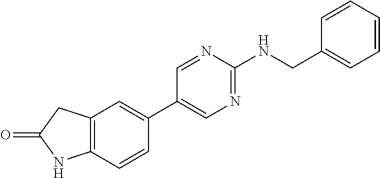 [SMILES: O=C1Cc2cc(-c3cnc(NCc4ccccc4)nc3)ccc2N1] | TBD | — |
| 18m | 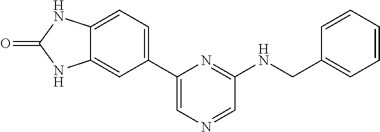 [SMILES: O=c1[nH]c2ccc(-c3cncc(NCc4ccccc4)n3)cc2[nH]1] | — | 1.13 |
| 29a | 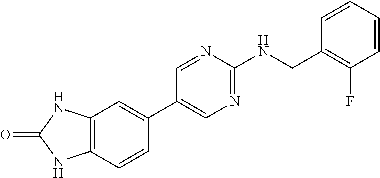 [SMILES: O=c1[nH]c2ccc(-c3cnc(NCc4ccccc4F)nc3)cc2[nH]1] | 69 | — |
| 29b | 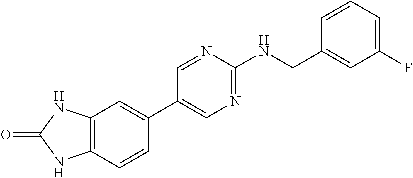 [SMILES: O=c1[nH]c2ccc(-c3cnc(NCc4cccc(F)c4)nc3)cc2[nH]1] | 91 | — |
| 29c | 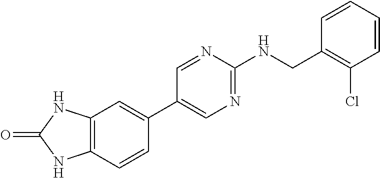 [SMILES: O=c1[nH]c2ccc(-c3cnc(NCc4ccccc4Cl)nc3)cc2[nH]1] | 250 | — |
| 29d | 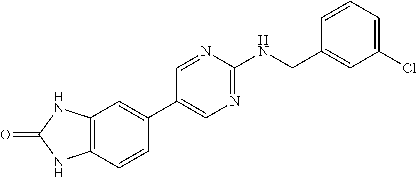 [SMILES: O=c1[nH]c2ccc(-c3cnc(NCc4cccc(Cl)c4)nc3)cc2[nH]1] | 280 | — |
| 29e | 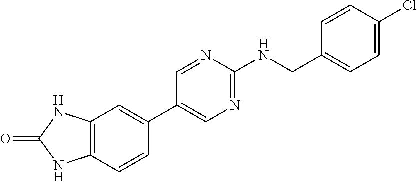 [SMILES: O=c1[nH]c2ccc(-c3cnc(NCc4ccc(Cl)cc4)nc3)cc2[nH]1] | 180 | — |
| 29f | 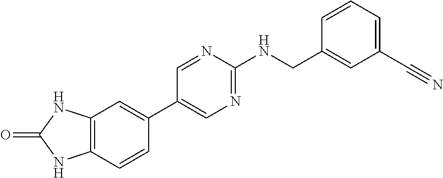 [SMILES: N#Cc1cccc(CNc2ncc(-c3ccc4[nH]c(=O)[nH]c4c3)cn2)c1] | 49 | — |
| 29g | 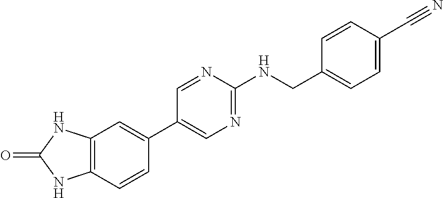 [SMILES: N#Cc1ccc(CNc2ncc(-c3ccc4[nH]c(=O)[nH]c4c3)cn2)cc1] | 120 | — |
| 29h | 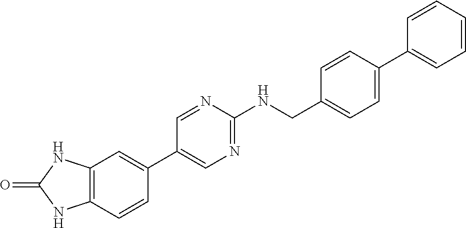 [SMILES: O=c1[nH]c2ccc(-c3cnc(NCc4ccc(-c5ccccc5)cc4)nc3)cc2[nH]1] | 1400 | — |
| 29i | 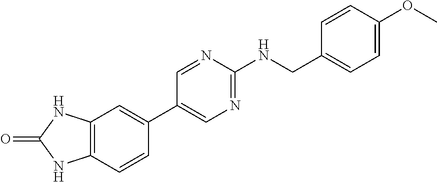 [SMILES: COc1ccc(CNc2ncc(-c3ccc4[nH]c(=O)[nH]c4c3)cn2)cc1] | TBD | — |
| 29j | 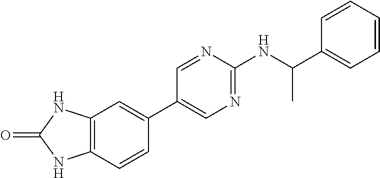 [SMILES: CC(Nc1ncc(-c2ccc3[nH]c(=O)[nH]c3c2)cn1)c1ccccc1] | 360 | — |
| 29k | 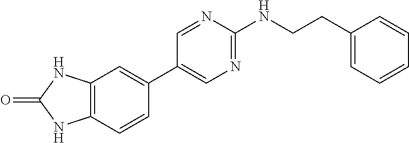 [SMILES: O=c1[nH]c2ccc(-c3cnc(NCCc4ccccc4)nc3)cc2[nH]1] | 200 | — |
| 29l | 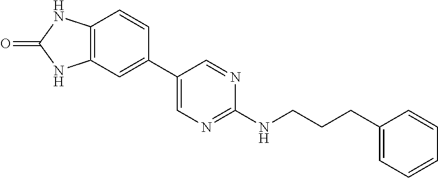 [SMILES: O=c1[nH]c2ccc(-c3cnc(NCCCc4ccccc4)nc3)cc2[nH]1] | — | 34 |
| 30b | 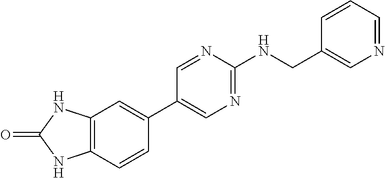 [SMILES: O=c1[nH]c2ccc(-c3cnc(NCc4cccnc4)nc3)cc2[nH]1] | 190 | — |
| 30c | 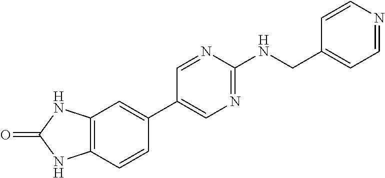 [SMILES: O=c1[nH]c2ccc(-c3cnc(NCc4ccncc4)nc3)cc2[nH]1] | 130 | — |
| 30d | 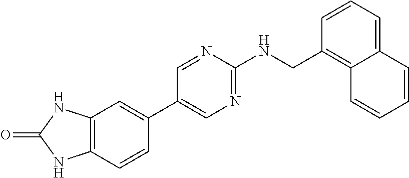 [SMILES: O=c1[nH]c2ccc(-c3cnc(NCc4cccc5ccccc45)nc3)cc2[nH]1] | 920 | — |
| 30e | 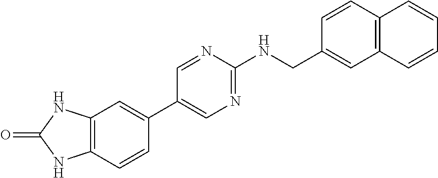 [SMILES: O=c1[nH]c2ccc(-c3cnc(NCc4ccc5ccccc5c4)nc3)cc2[nH]1] | 7100 | — |
| 32a | 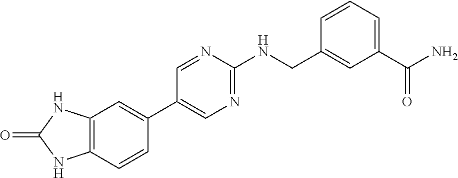 [SMILES: NC(=O)c1cccc(CNc2ncc(-c3ccc4[nH]c(=O)[nH]c4c3)cn2)c1] | 130 | — |
| 32b | 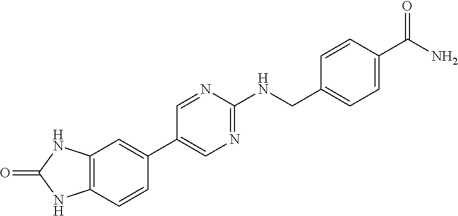 [SMILES: NC(=O)c1ccc(CNc2ncc(-c3ccc4[nH]c(=O)[nH]c4c3)cn2)cc1] | 46 | — |
| 37a | 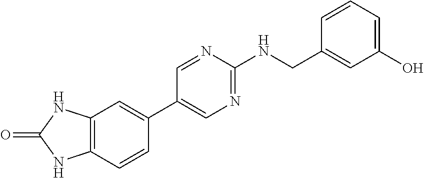 [SMILES: O=c1[nH]c2ccc(-c3cnc(NCc4cccc(O)c4)nc3)cc2[nH]1] | TBD | — |
| 37b | 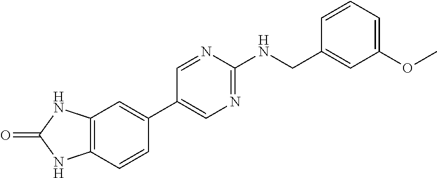 [SMILES: COc1cccc(CNc2ncc(-c3ccc4[nH]c(=O)[nH]c4c3)cn2)c1] | TBD | — |
| 37c | 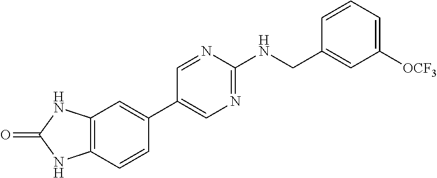 [SMILES: O=c1[nH]c2ccc(-c3cnc(NCc4cccc(OC(F)(F)F)c4)nc3)cc2[nH]1] | TBD | — |
| 38a | 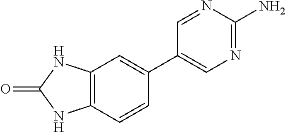 [SMILES: Nc1ncc(-c2ccc3[nH]c(=O)[nH]c3c2)cn1] | TBD | — |
| 38b | 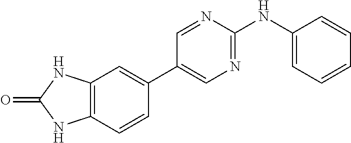 [SMILES: O=c1[nH]c2ccc(-c3cnc(Nc4ccccc4)nc3)cc2[nH]1] | TBD | — |
| 38c | 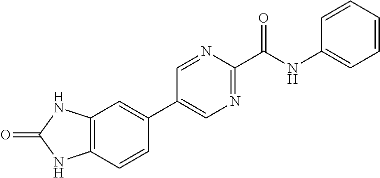 [SMILES: O=C(Nc1ccccc1)c1ncc(-c2ccc3[nH]c(=O)[nH]c3c2)cn1] | TBD | — |
| 38d | 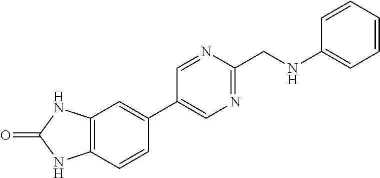 [SMILES: O=c1[nH]c2ccc(-c3cnc(CNc4ccccc4)nc3)cc2[nH]1] | TBD | — |
| 44a | 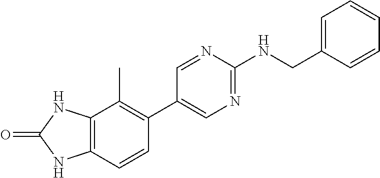 [SMILES: Cc1c(-c2cnc(NCc3ccccc3)nc2)ccc2[nH]c(=O)[nH]c12] | TBD | — |
| 44b | 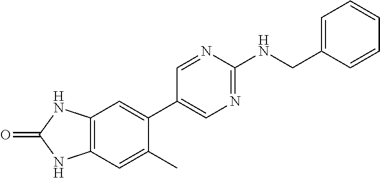 [SMILES: Cc1cc2[nH]c(=O)[nH]c2cc1-c1cnc(NCc2ccccc2)nc1] | TBD | — |
| 48a | 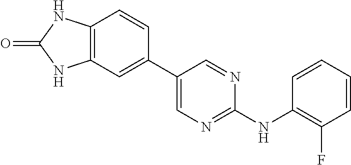 [SMILES: O=c1[nH]c2ccc(-c3cnc(Nc4ccccc4F)nc3)cc2[nH]1] | — | 78 |
| 48b | 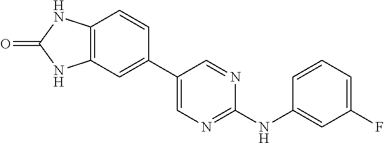 [SMILES: O=c1[nH]c2ccc(-c3cnc(Nc4cccc(F)c4)nc3)cc2[nH]1] | — | 47.7 |
| 48c | 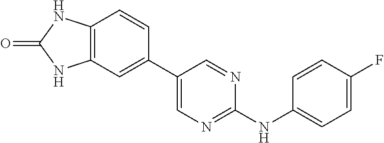 [SMILES: O=c1[nH]c2ccc(-c3cnc(Nc4ccc(F)cc4)nc3)cc2[nH]1] | — | 58 |
| 48d | 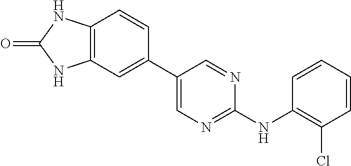 [SMILES: O=c1[nH]c2ccc(-c3cnc(Nc4ccccc4Cl)nc3)cc2[nH]1] | — | 59 |
| 48e | 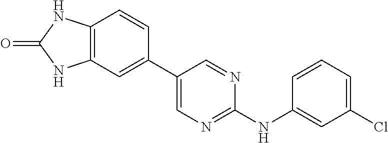 [SMILES: O=c1[nH]c2ccc(-c3cnc(Nc4cccc(Cl)c4)nc3)cc2[nH]1] | — | 54.9 |
| 48f | 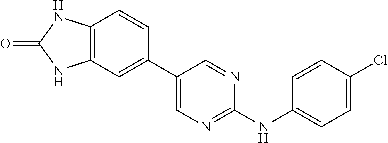 [SMILES: O=c1[nH]c2ccc(-c3cnc(Nc4ccc(Cl)cc4)nc3)cc2[nH]1] | — | 90.6 |
| 48g | 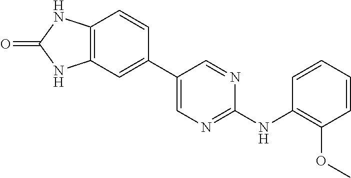 [SMILES: COc1ccccc1Nc1ncc(-c2ccc3[nH]c(=O)[nH]c3c2)cn1] | — | 497 |
| 48h | 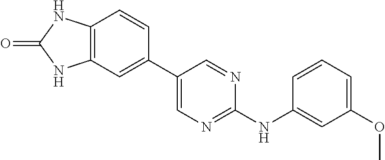 [SMILES: COc1cccc(Nc2ncc(-c3ccc4[nH]c(=O)[nH]c4c3)cn2)c1] | — | 23.5 |
| 48i | 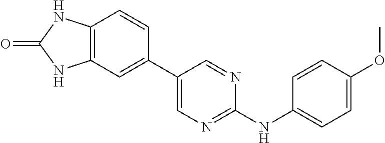 [SMILES: COc1ccc(Nc2ncc(-c3ccc4[nH]c(=O)[nH]c4c3)cn2)cc1] | — | 30.1 |
| 51a | 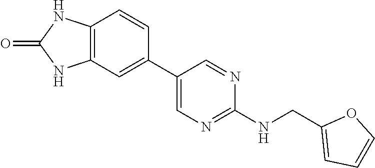 [SMILES: O=c1[nH]c2ccc(-c3cnc(NCc4ccco4)nc3)cc2[nH]1] | — | 73.2 |
| 51b | 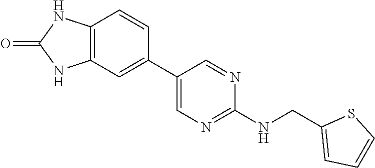 [SMILES: O=c1[nH]c2ccc(-c3cnc(NCc4cccs4)nc3)cc2[nH]1] | — | 32.7 |
| 51e | 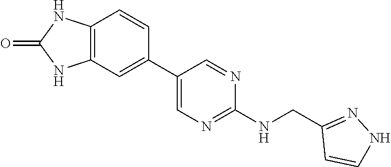 [SMILES: O=c1[nH]c2ccc(-c3cnc(NCc4cc[nH]n4)nc3)cc2[nH]1] | — | 337 |
| 51d | 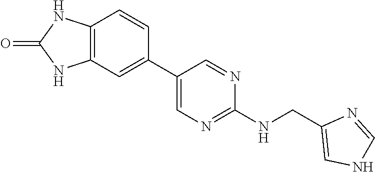 [SMILES: O=c1[nH]c2ccc(-c3cnc(NCc4c[nH]cn4)nc3)cc2[nH]1] | — | 326 |
| 51c | 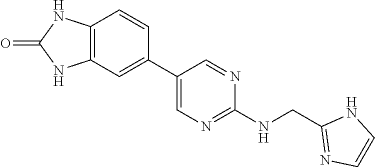 [SMILES: O=c1[nH]c2ccc(-c3cnc(NCc4ncc[nH]4)nc3)cc2[nH]1] | — | 960 |
| 51f | 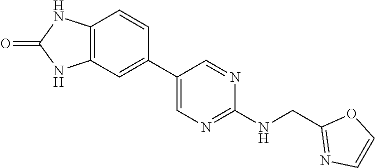 [SMILES: O=c1[nH]c2ccc(-c3cnc(NCc4ncco4)nc3)cc2[nH]1] | — | 427 |
| 57a | 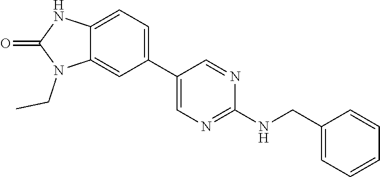 [SMILES: CCn1c(=O)[nH]c2ccc(-c3cnc(NCc4ccccc4)nc3)cc21] | — | 243 |
| 57b | 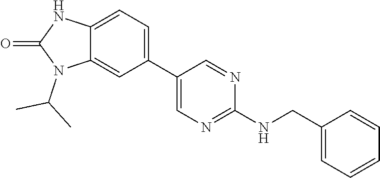 [SMILES: CC(C)n1c(=O)[nH]c2ccc(-c3cnc(NCc4ccccc4)nc3)cc21] | — | 4280 |
| 57c | 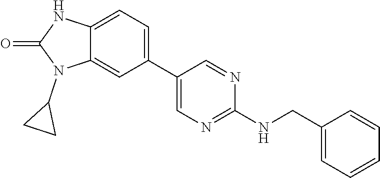 [SMILES: O=c1[nH]c2ccc(-c3cnc(NCc4ccccc4)nc3)cc2n1C1CC1] | — | 714 |
| 57d | 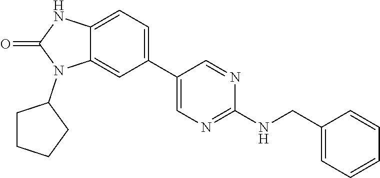 [SMILES: O=c1[nH]c2ccc(-c3cnc(NCc4ccccc4)nc3)cc2n1C1CCCC1] | — | NI |
| 57e | 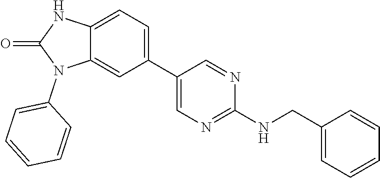 [SMILES: O=c1[nH]c2ccc(-c3cnc(NCc4ccccc4)nc3)cc2n1-c1ccccc1] | — | NI |
| 57f | 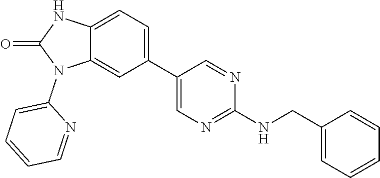 [SMILES: O=c1[nH]c2ccc(-c3cnc(NCc4ccccc4)nc3)cc2n1-c1ccccn1] | — | NI |
| 57g | 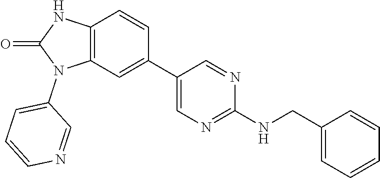 [SMILES: O=c1[nH]c2ccc(-c3cnc(NCc4ccccc4)nc3)cc2n1-c1cccnc1] | — | NI |
| 57h | 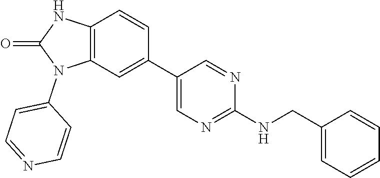 [SMILES: O=c1[nH]c2ccc(-c3cnc(NCc4ccccc4)nc3)cc2n1-c1ccncc1] | — | NI |
| 60a | 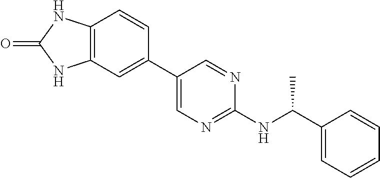 [SMILES: C[C@@H](Nc1ncc(-c2ccc3[nH]c(=O)[nH]c3c2)cn1)c1ccccc1] | — | 499 |
| 60b | 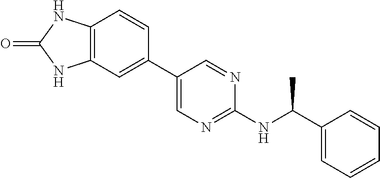 [SMILES: C[C@H](Nc1ncc(-c2ccc3[nH]c(=O)[nH]c3c2)cn1)c1ccccc1] | — | 177 |
| 62 | 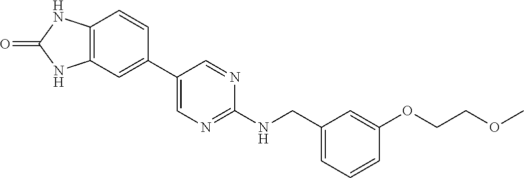 [SMILES: COCCOc1cccc(CNc2ncc(-c3ccc4[nH]c(=O)[nH]c4c3)cn2)c1] | — | 48.4 |

TBD: To be determined
NI: No inhibition

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A compound of formula (I) having the following structure: 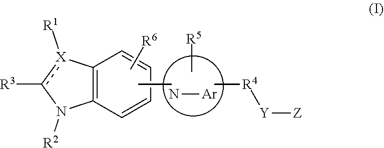
or a stereoisomer, pharmaceutically acceptable salt, oxide, or solvate thereof, wherein
R¹ and R⁶ are independently optionally present, and when present, each is independently selected from H, substituted or unsubstituted C₁-C₆ alkyl, halogen, —CF₃, —OCF₃, or a substituted or unsubstituted cycloalkyl;
R² is selected from H, substituted or unsubstituted C₁-C₆ alkyl, halogen, —CF₃, —OCF₃, substituted or unsubstituted cycloalkyl, or substituted or unsubstituted aryl or heteroaryl;
R³ is optionally present, and when present is an oxygen that forms a carbonyl, or a substituted or unsubstituted C₁-C₆ alkyl;
R⁴ is NH, carbonyl, or branched or unbranched C₁-C₆ alkyl;
R⁵ is optionally present, and when present is a substituted or unsubstituted C₁-C₆ alkyl, halogen, —CF₃, or —OCF₃; [symbol]
is a single or double bond;
X is C, CH, O, or N;
N—Ar is selected from pyridazine, pyrazine, and pyrimidine;
Y is a bond, NH, or branched or linear C₁-C₆ substituted or unsubstituted alkyl; and
Z is H or a substituted or unsubstituted aryl, biaryl, heteroaryl, cycloalkyl, heterocycle, or alkyl, wherein said substituent is selected from hydroxyl, —CF₃, —OCF₃, halogen, nitrile, aryl, C₁-C₆ alkoxy, amide, amino, alkyl, aminocarboxamide, substituted or unsubstituted carboxamide, or a C₁-C₆ alkyl ester.

2. The compound according to claim 1, wherein
R¹ and R² are H;
R³ is an oxygen that forms a carbonyl;
R⁴ is NH; [symbol]
is a single bond; and
X is N.

3. The compound according to claim 2, wherein Z is an unsubstituted phenyl ring or a phenyl ring substituted with hydroxyl, —OCF₃, a halogen, a nitrile, a benzene ring, C₁-C₆ alkoxy, or —CONH₂.

4. The compound according to claim 3, selected from the group consisting of

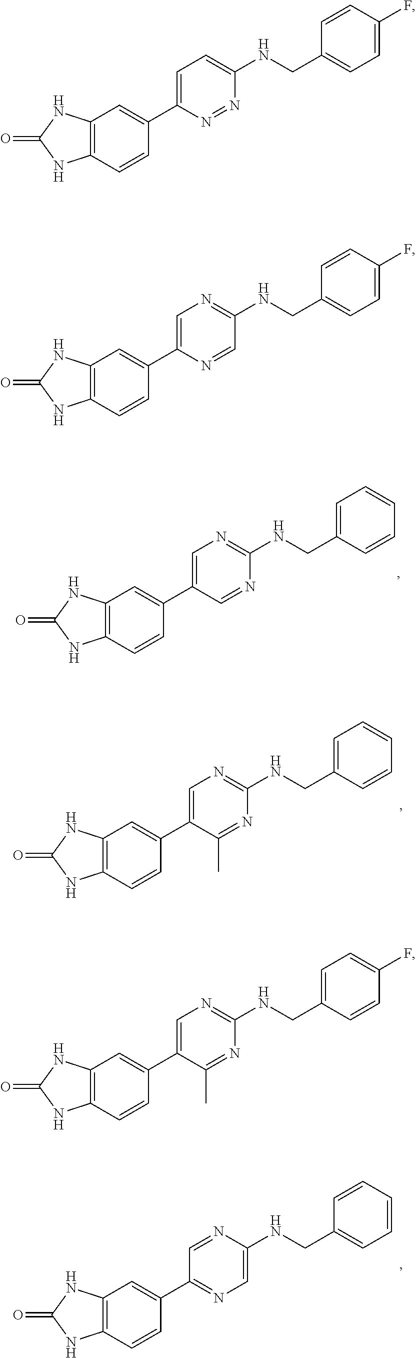 [SMILES: O=c1[nH]c2ccc(-c3ccc(NCc4ccc(F)cc4)nn3)cc2[nH]1 ; O=c1[nH]c2ccc(-c3cnc(NCc4ccc(F)cc4)cn3)cc2[nH]1 ; O=c1[nH]c2ccc(-c3cnc(NCc4ccccc4)nc3)cc2[nH]1 ; Cc1nc(NCc2ccccc2)ncc1-c1ccc2[nH]c(=O)[nH]c2c1 ; Cc1nc(NCc2ccc(F)cc2)ncc1-c1ccc2[nH]c(=O)[nH]c2c1 ; O=c1[nH]c2ccc(-c3cnc(NCc4ccccc4)cn3)cc2[nH]1]

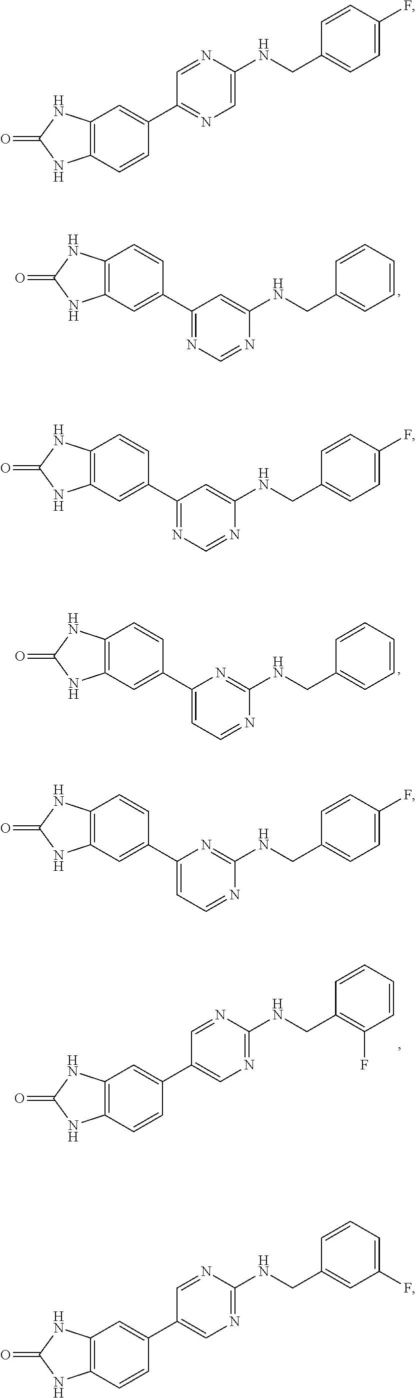

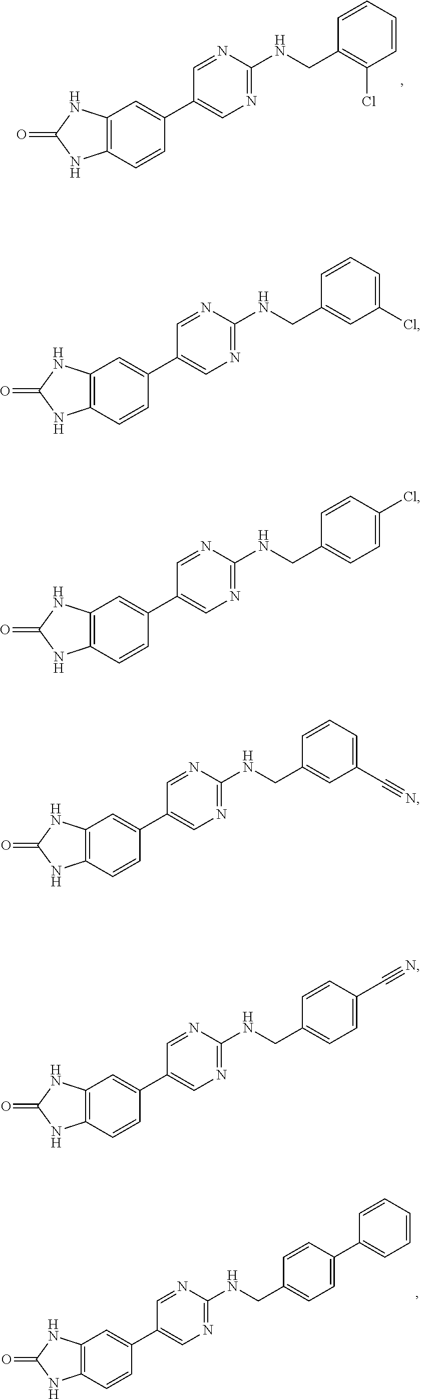 [SMILES: O=c1[nH]c2ccc(-c3cnc(NCc4ccccc4Cl)nc3)cc2[nH]1 ; O=c1[nH]c2ccc(-c3cnc(NCc4cccc(Cl)c4)nc3)cc2[nH]1 ; O=c1[nH]c2ccc(-c3cnc(NCc4ccc(Cl)cc4)nc3)cc2[nH]1 ; N#Cc1cccc(CNc2ncc(-c3ccc4[nH]c(=O)[nH]c4c3)cn2)c1 ; N#Cc1ccc(CNc2ncc(-c3ccc4[nH]c(=O)[nH]c4c3)cn2)cc1 ; O=c1[nH]c2ccc(-c3cnc(NCc4ccc(-c5ccccc5)cc4)nc3)cc2[nH]1]

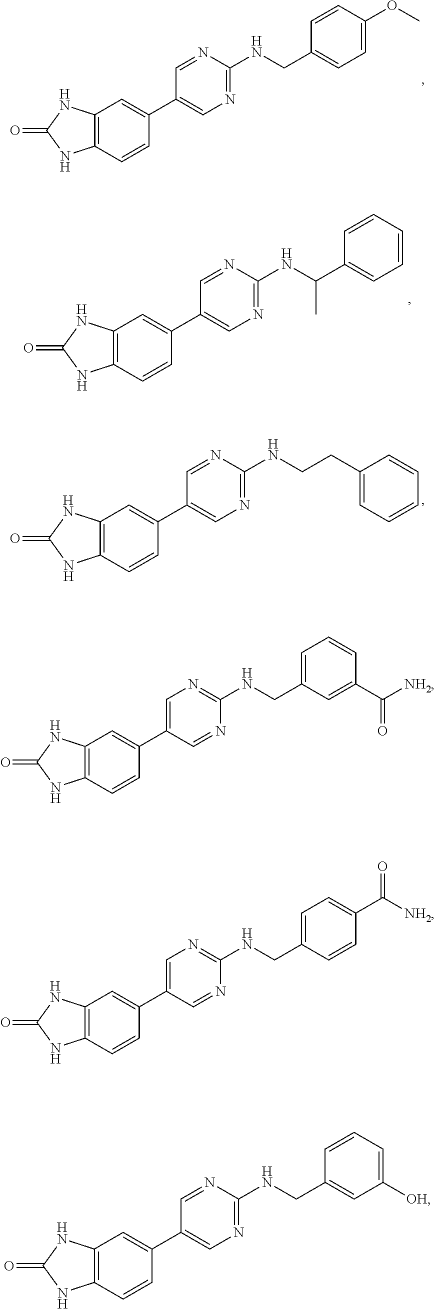 [SMILES: COc1ccc(CNc2ncc(-c3ccc4[nH]c(=O)[nH]c4c3)cn2)cc1 ; CC(Nc1ncc(-c2ccc3[nH]c(=O)[nH]c3c2)cn1)c1ccccc1 ; O=c1[nH]c2ccc(-c3cnc(NCCc4ccccc4)nc3)cc2[nH]1 ; NC(=O)c1cccc(CNc2ncc(-c3ccc4[nH]c(=O)[nH]c4c3)cn2)c1 ; NC(=O)c1ccc(CNc2ncc(-c3ccc4[nH]c(=O)[nH]c4c3)cn2)cc1 ; O=c1[nH]c2ccc(-c3cnc(NCc4cccc(O)c4)nc3)cc2[nH]1]

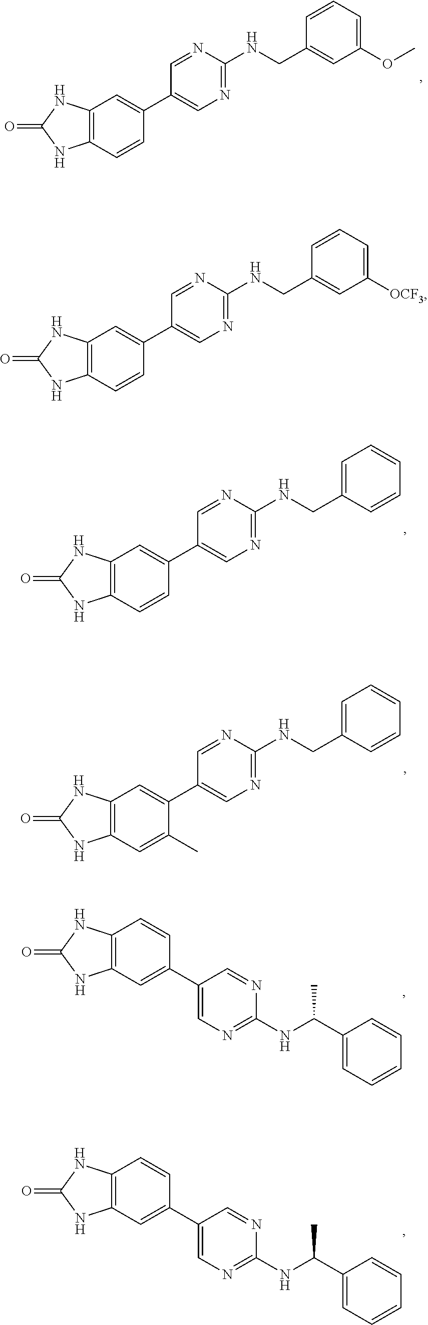 [SMILES: COc1cccc(CNc2ncc(-c3ccc4[nH]c(=O)[nH]c4c3)cn2)c1 ; O=c1[nH]c2ccc(-c3cnc(NCc4cccc(OC(F)(F)F)c4)nc3)cc2[nH]1 ; O=c1[nH]c2ccc(-c3cnc(NCc4ccccc4)nc3)cc2[nH]1 ; Cc1cc2[nH]c(=O)[nH]c2cc1-c1cnc(NCc2ccccc2)nc1 ; C[C@@H](Nc1ncc(-c2ccc3[nH]c(=O)[nH]c3c2)cn1)c1ccccc1 ; C[C@H](Nc1ncc(-c2ccc3[nH]c(=O)[nH]c3c2)cn1)c1ccccc1]

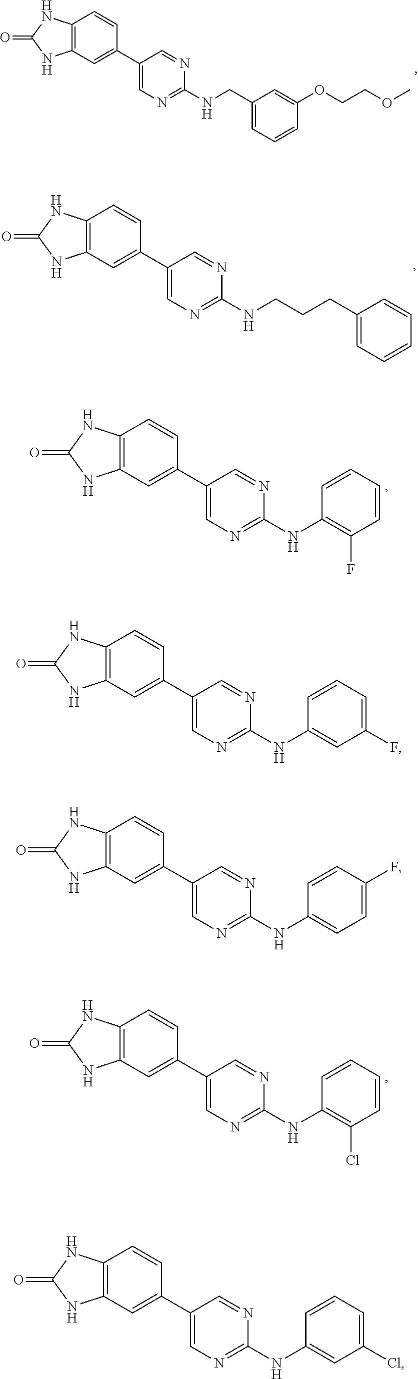

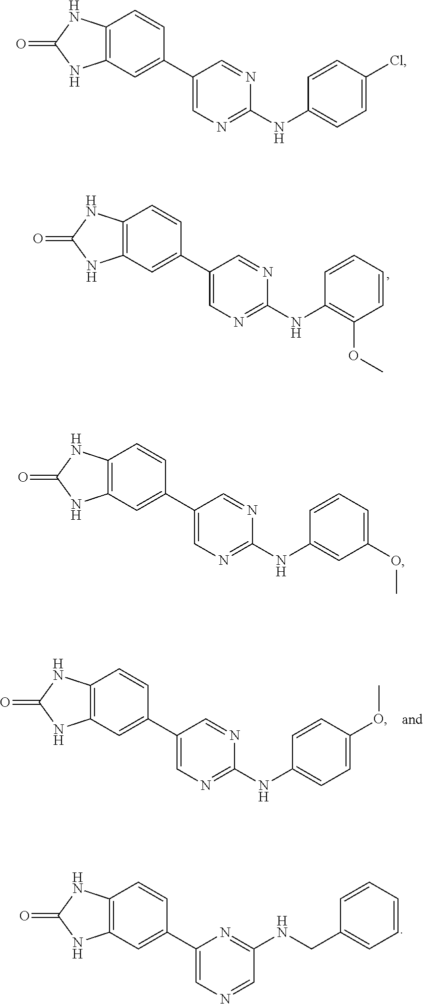 [SMILES: O=c1[nH]c2ccc(-c3cnc(Nc4ccc(Cl)cc4)nc3)cc2[nH]1 ; COc1ccccc1Nc1ncc(-c2ccc3[nH]c(=O)[nH]c3c2)cn1 ; COc1cccc(Nc2ncc(-c3ccc4[nH]c(=O)[nH]c4c3)cn2)c1 ; COc1ccc(Nc2ncc(-c3ccc4[nH]c(=O)[nH]c4c3)cn2)cc1 ; "and" ; O=c1[nH]c2ccc(-c3cncc(NCc4ccccc4)n3)cc2[nH]1]

5. The compound according to claim 2, wherein Z is selected from pyridinyl and naphthalene.

6. The compound according to claim 5, selected from the group consisting of

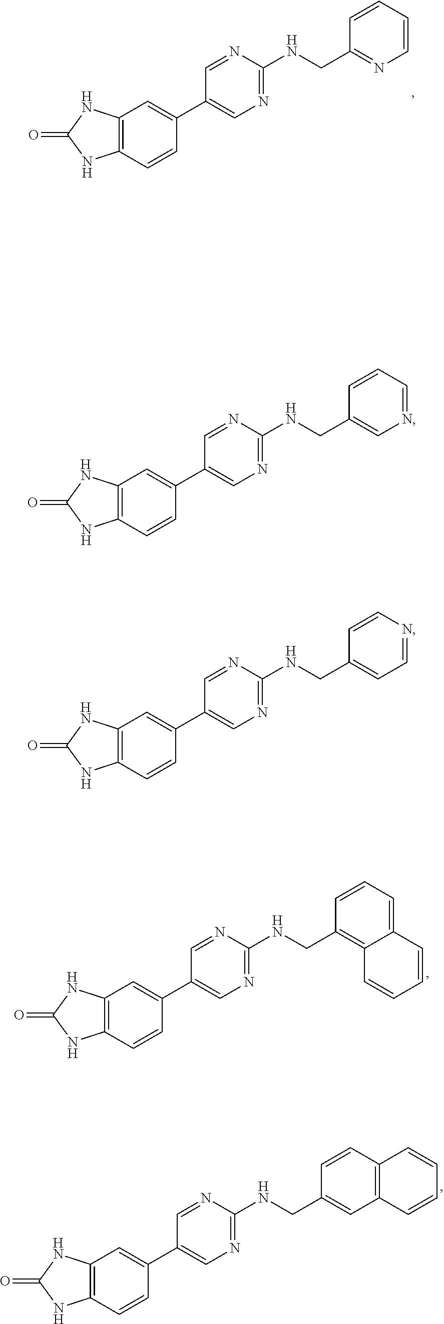 [SMILES: O=c1[nH]c2ccc(-c3cnc(NCc4ccccn4)nc3)cc2[nH]1 ; O=c1[nH]c2ccc(-c3cnc(NCc4cccnc4)nc3)cc2[nH]1 ; O=c1[nH]c2ccc(-c3cnc(NCc4ccncc4)nc3)cc2[nH]1 ; O=c1[nH]c2ccc(-c3cnc(NCc4cccc5ccccc45)nc3)cc2[nH]1 ; O=c1[nH]c2ccc(-c3cnc(NCc4ccc5ccccc5c4)nc3)cc2[nH]1]

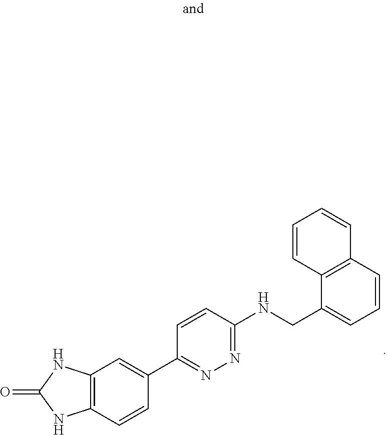 [SMILES: O=c1[nH]c2ccc(-c3ccc(NCc4cccc5ccccc45)nn3)cc2[nH]1]

7. The compound according to claim 1, wherein Y is selected from a bond, CH₂, CH(CH₃), CH₂CH₂ , CH₂CH(CH₃), and CH(CH₃)CH₂.

8. The compound according to claim 1, wherein
R² is H;
R⁴ is NH; [symbol]
is a double bond;
Y is CH₂; and
X is C or N.

9. The compound according to claim 8, selected from the group consisting of

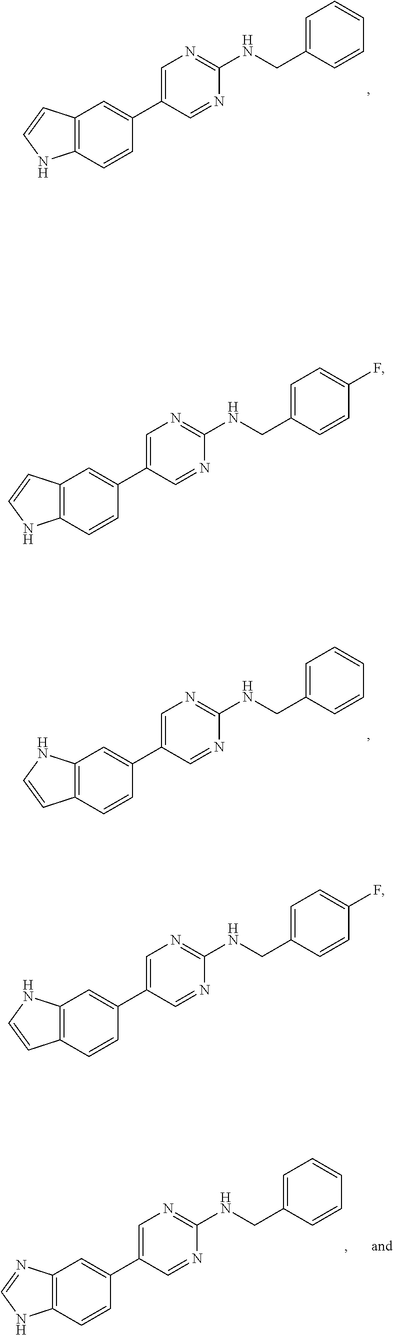 [SMILES: c1ccc(CNc2ncc(-c3ccc4[nH]ccc4c3)cn2)cc1 ; Fc1ccc(CNc2ncc(-c3ccc4[nH]ccc4c3)cn2)cc1 ; c1ccc(CNc2ncc(-c3ccc4cc[nH]c4c3)cn2)cc1 ; Fc1ccc(CNc2ncc(-c3ccc4cc[nH]c4c3)cn2)cc1 ; c1ccc(CNc2ncc(-c3ccc4[nH]cnc4c3)cn2)cc1]

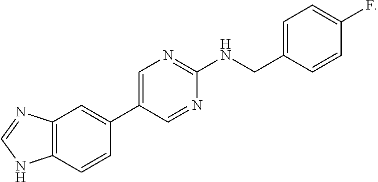 [SMILES: Fc1ccc(CNc2ncc(-c3ccc4[nH]cnc4c3)cn2)cc1]

10. The compound according to claim 1, wherein
R¹ and R² are H;
R³ is an oxygen that forms a carbonyl;
R⁵ is NH; [symbol]
is a single bond; and
X is CH.

11. The compound according to claim 10, selected from the group consisting of

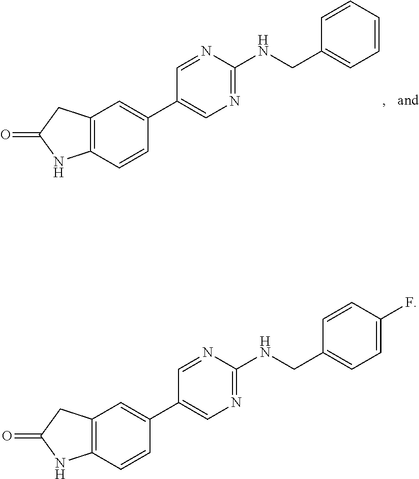 [SMILES: O=C1Cc2cc(-c3cnc(NCc4ccccc4)nc3)ccc2N1 ; O=C1Cc2cc(-c3cnc(NCc4ccc(F)cc4)nc3)ccc2N1]

12. The compound according to claim 1, wherein
R¹ is CH₃;
R² is H;
R³ is an oxygen that forms a carbonyl;
R⁴ is NH;
Y is CH₂; and
X is N.

13. The compound according to claim 12, selected from the group consisting of

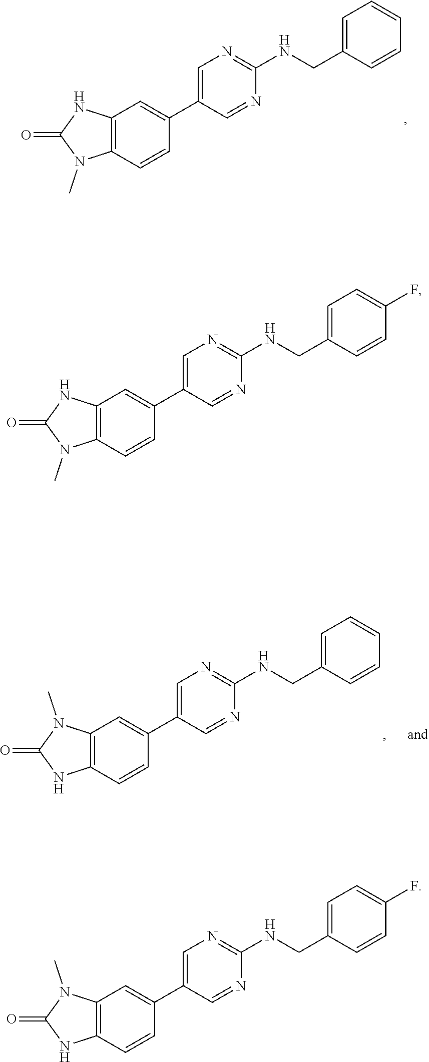 [SMILES: Cn1c(=O)[nH]c2cc(-c3cnc(NCc4ccccc4)nc3)ccc21 ; Cn1c(=O)[nH]c2cc(-c3cnc(NCc4ccc(F)cc4)nc3)ccc21 ; Cn1c(=O)[nH]c2ccc(-c3cnc(NCc4ccccc4)nc3)cc21 ; Cn1c(=O)[nH]c2ccc(-c3cnc(NCc4ccc(F)cc4)nc3)cc21]

14. The compound according to claim 1, wherein
R¹ is H;
R² is H;
R³ is an oxygen that forms a carbonyl;
R⁴ is NH;
Y is CH₂;
X is N; and
Z is a heteroaryl.

15. The compound according to claim 14, selected from the group consisting of

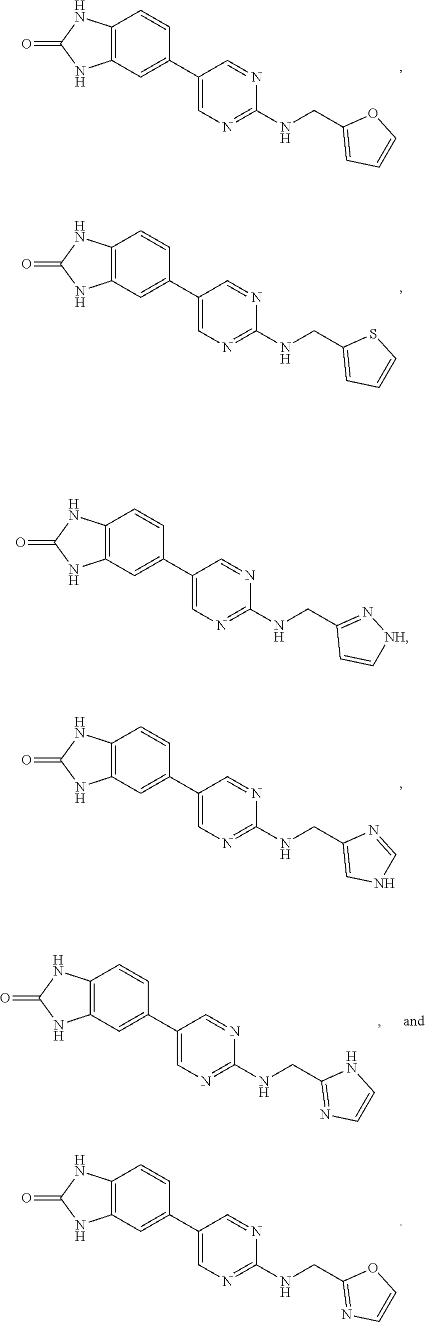 [SMILES: O=c1[nH]c2ccc(-c3cnc(NCc4ccco4)nc3)cc2[nH]1 ; O=c1[nH]c2ccc(-c3cnc(NCc4cccs4)nc3)cc2[nH]1 ; O=c1[nH]c2ccc(-c3cnc(NCc4cc[nH]n4)nc3)cc2[nH]1 ; O=c1[nH]c2ccc(-c3cnc(NCc4c[nH]cn4)nc3)cc2[nH]1 ; O=c1[nH]c2ccc(-c3cnc(NCc4ncc[nH]4)nc3)cc2[nH]1 ; O=c1[nH]c2ccc(-c3cnc(NCc4ncco4)nc3)cc2[nH]1]

16. The compound according to claim 1, wherein
R¹ is H;
R² is a C₂-C₃ alkyl, cycloalkyl, aryl, or heteroaryl;
R³ is an oxygen that forms a carbonyl;
R⁴ is NH;
Y is CH₂;
X is N; and
Z is a phenyl ring.

17. The compound according to claim 16, selected from the group consisting of

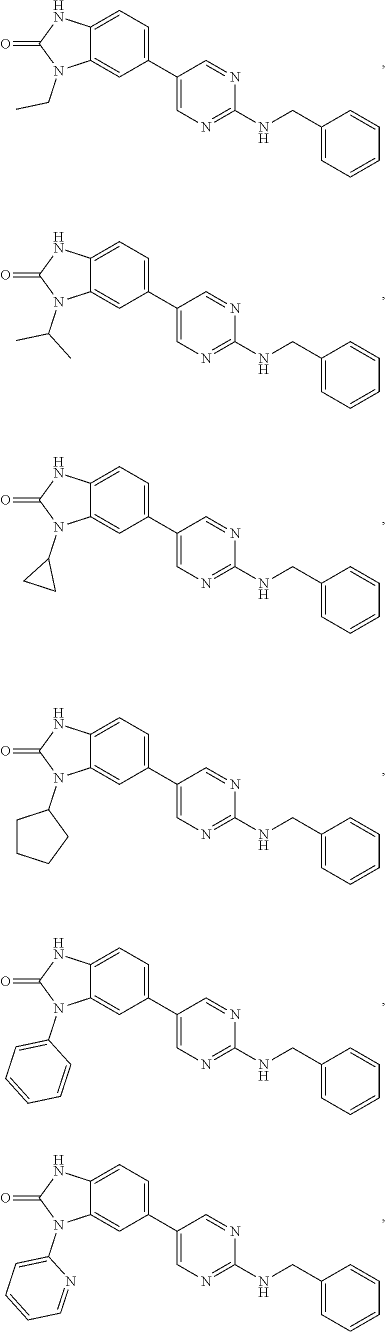 [SMILES: CCn1c(=O)[nH]c2ccc(-c3cnc(NCc4ccccc4)nc3)cc21 ; CC(C)n1c(=O)[nH]c2ccc(-c3cnc(NCc4ccccc4)nc3)cc21 ; O=c1[nH]c2ccc(-c3cnc(NCc4ccccc4)nc3)cc2n1C1CC1 ; O=c1[nH]c2ccc(-c3cnc(NCc4ccccc4)nc3)cc2n1C1CCCC1 ; O=c1[nH]c2ccc(-c3cnc(NCc4ccccc4)nc3)cc2n1-c1ccccc1 ; O=c1[nH]c2ccc(-c3cnc(NCc4ccccc4)nc3)cc2n1-c1ccccn1]

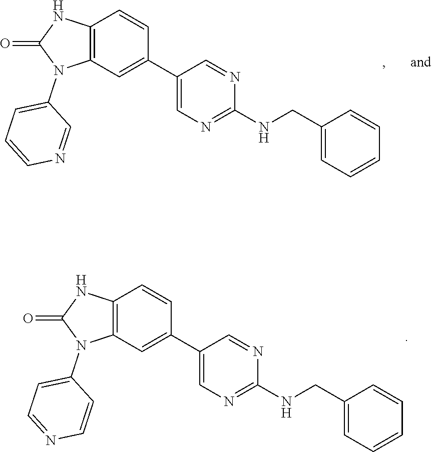 [SMILES: O=c1[nH]c2ccc(-c3cnc(NCc4ccccc4)nc3)cc2n1-c1cccnc1 ; O=c1[nH]c2ccc(-c3cnc(NCc4ccccc4)nc3)cc2n1-c1ccncc1]

18. A method of inhibiting activity of a kinase in a cell, said method comprising:
contacting the cell with a compound according to claim 1 under conditions effective to inhibit activity of the kinase in the cell.

19. The method according to claim 18, wherein the kinase is a dual-specificity tyrosine phosphorylation-regulated kinase (DYRK).

20. The method according to claim 19, wherein the kinase is dual-specificity tyrosine phosphorylation-regulated kinase 1A (DYRK1A).

21. The method according to claim 18, wherein said method is carried out ex vivo.

22. The method according to claim 18, wherein said method is carried out in vivo.

23. A method of increasing cell proliferation in a population of pancreatic beta cells, said method comprising:
contacting a population of pancreatic beta cells with a compound according to claim 1 under conditions effective to increase cell proliferation in the population of pancreatic beta cells.

24. The method according to claim 23 further comprising:
contacting the population of pancreatic beta cells with a transforming growth factor beta (TGFβ) superfamily signaling pathway inhibitor.

25. The method according to claim 23 or claim 24 further comprising:
contacting the population of pancreatic beta cells with a glucagon-like peptide-1 receptor (GLP1R) agonist, a Dipeptidyl Peptidase IV (DDP4) inhibitor, or a combination thereof.

26. The method according to any one of claims 23-25, wherein said method is carried out ex vivo.

27. The method according to any one of claims 23-25, wherein said method is carried out in vivo.

28. The method according to any one of claims 23-25, wherein said method is carried out with a composition comprising both the compound and the TGFβ superfamily signaling pathway inhibitor.

29. The method according to any one of claims 23-25, wherein the TGFβ superfamily signaling pathway inhibitor is selected from the group consisting of an inhibitor of TGFβ/TGFβ receptor binding, activin or inhibin/activin receptor binding, and bone morphogenetic protein (BMP)BMP receptor binding.

30. The method according to any one of claims 23-25, wherein the TGFβ superfamily signaling pathway inhibitor is an inhibitor of activin or inhibin/activin receptor binding selected from the group consisting of SB431542 and Alk5 inhibitor II.

31. The method according to any one of claims 23-25, wherein the TGFβ superfamily signaling pathway inhibitor is a SMAD signaling pathway inhibitor.

32. The method according to claim 25, wherein said method is carried out with a composition comprising the compound and the glucagon-like peptide-1 receptor (GLP1R) agonist, Dipeptidyl Peptidase IV (DDP4) inhibitor, or a combination of the GLP1R agonist and the DPP4 inhibitor.

33. The method according to claim 25 or claim 32, wherein the GLP1R agonist is selected from the group consisting of GLP1 analogs, extendin-4, liraglutide, lixisenatide, semaglutide, and combinations thereof.

34. The method according to claim 25 or claim 32, wherein the DDP4 is selected from the group consisting of sitagliptin, vildagliptin, saxagliptin, alogliptin, teneligliptin, and anagliptin.

35. The method according to any one of claims 23-34, wherein said pancreatic beta cells are primary human pancreatic beta cells.

36. The method according to any one of claims 23-35, wherein said contacting does not induce beta cell death or DNA damage.

37. The method according to any one of claims 23-36, wherein said contacting induces beta cell differentiation.

38. The method according to any one of claims 23-37, wherein said contacting increases glucose-stimulated insulin secretion.

39. A composition comprising:
a compound according to claim 1 and
a carrier.

40. The composition according to claim 39 further comprising:
a transforming growth factor beta (TGFβ) superfamily signaling pathway inhibitor.

41. The composition according to claim 39 or claim 40 further comprising:
a glucagon-like peptide-1 receptor (GLP1R) agonist, a Dipeptidyl Peptidase IV (DDP4) inhibitor, or a combination thereof.

42. The composition according to any one of claims 39-41, wherein the carrier is a pharmaceutically-acceptable carrier.

43. A method of treating a subject for a condition associated with insufficient insulin secretion, said method comprising:
administering to a subject in need of treatment for a condition associated with an insufficient level of insulin secretion a compound of claim 1 under conditions effective to treat the subject for the condition.

44. The method according to claim 43 further comprising:
administering a transforming growth factor beta (TGF(3) superfamily signaling pathway inhibitor.

45. The method according to claim 43 or claim 44 further comprising:
administering a glucagon-like peptide-1 receptor (GLP1R) agonist, a Dipeptidyl Peptidase IV (DDP4) inhibitor, or a combination thereof.

46. The method according to any one of claims 43-45, wherein said administering is carried out under conditions effective to increase pancreatic beta cell mass in the subject.

47. The method according to any one of claims 43-45, wherein the subject has been diagnosed as having one or more of type I diabetes (T1D), type II diabetes (T2D), gestational diabetes, congenital diabetes, maturity onset diabetes (MODY), cystic fibrosis-related diabetes, hemochromatosis-related diabetes, drug-induced diabetes, or monogenic diabetes.

48. The method according to any one of claims 43-45, wherein the subject has been diagnosed as having metabolic syndrome or insulin resistance.

49. The method according to any one of claims 43-45, wherein the subject has had a pancreatectomy, pancreas transplantation, or pancreatic islet transplantation.

50. The method according to any one of claims 43-45, wherein said administering is carried out orally, transdermally, parenterally, subcutaneously, intravenously, intramuscularly, or intraperitoneally.

51. The method according to any one of claims 43-45, wherein the subject is a mammalian subject.

52. The method according to any one of claims 43-45, wherein the subject is a human subject.

53. A method of treating a subject for a neurological disorder, said method comprising:
administering to a subject in need of treatment for a neurological disorder a compound of claim 1 under conditions effective to treat the subject for the condition.

54. The method according to claim 53 further comprising:
administering a transforming growth factor beta (TGFβ) superfamily signaling pathway inhibitor.

55. The method according to claim 53 or claim 54 further comprising:
administering a glucagon-like peptide-1 receptor (GLP1 R) agonist, a Dipeptidyl Peptidase IV (DDP4) inhibitor, or a combination thereof.

56. The method according to any one of claims 53-55, wherein the subject has been diagnosed as having one or more of diabetes, Down's Syndrome, or a neurodegenerative disease.

57. The method according to any one of claims 53-55, wherein said administering is carried out orally, transdermally, parenterally, subcutaneously, intravenously, intramuscularly, or intraperitoneally.

58. The method according to any one of claims 53-55, wherein the subject is a mammalian subject.

59. The method according to any one of claims 53-55, wherein the subject is a human subject.
